(19) United States
(12) Patent Application Publication
(10) Pub. No.: US 2024/0066148 A1
(43) Pub. Date: Feb. 29, 2024

(54) GLOBIN GENE THERAPY FOR TREATING HEMOGLOBINOPATHIES

(71) Applicants: MEMORIAL SLOAN-KETTERING CANCER CENTER, New York, NY (US); UNIVERSITY OF WASHINGTON, Seattle, WA (US)

(72) Inventors: Michel Sadelain, New York, NY (US); Isabelle Riviere, New York, NY (US); Jorge Mansilla-Soto, Forest Hills, NY (US); Xiuyan Wang, New York, NY (US); George Stamatoyannopoulos, Seattle, WA (US); John Stamatoyannopoulos, Seattle, WA (US); Mingdong Liu, Seattle, WA (US)

(73) Assignees: MEMORIAL SLOAN-KETTERING CANCER CENTER, New York, NY (US); UNIVERSITY OF WASHINGTON, Seattle, WA (US)

(21) Appl. No.: 18/160,083

(22) Filed: Jan. 26, 2023

Related U.S. Application Data
(63) Continuation of application No. 15/449,416, filed on Mar. 3, 2017, now Pat. No. 11,717,579.
Application No. 15/449,416 is a continuation of application No. PCT/US2015/048698, filed on Sep. 4, 2015.
(60) Provisional application No. 62/045,997, filed on Sep. 4, 2014.

(51) Int. Cl. A61K 48/00; C07K 14/805; C12N 9/22; C12N 15/85; C12N 15/86

(52) U.S. Cl. CPC A61K 48/0066; A61K 48/0058; C07K 14/805; C12N 9/22; C12N 15/85; C12N 15/86; C12N 2740/10043; C12N 2740/15043; C12N 2740/16043; C12N 2830/008; C12N 2830/15; C12N 2830/30; C12N 2830/40; C12N 2830/46; C12N 2830/48; C12N 2830/50

(57) ABSTRACT

The presently disclosed subject matter provides for expression cassettes that allow for expression of a globin gene or a functional portion thereof, vectors comprising thereof, and cells transduced with such expression cassettes and vectors. The presently disclosed subject matter further provides methods for treating a hemoglobinopathy in a subject comprising administering an effective amount of such transduced cells to the subject.

### CROSS-REFERENCE TO RELATED APPLICATIONS

This application is a continuation of U.S. patent application Ser. No. 15/449,416, filed Mar. 3, 2017, which is a continuation of International Patent Application No. PCT/US15/48698 filed Sep. 4, 2015, which claims priority to U.S. Provisional Application No. 62/045,997 filed Sep. 4, 2014, the contents of all of which are hereby incorporated by reference in their entireties herein, and to each of which priority is claimed.

### GRANT INFORMATION

This invention was made with government support under Grant No. HL053750 from National Heart, Lung and Blood Institute. The government has certain rights in the invention.

### SEQUENCE LISTING

The specification further incorporates by reference the Sequence Listing submitted on Jan. 26, 2023. The Sequence Listing file, identified as 072734.0477CON.xml, is 120,261 bytes in size and was created on Jan. 25, 2023. The Sequence Listing, electronically filed on Jan. 26, 2023, does not extend beyond the scope of the specification and thus does not contain new matter.

### INTRODUCTION

The presently disclosed subject matter provides expression cassettes and vectors comprising such expression cassettes that express a globin protein, e.g., a human β-globin protein. The presently disclosed subject matter further provides expression cassettes that comprise a globin gene or a functional portion thereof operably linked to a β-globin locus control region (LCR) comprising a plurality of Dnase I hypersensitive sites. The expression cassettes of the presently disclosed subject matter comprise one or more insulators that counteract the effect of enhancer elements. The insulators disclosed herein do not substantially adversely impact the titer of a vector that comprises the presently disclosed expression cassettes. The expression cassettes and vectors can be used for treating a hemoglobinopathy, e.g., β-thalassemia, and sickle cell anemia.

### BACKGROUND

β-thalassemia and sickle cell anemia are severe congenital anemias that are caused by defective production of the β chain of hemoglobin. In β-thalassemia, the β chain deficit leads to the intracellular precipitation of excess α-globin chains, causing ineffective erythropoiesis and hemolytic anemia (Weatherall and Clegg (1981), Stamatoyannopoulos et al., (1994), Weatherall (2001), Steinberg (2001)). In the most severe forms found in homozygotes or compound heterozygotes, anemia is lethal within the first years of life in the absence of any treatment (Cooley and Lee (1925)). Lifelong transfusion therapy is needed to correct anemia, suppress ineffective erythropoiesis and inhibit gastrointestinal iron absorption (Weatherall and Clegg (1981), Stamatoyannopoulos et al. (1994), Weatherall (2001), Steinberg (2001)). However, transfusion therapy itself leads to iron overload, which is lethal if untreated. The prevention and treatment of iron overload are the major goals of current patient management (Giardina (2001)). The only current curative treatment to cure β-thalassemia is to provide erythroid precursors harboring normal globin genes through allogeneic bone marrow transplantation (BMT) (Giardini and Lucarelli (1994), Boulad et al. (1998), Lucarelli et al. (1999), Tisdale and Sadelain (2001)).

In sickle cell anemia, the hemoglobin β chain is mutated at amino acid position 6 (Glu→Val), leading to the synthesis of β^{S} instead of the normal β^{A} chain (Steinberg (2001), Pauling et al. (1949)). The resulting hemoglobin, HbS, causes accelerated red cell destruction, erythroid hyperplasia and painful vaso-occlusive ‘crises’ (Steinberg (2001)). Vaso-occlusion can damage organs, eventually causing long-term disabilities (e.g. following stroke or bone necrosis), and sometimes sudden death. While a very serious disorder, the course of sickle cell disease is typically unpredictable (Steinberg (2001)). By increasing production of fetal hemoglobin (Swank and Stamatoyannopoulos (1998)) and suppressing hematopoiesis, hydroxyurea can produce a measurable clinical benefit (Platt et al. (1984)), Charache et al. (1992), Atweh and Loukopoulos (2001)). Since hydroxyurea is a cytotoxic agent, there is a great need for alternative, less toxic drugs to induce γ-globin gene expression (Perrine et al. (2005), Stamatoyannopoulos (2005)). As for β-thalassemia, allogeneic bone marrow transplantation (BMT) is at present the only curative therapy for sickle cell disease (Tisdale and Sadelain (2001), Vermylen et al. (1998), Luzzatto and Goodfellow (1989)).

BMT, however, is not available as a therapeutic option to most patients suffering from β-thalassemia or sickle cell disease, due to the lack of an HLA-matched bone marrow donor for most individuals. Furthermore, although potentially curative, allogeneic BMT is not devoid of complications. Safe transplantation requires the identification of a histo-compatible donor to minimize the risks of graft rejection and graft-versus-host disease (Tisdale and Sadelain (2001), Vermylen et al. (1998), Luzzatto and Goodfellow (1989)). Because of the greater risks associated with matched-unrelated or mismatched transplants, most patients have to settle for life-long transfusion therapy, which does not correct ineffective erythropoiesis and exacerbates systemic iron accumulation. Moreover, despite the considerable improvement in life expectancy in the last decades (Borgna-Pignatti et al. (2004), Telfer et al. (2009), Ladis et al. (2011)), the risk of some serious complications arising over the long term from viral infections, iron toxicity and liver cirrhosis, remain (Mancuso et al. (2006)). These medical risks, together with the socio-economic cost of chronic β-thalassemia, underscore the need for safe, effective and curative therapies.

The only means to cure rather than treat severe β-thalassemia is to provide the patient with healthy hematopoietic stem cells (HSCs). HSCs normally give rise to all blood cell types, including 20 billion RBCs per day in adults. HSCs can be harvested from a donor with wild-type β-globin genes to yield long-lived red blood cells (RBCs) with a normal content in hemoglobin. Alternatively, one may genetically correct the patient's own HSCs, which at once resolves the search for a donor and eliminates the risks of graft-versus-host disease and graft rejection associated with allogeneic BMT (Sadelain (1997), Sadelain et al. (2007)). Globin gene transfer aims to restore the capacity of the β-thalassemic subject's own blood-forming stem cells to generate RBCs with a sufficient hemoglobin content Sadelain et al. (2007), Persons and Tisdale (2004), Sadelain (2006)). The goal in patients with sickle cell anemia is to prevent sickling, which can be achieved by diluting the endogenous HbS with a non-sickling Hb that incorporates the vector-encoded globin chain. The patient's own HSCs are the cells that have to be genetically modified to ensure long-lasting therapeutic benefits and achieve a curative stem cell-based therapy.

The implementation of globin gene transfer for the treatment of severe β-thalassemia and sickle cell anemia requires the efficient introduction of a regulated human β- or β-like globin gene in HSCs. The β-globin gene (or β-like variant) must be expressed in erythroid-specific fashion and at high level, especially for the treatment of transfusion-dependent beta-zero thalassemias.

The globin vectors developed to date present shortcomings that may limit or even preclude their safe use in thalassemia and sickle cell patients. Some of the β-globin locus control region (LCR) components contained in the vectors, in particular Dnase I hypersensitive site-2 (HS2), may have non-erythroid activity, exposing patients to the risk of insertional oncogenesis as seen with non-specific expression vectors. Further, the use of large LCR segments can be detrimental to the production of high titer vectors and the efficient transduction of patients HSCs. Accordingly, there is a need for novel globin expression cassettes that allow for therapeutic expression of a globin gene (e.g., human β-globin gene) in erythroid-specific and differentiation stage-specific fashion with minimal risk of insertional oncogenesis, and that enable high level transduction, thus improving their safety when used in treating thalassemia and sickle cell patients.

### SUMMARY OF THE INVENTION

The presently disclosed subject matter generally provides enhancer blocking insulators, and certain insulators additionally possess barrier insulator activity. The presently disclosed subject matter also provides expression cassettes comprising one or more insulators and allows for expression of a globin gene (e.g., a human R globin gene). Also provided are vectors comprising such expression cassettes, cells transduced with such expression cassettes or such vectors, and uses of such expression cassettes for treating hemoglobinopathies (e.g., β-thalassemia and sickle cell anemia).

In certain non-limiting embodiments, the presently disclosed subject matter provides an insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO:1 (and see infra). The presently disclosed subject matter also provides expression cassettes comprising at least one insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO: 1. In a non-limiting embodiment, an expression cassette comprises at least one insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO:1, and a globin gene or a functional portion thereof operably linked to a β-globin locus control region (LCR). In certain embodiments, the β-globin LCR does not comprise a Dnase I hypersensitive site-2 (HS2) region. In certain embodiments, the β-globin LCR region does not comprise a core sequence of HS2. In one non-limiting embodiment, the core sequence of HS2 has the nucleotide sequence set forth in SEQ ID NO:20. In one non-limiting embodiment, the core sequence of HS2 has the nucleotide sequence set forth in SEQ ID NO:21. In certain embodiments, the β-globin LCR does not comprise a HS2 region that sustains the enhancer activity of HS2. In one non-limiting embodiment, the β-globin LCR comprises a Dnase I hypersensitive site-1 (HS1) region, a Dnase I hypersensitive site-3 (HS3) region, and a Dnase I hypersensitive site-4 (HS4) region. In certain embodiments, the HS3 region is positioned between the HS1 and the HS4 region.

In certain embodiments, the HS1 region is about 1.1 kb bp in length. In one non-limiting embodiment, the HS1 region is between about 500 bp and about 1000 bp in length. In one non-limiting embodiment, the HS1 region has the nucleotide sequence set forth in SEQ ID NO:2. In certain embodiments, the HS1 region is about 600 bp in length. In one non-limiting embodiment, the HS1 region is 602 bp in length. In certain embodiments, the HS1 region is between about 500 and about 600 bp in length. In one non-limiting embodiment, the HS1 region has the nucleotide sequence set forth in SEQ ID NO:3. In certain embodiments, the HS1 region is about 490 bp in length. In one non-limiting embodiment, the HS1 region is 489 bp in length. In one non-limiting embodiment, the HS1 region has the nucleotide sequence set forth in SEQ ID NO:4. In one non-limiting embodiment, the β-globin LCR comprises a HS1 region having a nucleotide sequence set forth in SEQ ID NO:2, a HS3 region having a nucleotide sequence set forth in SEQ ID NO:5, and a HS4 region having a nucleotide sequence set forth in SEQ ID NO:6, and the β-globin LCR region does not comprise a HS2 region. In one non-limiting embodiment, the β-globin LCR region comprises a HS1 region having a nucleotide sequence set forth in SEQ ID NO:3, a HS3 region having a nucleotide sequence set forth in SEQ ID NO:5, and a HS4 region having a nucleotide sequence set forth in SEQ ID NO:8, and the β-globin LCR does not comprise a HS2 region. In one non-limiting embodiment, the β-globin LCR comprises a HS1 region having a nucleotide sequence set forth in SEQ ID NO:4, a HS3 region having a nucleotide sequence set forth in SEQ ID NO:5, and a HS4 region having a nucleotide sequence set forth in SEQ ID NO:8, and the β-globin LCR does not comprise a HS2 region.

In certain embodiments, the β-globin LCR region does not comprise a HS1 region and/or does not comprise a HS2 region, and the β-globin LCR does not comprise a core sequence of HS2. In certain embodiments, the β-globin LCR does not comprise a core sequence of HS1. In one non-limiting embodiment, the core sequence of HS1 has the nucleotide sequence set forth in SEQ ID NO:22. In one non-limiting embodiment, the core sequence of HS1 has the nucleotide sequence set forth in SEQ ID NO:23. In certain embodiments, the β-globin LCR does not comprise a HS1 region that sustains the function of HS1. In certain embodiments, the β-globin LCR comprises a HS3 region and a HS4 region and does not comprise a core sequence of HS1. In certain embodiments, the HS3 region is positioned between a globin gene or functional portion thereof and the HS4 region. In certain embodiments, the HS3 region is between about 200 and about 1400 bp in length, e.g., between about 1300 and 1400 bp in length. In certain embodiments, the HS3 region is about 1300 bp in length. In one non-limiting embodiment, the HS3 region is 1301 bp in length. In one non-limiting embodiment, the HS3 region has the nucleotide sequence set forth in SEQ ID NO:5. In certain embodiments, the HS4 region is between about 200 and about 1200 bp in length, e.g., between about 400 and 1100 bp in length. In certain embodiments, the HS4 region is about 1.1 kb in length. In one non-limiting embodiment, the HS4 region is 1065 bp in length. In one non-limiting embodiment, the HS4 region has the nucleotide sequence set forth in SEQ ID NO:6. In one non-limiting embodiment, the HS4 region has the nucleotide sequence set forth in SEQ ID NO:7. In certain embodiments, the HS4 region is about 450 bp in length. In one non-limiting embodiment, the HS4 region is 446 bp in length. In one non-limiting embodiment, the HS4 region has the nucleotide sequence set forth in SEQ ID NO:8. In one non-limiting embodiment, the β-globin LCR region comprises a HS3 region having the nucleotide sequence set forth in SEQ ID NO:5 and a HS4 region having a nucleotide sequence set forth in SEQ ID NO:6, and the β-globin LCR region does not comprise a HS1 region or a HS2 region.

Alternatively, the β-globin LCR region can comprise a HS2 region, a HS3 region, and a HS4 region. In certain embodiments, the HS2 region is between about 400 and about 1000 bp in length, e.g., between about 800 and 900 bp in length. In certain embodiments, the HS2 region is about 860 bp in length. In one non-limiting embodiment, the HS2 region has the nucleotide sequence set forth in SEQ ID NO:9. In certain embodiments, the HS3 region is about 1300 bp in length. In one non-limiting embodiment, the HS3 region is 1301 bp in length. In one non-limiting embodiment, the HS3 region has the nucleotide sequence set forth in SEQ ID NO:5. In certain embodiments, the HS4 region is about 1.1 kb in length. In one non-limiting embodiment, the HS4 region is 1065 bp in length. In one non-limiting embodiment, the HS4 region has the nucleotide sequence set forth in SEQ ID NO:7. In one non-limiting embodiment, the β-globin LCR region comprises a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7. Additionally, the β-globin LCR region can further comprise a HS1 region.

In certain embodiments, the globin gene is selected from the group consisting of β-globin gene, γ-globin gene, and δ-globin gene. In one non-limiting embodiment, the globin gene is human β-globin gene. In non-limiting embodiments, the human β-globin gene is selected from the group consisting of a wild-type human β-globin gene, a deleted human β-globin gene comprising one or more deletions of intron sequences, and a mutated human β-globin gene encoding at least one anti-sickling amino acid residue. In one non-limiting embodiment, the human β-globin gene is human β^{A}-globin gene encoding a threonine to glutamine mutation at codon 87 (β^{A-T87Q}).

In certain embodiments, the expression cassette comprises one insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO:1. In certain embodiments, the expression cassette comprises two insulators, each comprising the CTCF binding site sequence set forth in SEQ ID NO:18, for example, but not limited to, where one or both insulators comprise SEQ ID NO: 24 or SEQ ID NO:25 and/or have the nucleotide sequence set forth in SEQ ID NO:1.

In certain embodiments, the expression cassette further comprises a β-globin promoter. In certain embodiments, the β-globin promoter is positioned between the globin gene or functional portion thereof and β-globin LCR region. In certain embodiments, the β-globin promoter is between about 200 and about 700 bp in length. In one non-limiting embodiment, the β-globin promoter is a human β-globin promoter that is about 613 bp in length. In one non-limiting embodiment, the human β-globin promoter has the nucleotide sequence set forth in SEQ ID NO:10. In another non-limiting embodiment, the β-globin promoter is a human β-globin promoter that is about 265 bp in length. In one non-limiting embodiment, the R human β-globin promoter has the nucleotide sequence set forth in SEQ ID NO:11.

In certain embodiments, the expression cassette further comprises a human β-globin 3′ enhancer. In certain embodiments, the human β-globin 3′ enhancer is positioned in the upstream of the globin gene or functional portion thereof. In certain embodiments, the β-globin 3′ enhancer is between about 700 and about 900 bp in length, e.g., between about 800 and 900 bp in length. In one non-limiting embodiment, the human β-globin 3′ enhancer is about 879 bp in length. In one non-limiting embodiment, the human β-globin 3′ enhancer has the nucleotide sequence set forth in SEQ ID NO:12.

In certain embodiments, the expression cassette further comprises at least one erythroid-specific enhancer. In certain embodiments, the at least one erythroid-specific enhancer is positioned between the globin gene or functional portion thereof and the β-globin LCR region. In certain embodiments, the at least one erythroid-specific enhancer has a nucleotide sequence selected from the group consisting of SEQ ID NOS: 13, 14, 15, 16 and 17. In certain embodiments, the at least one erythroid-specific enhancer is between about 100 and about 200 bp in length. In certain embodiments, the expression cassette comprises one, two or three erythroid-specific enhancers.

In certain embodiments, the expression cassette allows for expression of the globin gene or functional portion thereof in a mammal. In one non-limiting embodiment, the expression cassette allows for expression of a human β-globin gene. In certain embodiments, the expression of the globin gene or functional portion thereof is restricted to erythroid tissue.

The presently disclosed subject matter also provides recombinant vectors comprising the above-described expression cassettes. In certain embodiments, the recombinant vector is a retroviral vector. In one non-limiting embodiment, the retroviral vector is a lentivirus vector. In certain embodiments, the expression cassette comprised in the recombinant vector comprises one insulator. In certain embodiments, the recombinant vector further comprises a Woodchuck hepatitis post-regulatory element (WPRE) in the 3′ long terminal repeat (LTR) of the vector. In certain embodiments, the recombinant vector further comprises a bovine growth hormone polyadenylation signal in the 3′ long terminal repeat (LTR) of the vector.

In addition, the presently disclosed subject matter provides non-naturally occurring or engineered nucleases comprising the above-described expression cassettes. In certain embodiments, the nuclease is selected from the group consisting of a non-naturally occurring or engineered zinc-finger nuclease (ZFN), a non-naturally occurring or engineered meganuclease, and a non-naturally occurring or engineered transcription activator-like effector nuclease (TALEN). In certain embodiments, the nuclease comprises a DNA binding domain and a nuclease cleavage domain. In certain embodiments, the nuclease binds to a genomic safe harbor site. In certain embodiments, the nuclease generates a double strand break (DSB) at the genomic safe harbor site. In certain embodiments, the expression cassette comprised in the nuclease comprises two of the insulator having the nucleotide sequence set forth in SEQ ID NO:1. In certain embodiments, the nuclease allows for targeted delivery of the expression cassette. The presently disclosed subject matter also provides polynucleotides encoding the above-described nucleases, and vectors comprising the polynucleotides. In one non-limiting embodiment, the vector is a lentiviral vector.

Furthermore, the presently disclosed subject matter provides non-naturally occurring or engineered CRISPR-Cas systems comprising the above-described expression cassettes. In certain embodiments, the CRISPR-Cas system comprises a CRISPR-Cas nuclease and single-guide RNA. In certain embodiments, the CRISPR-Cas system binds to a genomic safe harbor site. In certain embodiments, the CRISPR-Cas system generates a double strand break (DSB) at the genomic safe harbor site. In certain embodiments, the expression cassette comprised in the CRISPR-Cas system comprises two of the insulator having the nucleotide sequence set forth in SEQ ID NO:1. In certain embodiments, the CRISPR-Cas allows for targeted delivery of the expression cassette. The presently disclosed subject matter also provides polynucleotides encoding the above-described CRISPR-Cas systems, and vectors comprising the polynucleotides. In one non-limiting embodiment, the vector is a lentiviral vector.

In some embodiments, the genomic safe harbor site is an extragenic genomic safe harbor site. In certain embodiments, the genomic safe harbor site is located on chromosome 1. In some embodiments, the genomic safe harbor meets all of the following five criteria: (1) distance of at least 50 kb from the 5′ end of any gene (e.g., from the 5′ end of the gene), (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the human genome.

Additionally, the presently disclosed subject matter provides cells transduced with the above-described expression cassettes, cells transduced with the above-described recombinant vectors, cells transduced with the above-described nucleases, cells transduced with the above-described CRISPR-Cas systems. In addition, the presently disclosed subject matter provides cells transduced with the above-described vectors. In certain embodiments, the cell is selected from the group consisting of a hematopoietic stem cell, an embryonic stem cell, an induced pluripotent stem cell, and a hemogenic endothelium cell. In one non-limiting embodiment, the hematopoietic stem cell is a CD34⁺ hematopoietic stem cell. In certain embodiments, the cell is transduced ex vivo.

Also provided are pharmaceutical compositions comprising an effective amount of the above-described cells and a pharmaceutically acceptable carrier. The presently disclosed subject matter also provides pharmaceutical compositions for treating a hemoglobinopathy comprising an effective amount of the above-described cells and a pharmaceutically acceptable carrier.

Furthermore, the presently disclosed subject matter provides kits for treating a hemoglobinopathy comprising the above-described cells. In certain embodiments, the kits further comprise written instructions for using the cell for treating a subject having a hemoglobinopathy.

In addition, the presently disclosed subject matter provides methods of treating a hemoglobinopathy in a subject, comprising administering an effective amount of the above-described cells to the subject, thereby restoring the subject's ability to produce red blood cells containing normal hemoglobin. In certain embodiments, a therapeutically relevant level of hemoglobin is produced in the subject following administering the cell to the subject. In certain amendments, the method comprises administering an effective amount of the cell transduced with the above-described recombinant vector. In some embodiments, the vector copy number of the recombinant vector in the cell that provides for the therapeutically relevant level of hemoglobin in the subject is about 0.5-2 vector copy number per cell. In certain embodiments, the method corrects ineffective erythropoiesis in the subject. In certain embodiments, the method does not incur the risk of graft-versus-host disease in the subject. In certain embodiments, the method does not comprise administering an immunosuppressive agent. In certain embodiments, the cell is selected from the group consisting of a hematopoietic stem cell, an embryonic stem cell, an induced pluripotent stem cell, and a hemogenic endothelium cell. In one non-limiting embodiment, the subject is a human. In certain embodiments, the cell is from the subject. In one non-limiting embodiment, the cell is from bone marrow of the subject.

In accordance with the presently disclosed subject matter, the hemoglobinopathy can be selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In one non-limiting embodiment, the hemoglobinopathy is β-thalassemia. In another non-limiting embodiment, the hemoglobinopathy is sickle cell anemia.

### BRIEF DESCRIPTION OF THE FIGURES

The following Detailed Description, given by way of example, but not intended to limit the invention to specific embodiments described, may be understood in conjunction with the accompanying drawings.

FIG. 1 depicts a recombinant vector comprising an expression cassette in accordance with one non-limiting embodiment of the presently disclosed subject matter.

FIG. 2 depicts a recombinant vector an expression cassette in accordance with one non-limiting embodiment of the presently disclosed subject matter.

FIG. 3 depicts a recombinant vector an expression cassette in accordance with one non-limiting embodiment of the presently disclosed subject matter.

FIG. 4 depicts a recombinant vector an expression cassette in accordance with one non-limiting embodiment of the presently disclosed subject matter.

FIGS. 5A-C represent the genotoxicity of insulator A1. FIG. 5A demonstrate the gammaretroviral vector genotoxicity assay used. FIG. 5B notice the increased survival of mice receiving 32D cells transduced with insulated gammaretroviral vector. Also notice the results obtained with cHS4 and with the uninsulated control. FIG. 5C show that insulator A1 decreased the risk of genotoxicity.

FIG. 6 represents normalized β chain expression in thalassemic Hbb^{th3/+} mice 8 and 44 weeks post-treatment.

FIG. 7 represents the evaluation of enhancer activity in non-erythroid K562 cells.

FIG. 8 represents the erythroid-specific enhancers in accordance with certain embodiments of the presently disclosed subject matter.

FIG. 9 represents the erythroid-specific enhancers in accordance with certain embodiments of the presently disclosed subject matter.

FIGS. 10A-B depict various recombinant vectors comprising the presently disclosed expression cassettes.

FIG. 11 represents the titer of the recombinant vectors comprising the presently disclosed expression cassettes.

FIG. 12 represents the titer of the recombinant vectors comprising the presently disclosed expression cassettes.

### DETAILED DESCRIPTION OF THE INVENTION

The presently disclosed subject matter generally provides expression cassettes that allow for expression of a globin gene (e.g., human β-globin gene). In one non-limiting example, the expression cassette comprises at least one insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO:1 and a globin gene or a functional portion thereof operably linked to a β-globin locus control region (LCR) region. The expression of the globin gene induced by the presently disclosed expression cassettes is erythroid-specific, differentiation stage-specific, high-level, and sustained. The presently disclosed subject matter also provides recombinant vectors, non-naturally occurring or engineered nucleases, and non-naturally occurring or engineered CRISPR-Cas systems comprising such expression cassettes, and cells transduced with such expression cassettes, recombinant vectors, nucleases and CRISPR-Cas systems. The presently disclosed expression cassettes and vectors comprising thereof provide for a safe gene transfer therapy as therapeutic transgene expression is achieved (e.g., a therapeutically relevant level of hemoglobin is produced) with a low vector copy number per cell (e.g., 0.5-2, 1-2, or even 0.5-1). In addition, the presently disclosed subject matter provides methods of using such transduced cells for treating a hemoglobinopathy (e.g., β-thalassemia and sickle cell anemia).

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “expression cassette” refers to a nucleic acid construct, generated recombinantly or synthetically, with a series of specified nucleic acid elements, which permit transcription of a particular nucleic acid in a target cell. The expression cassette can be incorporated into a plasmid, chromosome, mitochondrial DNA, plastid DNA, virus or nucleic acid region. The expression cassette portion can include a gene to be transcribed and elements that control the expression of the gene (e.g., a promoter).

As used herein, the term “β-globin locus control region (LCR) region” refers to a polynucleotide composed of one or more Dnase I hypersensitive site (HS) regions, including a HS1 region, a HS2 region, a HS3 region, and a HS4 region. The structure of many LCRs of the β-globin genes have been published, e.g., human (Li et al., J. Biol. Chem. (1985); 260:14, 901; Li et al., Proc. Natl. Acad. Sci. (1990) 87:8207); mouse (Shehee et al., J. Mol. Biol. (1989); 205:41); rabbit (Margot et al., J. Mol. Biol. (1989); 205:15); and goat (Li, Q., et al., Genomics (1991); 9:488), each of which are incorporated by reference herein. In certain embodiments, the β-globin LCR region comprises a HS2 region (e.g., a β-globin LCR region comprising a HS2 region, a HS3 region and a HS4 region; and a β-globin LCR region comprising a HS1 region, a HS2 region, a HS3 region and a HS4 region). In certain embodiments, the β-globin LCR region does not comprise a HS2 region (e.g., a β-globin LCR region comprising a HS1 region, a HS3 region, a HS4 region). In certain embodiments, the β-globin LCR region does not comprise a HS2 region or a HS1 region (e.g., a β-globin LCR region comprising a HS3 region and a HS4 region).

As used herein, the term “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene.

As used herein, the term “globin” refers to a family of heme-containing proteins that are involved in the binding and transport of oxygen. Subunits of vertebrate and invertebrate hemoglobins, vertebrate and invertebrate myoglobins or mutants thereof are included by the term globin.

As used herein, the term “wild-type” refers to the normal gene, virus, or organism found in nature without any mutation or modification.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene region, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide can comprise one or more modified nucleotides, such as methylated nucleotides and nucleotide analogs. In particular embodiments, the presently disclosed subject matter provides polynucleotides encoding one or more globin genes or functional portions thereof. If present, modifications to the nucleotide structure may be imparted before or after assembly of the polymer. The sequence of nucleotides may be interrupted by non nucleotide components. A polynucleotide may be further modified after polymerization, such as by conjugation with a labeling component. Such polynucleotides need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, the terms “polypeptide” and “protein” are used interchangeably to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. Particular embodiments of the presently disclosed subject matter also include polypeptide “variants.” Polypeptide “variant” refers to polypeptides that are distinguished from a reference polypeptide by the addition, deletion, truncations, and/or substitution of at least one amino acid residue, and that retain a biological activity. In certain embodiments, a polypeptide variant is distinguished from a reference polypeptide by one or more substitutions, which may be conservative or non-conservative, as known in the art. In certain embodiments, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain embodiments, the amino acid additions or deletions occur at the C-terminal end and/or the N-terminal end of the reference polypeptide. In certain embodiments, the amino acid deletions include C-terminal truncations of about 1, about 2, about 3, about 4, about 5, about 6, about 7, about 8, about 9, about 10, about 15, about 20, about 25, about 30, about 35, about 40, about 45, 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, or about 175 or more amino acids, including all intervening numbers of amino acids, e.g., 25, 26, 27, 29, 30 . . . 100, 101, 102, 103, 104, 105 . . . 170, 171, 172, 173, 174, etc.

As noted above, polypeptides of the presently disclosed subject matter may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., Molecular Biology of the Gene, Fourth Edition, Benjamin/Cummings, Menlo Park, Calif, 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

As used herein, the term “substantially identical” refers to a polypeptide or a polynucleotide exhibiting at least 50% identity to a reference amino acid sequence (for example, any one of the amino acid sequences described herein) or a nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity or homology is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity or homology, a BLAST program may be used, with a probability score between e-3 and e-100 indicating a closely related sequence. The percentage of identity between two sequences can also be determined with programs such as DNAMAN (Lynnon Biosoft, version 3.2). Using this program two sequences can be. aligned using the optimal alignment algorithm (Smith and Waterman, 1981). After alignment of the two sequences the percentage identity can be calculated by dividing the number of identical nucleotides between the two sequences by the length of the aligned sequences minus the length of all gaps.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation.

As used herein, a “single guide RNA” or a “synthetic guide RNA” refers to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. The term “tracr mate sequence” may also be used interchangeably with the term “direct repeat(s)”.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

As used herein, the term “isolated cell” refers to a cell that is separated from the molecular and/or cellular components that naturally accompany the cell. As used herein, the term “isolated” refers to material that is free, substantially free, or essentially free to varying degrees from components which normally accompany it as found in its native state. “Isolate” denotes a degree of separation from original source or surroundings.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 10³ cells, at least about 10⁴ cells, at least about 10⁵ cells, at least about 10⁶ cells, at least about 10⁷ cells, or at least about 10⁸ cells expressing similar or different phenotypes.

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

As used herein, the term “cleavage half-domain” refers to a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

As used herein, the term “chromosome” refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes.

As used herein, the term “gene” includes a DNA region encoding a gene product, as well as all DNA regions which regulate the production of the gene product, whether or not such regulatory sequences are adjacent to coding and/or transcribed sequences. Accordingly, a gene includes, but is not limited to, promoter sequences, terminators, translational regulatory sequences such as ribosome binding sites and internal ribosome entry sites, enhancers, silencers, insulators, boundary elements, replication origins, matrix attachment sites and locus control regions

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional region” or “functional portion” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional region can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. The ability of a protein to interact with another protein can be determined, for example, by co-immunoprecipitation, two-hybrid assays or complementation, both genetic and biochemical.

As used herein, the term “promoter” refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “modulate” refers to altering positively or negatively. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

### II. Insulators

Several cases of vector-related malignant transformation have been reported in clinical settings, associated with the activation of cellular oncogenes by vector-encoded enhancers (Baum et al. (2006), Nienhuis et al. (2006), Ramezani et al. (2006)) and various vector modifications have been performed or proposed to reduce vector genotoxicity (Baum et al. (2006), Nienhuis et al. (2006), Ramezani et al. (2006)). A class of DNA elements known as chromatin insulators has been recognized as one approach to improve vector safety and performance (Emery (2011)).

Insulators are naturally occurring DNA elements that help from the functional boundaries between adjacent chromatin domains. Insulators bind proteins that modify chromatin and alter regional gene expression. The placement of insulators in the vectors described herein offer various potential benefits including, but not limited to, 1) shielding of the vector from positional effect variegation of expression by flanking chromosomes (i.e., barrier activity, which may decrease position effects and vector silencing); and 2) shielding flanking chromosomes from insertional transactivation of endogenous gene expression by the vector (enhancer blocking). There are two basic classes of chromatin insulators: (a) barrier insulators that block the encroachment of silencing heterochromatin into adjoining regions of open chromatin that are transcriptionally permissive, and (b) enhancer blocking insulators that prevent enhancer-mediated transcriptional activation of adjoining regions. The sequences that mediate these activities are physically separable and mechanistically distinct (Recillas-Targa et al. (2002)). Chromatin insulators do not exhibit inherent transcriptional enhancing or repressing activities on their own. As such, they make ideal elements for reducing the interaction between gene transfer vectors and the target cell genome. Insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Nat'l Acad. Sci. USA, 99: 16433; and Zhan et al. (2001) Hum. Genet., 109: 471).

The problems created by insertional mutagenesis of viral vectors are widely known (Nienhuis (2013), Baum et al. (2006), Nienhuis et al. (2006)) as is the evidence that the risks of genotoxicity can be reduced by the use of chromatin insulators (Arumugam et al. (2007), Emery (2011), Evans-Galea et al. (2007), Rivella et al. (2000), Emery et al. (2000), Emery et al. (2002), Yannaki et al. (2002), Hino et al. (2004), Ramezani et al. (2003), Ramezani et al. (2008)). The presently disclosed subject matter provides novel insulators that are powerful enhancer blocking insulators, and certain insulators additionally possess barrier insulator activity. In vertebrates, the function of enhancer blocking insulators is mediated through the zinc-finger DNA-binding factor CTCF (Gaszner and Felsenfeld (2006), Wallace and Felsenfeld (2007)). In general, these elements are thought to function through physical loop structures, which are established by CTCF-mediated interactions between adjacent insulator elements or through CTCF-mediated tethering of the chromatin fiber to structural elements within the nucleus. The first characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb region containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp region. Within this stretch is a 49-bp cHS4 element (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

Insulators, such as cHS4, can block the interaction between enhancers and promoters when placed between these elements (Evans-Galea et al. (2007), Chung et al. (1997), Bell et al. (1999), Ryu et al. (2007), Ryu et al. (2008)). Several studies have demonstrated the ability of the cHS4 insulator to reduce position-effect silencing of gammaretroviral vectors (Evans-Galea et al. (2007), Rivella et al. (2000), Emery et al. (2000), Emery et al. (2002), Yannaki et al. (2002), Hino et al. (2004), Ramezani et al. (2006), Yao et al. (2003), Nishino et al. (2006), Aker et al. (2007), Li and Emery (2008)), and lentiviral vectors (Bank et al. (2005), Arumugam et al. (2007), Puthenveetil et al. (2004), Evans-Galea et al. (2007), Ramezani et al. (2003), Aker et al. (2007), Ma et al. (2003), Chang et al. (2005), Pluta et al. (2005)). Those appropriately designed studies demonstrated that inclusion of the 1.2 kb version of the cHS4 insulator increased the likelihood and/or consistency of vector transgene expression in at least some settings (Arumugam et al. (2007), Emery (2011), Evans-Galea et al. (2007), Emery et al. (2002), Yannaki et al. (2002), Hino et al. (2004), Ramezani et al. (2006), Aker et al. (2007), Li and Emery (2008), Pluta et al. (2005). Jakobsson et al. (2004)). Nevertheless, the degree of protection afforded by the cHS4 insulator is far from complete. In addition, the inclusion of the 1.2 Kb cHS4 can adversely affect vector titers while the smallest cHS4 core has been proven ineffective (Aker et al. (2007), Jakobsson et al. (2004)). By contrast, the insulators of the presently disclosed subject matter do not affect adversely the titers of viral vectors, and are more powerful and effective than the cHS4 insulator.

The presently disclosed insulators are identified through genomic approaches, e.g., using genomic approaches to identify insulators that are powerful enhancer blockers as well as barrier insulators of the human genome. The presently disclosed insulators enhance the safety of gene therapy (e.g., stem cell gene therapy, globin gene therapy). For gene therapy of the hemoglobinopathies, powerful enhancers are required to achieve therapeutic levels of globin gene expression. Powerful insulators therefore represent one means to protect the genomic environment from the powerful enhancers of the integrating vectors.

The presently disclosed insulators possess powerful enhancer blocking activity. For example, and not by way of limitation, an insulator of the present disclosure can reduce the activity of an enhancer element by at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%. In certain embodiments, the insulators possess barrier activity in addition to enhancer blocking activity. The presently disclosed insulators substantially decrease the risks of insertional mutagenesis and genotoxicity associated with viral vectors. Furthermore, when a presently disclosed insulator is incorporated into a vector, the insulator does not adversely effect vector titers of the vector. In certain embodiments, the insulators (e.g., insulator A1) increase the in vivo expression of the globin gene or functional portion thereof.

In certain embodiments, the insulator comprises a Transcriptional repressor CTCF binding site, which has the nucleotide sequence set forth in SEQ ID NO: 18, which is provided below:

[SEQ ID NO: 18]
CACCAGGTGGCGCT.

In one non-limiting embodiment, the insulator has the nucleotide sequence set forth in SEQ ID NO:1, which is provided below, or a sequence which is at least about 95 percent homologous, or at least about 98 percent identical (homologous), to SEQ ID NO:1. This insulator having the nucleotide sequence set forth in SEQ ID NO:1 is designated as insulator A1.

[SEQ ID NO: 1]
TCCTTCCTTTCTAAATGACGAGAGAGACAGAAGAATTCTTCAAGGTTAGT
GTGTCCAGCATGCAACCTTTCCTTCCTGGATGAGCATCCCTGGAGTAGGA
GAGCCAGCCTGCCTCCTGCGCTGGCACAGAGCCCGGTTCCCTAGACAACT
GCCTCTCCAAATCTGATGTCCAGCGCCACCTGGTGTCCACATCAAGCAGA
CACAATTAATAGTCAACCTGTTCAGGAAAACTGTGAGGGGGAAAAAAAAG
AAAGAGGATTTATGAAGGGAAAAGAAAGTTTAGAGGATATGCCACGATTG
GCTAG

In certain embodiments, the insulator comprises a nucleotide sequence as set forth in SEQ ID NO:24, or a sequence which is at least about 95 percent identical, or at least about 98 percent identical, to SE ID NO: 24.

[SEQ ID NO: 24]
CCAATC GTGGCATATC CTCTAAACTT TCTTTTCCCT TCATAAATCC
TCTTTCTTTT TTTTCCCCCT CACAGTTTTC CTGAACAGGT
TGACTATTAA TTGTGTCTGC TTGATGTGGA CACCAGGTGG
CGCTGGACAT CAGATTTGGA GAGGCAGTTG TCTAGGGAAC
CGGGCTCTGT GCCAGCGCAG GAGGCAGGCT GGCTCTCCTA
TTCCAGGGAT GCTCATCCAG GAAGGAAAGG TTGCATGCTG
GACACACTAA CCTTGAAGAA TTCTTCTGTC TCTCTCGTCA
TTTAGAAAGG AAGGA.

In certain embodiments, the insulator comprises a nucleotide sequence as set forth in SEQ ID NO:25 (which is the reverse complement of SEQ ID NO:1), or a sequence which is at least about 95 percent identical, or at least about 98 percent identical, to SEQ ID NO: 25.

[SEQ ID NO: 25]
CTAGCCAATCGTGGCATATCCTCTAAACTTTCTTTTCCCTTCATAAATCC
TCTTTCTTTTTTTTCCCCCTCACAGTTTTCCTGAACAGGTTGACTATTAA
TTGTGTCTGCTTGATGTGGACACCAGGTGGCGCTGGACATCAGATTTGGA
GAGGCAGTTGTCTAGGGAACCGGGCTCTGTGCCAGCGCAGGAGGCAGGCT
GGCTCTCCTACTCCAGGGATGCTCATCCAGGAAGGAAAGGTTGCATGCTG
GACACACTAACCTTGAAGAATTCTTCTGTCTCTCTCGTCATTTAGAAAGG
AAGGA

In certain embodiments, the insulator comprises a nucleotide sequence as set forth in hg18 coordinates 76229933 to 76230115 of chromosome 1.

In certain embodiments, the insulator comprises a nucleotide sequence between residues 68041 and 68160, or between residues and 68041 and 68210, or between residues 68041 and 68280, or between residues 68005 and 68305, of Homo sapiens chromosome 1 clone RP11-550H2, GenBank Accession No. AC092813.2, or a sequence at least 95 or 98 percent identical thereto.

### III. Expression Cassettes

The presently disclosed subject matter provides expression cassettes comprising one or more the above-disclosed insulators (e.g., insulator A1). In certain embodiments, an expression cassette comprises at least one insulator having the nucleotide sequence set forth in SEQ ID NO:1, and a globin gene or a functional portion thereof operably linked to a β-globin LCR region.

β-Globin LCR Region

The human β-globin gene cluster consists of five genes embedded within one of many olfactory receptor gene arrays (Bulger et al., PNAS (1999); 96:5129-5134). The cluster spans over 80 kb on chromosome 11p15.4, and includes the five expressed β-like genes and cis-acting regulatory elements that direct their stage-specific expression during ontogeny (Forget (2001), Molecular Mechanism of Beta Thalassemia. Steinberg M H et al., Eds. Disorders of Hemoglobin. Genetics, Pathophysiology and Clinical Management, Cambridge University Press, Cambridge). The genes are arranged in the order of their developmental expression (Stamatoyannopoulos et al., (2001) Hemoglobin Switching. In: Stamatoyannopoulos G, et al., Eds. Molecular Basis of Blood Disorders, W. B. Saunders, Philadelphia, PA), 5′-ε-^{G}γ-^{A}γ-ψη-δ-β-3′. The α-like globin gene cluster (5′-ξ2-ψξ1-ψα2-ψα1-α2-α1-θ-3′) is located very close to the telomere of the short arm of chromosome 16 and spans about 40 kb. The expression of genes encoded within these two independent clusters is limited to erythroid cells and balanced so that the output of the β-globin-like chains matches that of the α-chains. This fine tuned balance is regulated at the transcriptional, posttranscriptional and posttranslational levels.

Developmental stage-specific expression is controlled by a number of proximal or distal cis-acting elements and the transcriptional factors that bind to them. In the case of the β-globin gene (HBB), the proximal regulatory elements comprise the β-globin promoter and two downstream enhancers, one located in the second intron of β-globin and the other approximately 800 bp downstream of the gene (Antoniou et al., EMBO J. (1988); 7:377-384; Trudel et al., Genes Dev. (1987); 1:954-961; Trudel et al., Mol. Cell. Biol. (1987); 7:4024-4029). The most prominent distal regulatory element is the β-globin LCR, located 50-60 kb upstream of the HBB and composed of several sub-regions with heightened sensitivity to DNaseI in erythroid cells (Forget (2001); Grosveld et al., Cell (1987); 51:975-985; Talbot et al., Nature (1989); 338:352). The most prominent property of the LCR is its strong, transcription-enhancing activity. An exemplary nucleotide sequence of the human β-globin region on chromosome 11 is set forth in SEQ ID NO: 19 (GenBank Access No.: NG_000007.3), which is provided below:

[SEQ ID NO: 19]
ggatcctcacatgagttcagtatataattgtaacagaataaaaaatcaattatgtattcaagttgctagtgtcttaagaggttcac
atttttatctaactgattatcacaaaaatacttcgagttacttttcattataattcctgactacacatgaagagactgacacgtag
gtgccttacttaggtaggttaagtaatttatccaaaaccacacaatgtagaacctaagctgattcggccatagaaacacaatatgt
ggtataaatgagacagagggatttctctccttcctatgctgtcagatgaatactgagatagaatatttagttcatctatcacacat
taaacgggactttacatttctgtctgttgaagatttgggtgtggggataactcaaggtatcatatccaagggatggatgaaggcag
gtgactctaacagaaagggaaaggatgttggcaaggctatgttcatgaaagtatatgtaaaatccacattaagcttctttctgcat
gcattggcaatgtttatgaataatgtgtatgtaaaagtgtgctgtatattcaaaagtgtttcatgtgcctaggggtgtcaaatact
ttgagtttgtaagtatatacttctctgtaatgtgtctgaatatctctatttacttgattctcaataagtaggtatcatagtgaaca
tctgacaaatgtttgaggaacaatttagtgtttacctattcaccaaaatttattaaatgcctaatctgtatcagatatacaattat
ctggcgaaatctgtaattcctaatttaaacagctgtgtagcctaattagggataaaggcatgcaaacccataatttgtgtaggttg
aaatgagctatagaaaaatgcagtatatttatcagaagtctttagggtcatgaaaaggaatggtcaactgacactgccagggactc
atatgtaagagataactaatgtgaagtgactttaaaggagaaattagcagaagttttctttccatgtctcctcatcatgttacaat
aacggaagagattaaaacaacaaatacatttagacagcaatgtttatcctggttagatgttttaatctaaatctatcttggagtgt
taaaatgcatttgctcacctactttaaaatataaatgaaggtaggaacctgtagatacaaaaagttggagaaaaaaagacaataaa
gatgacaaaaatctattaatccttgatagaaaatgagaagagataaaacactggtttacataaagaaaataagatggatagatagc
agatccttataaaagtgataatttgagaaaaaaaatactccatattctgagtttcttcacataaaataatacaaatctgctgtggt
aagttacaaagagatagattttttatcattatataaaagatattttaaacagagttatacaacaaaggaacagactatgtcatata
ttctcacttatcactataaacatctcagaaaaatctgcaaaatcatttcatagcattttaaatagttaggaataatgtagaaaact
gaaacagttctaagtttcccacaaacttagagtctcaaatgttgcattacctaacttacctgcaaatattttatacaaatttgcac
atgctactctagtcaaaaatatatgtacattatgggtattttctgtgtgtaacttggttctagttgcttctttcagaaatagcctc
tatttttgatttacctgataaaatcacattcctctccaaagccttctaaatacttccagactaactactttttagtacatctaaga
agaaaagagttttgtctcttatccacctctgagtcaaaaagcagcatgtccatcaattggtacatagttcccacagccccacttag
ctctggattggagttctacttggcattgtttgcaactacatggacgtaaaatgcatggattctcttgaaaaaatgtttctgccatg
atgttctctgaaagagactaaccttccctcgctttgcagagaaagactcgtgtaatccttgacaatgtcatctcatctatttattc
ccatgtctacccatatgtgaccttcatgtctttgctctaagcccctacatcctcaatctacacactaggatagtataaaagtaata
gtaataatagtagtaatagtaataacaatacaatgattatggcttatactatacacaagacactgttgatatattatttcatttag
tattcacagtaactctgtgcctcaagtactattgtaataccctttaagaggaggaaactgaggcacagggccctaaagtaatattc
caagatgaagtggctactaactgacagagggcataattcaactcatgatatttggctctagaatacatgctctgaatcattataca
ataataattcatgaggaaacattttttaaagcctaagttatttgctctgaaataagacataatttggggtgagaaagcttagattc
catgaagtattacagcatttggtagtctttttgcactccaggtcttatttttactgcttaaacataataaaacatatggttcagta
tgcctttgattttacaataatattcctgttatttttggaagcacagggtgtgggataatgctaattactagtgattagtattgaga
ggtgacagcgtgctggcagtcctcacagccctcgctcgctcttggcgcctcctctgcctgggctcccacattggtggcacttgagg
agcccttcagccggccgctgcactgtgggagcccttttctgggctggccaaggccagagccggctccctcagcttgccaggaggtg
tggagggacagacgcgggcaggaaccgggctgtgcgccgtgcttgagggagttccgggtgggcatgggctccgaggaccccgcact
cggagccgccagccggccccaccggccgcgggcagtgaggggcttagcacctgggccagcagctgctgtgctcaattcctcgccgg
gccttagctgccttcctgcggggcagggctcgggacctgcagcgcgccatgcctgagcctccccaccttcatgggctcctgtgcgg
cccgagcctcgccgacgagcgccgccccctgctccagggcacccagtcccatcgaccacccaagggctgaagagtgcgggcgcacg
gcaggggactggcaggcagctccccctgcagcccaggtgcgggatccactgggtgaagccggctaggctcctgagtttgctgggga
tgcgaagaacccttatgtctagataagggattgtaaatacaccaattggcactctgtatctagctcaaggtttgtaaacacaccaa
tcagcaccctgtgtctagctcagggtttgtgaatgcaccaatcaacactctatctagctactctggtggggccttggagaaccttt
atgtctagctcagggattgtaaatacaccaatcggcagtctgtatctagctcaaggtttgtaaacacaccaatcagcaccctgtgt
ctagctcagggtttgtgaatgcaccaatcaacactctgtatctagctactctggtggggacgtggagaacctttatgtctagctca
gggattgtaaatacaccactcggcagtctgtatctagctcaaggtttgtaaacacaccaatcagcaccctgtgtctagctcagggt
ttgtgaatgcaccaatcaacactctgtatctagctactctggtggggacttggagaacctttgtgtggacactctgtatctagcta
atctggtggggacgtggagaacctttgtgtctagctcatggattgtaaatgcaccaatcagtgccctgtcaaaacagaccactggg
ctctaccaatcagcaggatgtgggtggggccagataagagaataaaagcaggctgcccgagccagcagtggcaacccgctcgggtc
cccttccacactgtggaagctttgttctttcgctctttgcaataaatcttgctgctgctcactgtttgggtctacactgcctttat
gagctgtaacgctcaccgcgaaggtctgcagcttcactcttgaagccagcgagaccacgaacccaccgggaggaacgaacaactcc
agaggcgccgccttaagagctggaacgttcactgtgaaggtctgcagcttcactcctgagccagcgagaccacgaacccatcagaa
ggaagaaactccgaacacatccaaacatcagaacgaacaaactccacacacgcagcctttaagaactgtaacactcaccacgaggg
tccccggcttcattcttgaagtcagtgaaaccaagaacccaccaattccggacacagtatgtcagaaacaatatgagtcactaaat
caatatacttctcaacaatttccaacagcccttgcaattaacttggccatgtgactggttgtgactaaaataatgtggagataata
atgtgttactccctaaggcagagtgcccttctatcattctctttcccttcctctatgtggcagaaagtaaaagattctgaaatgat
aaagtcaatcacaggaaggcacctggactcctggcccactgcttggaggagagcactcaggaccatgaacatctgactgtgacgta
gcaataaagaaacccacgtttcatatgaaactgcttaaaattaatggcacaagtcatgtttttgatgttgcacatttgtctttatt
tgtggcttgttttgcttccacatcaatccactcaaggcctacattctgctataatgcaatttcaagttctttacaggccgagaaaa
atgaatctgaattcctgacctccaaaagtgatcaagatatttttagttcaggctccaaaattttctcattttcataggttttcctc
gattgatcattattcatgatttgcaaggaatcattcaatgttttctaaatctattactgcatcctgacacatatgacattttaact
atgttccagatttttgaatgaagagtgtaaattttaaatgttttcaccacaaaaaataagtatgtgaagtggtggatttgttaatt
agccttatttaaccatttaatattgtacacgtacaccaaagcatcatgttgtaccccatgaatacacacaattattatttgtcaat
ttaaaatgaaataataaaaaataacaaaggcattagcctctgcattgcctttaccggtcatcctcacggtgactaacgcaaaaaac
gttctatttcatccttacaaacatccctatctttgatgcctctttgtctagatctctatcccctcctgttttctctacgttattta
tatgggtatcatcaccatcctggacaacatcaggacagatatccctcaccaagccaatgttcctctctatgttggctcaaatgtcc
ttgaactttcctttcaccaccctttccacagtcaaaaggatattgtagtttaatgcctcagagttcagcttttaagcttctgacaa
attattcttcctctttaggttctcctttatggaatcttctgtactgatggccatgtcctttaactactatgtagatatctgctact
acctgtattatgcctctacctttattagcagagttatctgtactgttggcatgacaatcatttgttaatatgacttgcctttcctt
tttctgctattcttgatcaaatggctcctctttcttgctcctctcatttctcctgccttcacttggacgtgcttcacgtagtctgt
gcttatgactggattaaaaattgatatggacttatcctaatgttgttcgtcataatatgggttttatggtccattattatttccta
tgcattgatctggagaaggcttcaatccttttactctttgtggaaaatatctgtaaaccttctggttcactctgctatagcaattt
cagtttaggctagtaagcatgaggatgcctccttctctgatttttcccacagtctgttggtcacagaataacctgagtgattactg
atgaaagagtgagaatgttattgatagtcacaatgacaaaaaacaaacaactacagtcaaaatgtttctctttttattagtggatt
atatttcctgacctatatctggcaggactctttagagaggtagctgaagctgctgttatgaccactagagggaagaagatacctgt
ggagctaatggtccaagatggtggagccccaagcaaggaagttgttaaggagcccttttgattgaaggtgggtgcccccaccttac
agggacaggacatctggatactcctcccagtttctccagtttccctttttcctaatatatctcctgataaaatgtctatactcact
tccccatttctaataataaagcaaaggctagttagtaagacatcaccttgcattttgaaaatgccatagactttcaaaattatttc
atacatcggtctttctttatttcaagagtccagaaatggcaacattacctttgattcaatgtaatggaaagagctctttcaagaga
cagagaaaagaataatttaatttctttccccacacctccttccctgtctcttaccctatcttccttccttctaccctccccatttc
tctctctcatttctcagaagtatattttgaaaggattcatagcagacagctaaggctggttttttctaagtgaagaagtgatattg
agaaggtagggttgcatgagccctttcagttttttagtttatatacatctgtattgttagaatgttttataatataaataaaatta
tttctcagttatatactagctatgtaacctgtggatatttccttaagtattacaagctatacttaactcacttggaaaactcaaat
aaatacctgcttcatagttattaataaggattaagtgagataatgcccataagattcctattaataacagataaatacatacacac
acacacacattgaaaggattcttactttgtgctaggaactataataagttcattgatgcattatatcattaagttctaatttcaac
actagaaggcaggtattatctaaatttcatactggatacctccaaactcataaagataattaaattgccttttgtcatatatttat
tcaaaagggtaaactcaaactatggcttgtctaattttatatatcaccctactgaacatgaccctattgtgatattttataaaatt
attctcaagttattatgaggatgttgaaagacagagaggatggggtgctatgccccaaatcagcctcacaattaagctaagcagct
aagagtcttgcagggtagtgtagggaccacagggttaagggggcagtagaattatactcccactttagtttcatttcaaacaatcc
atacacacacagccctgagcacttacaaattatactacgctctatactttttgtttaaatgtataaataagtggatgaaagaatag
atagatagatagacagatagatgatagatagaataaatgcttgccttcatagctgtctccctaccttgttcaaaatgttcctgtcc
agaccaaagtaccttgccttcacttaagtaatcaattcctaggttatattctgatgtcaaaggaagtcaaaagatgtgaaaaacaa
tttctgacccacaactcatgctttgtagatgactagatcaaaaaatttcagccatatcttaacagtgagtgaacaggaaatctcct
cttttccctacatctgagatcccagcttctaagaccttcaattctcactcttgatgcaacagaccttggaagcatacaggagagct
gaacttggtcaacaaaggagaaaagtttgttggcctccaaaggcacagctcaaacttttcaagccttctctaatcttaaaggtaaa
caagggtctcatttctttgagaacttcagggaaaatagacaaggacttgcctggtgcttttggtaggggagcttgcactttccccc
tttctggaggaaatatttatccccaggtagttccctttttgcaccagtggttctttgaagagacttccacctgggaacagttaaac
agcaactacagggccttgaactgcacactttcagtccggtcctcacagttgaaaagacctaagcttgtgcctgatttaagcctttt
tggtcataaaacattgaattctaatctccctctcaaccctacagtcacccatttggtatattaaagatgtgttgtctactgtctag
tatccctcaagtagtgtcaggaattagtcatttaaatagtctgcaagccaggagtggtggctcatgtctgtaattccagcacttga
gaggtagaagtgggaggactgcttgagctcaagagtttgatattatcctggacaacatagcaagacctcgtctctacttaaaaaaa
aaaaaaaaattagccaggcatgtgatgtacacctgtagtcccagctactcaggaggccgaaatgggaggatcccttgagctcagga
ggtcaaggctgcagtgagacatgatcttgccactgcactccagcctggacagcagagtgaaaccttgcctcacgaaacagaataca
aaaacaaacaaacaaaaaactgctccgcaatgcgcttccttgatgctctaccacataggtctgggtactttgtacacattatctca
ttgctgttcataattgttagattaattttgtaatattgatattattcctagaaagctgaggcctcaagatgataacttttattttc
tggacttgtaatagctttctcttgtattcaccatgttgtaactttcttagagtagtaacaatataaagttattgtgagtttttgca
aacacagcaaacacaacgacccatatagacattgatgtgaaattgtctattgtcaatttatgggaaaacaagtatgtactttttct
actaagccattgaaacaggaataacagaacaagattgaaagaatacattttccgaaattacttgagtattatacaaagacaagcac
gtggacctgggaggagggttattgtccatgactggtgtgtggagacaaatgcaggtttataatagatgggatggcatctagcgcaa
tgactttgccatcacttttagagagctcttggggaccccagtacacaagaggggacgcagggtatatgtagacatctcattctttt
tcttagtgtgagaataagaatagccatgacctgagtttatagacaatgagcccttttctctctcccactcagcagctatgagatgg
cttgccctgcctctctactaggctgactcactccaaggcccagcaatgggcagggctctgtcagggctttgatagcactatctgca
gagccagggccgagaaggggtggactccagagactctccctcccattcccgagcagggtttgcttatttatgcatttaaatgatat
atttattttaaaagaaataacaggagactgcccagccctggctgtgacatggaaactatgtagaatattttgggttccattttttt
ttccttctttcagttagaggaaaaggggctcactgcacatacactagacagaaagtcaggagctttgaatccaagcctgatcattt
ccatgtcatactgagaaagtccccacccttctctgagcctcagtttctctttttataagtaggagtctggagtaaatgatttccaa
tggctctcatttcaatacaaaatttccgtttattaaatgcatgagcttctgttactccaagactgagaaggaaattgaacctgaga
ctcattgactggcaagatgtccccagaggctctcattcagcaataaaattctcaccttcacccaggcccactgagtgtcagatttg
catgcactagttcacgtgtgtaaaaaggaggatgcttctttcctttgtattctcacatacctttaggaaagaacttagcacccttc
ccacacagccatcccaataactcatttcagtgactcaacccttgactttataaaagtcttgggcagtatagagcagagattaagag
tacagatgctggagccagaccacctgagtgattagtgactcagtttctcttagtagttgtatgactcagtttcttcatctgtaaaa
tggagggttttttaattagtttgtttttgagaaagggtctcactctgtcacccaaatgggagtgtagtggcaaaatctcggctcac
tgcaacttgcacttcccaggctcaagcggtcctcccacctcaacatcctgagtagctggaaccacaggtacacaccaccatacctc
gctaattttttgtatttttggtagagatggggtttcacatgttacacaggatggtctcagactccggagctcaagcaatctgccca
cctcagccttccaaagtgctgggattataagcatgattacaggagttttaacaggctcataagattgttctgcagcccgagtgagt
taatacatgcaaagagtttaaagcagtgacttataaatgctaactactctagaaatgtttgctagtattttttgtttaactgcaat
cattcttgctgcaggtgaaaactagtgttctgtactttatgcccattcatctttaactgtaataataaaaataactgacatttatt
gaaggctatcagagactgtaattagtgctttgcataattaatcatatttaatactcttggattctttcaggtagatactattatta
tccccattttactacagttaaaaaaactacctctcaacttgctcaagcatacactctcacacacacaaacataaactactagcaaa
tagtagaattgagatttggtcctaattatgtctttgctcactatccaataaatatttattgacatgtacttcttggcagtctgtat
gctggatgctggggatacaaagatgtttaaatttaagctccagtctctgcttccaaaggcctcccaggccaagttatccattcaga
aagcattttttactctttgcattccactgtttttcctaagtgactaaaaaattacactttattcgtctgtgtcctgctctgggatg
atagtctgactttcctaacctgagcctaacatccctgacatcaggaaagactacaccatgtggagaaggggtggtggttttgattg
ctgctgtcttcagttagatggttaactttgtgaagttgaaaactgtggctctctggttgactgttagagttctggcacttgtcact
atgcctattatttaacaaatgcatgaatgcttcagaatatgggaatattatcttctggaatagggaatcaagttatattatgtaac
ccaggattagaagattcttctgtgtgtaagaatttcataaacattaagctgtctagcaaaagcaagggcttggaaaatctgtgagc
tcctcaccatatagaaagcttttaacccatcattgaataaatccctataggggatttctaccctgagcaaaaggctggtcttgatt
aattcccaaactcatatagctctgagaaagtctatgctgttaacgttttcttgtctgctaccccatcatatgcacaacaataaatg
caggcctaggcatgactgaaggctctctcataattcttggttgcatgaatcagattatcaacagaaatgttgagacaaactatggg
gaagcagggtatgaaagagctctgaatgaaatggaaaccgcaatgcttcctgcccattcagggctccagcatgtagaaatctgggg
ctttgtgaagactggcttaaaatcagaagccccattggataagagtagggaagaacctagagcctacgctgagcaggtttccttca
tgtgacagggagcctcctgccccgaacttccagggatcctctcttaagtgtttcctgctggaatctcctcacttctatctggaaat
ggtttctccacagtccagcccctggctagttgaaagagttacccatgcagaggccctcctagcatccagagactagtgcttagatt
cctactttcagcgttggacaacctggatccacttgcccagtgttcttccttagttcctaccttcgaccttgatcctcctttatctt
cctgaaccctgctgagatgatctatgtggggagaatggcttctttgagaaacatcttcttcgttagtggcctgcccctcattccca
ctttaatatccagaatcactataagaagaatataataagaggaataactcttattataggtaagggaaaattaagaggcatacgtg
atgggatgagtaagagaggagagggaaggattaatggacgataaaatctactactatttgttgagaccttttatagtctaatcaat
tttgctattgttttccatcctcacgctaactccataaaaaaacactattattatctttattttgccatgacaagactgagctcaga
agagtcaagcatttgcctaaggtcggacatgtcagaggcagtgccagacctatgtgagactctgcagctactgctcatgggccctg
tgctgcactgatgaggaggatcagatggatggggcaatgaagcaaaggaatcattctgtggataaaggagacagccatgaagaagt
ctatgactgtaaatttgggagcaggagtctctaaggacttggatttcaaggaattttgactcagcaaacacaagaccctcacggtg
actttgcgagctggtgtgccagatgtgtctatcagaggttccagggagggtggggtggggtcagggctggccaccagctatcaggg
cccagatgggttataggctggcaggctcagataggtggttaggtcaggttggtggtgctgggtggagtccatgactcccaggagcc
aggagagatagaccatgagtagagggcagacatgggaaaggtgggggaggcacagcatagcagcatttttcattctactactacat
gggactgctcccctatacccccagctaggggcaagtgccttgactcctatgttttcaggatcatcatctataaagtaagagtaata
attgtgtctatctcatagggttattatgaggatcaaaggagatgcacactctctggaccagtggcctaacagttcaggacagagct
atgggcttcctatgtatgggtcagtggtctcaatgtagcaggcaagttccagaagatagcatcaaccactgttagagatatactgc
cagtctcagagcctgatgttaatttagcaatgggctgggaccctcctccagtagaaccttctaaccagctgctgcagtcaaagtcg
aatgcagctggttagactttttttaatgaaagcttagctttcattaaagattaagctcctaagcagggcacagatgaaattgtcta
acagcaactttgccatctaaaaaaatctgacttcactggaaacatggaagcccaaggttctgaacatgagaaatttttaggaatct
gcacaggagttgagagggaaacaagatggtgaagggactagaaaccacatgagagacacgaggaaatagtgtagatttaggctgga
ggtaaatgaaagagaagtgggaattaatacttactgaaatctttctatatgtcaggtgccattttatgatatttaataatctcatt
acatatggtaattctgtgagatatgtattattgaacatactataattaatactaatgataagtaacacctcttgagtacttagtat
atgctagaatcaaatttaagtttatcatatgaggccgggcacggtggctcatatatgggattacatgcctgtaatcccagcacttt
gggaggccaaggcaattggatcacctgaggtcaggagttccagaccagcctggccaacatggtgaaaccccttctctactaaaaaa
tacaaaaaatcagccaggtgtggtggcacgcgtctataatcccagctactcaggaggctgaggcaggagaatcacttgaacccagg
aggtggaggttgcagtgagctaagattgcaccactgcactccagcctaggcgacagagtgagactccatctcaaaaaaaaaaaaag
aagtttattatatgaattaacttagttttactcacaccaatactcagaagtagattattacctcatttattgatgaggagcccaat
gtacttgtagtgtagatcaacttattgaaagcacaagctaataagtagacaattagtaattagaagtcagatggtctgagctctcc
tactgtctacattacatgagctcttattaactggggactcgaaaatcaaagacatgaaataatttgtccaagcttacagaaccacc
aagtagtaaggctaggatgtagacccagttctgctacctctgaagacagtgttttttccacagcaaaacacaaactcagatattgt
ggatgcgagaaattagaagtagatattcctgccctgtggcccttgcttcttacttttacttcttgtcgattggaagttgtggtcca
agccacagttgcagaccatacttcctcaaccataattgcatttcttcaggaaagtttgagggagaaaaaggtaaagaaaaatttag
aaacaacttcagaataaagagattttctcttgggttacagagattgtcatatgacaaattataagcagacacttgagaaaactgaa
ggcccatgcctgcccaaattaccctttgaccccttggtcaagctgcaactttggttaaagggagtgtttatgtgttatagtgttca
tttactcttctggtctaacccattggctccgtcttcatcctgcagtgacctcagtgcctcagaaacatacatatgtttgtctagtt
taagtttgtgtgaaattctaactagcgtcaagaactgagggccctaaactatgctaggaatagtgctgtggtgctgtgataggtac
acaagaaatgagaagaaactgcagattctctgcatctccctttgccgggtctgacaacaaagtttccccaaattttaccaatgcaa
gccatttctccatatgctaactactttaaaatcatttggggcttcacattgtctttctcatctgtaaaaagaatggaagaactcat
tcctacagaactccctatgtcttccctgatgggctagagttcctctttctcaaaaattagccattattgtatttccttctaagcca
aagctcagaggtcttgtattgcccagtgacatgcacactggtcaaaagtaggctaagtagaagggtactttcacaggaacagagag
caaaagaggtgggtgaatgagagggtaagtgagaaaagacaaatgagaagttacaacatgatggcttgttgtctaaatatctccta
gggaattattgtgagaggtctgaatagtgttgtaaaataagctgaatctgctgccaacattaacagtcaagaaatacctccgaata
actgtacctccaattattctttaaggtagcatgcaactgtaatagttgcatgtatatatttatcataatactgtaacagaaaacac
ttactgaatatatactgtgtccctagttctttacacaataaactaatctcatcctcataattctattagctaatacatattatcat
cctatatttcagagacttcaagaagttaagcaacttgctcaagatcatctaagaagtaggtggtatttctgggctcatttggcccc
tcctaatctctcatggcaacatggctgcctaaagtgttgattgccttaattcatcagggatgggctcatactcactgcagacctta
actggcatcctcttttcttatgtgatctgcctgaccctagtagacttatgaaatttctgatgagaaaggagagaggagaaaggcag
agctgactgtgatgagtgatgaaggtgccttctcatctgggtaccagtggggcctctaagactaagtcactctgtctcactgtgtc
ttagccagttccttacagcttgccctgatgggagatagagaatgggtatcctccaacaaaaaaataaattttcatttctcaaggtc
caacttatgttttcttaatttttaaaaaaatcttgaccattctccactctctaaaataatccacagtgagagaaacattcttttcc
cccatcccataaatacctctattaaatatggaaaatctgggcatggtgtctcacacctgtaatcccagcactttgggaggctgagg
tgggtggactgcttggagctcaggagttcaagaccatcttggacaacatggtgataccctgcctctacaaaaagtacaaaaattag
cctggcatggtggtgtgcacctgtaatcccagctattagggtggctgaggcaggagaattgcttgaacccgggaggcggaggttgc
agtgagctgagatcgtgccactgcactccagcctgggggacagagcacattataattaactgttattttttacttggactcttgtg
gggaataagatacatgttttattcttatttatgattcaagcactgaaaatagtgtttagcatccagcaggtgcttcaaaaccattt
gctgaatgattactatactttttacaagctcagctccctctatcccttccagcatcctcatctctgattaaataagcttcagtttt
tccttagttcctgttacatttctgtgtgtctccattagtgacctcccatagtccaagcatgagcagttctggccaggcccctgtcg
gggtcagtgccccacccccgccttctggttctgtgtaaccttctaagcaaaccttctggctcaagcacagcaatgctgagtcatga
tgagtcatgctgaggcttagggtgtgtgcccagatgttctcagcctagagtgatgactcctatctgggtccccagcaggatgctta
cagggcagatggcaaaaaaaaggagaagctgaccacctgactaaaactccacctcaaacggcatcataaagaaaatggatgcctga
gacagaatgtgacatattctagaatatattatttcctgaatatatatatatatatacacatatacgtatatatatatatatatata
tatttgttgttatcaattgccatagaatgattagttattgtgaatcaaatatttatcttgcaggtggcctctatacctagaagcgg
cagaatcaggctttattaatacatgtgtatagatttttaggatctatacacatgtattaatatgaaacaaggatatggaagaggaa
ggcatgaaaacaggaaaagaaaacaaaccttgtttgccattttaaggcacccctggacagctaggtggcaaaaggcctgtgctgtt
agaggacacatgctcacatacggggtcagatctgacttggggtgctactgggaagctctcatcttaaggatacatctcaggccagt
cttggtgcattaggaagatgtaggcaactctgatcctgagaggaaagaaacattcctccaggagagctaaaagggttcacctgtgt
gggtaactgtgaaggactacaagaggatgaaaaacaatgacagacagacataatgcttgtgggagaaaaaacaggaggtcaagggg
atagagaaggcttccagaagaatggctttgaagctggcttctgtaggagttcacagtggcaaagatgtttcagaaatgtgacatga
cttaaggaactatacaaaaaggaacaaatttaaggagaggcagataaattagttcaacagacatgcaaggaattttcagatgaatg
ttatgtctccactgagcttcttgaggttagcagctgtgagggttttgcaggcccaggacccattacaggacctcacgtatacttga
cactgttttttgtattcatttgtgaatgaatgacctcttgtcagtctactcggtttcgctgtgaatgaatgatgtcttgtcagcct
acttggtttcgctaagagcacagagagaagatttagtgatgctatgtaaaaacttcctttttggttcaagtgtatgtttgtgatag
aaatgaagacaggctacatgatgcatatctaacataaacacaaacattaagaaaggaaatcaacctgaagagtatttatacagata
acaaaatacagagagtgagttaaatgtgtaataactgtggcacaggctggaatatgagccatttaaatcacaaattaattagaaaa
aaaacagtggggaaaaaattccatggatgggtctagaaagactagcattgttttaggttgagtggcagtgtttaaagggtgatatc
agactaaacttgaaatatgtggctaaataactagaatactctttattttttcgtatcatgaatagcagatatagcttgatggcccc
atgcttggtttaacatccttgctgttcctgacatgaaatccttaatttttgacaaaggggctattcattttcattttatattgggc
ctagaaattatgtagatggtcctgaggaaaagtttatagcttgtctatttctctctctaacatagttgtcagcacaatgcctaggc
tataggaagtactcaaagcttgttaaattgaattctatccttcttattcaattctacacatggaggaaaaactcatcagggatgga
ggcacgcctctaaggaaggcaggtgtggctctgcagtgtgattgggtacttgcaggacgaagggtggggtgggagtggctaacctt
ccattcctagtgcagaggtcacagcctaaacatcaaattccttgaggtgcggtggctcactcctgtaatcacagcagtttgggacg
ccaaggtgggcagatcacttgaggtcaggagttggacaccagcccagccaacatagtgaaacctggtctctgcttaaaaatataaa
aattagctggacgtggtgacgggagcctgtaatccaactacttgggaggctgaggcaggagaatcgcttgaaccggggaggtggag
tttgcactgagcagagatcatgccattgcactccagcctccagagcgagactctgtctaaagaaaaacgaaaacaaacaaacaaac
aaacaaacaaaacccatcaaattccctgaccgaacagaattctgtctgattgttctctgacttatctaccattttccctccttaaa
gaaactgtgaacttccttcagctagaggggcctggctcagaagcctctggtcagcatccaagaaatacttgatgtcactttggcta
aaggtatgatgtgtagacaagctccagagatggtttctcatttccatatccacccacccagctttccaattttaaagccaattctg
aggtagagactgtgatgaacaaacaccttgacaaaattcaacccaaagactcactttgcctagcttcaaaatccttactctgacat
atactcacagccagaaattagcatgcactagagtgtgcatgagtgcaacacacacacacaccaattccatattctctgtcagaaaa
tcctgttggtttttcgtgaaaggatgttttcagaggctgaccccttgccttcacctccaatgctaccactctggtctaagtcactg
tcaccaccacctaaattatagctgttgactcataacaatcttcctgcttctaccactgccccactacaatttcttcccaatatact
atccaaattagtcttttcaaaatgtaagtcatatatggtcacctctttgttcaaagtcttctgatagtttcctatatcatttataa
taaaaccaaatccttacaattctctacaatagttgttcatgcatatattatgtttattacagatacatatatatagctctcatata
aataaatatatatatttatgtgtatgtgtgtagagtgttttttcttacaactctatgatgtaggtattattagtgtcccaaatttt
ataatttaggacttctatgatctcatcttttattctccccttcaccgaatctcatcctacattggccttattgatattccttgaaa
attctaagcatcttacatctttagggtatttacatttgccattccctatgccctaaatatttaatcatagtttcatataaatgggt
tcctcatcatctatgggtactctctcaggtgttaactttatagtgaggactttcctgccatactacttaaagtagcgatacccttt
caccctgtcctaatcacactctggccttcatttcagttttttttttttctccatagcacctaatctcattggtatataacatgttt
catttgcttatttaatgtcaagctctttccactatcaagtccatgaaaacaggaactttattcctctattctgtttttgtgctgta
ttcttagcaattttacaattttgaatgaatgaatgagcagtcaaacacatatacaactataattaaaaggatgtatgctgacacat
ccactgctatgcacacacaaagaaatcagtggagtagagctggaagtgctaagcctgcatagagctagttagccctccgcaggcag
agccttgatgggattactgagttctagaattggactcatttgttttgtaggctgagatttgctcttgaaaacttgttctgaccaaa
ataaaaggctcaaaagatgaatatcgaaaccagggtgttttttacactggaatttataactagagcactcatgtttatgtaagcaa
ttaattgtttcatcagtcaggtaaaagtaaagaaaaactgtgccaaggcaggtagcctaatgcaatatgccactaaagtaaacatt
atttcataggtgtcagatatggcttattcatccatcttcatgggaaggatggccttggcctggacatcagtgttatgtgaggttca
aaacacctctaggctataaggcaacagagctccttttttttttttctgtgctttcctggctgtccaaatctctaatgataagcata
cttctattcaatgagaatattctgtaagattatagttaagaattgtgggagccattccgtctcttatagttaaatttgagcttctt
ttatgatcactgtttttttaatatgctttaagttctggggtacatgtgccatggtggtttgctgcacccatcaacccgtcatctac
attaggtatttctcctaatgctatccttcccctagccccccacccccaacaggccccagtgtgtgatgttcccctccctgtgtcca
tggatcactggtttttttttgttttttttttttttttaaagtctcagttaaatttttggaatgtaatttattttcctggtatccta
ggacttgcaagttatctggtcactttagccctcacgttttgatgataatcacatatttgtaaacacaacacacacacacacacaca
cacacatatatatatatataaaacatatatatacataaacacacataacatatttatcgggcatttctgagcaactaatcatgcag
gactctcaaacactaacctatagccttttctatgtatctacttgtgtagaaaccaagcgtggggactgagaaggcaatagcaggag
cattctgactctcactgcctttagctaggcccctccctcatcacagctcagcatagtcctgagctcttatctatatccacacacag
tttctgacgctgcccagctatcaccatcccaagtctaaagaaaaaaataatgggtttgcccatctctgttgattagaaaacaaaac
aaaataaaataagcccctaagctcccagaaaacatgactaaaccagcaagaagaagaaaatacaataggtatatgaggagactggt
gacactagtgtctgaatgaggcttgagtacagaaaagaggctctagcagcatagtggtttagaggagatgtttctttccttcacag
atgccttagcctcaataagcttgcggttgtggaagtttactttcagaacaaactcctgtggggctagaattattgatggctaaaag
aagcccgggggagggaaaaatcattcagcatcctcacccttagtgacacaaaacagagggggcctggttttccatatttcctcatg
atggatgatctcgttaatgaaggtggtctgacgagatcattgcttcttccatttaagccttgctcacttgccaatcctcagtttta
accttctccagagaaatacacattttttattcaggaaacatactatgttatagtttcaatactaaataatcaaagtactgaagata
gcatgcataggcaagaaaaagtccttagctttatgttgctgttgtttcagaatttaaaaaagatcaccaagtcaaggacttctcag
ttctagcactagaggtggaatcttagcatataatcagaggtttttcaaaatttctagacataagattcaaagccctgcacttaaaa
tagtctcatttgaattaactctttatataaattgaaagcacattctgaactacttcagagtattgttttatttctatgttcttagt
tcataaatacattaggcaatgcaatttaattaaaaaaacccaagaatttcttagaattttaatcatgaaaataaatgaaggcatct
ttacttactcaaggtcccaaaaggtcaaagaaaccaggaaagtaaagctatatttcagcggaaaatgggatatttatgagttttct
aagttgacagactcaagttttaaccttcagtgcccatcatgtaggaaagtgtggcataactggctgattctggctttctactcctt
tttcccattaaagatccctcctgcttaattaacattcacaagtaactctggttgtactttaggcacagtggctcccgaggtcagtc
acacaataggatgtctgtgctccaagttgccagagagagagattactcttgagaatgagcctcagccctggctcaaactcacctgc
aaacttcgtgagagatgaggcagaggtacactacgaaagcaacagttagaagctaaatgatgagaacacatggactcatagaggga
aacaacgcatactggggcctatcagagggtggagggtgagagaaggagaggatcaggaaaaatcactaatggatgctaagcgtaat
acctgagtgatgagatcatctatacaacaaacccccttgacattcatttatctatgtaacaaacctgcacatcctgtacatgtacc
cctgaacttaaaataaaagttgaaaacaagaaagcaacagtttgaacacttgttatggtctattctctcattctttacaattacac
tagaaaatagccacaggcttcctgcaaggcagccacagaatttatgacttgtgatatccaagtcattcctggataatgcaaaatct
aacacaaaatctagtagaatcatttgcttacatctatttttgttctgagaatatagatttagatacataatggaagcagaataatt
taaaatctggctaatttagaatcctaagcagctcttttcctatcagtggtttacaagccttgtttatatttttcctattttaaaaa
taaaaataaagtaagttatttgtggtaaagaatattcattaaagtatttatttcttagataataccatgaaaaacattcagtgaag
tgaagggcctactttacttaacaagaatctaatttatataatttttcatactaatagcatctaagaacagtacaatatttgactct
tcaggttaaacatatgtcataaattagccagaaagatttaagaaaatattggatgtttccttgtttaaattaggcatcttacagtt
tttagaatcctgcatagaacttaagaaattacaaatgctaaagcaaacccaaacaggcaggaattaatcttcatcgaatttgggtg
tttctttctaaaagtcctttatacttaaatgtcttaagacatacatagattttattttactaattttaattatatagacaataaat
gaatattcttactgattactttttctgactgtctaatctttctgatctatcctggatggccataacacttatctctctgaactttg
ggcttttaatataggaaagaaaagcaataatccatttttcatggtatctcatatgataaacaaataaaatgcttaaaaatgagcag
gtgaagcaatttatcttgaaccaacaagcatcgaagcaataatgagactgcccgcagcctacctgacttctgagtcaggatttata
agccttgttactgagacacaaacctgggcctttcaatgctataacctttcttgaagctcctccctaccacctttagccataaggaa
acatggaatgggtcagatccctggatgcaagccaggtctggaaccataggcagtaaggagagaagaaaatgtgggctctgcaactg
gctccgagggagcaggagaggatcaaccccatactctgaatctaagagaagactggtgtccatactctgaatgggaagaatgatgg
gattacccatagggcttgttttagggagaaacctgttctccaaactcttggccttgagatacctggtccttattccttggactttg
gcaatgtctgaccctcacattcaagttctgaggaagggccactgccttcatactgtggatctgtagcaaattccccctgaaaaccc
agagctgtatcttaattggttaaaaaaaattatattatctcaacgactgttcttctctgagtagccaagctcagcttggttcaagc
tacaagcagctgagctgctttttgtctagtcattgttcttttatttcagtggatcaaatacgttctttccaaacctaggatcttgt
cttcctaggctatatattttgtcccaggaagtcttaatctggggtccacagaacactagggggctggtgaagtttatagaaaaaaa
atctgtatttttacttacatgtaactgaaatttagcattttcttctactttgaatgcaaaggacaaactagaatgacatcatcagt
acctattgcatagttataaagagaaaccacagatattttcatactacaccataggtattgcagatctttttgtttttgtttttgtt
tgagatggagtttcgctcttattgcccaggctggagtgcagtggcatgatttcggctcactgcaacctccccttcctgcattcaag
caattctcctgccttggcctcctgagtagctggggattacaggcacctgccaccatgccagtctaatttttgtatttttagtagag
atggggtttcgccatgttggccaggctggtcttgaactcctgacctcagatgatctgcccgccttggcctcctgaagtgctgggat
tataggtgtgagccaccacgcctggcccattgcagatatttttaattcacatttatctgcatcactacttggatcttaaggtagct
gtagacccaatcctagatctaatgctttcataaagaagcaaatataataaatactataccacaaatgtaatgtttgatgtctgata
atgatatttcagtgtaattaaacttagcactcctatgtatattatttgatgcaataaaaacatatttttttagcacttacagtctg
ccaaactggcctgtgacacaaaaaaagtttaggaattcctggttttgtctgtgttagccaatggttagaatatatgctcagaaaga
taccattggttaatagctaaaagaaaatggagtagaaattcagtggcctggaataataacaatttgggcagtcattaagtcaggtg
aagacttctggaatcatgggagaaaagcaagggagacattcttacttgccacaagtgtttttttttttttttttttttatcacaaa
cataagaaaatataataaataacaaagtcaggttatagaagagagaaacgctcttagtaaacttggaatatggaatccccaaaggc
acttgacttgggagacaggagccatactgctaagtgaaaaagacgaagaacctctagggcctgaacatacaggaaattgtaggaac
agaaattcctagatctggtggggcaaggggagccataggagaaagaaatggtagaaatggatggagacggaggcagaggtgggcag
atcatgaggtcaagagatcgagaccatcctggcaaacatggtgaaatcccgtctctactaaaaataaaaaaattagctgggcatgg
tggcatgcgcctgtagtcccagctgctcgggaggctgaggcaggagaatcgtttgaacccaggaggcgaaggttgcagtgagctga
gatagtgccattgcactccagtctggcaacagagtgagactccgtctcaaaaaaaaaaaaaaaagaaagaaagaaaagaaaaagaa
aaaagaaaaaataaatggatgtagaacaagccagaaggaggaactgggctggggcaatgagattatggtgatgtaagggactttta
tagaattaacaatgctggaatttgtggaactctgcttctattattcccccaatcattacttctgtcacattgatagttaaataatt
tctgtgaatttattccttgattctaaaatatgaggataatgacaatggtattataagggcagattaagtgatatagcatgagcaat
attcttcaggcacatggatcgaattgaatacactgtaaatcccaacttccagtttcagctctaccaagtaaagagctagcaagtca
tcaaaatggggacatacagaaaaaaaaaaggacactagaggaataatataccctgactcctagcctgattaatatatcgattcact
tttttctctgtttgatgacaaattctggctttaaataattttaggattttaggcttctcagctcccttcccagtgagaagtataag
caggacagacaggcaagcaagaagagagccccaggcaatactcacaaagtagccaatgtcccctgtggtcatagagaaatgaaaag
agagaggattctctggaagcactggatgtaatcttttctgtctgtcctctctagggaatcaccccaaggtactgtactttgggatt
aaggctttagtcccactgtggactacttgctattctgttcagtttctagaaggaactatgtacggtttttgtctccctagagaaac
taaggtacagaagttttgtttacaatgcactccttaagagagctagaactgggtgagattctgttttaacagctttattttctttt
ccttggccctgtttttgtcactgtcaccacctttaaggcaaatgttaaatgcgctttggctgaaactttttttcctattttgagat
ttgctcctttatatgaggctttcttggaaaaggagaatgggagagatggatatcattttggaagatgatgaagagggtaaaaaagg
ggacaaatggaaatttgtgttgcagatagatgaggagccaacaaaaaagagcctcaggatccagcacacattatcacaaacttagt
gtccatccatcactgctgaccctctccggacctgactccacccctgagggacacaggtcagccttgaccaatgacttttaagtacc
atggagaacagggggccagaacttcggcagtaaagaataaaaggccagacagagaggcagcagcacatatctgcttccgacacagc
tgcaatcactagcaagctctcaggcctggcatcatggtgcattttactgctgaggagaaggctgccgtcactagcctgtggagcaa
gatgaatgtggaagaggctggaggtgaagccttgggcaggtaagcattggttctcaatgcatgggaatgaagggtgaatattaccc
tagcaagttgattgggaaagtcctcaagattttttgcatctctaattttgtatctgatatggtgtcatttcatagactcctcgttg
tttacccctggacccagagattttttgacagctttggaaacctgtcgtctccctctgccatcctgggcaaccccaaggtcaaggcc
catggcaagaaggtgctgacttcctttggagatgctattaaaaacatggacaacctcaagcccgcctttgctaagctgagtgagct
gcactgtgacaagctgcatgtggatcctgagaacttcaaggtgagttcaggtgctggtgatgtgattttttggctttatattttga
cattaattgaagctcataatcttattggaaagaccaacaaagatctcagaaatcatgggtcgagcttgatgttagaacagcagact
tctagtgagcataaccaaaacttacatgattcagaactagtgacagtaaaggactactaacagcctgaattggcttaacttttcag
gaaatcttgccagaacttgatgtgtttatcccagagaattgtattatagaattgtagacttgtgaaagaagaatgaaatttggctt
ttggtagatgaaagtccatttcaaggaaatagaaatgccttattttatgtgggtcatgataattgaggtttagaaagagatttttg
caaaaaaaataaaagatttgctcaaagaaaaataagacacattttctaaaatatgttaaatttcccatcagtattgtgaccaagtg
aaggcttgtttccgaatttgttggggattttaaactcccgctgagaactcttgcagcactcacattctacatttacaaaaattaga
caattgcttaaagaaaaacagggagagagggaacccaataatactggtaaaatggggaagggggtgagggtgtaggtaggtagaat
gttgaatgtagggctcatagaataaaattgaacctaagctcatctgaattttttgggtgggcacaaaccttggaacagtttgaggt
cagggttgtctaggaatgtaggtataaagccgtttttgtttgtttgtttgttttttcatcaagttgttttcggaaacttctactca
acatgcctgtgtgttattttgtcttttgcctaacagctcctgggtaacgtgatggtgattattctggctactcactttggcaagga
gttcacccctgaagtgcaggctgcctggcagaagctggtgtctgctgtcgccattgccctggcccataagtaccactgagttctct
tccagtttgcaggtgttcctgtgaccctgacaccctccttctgcacatggggactgggcttggccttgagagaaagccttctgttt
aataaagtacattttcttcagtaatcaaaaattgcaattttatcttctccatcttttactcttgtgttaaaaggaaaaagtgttca
tgggctgagggatggagagaaacataggaagaaccaagagcttccttaagaaatgtatgggggcttgtaaaattaatgtggatgtt
atgggagaattccaggattccaaggaggatgatatgatggagaaaaatctttatcggggtgggaaaatggttaattaagtggacag
agactcctaggcagtttttactgcaccggggaaagaaggagctgttagtggtacctgagaaagcagatttgtggtacatgtcactt
ttcattaaaaacaaaaacaaaacaaaacaaaacttcatagatatccaagatataggctagaattactattttaatttactcttatt
tacattttgaagtagctagcttgtcacatgttttatgaaattgatttggagataagatgagtgtgtatcaacaatagcctgctctt
tccatgaaggattccattatttcatgggttagctgaagctaagacacatgatatcattgtgcattatcttctgatagaatgtaaca
tgcactaaaataaagttagagttaggacctgagtgggaaagtttttggagagtgtgatgaagactttccgtgggagatagaatact
aataaaggcttaaattctaaaaccagcaagctagggcttcgtgacttgcatgaaactggctctctggaagtagaagggagagtaag
acatacgtagaggactaggaaagaccagatagtacagggcctggctacaaaaatacaagcttttactatgctattgcaatactaaa
cgataagcattaggatgttaagtgactcaggaaataagattttgggaaaaagtaatctgcttatgtgcacaaaatggattcaagtt
tgcagataaaataaaatatggatgatgattcaaggggacagatacaatggttcaaacccaagaggagcagtgagtctgtggaattt
gaaggatggacaaaggtggggtgagaaagacatagtattcgactgactgtgggagatgagaaggaagaaggaggtgataaatgact
gaaagctcccagactggtgaagataacaggaggaaaccatgcactgacctggtgactctcatgtgtgaagggtagagggatattaa
cagatttactttttaggaagtgctagattggtcagggagttttgaccttcaggtcttgtgtctttcatatcaaggaacctttgcat
tttccaagttagagtgccatattttggcaaatataactttattagtaattttatagtgctctcacattgatcagactttttcctgt
gaattacttttgaatttggctgtatatatccagaatatgggagagagacaaataattattgtagttgcaggctatcaacaatactg
gtctctctgagccttataacctttcaatatgcccataaacagagtaaacagggattattcatggcactaaatattttcacctagtc
agtcaacaaatgggagcaatgtgcattttttgatacatatttttatatatttatggggtacatgtgatacttacatgcctagaaca
tgtgatgattaagtctagatatttaggatatccattgctttgagcatttatcatttctatgtattgagaaaatttcaaatcctcat
ttctagccattttgaaatatataataaatagtaattaactatagtcaccctactcaaatatcaaacattatggcttaatccttcta
tccaactgtgtttgtacctattaaccaacatctcttaaatcccctcccatacacactcacactttttccagcctctgataactatc
attctactctctaccaccatgagacccacttttttagctcccacagatgaataaaaacatgtgatatttgactttctgtatctggc
ttattttattatctatctctttggcataccaagagtttgtttttgttctgcttcagggctttcaattaacataatgacctctggtt
ccatccatgttgctacaaatgacaagatttcattctttttcatggcaaaatagtactgtgcaaaaatacaattttttaatccgttc
atctgttgatagacacttaggttgatcccaaaccttaactattgtgaatagtgcttcaataaacatgagtgtaatgtgtccattgg
atatactgatttcctttcttttggataaataaccactagtgagattgctggattgtatgatagttctgtttttagtttactgagaa
atcttcatactgttttccataatggttgtactattttacattcccaccaacagtgtgtaagaaagagttcccttttctccatatcc
tcacaaggatctgttattttttgtcttttttgttaatagccgttttaactagagtaagtagatatctcattgtagttttgatttgc
atttccctgatcattagtgatgttgagaattttttcatatgtttgttggtcatttgtatatctttttctgagaattgtctgttcat
gtccttagcctactttttattgggattgtttgttattttcttgataatctatttgtgttcattttagagcctggatattattcttt
tgtcagatgtatagattgtgaagattttctcccactctgtgggttgtctgtttattctgcagactcttccttttgccatgcaaaag
ctctttagtttaatttagtcccagatattttctttgtttttatgtatttgcatttgtgttcttggtcatgaaatcctttcctaagc
caatgtgtagaagggtttttccgatgttattttctagaattgttacagtttcagggcttagatttaagtccttgatccatcttgag
ttgatttttgtataaggtgagagatgaagatccagtttcattctcctacatgtagcttgccagctatccccgcaccatttgttgaa
tagggtgccctttccccactttatgtttttgtttgctttgtcaaagatcagttggatgtaagtatttgagtttatttctgggttct
ctattctgttccattggtcgatgtgcctatttgtacaccagcatcatgctgttttggtgactatggccttattgtatagtttgaaa
tgaggtaatgtaatgccttcagatttgttcttttttttagacttgcttgtttattgggctcttttttggttccataagaattttag
gattgttttttctagttctgtgaagactaatggtggtattttgatgggaattgcaatgaatttgtaggttgcttctggcattatgg
ccattttcacaatattgattctacccatctatgagaatggcatgtgtttccatttgtttgtgtcttatatgattactttcagccgt
gttttgtagttttccttgtagatgtctttcacctccttggttaggtatatattcctaagtttttgttttgttttgttttgtttttt
gcagctattgtaaaaggggttgagttcttgattttattctcagcttggtcattgctggtatgtaagaaagcaactcattggtgtac
gttaattttgtatccagaaactttgctgaattattttatcagttctagggggttttggaggagtctttagagttttctacatacac
aatcatatcatcagcaaacagtgacagtttgactttctctttaacaatttggatgtgctttacttgtttctcttgtctgattgctc
ttgctaggacttccagtaatatgttaaagagaagtggtgagagtgggtatccttgtctcattccagttttcagacagaatgctttt
aactttttcccattcaatataatgttggctgtgtgtttaccatagctggcttttattacattgaggtatgtcctttgtaaaccgat
tttgctgagttttagtcataaagtgatgttgaattttgttgaatgcagtttctgtggctattgagataatcacatgatttttgttt
ccaattctctttatgttgtgtatcacacttattgacttgcgtatgttaaaccatccgtgcatccctcgcatgaaacccacttgatc
atgggttttgatatgctgtcggatgctattagctagtattttgtcaaggatgttggcatctatgttcatcagggatattgatctgt
agtgttttttttttttggttatgttctttcccagttttggtattaaggtgatactggcttcatagaatgatttagggaggattctc
tctttctctatcttgtagaatactgtcaataggattggtatcaattcttctttgaatgtctggtagaattcagctgtgaatctatc
tggtcctggacttttttgttgttggtaaatttttattatcatttcagtcttgctgcttattactggtctgttcagggtatctaatt
cttcctgacttaagctagagccctgtatctttccaggaattcgaacgtctcctttaggttttctagtttatgcatgtaaaggtgtt
catagtagccttgaataatcttttgtatttctgtggtatcagtaatagtatctcctgttttgtttctaattgagtttatttgcact
tctctcctcttttcttggttaatcttgctaatggtctatcagttttatttatcttttcaaagaaccagctttttatttcatttagc
ttttgtatttttttgcagttgttttaatttcatttagttctcctcttatcttagttattccctttcttttgctgggttttggttct
gtttgtttttgtttctctagtttcttgtggtgtgaccttatattgtctgtctgtcctctttcagactctttgacatcgacatttag
ggctgtgaactttccttttagcaccatctttgctgtatcctagaggttttgataggttgtgtcactattgtcggtcagttcaagta
attttgttgttcttattatactttaagttctgggatacatgtgcagaatgtgcaggtttgttacataggtatagatgtgccatggt
ggtttgctgcacccatcaacctgtcatctacattaggtatttcttttaatgttatccctctcctaaccccctcaccccccgacagg
ccctggtgtgtgatgttcccctccctgtgtccatgtgttctcattgttcaactcccacttatgagtgagaacgtgtggtgtttggt
ttctctgttcctgtgttagtttgctcagaatgatggtttccaccttcatccatgtccctgcaaagacatgaactcatcatttttat
ggctgcatagtattccatggtgtatatgtgccacattttctttatccattatatcgctgatggccatttgggttggttccaagtct
ttgctattgtgaatagtgccacaataaacatacgtgtgcacgtgtctttatagtagaatgatttctaattctttgggtatataccc
agtaatgggattgctgggtcaaacagtatttctggttctagatccttgaggaatcgccacactgtcttccacaatggttgaactaa
tttacacacccatcaacagtgtaaaatttttcctattcttccacatcctctccagcaccttttgtttcctgactttttaataattg
ccattctaactggcatgagatggtatctcattgtggttttgatttgcatttctctaatgaccagtgatgatgagcttcttttcatg
tgtttcttggccacataaatgacttctttagagaagcatctgttcatatcctttgtccactttttgatggggtcgttaggtttttt
cttgtaaatttgttgaagttctttgtagattttggatgttagccctttgtcagatggatagattgcaaaaattttctcccattctg
taggttgcctgttcactctgatgatagtcttttgctgtgcagaagctctttagtttaattagatcccatatgtcaattttggcctt
tgttgtcattgcttttgatgttttagtcgtgaatttttgcccatgcctatgtcctgaatggtattgcctaggttatcttctaggat
ttttatggttttaggttgcacatttaagtctttaatccaccttgagttaatttttgtataaggtgtaaggaaggggtacagtttca
gttttatgcatattgctagccagtttttccagcaccatttattaaatagggaattctttctccattgcttttgtgatgtttgtcaa
agatcagatggtcgtagatgtgtggcattatttctgaggcttctgttctgttccactggtctatatatctgttttggtaccagtac
catgctgtttttgttactgtagccttgtagtatagtttgaagtcaggtagcatcatgcctccagctttgttctttttgtttaggat
tgtcttggctatatgggctcttttttgattccatatgacatttaaagtagttttttctaattctttgaaaaaagtcagtggtagct
tgatggggatagcattgaatctataaattactttgggcagtatggccattttaaagatattgattctttctatctatgagcatgga
atgtttttccatttgtttgtgtcctctcttatttccttgagcagtgagtggtttgtagctctccttgaagaggttcttcacatccc
ttagaagttgtatttctaggtattttattttattctctttgcagcaattgtgaatgggagttcacccatgatttggctctctgctt
gtctattattggtgtataggaacgcttgtgatttctgcacactgattttgtatcttgagactttgctgaagctgtttatcagctta
agattttgggctgagatgacagggtcttctaaatatacaatcatgtcatctgcaaacagagacaatttgacttcctctcttcctat
ttgaatatgctttatttctttctcttgcctgattgtcctggcgagaacttccaatactatgttgagtaagagtggcgagagggcat
ccttgtcttgtgccggttttcaaagcaaatgatttttaaatttccatcttgatttcattgttgacccaatgatcattcaggagcag
gttatttaatttccctgtatttgcatggttttgaaggttccttttgtagttgatttccaattttattctactgtggtctgagagag
tgcttgatataatttcaatttttaaaaatttattgaggcttgttttgtggcatatcatatggcctatcttggagaaagttccatgt
gctgatgaatagaatgtgtattctgcagttgttgggtagaatgtcctgtaaatatctgttaagtccatttgttctttaaatccatt
gtttctttgtagactgtcttgatgacctgcctagtgcagtcagtggagtattgaagtcccccactattattatgttgctgtctagt
ctagtagtaattgttttataaatttgggatctccagtattagatgcatatatattaagaattgtaatattctcccattggacaagg
gcttttatcattatatgatgtccctctttgtcttttttaactgctgtttctttaaagtttgttttgtctgacataagaatagctgc
tttggctcgcttttggtgtccatttgtgtggaatgtcattttccacccctttaccttaagtttatgtgagtccttatgtgttaggt
gagtctcctgaaggcggcagataactggttggtgaattcttattcattctgcaattctgtatcttttaagtggagcatttagtcca
tttacattcaacatcagtattgaggtgtgaggtactattccattcttcgtggtatttgttgcctgtgtatctttttatctgtattt
ttgttgtatatgtcctatgggatttatgctttaaagaggttctgttttgatgtgcttccagggtttatttcaagatttagagctcc
ttttatcagttcttgtagtgttggcttggtagtgccgaattctctcagcatttgtttttctgaaaaacactgtgtattttcttcat
ttgtgaagcttagtttcactggatataaaattcttggctgataattgttttgtttaagaaggctgaagatagggccatattcactt
ctagcttttacggtttctgctgagaaatctgctgttaatctgataggttttctttcataggttacctggtagtttcacctcacagc
tcttaagattctctttgtctttagataactttggatactctgatgacaatgtacctaggcaatgatatttttgcaatgaatttccc
aggtgtttattgagcttcttgtatttggatatctaggtctctagcaaggtgggggaagttttccttgattatttccctggataagt
tttccaaacttttagatttctcttctttctcaggaatgctgattattcttaggtttgattgtttaacataatcccagatttcttgg
aggctttgttcatattttcttattcttttttctttgtctttgttggattgggttaattcaaaaactttgtcttcaagctctgaatt
tcttctgcttggattctattgctgagactttctagagcattttgcatttctataagtgcatccattcatccattgtttcctgaagt
tttgaatgttttttatttatgctatctctttaactgaagatttctcccctcatttcttgtatcatatttttggtttttttaaaatt
ggacttcaccttcctcggatgcctccttgattagcttaataactgaccttctgaattatttttcaggtaaatcagggatttcttct
tggtttggatgcattgctggtgagctagtatgattttttggggggtgttaaagaaccttgtttttcatattaccagagttagtttt
ctggttccttctcacttgggtaggctctgtcagagggaaagtctaggcctcaaggctgagacttttgtcccatgaggtgttccctt
gatgtagcacagtcccccttttcctaggcgtggggcttcctgagagccgaactgtagtgattgttatctctcttctggatctagcc
acccatcaggtctaccagactccaggctggtactggggtttgtctgcacagagtcttgtgacgtgaaccatctgtgggtctctcag
ccatagatacaaccacctgctccaatggaggtggcagaggatgaaatggactctgtgagggtccttacttttggttgttcaatgca
ctatttttgtgctggttggcctcctgccaggaggtggcactttctagaaagcatcagcagaggcagtcaggtggtggtggctgggg
gggctggggcaccctagaactcccaagaatatatgccctttgtcttcagctaccagggtgagtaaggaaggaccatcaggtggggg
caggactagtcgtgtctgagctcagagtctccttgggcaggtctttctgtggctactgtgggaggatgggggtgtagtttccaggt
caatggatttatgttcctaggacaattatggctgcctctgctgtgtcatgcaggtcatcaggaaagtgggggaaagcaagcagtca
cgtgacttgcccagctcccatgcaactcaaaaggttggtctcacttccagcgtgcaccctcccccgcaacagcaccgaatctgttt
ccatgcagtcagtgagcaaggctgagaacttgccccaggctaccagctgcgaaaccaagtagggctgtcctacttccctgccagtg
gagtctgcacaccaaattcatgtccccccaccaacccccccactgcccagcccctagatctggccaggtggagattttctttttcc
tgtcatcttttcccagttcctctggcagccctcccaaatgacccctgtgaggcaaggcagaaatggcttcctaggggacccagaga
gcccacagggcttttcccgctgcttcctctacccctgtattttgcttggccctctaaattgactcagctccaggtaaggtcagaat
cttctcctgtggtctagatcttcaggttccccagtgaggatgtgtgtttgggggtagacggtcccccttttccacttccacagttt
gggcactcacaatatttggggtgtttcccgggtcctgcaggagcaatctgcttctttcagagggtgtgtgcgttctctcagctttc
ttgatttatttctgcaggtggttctgcaaaaaaaattcctgatgggagacttcacatgctgctctgtgcatccgagtgggagctgc
aatgtacttctgctgcctcccatctgccatcaccctctaatttgtcggtaatatgcatttttaatcaatctttttttctctctctc
tctttttcttctcccccaaaactatactgccctttgatatcaaggaatcaaggacgtgatgttgaggggtgggcagtggatacact
ctttaccccttagggagctatatctagatttagatattgccaattcaagataacttaattgaaagcaaattcataatgaatacaca
cacacacacacacatctgcatgacaagatttttaatagttgaaagaataactaataattgtccacaggcaataagggctttttaag
caaaacagttgtgataaacaggtcattcttagaatagtaatccagccaatagtacaggttgcttagagattatgtcattaccagag
ttaaaattctataatggcttctcactccctaccactgaggacaagtttatgtccttaggtttatgcttccctgaaacaataccacc
tgctattctccactttacatatcaacggcactggttctttatctaactctctggcacagcaggagtttgttttcttctgcttcaga
gctttgaatttactatttcagcttctaaactttatttggcaatgccttcccatggcagattccttctgtcattttgcctctgttcg
aatactttctccttaatttcattcttagttaataatatctgaaattattttgttgtttaacttaattattaattttatgtatgttc
tacctagattataatcttcagaggaaagttttattctctgacttatttaacttaaatgcccactactttaaaaattatgacattta
tttaacagatatttgctgaacaaatgtttgaaaatacatgggaaagaatgcttgaaaacacttgaaattgcttgtgtaaagaaaca
gttttatcagttaggatttaatcaatgtcagaagcaatgatataggaaaaatcgaggaataagacagttatggataaggagaaatc
aacaaactcttaaaagatattgcctcaaaagcataagaggaaataagggtttatacatgacttttagaacactgccttggtttttg
gataaatggggaagttgtttgaaaacaggagggatcctagatattccttagtctgaggaggagcaattaagattcacttgtttaga
ggctgggagtggtggctcacgcctgtaatcccagaattttgggaggccaaggcaggcagatcacctgaggtcaagagttcaagacc
aacctggccaacatggtgaaatcccatctctacaaaaatacaaaaattagacaggcatgatggcaagtgcctgtaatcccagctac
ttgggaggctgaggaaggagaattgcttgaacctggaaggcaggagttgcagtgagccgagatcataccactgcactccagcctgg
gtgacagaacaagactctgtctcaaaaaaaaaaaagagagattcaaaagattcacttgtttaggccttagcgggcttagacaccag
tctctgacacattcttaaaggtcaggctctacaaatggaacccaaccagactctcagatatggccaaagatctatacacacccatc
tcacagatcccctatcttaaagagaccctaatttgggttcacctcagtctctataatctgtaccagcataccaataaaaatctttc
tcacccatccttagattgagagaagtcacttattattatgtgagtaactggaagatactgataagttgacaaatctttttctttcc
tttcttattcaacttttattttaacttccaaagaacaagtgcaatatgtgcagctttgttgcgcaggtcaacatgtatctttctgg
tcttttagccgcctaacactttgagcagatataagccttacacaggattatgaagtctgaaaggattccaccaatattattataat
tcctatcaacctgataggttaggggaaggtagagctctcctccaataagccagatttccagagtttctgacgtcataatctaccaa
ggtcatggatcgagttcagagaaaaaacaaaagcaaaaccaaacctaccaaaaaataaaaatcccaaagaaaaaataaagaaaaaa
acagcatgaatacttcctgccatgttaagtggccaatatgtcagaaacagcactgagttacagataaagatgtctaaactacagtg
acatcccagctgtcacagtgtgtggactattagtcaataaaacagtccctgcctcttaagagttgttttccatgcaaatacatgtc
ttatgtcttagaataagattccctaagaagtgaacctagcatttatacaagataattaattctaatccatagtatctggtaaagag
cattctaccatcatctttaccgagcatagaagagctacaccaaaaccctgggtcatcagccagcacatacacttatccagtgataa
atacacatcatcgggtgcctacatacatacctgaatataaaaaaaatacttttgctgagatgaaacaggcgtgatttatttcaaat
aggtacggataagtagatattgaagtaaggattcagtcttatattatattacataacattaatctattcctgcactgaaactgttg
ctttataggatttttcactacactaatgagaacttaagagataatggcctaaaaccacagagagtatattcaaagataagtatagc
acttcttatttggaaaccaatgcttactaaatgagactaagacgtgtcccatcaaaaatcctggacctatgcctaaaacacatttc
acaatccctgaacttttcaaaaattggtacatgctttaactttaaactacaggcctcactggagctacagacaagaaggtgaaaaa
cggctgacaaaagaagtcctggtatcttctatggtgggagaagaaaactagctaaagggaagaataaattagagaaaaattggaat
gactgaatcggaacaaggcaaaggctataaaaaaaattaagcagcagtatcctcttgggggccccttccccacactatctcaatgc
aaatatctgtctgaaacggtccctggctaaactccacccatgggttggccagccttgccttgaccaatagccttgacaaggcaaac
ttgaccaatagtcttagagtatccagtgaggccaggggccggcggctggctagggatgaagaataaaaggaagcacccttcagcag
ttccacacactcgcttctggaacgtctgaggttatcaataagctcctagtccagacgccatgggtcatttcacagaggaggacaag
gctactatcacaagcctgtggggcaaggtgaatgtggaagatgctggaggagaaaccctgggaaggtaggctctggtgaccaggac
aagggagggaaggaaggaccctgtgcctggcaaaagtccaggtcgcttctcaggatttgtggcaccttctgactgtcaaactgttc
ttgtcaatctcacaggctcctggttgtctacccatggacccagaggttctttgacagctttggcaacctgtcctctgcctctgcca
tcatgggcaaccccaaagtcaaggcacatggcaagaaggtgctgacttccttgggagatgccataaagcacctggatgatctcaag
ggcacctttgcccagctgagtgaactgcactgtgacaagctgcatgtggatcctgagaacttcaaggtgagtccaggagatgtttc
agcactgttgcctttagtctcgaggcaacttagacaactgagtattgatctgagcacagcagggtgtgagctgtttgaagatactg
gggttgggagtgaagaaactgcagaggactaactgggctgagacccagtggcaatgttttagggcctaaggagtgcctctgaaaat
ctagatggacaactttgactttgagaaaagagaggtggaaatgaggaaaatgacttttctttattagatttcggtagaaagaactt
tcacctttcccctatttttgttattcgttttaaaacatctatctggaggcaggacaagtatggtcattaaaaagatgcaggcagaa
ggcatatattggctcagtcaaagtggggaactttggtggccaaacatacattgctaaggctattcctatatcagctggacacatat
aaaatgctgctaatgcttcattacaaacttatatcctttaattccagatgggggcaaagtatgtccaggggtgaggaacaattgaa
acatttgggctggagtagattttgaaagtcagctctgtgtgtgtgtgtgtgtgtgtgcgcgcgtgtgtttgtgtgtgtgtgagagc
gtgtgtttcttttaacgttttcagcctacagcatacagggttcatggtggcaagaagataacaagatttaaattatggccagtgac
tagtgctgcaagaagaacaactacctgcatttaatgggaaagcaaaatctcaggctttgagggaagttaacataggcttgattctg
ggtggaagcttggtgtgtagttatctggaggccaggctggagctctcagctcactatgggttcatctttattgtctcctttcatct
caacagctcctgggaaatgtgctggtgaccgttttggcaatccatttcggcaaagaattcacccctgaggtgcaggcttcctggca
gaagatggtgactggagtggccagtgccctgtcctccagataccactgagctcactgcccatgatgcagagctttcaaggataggc
tttattctgcaagcaatcaaataataaatctattctgctaagagatcacacatggttgtcttcagttctttttttatgtcttttta
aatatatgagccacaaagggttttatgttgagggatgtgtttatgtgtatttatacatggctatgtgtgtttgtgtcatgtgcaca
ctccacacttttttgtttacgttagatgtgggttttgatgagcaaataaaagaactaggcaataaagaaacttgtacatgggagtt
ctgcaagtgggagtaaaaggtgcaggagaaatctggttggaagaaagacctctataggacaggactcctcagaaacagatgttttg
gaagagatggggaaaggttcagtgaagggggctgaacccccttccctggattgcagcacagcagcgaggaaggggctcaacgaaga
aaaagtgttccaagctttaggaagtcaaggtttaggcagggatagccattctattttattaggggcaatactatttccaacggcat
ctggcttttctcagcccttgtgaggctctacagggaggttgaggtgttagagatcagagcaggaaacaggtttttctttccacggt
aactacaatgaagtgatccttactttactaaggaacttttcattttaagtgttgacgcatgcctaaagaggtgaaattaatcccat
acccttaagtctacagactggtcacagcatttcaaggaggagacctcattgtaagcttctagggaggtggggacttaggtgaagga
aatgagccagcagaagctcacaagtcagcatcagcgtgtcatgtctcagcagcagaacagcacggtcagatgaaaatatagtgtga
agaatttgtataacattaattgagaaggcagattcactggagttcttatataattgaaagttaatgcacgttaataagcaagagtt
tagtttaatgtgatggtgttatgaacttaacgcttgtgtctccagaaaattcacatgctgaatccccaactcccaattggctccat
ttgtgggggaggctttggaaaagtaatcaggtttagaggagctcatgagagcagatccccatcatagaattattttcctcatcaga
agcagagagattagccatttctcttccttctggtgaggacacagtgggaagtcagccacctgcaacccaggaagagagccctgacc
aggaaccagcagaaaagtgagaaaaaatcctgttgttgaagtcacccagtctatgctattttgttatagcaccttgcactaagtaa
ggcagatgaagaaagagaaaaaaataagcttcggtgttcagtggattagaaaccatgtttatctcaggtttacaaatctccacttg
tcctctgtgtttcagaataaaataccaactctactactctcatctgtaagatgcaaatagtaagcctgagcccttctgtctaactt
tgaattctattttttcttcaacgtactttaggcttgtaatgtgtttatatacagtgaaatgtcaagttctttctttatatttcttt
ctttcttttttttcctcagcctcagagttttccacatgcccttcctactttcaggaacttctttctccaaacgtcttctgcctggc
tccatcaaatcataaaggacccacttcaaatgccatcactcactaccatttcacaattcgcactttctttctttgtcctttttttt
tttagtaaaacaagtttataaaaaattgaaggaataaatgaatggctacttcataggcagagtagacgcaagggctactggttgcc
gatttttattgttatttttcaatagtatgctaaacaaggggtagattatttatgctgcccatttttagaccataaaagataacttc
ctgatgttgccatggcatttttttccttttaattttatttcatttcattttaatttcgaaggtacatgtgcaggatgtgcaggctt
gttacatgggtaaatgtgtgtctttctggccttttagccatctgtatcaatgagcagatataagctttacacaggatcatgaagga
tgaaagaatttcaccaatattataataatttcaatcaacctgatagcttaggggataaactaatttgaagatacagcttgcctccg
ataagccagaattccagagcttctggcattataatctagcaaggttagagatcatggatcactttcagagaaaaacaaaaacaaac
taaccaaaagcaaaacagaaccaaaaaaccaccataaatacttcctaccctgttaatggtccaatatgtcagaaacagcactgtgt
tagaaataaagctgtctaaagtacactaatattcgagttataatagtgtgtggactattagtcaataaaaacaacccttgcctctt
tagagttgttttccatgtacacgcacatcttatgtcttagagtaagattccctgagaagtgaacctagcatttatacaagataatt
aattctaatccacagtacctgccaaagaacattctaccatcatctttactgagcatagaagagctacgccaaaaccctgggtcatc
agccagcacacacacttatccagtggtaaatacacatcatctggtgtatacatacatacctgaatatggaatcaaatatttttcta
agatgaaacagtcatgatttatttcaaataggtacggataagtagatattgaggtaagcattaggtcttatattatgtaacactaa
tctattactgcgctgaaactgtggctttatagaaattgttttcactgcactattgagaaattaagagataatggcaaaagtcacaa
agagtatattcaaaaagaagtatagcactttttccttagaaaccactgctaactgaaagagactaagatttgtcccgtcaaaaatc
ctggacctatgcctaaaacacatttcacaatccctgaacttttcaaaaattggtacatgctttagctttaaactacaggcctcact
ggagctagagacaagaaggtaaaaaacggctgacaaaagaagtcctggtatcctctatgatgggagaaggaaactagctaaaggga
agaataaattagagaaaaactggaatgactgaatcggaacaaggcaaaggctataaaaaaaattagcagtatcctcttgggggccc
cttccccacactatctcaatgcaaatatctgtctgaaacggtccctggctaaactccacccatgggttggccagccttgccttgac
caatagccttgacaaggcaaacttgaccaatagtcttagagtatccagtgaggccaggggccggcggctggctagggatgaagaat
aaaaggaagcacccttcagcagttccacacactcgcttctggaacgtctgaggttatcaataagctcctagtccagacgccatggg
tcatttcacagaggaggacaaggctactatcacaagcctgtggggcaaggtgaatgtggaagatgctggaggagaaaccctgggaa
ggtaggctctggtgaccaggacaagggagggaaggaaggaccctgtgcctggcaaaagtccaggtcgcttctcaggatttgtggca
ccttctgactgtcaaactgttcttgtcaatctcacaggctcctggttgtctacccatggacccagaggttctttgacagctttggc
aacctgtcctctgcctctgccatcatgggcaaccccaaagtcaaggcacatggcaagaaggtgctgacttccttgggagatgccac
aaagcacctggatgatctcaagggcacctttgcccagctgagtgaactgcactgtgacaagctgcatgtggatcctgagaacttca
aggtgagtccaggagatgtttcagccctgttgcctttagtctcgaggcaacttagacaacggagtattgatctgagcacagcaggg
tgtgagctgtttgaagatactggggttgggggtgaagaaactgcagaggactaactgggctgagacccagtggtaatgttttaggg
cctaaggagtgcctctaaaaatctagatggacaattttgactttgagaaaagagaggtggaaatgaggaaaatgacttttctttat
tagattccagtagaaagaactttcatctttccctcatttttgttgttttaaaacatctatctggaggcaggacaagtatggtcgtt
aaaaagatgcaggcagaaggcatatattggctcagtcaaagtggggaactttggtggccaaacatacattgctaaggctattccta
tatcagctggacacatataaaatgctgctaatgcttcattacaaacttatatcctttaattccagatgggggcaaagtatgtccag
gggtgaggaacaattgaaacatttgggctggagtagattttgaaagtcagctctgtgtgtgtgtgtgtgtgtgcgcgcgcgcgtgt
gtgtgtgtgtgtcagcgtgtgtttcttttaacgtcttcagcctacaacatacagggttcatggtggcaagaagatagcaagattta
aattatggccagtgactagtgcttgaaggggaacaactacctgcatttaatgggaaggcaaaatctcaggctttgagggaagttaa
cataggcttgattctgggtggaagcttggtgtgtagttatctggaggccaggctggagctctcagctcactatgggttcatcttta
ttgtctcctttcatctcaacagctcctgggaaatgtgctggtgaccgttttggcaatccatttcggcaaagaattcacccctgagg
tgcaggcttcctggcagaagatggtgactgcagtggccagtgccctgtcctccagataccactgagctcactgcccatgattcaga
gctttcaaggataggctttattctgcaagcaatacaaataataaatctattctgctgagagatcacacatgattttcttcagctct
tttttttacatctttttaaatatatgagccacaaagggtttatattgagggaagtgtgtatgtgtatttctgcatgcctgtttgtg
tttgtggtgtgtgcatgctcctcatttatttttatatgagatgtgcattttgatgagcaaataaaagcagtaaagacacttgtaca
cgggagttctgcaagtgggagtaaatggtgtaggagaaatccggtgggaagaaagacctctataggacaggacttctcagaaacag
atgttttggaagagatgggaaaaggttcagtgaagacctgggggctggattgattgcagctgagtagcaaggatggttcttaagga
agggaaagtgttccaagctttaggaattcaaggtttagtcaggtgtagcaattctattttattaggaggaatactatttctaatgg
cacttagcttttcacagcccttgtggatgcctaagaaagtgaaattaatcccatgccctcaagtgtgcagattggtcacagcattt
caagggagagacctcattgtaagactctgggggaggtggggacttaggtgtaagaaatgaatcagcagaggctcacaagtcagcat
gagcatgttatgtctgagaaacagaccagcactgtgagatcaaaatgtagtgggaagaatttgtacaacattaattggaaggctta
cttaatggaatttttgtatagttggatgttagtgcatctctataagtaagagtttaatatgatggtgttacggacctaatgtttgt
gtctcctcaaaattcacatgctgaatccccaactcccaactgaccttatctgtgggggaggcttttgaaaagtaattaggtttaga
tgagctcataagagcagatccccatcataaaattattttccttatcagaagcagagagacaagccatttctctttcctcccggtga
ggacacagtgagaagtccgccatctgcaatccaggaagagaaccctgaccacgagtcagccttcagaaatgtgagaaaaaactctg
ttgttgaagccacccagtcttttgtattttgttatagcaccttgcactgagtaaggcagatgaagaaggagaaaaaaataagcttg
ggttttgagtggactacagaccatgtttatctcaggtttgcaaagctcccctcgtcccctatgtttcagtataaaatacctactct
actactctcatctataagacccaaataataagcctgcgcccttctctctaactttgatttctcctatttttacttcaacatgcttt
actctagccttgtaatgtctttacatacagtgaaatgtaaagttctttattctttttttctttctttcttttttctcctcagcctc
agaatttggcacatgcccttccttctttcaggaacttctccaacatctctgcctggctccatcatatcataaaggtcccacttcaa
atgcagtcactaccgtttcagaatatgcactttctttcttttttgttttttgttttttttaagtcaaagcaaatttcttgagagag
taaagaaataaacgaatgactactgcataggcagagcagccccgagggccgctggttgttccttttatggttatttcttgatgata
tgttaaacaagttttggattatttatgccttctctttttaggccatatagggtaactttctgacattgccatggcatttttctttt
aatttaatttactgttaccttaaattcaggggtacacgtacaggatatgcaggtttgttttataggtaaaagtgtgccatggtttt
aatgggttttttttttcttgtaaagttgtttaagtttcttgtttactctggatattaggcctttgtcagaagaatagattggaaaa
tctttttcccattctgtagattgtctttcgctctgatggtagtttcttttgctgagcaggagctctttagtttaattagattccat
tggtcaatttttgcttttgctgcaattgcttttcacgctttcatcatgaaatctgtgcccgtgtttatatcatgaatagtattgcc
ttgatttttttctaggctttttatagtttggggtttttcatttaagtctctaatccatctggagttaattttggataaggtataag
gaaggagtccagtttcatttttcagcatatggctagccagttctcccccatcatttattaaattgaaaatcctttccccattgctt
gcttttgtcaggtttctaaaagaccagatggttgtaggtacaatatgcagtttcttcaagtcatataataccatctgaaatctctt
attaattcatttcttttagtatgtatgctggtctcctctgctcactatagtgagggcaccattagccagagaatctgtctgtctag
ttcatgtaagattctcagaattaagaaaaatggatggcatatgaatgaaacttcatggatgacatatggaatctaatatgtatttg
ttgaattaatgcataagatgcaacagagagaagttgacaactgcaatgataacctggtattgatgatataagagtctatagatcac
agtagaagcaataatcatggaaaacaattggaaatggggaacagccacaaacaagaaagaatcaatacttccaggaaagtgactgc
aggtcacttttcctggagcgggtgagagaaaagtggaagttagcagtaactgctgaattcctggttggctgatggaaagatggggc
agctgttcactggtacgcagggttttagatgtatgtacctaaggatatgaggtatggcaatgaacagaaattcttttgggaatgag
ttttagggccattaaaggacatgacctgaagtttcctctgaggccagtccccacaactcaatataaatgtgtttcctgcatatagt
caaagttgccacttctttttcttcatatcatcgatctctgctcttaaagataatcttggttttgcctcaaactgtttgtcactaca
aactttccccatgttcctaagtaaaacaggtaactgcctctcaactatatcaagtagactaaaatattgtgtctctaatatcagaa
attcagctttaatatattgggtttaactctttgaaatttagagtctccttgaaatacacatgggggtgatttcctaaactttattt
cttgtaaggatttatctcaggggtaacacacaaaccagcatcctgaacctctaagtatgaggacagtaagccttaagaatataaaa
taaactgttcttctctctgccggtggaagtgtgccctgtctattcctgaaattgcttgtttgagacgcatgagacgtgcagcacat
gagacacgtgcagcagcctgtggaatattgtcagtgaagaatgtctttgcctgattagatataaagacaagttaaacacagcatta
gactatagatcaagcctgtgccagacacaaatgacctaatgcccagcacgggccacggaatctcctatcctcttgcttgaacagag
cagcacacttctcccccaacactattagatgttctggcataattttgtagatatgtaggatttgacatggactattgttcaatgat
tcagaggaaatctcctttgttcagataagtacactgactactaaatggattaaaaaacacagtaataaaacccagttttcccctta
cttccctagtttgtttcttattctgctttcttccaagttgatgctggatagaggtgtttatttctattctaaaaagtgatgaaatt
ggccgggcgcggtggctcacacctgtaatcccagcactttgggaggctgaggtgggcggatcacgaggtcaggagatcaagaccat
cctggctaacatggtgaaaccccatctctactaaaaatacaaaaaattagccagagacagtggcgggtgcctgtagtcccagctac
tcgggaggctgaggcaggagaatggcgtgaacctgggaggcagagcttgcggtgagcagagatcgcgccactgcacactccagcct
gggtgacaaagcgagactccatctcaaaaaaaaaaaaaaaaaaaagaaaaagaaagaaagaaagaaaaaaaaactgatgaaattgt
gtattcaatgtagtctcaagagaattgaaaaccaagaaaggctgtggcttcttccacataaagcctggatgaataacaggataaca
cgttgttacattgtcacaactcctgatccaggaattgatggctaagatattcgtaattcttatccttttcagttgtaacttattcc
tatttgtcagcattcaggttattagcggctgctggcgaagtccttgagaaataaactgcacactggatggtgggggtagtgtagga
aaatggaggggaaggaagtaaagtttcaaattaagcctgaacagcaaagttcccctgagaaggccacctggattctatcagaaact
cgaatgtccatcttgcaaaacttccttgcccaaaccccacccctggagtcacaacccacccttgaccaatagattcattttactga
gggaggcaaagggctggtcaatagattcatttcactgggagaggcaaagggctgggggccagagaggagaagtaaaaagccacaca
tgaagcagcaatgcaggcatgcttctggctcatctgtgatcaccaggaaactcccagatctgacactgtagtgcatttcactgctg
acaagaaggctgctgccaccagcctgtgaagcaaggttaaggtgagaaggctggaggtgagattctgggcaggtaggtactggaag
ccgggacaaggtgcagaaaggcagaaagtgtttctgaaagagggattagcccgttgtcttacatagtctgactttgcacctgctct
gtgattatgactatcccacagtctcctggttgtctacccatggacctagaggtactttgaaagttttggatatctgggctctgact
gtgcaataatgggcaaccccaaagtcaaggcacatggcaagaaggtgctgatctccttcggaaaagctgttatgctcacggatgac
ctcaaaggcacctttgctacactgagtgacctgcactgtaacaagctgcacgtggaccctgagaacttcctggtgagtagtaagta
cactcacgctttcttctttacccttagatatttgcactatgggtacttttgaaagcagaggtggctttctcttgtgttatgagtca
gctatgggatatgatatttcagcagtgggattttgagagttatgttgctgtaaataacataactaaaatttggtagagcaaggact
atgaataatggaaggccacttaccatttgatagctctgaaaaacacatcttataaaaaattctggccaaaatcaaactgagtgttt
ttggatgagggaacagaagttgagatagagaaaataacatctttcctttggtcagcgaaattttctataaaaattaatagtcactt
ttctgcatagtcctggaggttagaaaaagatcaactgaacaaagtagtgggaagctgttaaaaagaggattgtttccctccgaatg
atgatggtatacttttgtacgcatggtacaggattctttgttatgagtgtttgggaaaattgtatgtatgtatgtatgtatgtatg
tgatgactggggacttatcctatccattactgttccttgaagtactattatcctactttttaaaaggacgaagtctctaaaaaaaa
aatgaaacaatcacaatatgttggggtagtgagttggcatagcaagtaagagaaggataggacacaatgggaggtgcagggctgcc
agtcatattgaagctgatatctagcccataatggtgagagttgctcaaactctggtgaaaaaggatgtaagtgttatatctattta
ctgcaagtccagcttgaggccttctattcactatgtaccattttcttttttatcttcactccctccccagctcttaggcaacgtga
tattgattgttttggcaacccacttcagcgaggattttaccctacagatacaggcttcttggcagtaactaacaaatgctgtggtt
aatgctgtagcccacaagaccactgagttccctgtccactatgtttgtacctatggtccactatgtttgtacctatgtcccaaaat
ctcatctcctttagatgggggaggttggggagaagagcagtatcctgcctgctgattcagttcctgcatgataaaaatagaataaa
gaaatatgctctctaagaaatatcattgtactctttttctgtctttatattttaccctgattcagccaaaaggacgcactatttct
gatggaaatgagaatgttggagaatgggagtttaaggacagagaagatactttcttgcaatcctgcaagaaaagagagaactcgtg
ggtggatttagtggggtagttactcctaggaaggggaaatcgtctctagaataagacaatgtttttacagaaagggaggtcaatgg
aggtactctttggaggtgtaagaggattgttggtagtgtgtagaggtatgttaggactcaaattagaagttctgtataggctatta
tttgtatgaaactcaggatatagctcatttggtgactgcagttcacttctacttattttaaacaacatattttttattatttataa
tgaagtggggatggggcttcctagagaccaatcaagggccaaaccttgaactttctcttaacgtcttcaatggtattaatagagaa
ttatctctaaggcatgtgaactggctgtcttggttttcatctgtacttcatctgctacctctgtgacctgaaacatatttataatt
ccattaagctgtgcatatgatagatttatcatatgtattttccttaaaggatttttgtaagaactaattgaattgatacctgtaaa
gtctttatcacactacccaataaataataaatctctttgttcagctctctgtttctataaatatgtacaagttttattgtttttag
tggtagtgattttattctctttctatatatatacacacacatgtgtgcattcataaatatatacaatttttatgaataaaaaatta
ttagcaatcaatattgaaaaccactgatttttgtttatgtgagcaaacagcagattaaaaggctgagatttaggaaacagcacgtt
aagtcaagttgatagaggagaatatggacatttaaaagaggcaggatgatataaaattagggaaactggatgcagagaccagatga
agtaagaaaaatagctatcgttttgagcaaaaatcactgaagtttcttgcatatgagagtgacataataaatagggaaacgtagaa
aattgattcacatgtatatatatatatagaactgattagacaaagtctaacttgggtatagtcagaggagcttgctgtaattatat
tgaggtgatggataaagaactgaagttgatggaaacaatgaagttaagaaaaaaaatcgagtaagagaccattgtggcagtgattg
cacagaactggaaaacattgtgaaacagagagtcagagatgacagctaaaatccctgtctgtgaatgaaaagaaggaaatttattg
acagaacagcaaatgcctacaagccccctgtttggatctggcaatgaacgtagccattctgtggcaatcacttcaaactcctgtac
ccaagacccttaggaagtatgtagcaccctcaaacctaaaacctcaaagaaagaggttttagaagatataataccctttcttctcc
agtttcattaatcccaaaacctctttctcaaagtatttcctctatgtgtccaccccaaagagctcacctcaccatatctcttgagt
gggagcacatagataggcggtgctaccatctaacagcttctgaaattcctttgtcatatttttgagtccccactaataacccacaa
agcagaataaataccagttgctcatgtacaataatcactcaactgctgtcttgtagcatacattaattaagcacattctttgaata
attactgtgtccaaacaatcacactttaaaatctcacacttgtgctatcccttgcccttctgaatgtcactctgtattttaaatga
agagatgagggttgaatttcctgtgttacttattgttcatttctcgatgaggagttttcacattcacctttagtggaaaacacata
agtacacatcttacaggaaaaatataccaaactgacatgtagcatgaatgcttgtgcatgtagtcatataaaatcttgtagcaatg
taaacattctctgatatacacatacagatgtgtctatatgtctacacaatttcttatgctccatgaacaaacattccatgcacaca
taagaacacacactgttacagatgcatacttgagtgcattgacaaaattaccccagtcaatctagagaatttggatttctgcattt
gactctgttagctttgtacatgctgttcatttactctgggtgatgtctttccctcattttgccttgtctatcttgtactcatactt
taagtcctaacttatatgttatctcaactaagaagctatttttttttaattttaactgggcttaaagccctgtctataaactctgc
tacaattatgggctctttcttataatatttagtgtttttcctactaatgtacttaatctgctcattgtatattcctaccactaaat
tttaacctcttttatggtagagacattgtcttgtaaactcttatttccctagtatttggagatgaaaaaaaagattaaattatcca
aaattagatctctcttttctacattatgagtattacactatccatagagaagtttgtttgagacctaaactgaggaacctttggtt
ctaaaatgactatgtgatatcttagtatttataggtcatgaggttccttcctctgcctctgctatagtttgattagtcaacaagca
tgtgtcatgcatttattcacatcagaatttcatacactaataagacatagtatcagaagtcagtttattagttatatcagttaggg
tccatcaaggaaaggacaaaccattatcagttactcaacctagaattaaatacagctcttaatagttaattatccttgtattggaa
gagctaaaatatcaaataaaggacagtgcagaaatctagatgttagtaacatcagaaaacctcttccgccattaggcctagaaggg
cagaaggagaaaatgtttataccaccagagtccagaaccagagcccataaccagaggtccactggattcagtgagctagtgggtgc
tccttggagagagccagaactgtctaatgggggcatcaaagtatcagccataaaaaaccataaaaaagactgtctgctgtaggaga
tccgttcagagagagagagagaccagaaataatcttgcttatgctttccctcagccagtgtttaccattgcagaatgtacatgcga
ctgaaagggtgaggaaacctgggaaatgtcagttcctcaaatacagagaacactgagggaaggatgagaaataaatgtgaaagcag
acatgaatggtaattgacagaaggaaactaggatgtgtccagtaaatgaataattacagtgtgcagtgattattgcaatgattaat
gtattgataagataatatgaaaacacagaattcaaacagcagtgaactgagattagaattgtggagagcactggcatttaagaatg
tcacacttagaatgtgtctctaggcattgttctgtgcatatatcatctcaatattcattatctgaaaattatgaattaggtacaaa
gctcaaataatttattttttcaggttagcaagaactttttttttttttttctgagatagagcattgctatggttgcccaggctgga
gtgcaatggcatgatccaggctcactgcaacatctgcctcccaggttcaagcgattctcctgcctcagcctcccaagtagctggca
ctacaggcatgtgccaccaccatgcctggctaattttctatttttagtagatagggggtttcaccatgttggtcaggctgatctcg
aactcctaacatcaggtgatccaccctcctcggcctctgaaagtgctgggatcacaggcgtgagccaccacacccagccaagaatg
tgaattttgtagaaggatataacccatatttctctgaccctagagtccttagtatacctcccataccatgtggctcatcctcctta
catacatttcccatctttcaccctaccttttcctttttgtttcagcttttcactgtgtcaaaatctagaaccttatctcctacctg
ctctgaaaccaacagcaagttgacttccattctaacccacattggcattacactaattaaaatcgatactgagttctaaaatcatc
ggggattttggggactatgtcttacttcatacttccttgagatttcacattaaatgttggtgttcattaaaggtccttcatttaac
tttgtattcatcacactcttggattcacagttatatctaaactcttaaatacagcctgtataatcccaattcccaactctgatttc
taacctctgacctccaacctcagtgccaaacccatatatcaaacaatgtactgggcttatttatatagatgtcctataggcacctc
agactcagcatgggtatttcacttgttatactaaaactgtttctcttccagtgttttccattttagtcattagatagctacttgcc
cattcaccaaggtcacagattaaaatcatttccctacctctaatcaacagttcgattctgcttcaatttgtccctatctattaatc
accactcttactgcccagtcaggtcctcattgtttcctgaacaagagtagatgctattctttccacttttagaccttatcctggct
ggatgcggtggctcaggcttgtaaacccagcactttgggaggccaaggcaggcagatcacttgaggtcaggagttcaagaccagcc
tgaccaacatggtgaaaccccatctctactaaaaatacaaaatcagccgggcgtgtggtgcatgcctgcagtcccagctattcagg
tggctgaggcaggagaattgcttgaacccaggaggcagaggttgcggtgagcctagattgcaccattgcactctagcttgggcaat
agggatgaaactccatctcagaagagaaaagaaaaaaagaccttattctgttatacaaatcctctcaatgcaatccatatagaata
aacatgtaaccagatctcccaatgtgtaaaatcatttcaggtagaacagaattaaagtgaaaagccaagtctttggaattaacaga
caaagatcaaataacagtcctcatggccttaagaatttacctaacattttttttagaatcaattttcttatatatgaattggaaac
ataattcctccctcacaaacacattctaagattttaaggagatattgatgaagtacatcatctgtcatttttaacaggtagtggta
gtgattcacacagcacattatgatctgttcttgtatgttctgttccattctgtattcttgacctggttgtattctttctgagctcc
agatccacatatctaagtacatctttttgcattttacaagagtgcatacaatacaatgtatccaagactgtatttctgattttatc
gtaccactaaactcacaaatgtggccctattcttgtgttcacgactgacatcaccgtcatggtccaagtctgataatagaaatggc
attgtcactttcttccctactgcaacagaagcccagctatttgtctcccattttctctacttctaaaatacatttcttcactaagt
gagaataatcttttaaagacacaaatcaaaccatgccaccacctttcttgaattattcaatatctttcgttggcttccaggttaca
gaaaaataacttgtaacaaagtttaaaggtcattcatggctcctctctaccctattttataacatttccccttgtgatcagaatct
caggcacatcatccatctttctatatacaaataaagtcatatagtttgaactcacctctggttacttttaatcaaccaaatgctgt
aaaatgcatttgtatcgctacgtgttaagcagtagttgattcttttcatttctgtgtaatattctattctttgactataccgtaat
ttatcaattctactgttggtaagcatttaagtggctaccggtttgaggtttttatgattattgctgtcataagcatttctatacat
gtctttggatacacacatgcatgtgtttctgaatatctaaaaatgtaattgctaggtaatagacttatcaagcatccagcatttgt
ggatactattaaaggttttccaaaggggttatactattgtacagtgtcaccaacagagtttgagtttctattgatccatatcacca
ccaaaatttgaactgtcagtcttatctcttctcttgtctcttttttcctcttttttttccttcccttcccctctcttcgtttcttt
tctctcctcttctcttctttcctctcttcccttccctttctctttctcttccctatcccttctcctctcctctcccctcctttttt
ctcctctcctctccattatttatttttccttcttctcctccatcccttccatcctctctcttcccctcttccttccttcctttctc
catttcttcctcctctttccttcaatccttccttttggatatgctcatgggtgtgtatttgtctgccattgtggcattatttgaat
tcagaaaagagtgaaaaactactgggatcttcattcctgggtctaattccacatttttttttaagaacacatctgtaaaaatgttc
tgtactagcatattcccaggaacttcgttaaatttaatctggctgaatatggtaaatctacttttcactttgcattctttctttag
tcataccataattttaaacattcaaaatatttgtatataatatttgattttatctgtcattaaaatgttaaccttaaaattcatgt
ttccagaacctatttcaataactggtaaataaacactattcattttttaaatattcttttaatggatatttatttcaatataataa
aaaattagagttttattataggaagaatttaccaaaagaaggaggaagcaagcaagtttaaactgcagcaatagatttgtccattc
caacctctcaaaattcccttggagacaaaaatctctagaggcaaagaagaactttatattgagtcaacttgttaaaacatctgctt
ttagataagttttcttagtataaagtgacagaaacaaataagttaaactctaagatacattccactatattagcctaaaacacttc
tgcaaaaatgaaactaggaggatatttttagaaacaactgctgaaagagatgcggtggggagatatgtagaggagaacagggtttc
tgagtcaagacacacatgacagaacagccaatctcagggcaagttaagggaatagtggaatgaaggttcatttttcattctcacaa
actaatgaaaccctgcttatcttaaaccaacctgctcactggagcagggaggacaggaccagcataaaaggcagggcagagtcgac
tgttgcttacactttcttctgacataacagtgttcactagcaacctcaaacagacaccatggtgcatctgactcctgaggagaaga
ctgctgtcaatgccctgtggggcaaagtgaacgtggatgcagttggtggtgaggccctgggcaggttggtatcaaggttataagag
aggctcaaggaggcaaatggaaactgggcatgtgtagacagagaagactcttgggtttctgataggcactgactctctgtcccttg
ggctgttttcctaccctcagattactggtggtctacccttggacccagaggttctttgagtcctttggggatctgtcctctcctga
tgctgttatgggcaaccctaaggtgaaggctcatggcaagaaggtgctaggtgcctttagtgatggcctggctcacctggacaacc
tcaagggcactttttctcagctgagtgagctgcactgtgacaagctgcacgtggatcctgagaacttcagggtgagtccaggagat
gcttcacttttctctttttactttctaatcttacattttggttcttttacctacctgctcttctcccacatttttgtcattttact
atattttatcatttaatgcttctaaaattttgttaattttttatttaaatattctgcattttttccttcctcacaatcttgctatt
ttaaattatttaatatcctgtctttctctcccaaccccctcccttcatttttccttctctaacaacaactcaaattatgcatacca
gctctcacctgctaattctgcacttagaataatccttttgtctctccacatgggtatgggagaggctccaactcaaagatgagagg
catagaatactgttttagaggctataaatcattttacaataaggaataattggaattttataaattctgtagtaaatggaatggaa
aggaaagtgaatatttgattatgaaagactaggcagttacactggaggtggggcagaagtcgttgctaggagacagcccatcatca
cactgattaatcaattaatttgtatctattaatctgtttatagtaattaatttgtatatgctatatacacatacaaaattaaaact
aatttggaattaatttgtatatagtattatacagcatatatagcatatatgtacatatatagactacatgctagttaagtacatag
aggatgtgtgtgtatagatatatgttatatgtatgcattcatatatgtacttatttatgctgatgggaataacctggggatcagtt
ttgtctaagatttgggcagaaaaaaatgggtgttggctcagtttctcagaagccagtctttatttctctgttaaccatatgcatgt
atctgcctacctcttctccgcagctcttgggcaatgtgctggtgtgtgtgctggcccgcaactttggcaaggaattcaccccacaa
atgcaggctgcctatcagaaggtggtggctggtgtggctaatgccctggctcacaagtaccattgagatcctggactgtttcctga
taaccataagaagaccctatttccctagattctattttctgaacttgggaacacaatgcctacttcaagggtatggcttctgccta
ataaagaatgttcagctcaacttcctgattaatttcacttatttcatttttttgtccaggtgtgtaagaaggttcctgaggctcta
cagatagggagcacttgtttattttacaaagagtacatgggaaaagagaaaagcaagggaaccgtacaaggcattaatgggtgaca
cttctacctccaaagagcagaaattatcaagaactcttgatacaaagataatactggcactgcagaggttctagggaagacctcaa
ccctaagacatagcctcaagggtaatgctacgattaaactccaacaattactgagaaaataatgtgctcaattaaaggcataatga
ttactcaagacaatgttatgttgtctttcttcctccttcctttgcctgcacattgtagcccataatactataccccatcaagtgtt
cctgctccaagaaatagcttcctcctcttacttgccccagaacatctctgtaaagaatttcctcttatcttcccatatttcagtca
agattcattgctcacgtattacttgtgacctctcttgaccccagccacaataaacttctctatactacccaaaaaatctttccaaa
ccctcccccacaccattttttatatttttatatttttcttatttatttcatgcacacacacacactccgtgctttataagcaattc
tgcctattctctaccttcttacatgcctactgtgcctcatattaaattcatcaatgggcagaaagaaaatatttattcaagaaaac
agtgaatgaatgaacgaatgagtaaatgagtaaatgaaggaatgattattccttgctttagaacttctggaattagaggacaatat
taataataccatcgcacagtgtttctttgttgttaatgctacaacatacaaagaggaagcatgcagtaaacaaccgaacagttatt
tcctttctgatcataggagtaatatttttttccttgagcaccatttttgccataggtaaaattagaaggatttttagaactttctc
agttgtatacatttttaaaaatctgtattatatgcatgttgattaattttaaacttacttgaatacctaaacagaatctgttgttt
ccttgtgtttgaaagtgctttcacagtaactctgtctgtactgccagaatatactgacaatgtgttatagttaactgttttgatca
caacattttgaattgactggcagcagaagctcttttatatccatgtgttttccttaagtcattatacatagtaggcactgagaact
ctttatatctgaataagatatttaggaaccactggtttacatatcagaagcagagctactcagggcattttggggaagatcacttt
cacattcctgagcatagggaagttctcataagagtaagatattaaaaggagatacttgtgtggtattcgaaagacagtaagagaga
ttgtagaccttatgatcttgatagggaaaacaaactacattcctttctccaaaagtcaaaaaaaaagagcaaatatagcttactat
accttctattcctacaccattagaagtagtcagtgagtctaggcaagatgttggccctaaaaatccaaataccagagaattcatga
gaacatcacctggatgggacatgtgccgagcacacacaattactatatgctaggcattgctatcttcatattgaagatgaggaggt
caagagatgaaaaaagacttggcaccttgttgttatattaaaattatttgttagagtagagcttttgtaagagtctaggagtgtgg
gagctaaatgatgatacacatggacacaaaaaatagatcaacagacacccaggcctacttgagggttgagggtgggaagagggaga
cgatgaaaaagaacctattgggtattaagttcatcactgagtgatgaaataatctgtacatcaagacccagtgatatgcaatttac
ctatataacttgtacatgtacccccaaatttaaaatgaaagttaaaacaaagtataggaatggaattaattcctcaagatttggct
ttaattttatttgataatttatcaaatggttgtttttcttttctcactatggcgttgctttataaactatgttcagtatgtctgaa
tgaaagggtgtgtgtgtgtgtgaaagagagggagagaggaagggaagagaggacgtaataatgtgaatttgagttcatgaaaattt
ttcaataaaataatttaatgtcaggagaattaagcctaatagtctcctaaatcatccatctcttgagcttcagagcagtcctctga
attaatgcctacatgtttgtaaagggtgttcagactgaagccaagattctacctctaaagagatgcaatctcaaatttatctgaag
actgtacctctgctctccataaattgacaccatggcccacttaatgaggttaaaaaaaagctaattctgaatgaaaatctgagccc
agtggaggaaatattaatgaacaaggtgcagactgaaatataaatttttctgtaataattatgcatatactttagcaaagttctgt
ctatgttgactttattgctttttggtaagaaatacaactttttaaagtgaactaaactatcctatttccaaactattttgtgtgtg
tgcggtttgtttctatgggttctggttttcttggagcatttttatttcattttaattaattaattctgagagctgctgagttgtgt
ttactgagagattgtgtatctgcgagagaagtctgtagcaagtagctagactgtgcttgacctaggaacatatacagtagattgct
aaaatgtctcacttggggaattttagactaaacagtagagcatgtataaaaatactctagtcaagtgctgcttttgaaacaaatga
taaaaccacactcccatagatgagtgtcatgattttcatggaggaagttaatattcatcctctaagtatacccagactagggccat
tctgatataaaacattaggacttaagaaagattaatagactggagtaaaggaaatggacctctgtctctctcgctgtctctttttt
gaggacttgtgtgtgtgtgtgtgtgtgtgtgtgtgtgtgtgttgtggtcagtggggctggaataaaagtagaatagacctgcacct
gctgtggcatccattcacagagtagaagcaagctcacaatagtgaagatgtcagtaagcttgaatagtttttcaggaactttgaat
gctgatttagatttgaaactgaggctctgaccataaccaaatttgcactatttattgcttcttgaaacttatttgcctggtatgcc
tgggcttttgatggtcttagtatagcttgcagccttgtccctgcagggtattatgggtaatagaaagaaaagtctgcgttacactc
tagtcacactaagtaactaccattggaaaagcaacccctgccttgaagccaggatgatggtatctgcagcagttgccaacacaaga
gaaggatccatagttcatcatttaaaaaagaaaacaaaatagaaaaaggaaaactatttctgagcataagaagttgtagggtaagt
ctttaagaaggtgacaatttctgccaatcaggatttcaaagctcttgctttgacaattttggtctttcagaatactataaatataa
cctatattataatttcataaagtctgtgcattttctttgacccaggatatttgcaaaagacatattcaaacttccgcagaacactt
tatttcacatatacatgcctcttatatcagggatgtgaaacagggtcttgaaaactgtctaaatctaaaacaatgctaatgcaggt
ttaaatttaataaaataaaatccaaaatctaacagccaagtcaaatctgcatgttttaacatttaaaatattttaaagacgtcttt
tcccaggattcaacatgtgaaatcttttctcagggatacacgtgtgcctagatcctcattgctttagttttttacagaggaatgaa
tataaaaagaaaatacttaaattttatccctcttacctctataatcatacataggcataattttttaacctaggctccagatagcc
atagaagaaccaaacactttctgcgtgtgtgagaataatcagagtgagattttttcacaagtacctgatgagggttgagacaggta
gaaaaagtgagagatctctatttatttagcaataatagagaaagcatttaagagaataaagcaatggaaataagaaatttgtaaat
ttccttctgataactagaaatagaggatccagtttcttttggttaacctaaattttatttcattttattgttttattttattttat
tttattttattttgtgtaatcgtagtttcagagtgttagagctgaaaggaagaagtaggagaaacatgcaaagtaaaagtataaca
ctttccttactaaaccgacatgggtttccaggtaggggcaggattcaggatgactgacagggcccttagggaacactgagacccta
cgctgacctcataaatgcttgctacctttgctgttttaattacatcttttaatagcaggaagcagaactctgcacttcaaaagttt
ttcctcacctgaggagttaatttagtacaaggggaaaaagtacagggggatgggagaaaggcgatcacgttgggaagctatagaga
aagaagagtaaattttagtaaaggaggtttaaacaaacaaaatataaagagaaataggaacttgaatcaaggaaatgattttaaaa
cgcagtattcttagtggactagaggaaaaaaataatctgagccaagtagaagaccttttcccctcctacccctactttctaagtca
cagaggctttttgttcccccagacactcttgcagattagtccaggcagaaacagttagatgtccccagttaacctcctatttgaca
ccactgattaccccattgatagtcacactttgggttgtaagtgactttttatttatttgtatttttgactgcattaagaggtctct
agttttttatctcttgtttcccaaaacctaataagtaactaatgcacagagcacattgatttgtatttattctatttttagacata
atttattagcatgcatgagcaaattaagaaaaacaacaacaaatgaatgcatatatatgtatatgtatgtgtgtatatatacacac
atatatatatatattttttcttttcttaccagaaggttttaatccaaataaggagaagatatgcttagaaccgaggtagagttttc
atccattctgtcctgtaagtattttgcatattctggagacgcaggaagagatccatctacatatcccaaagctgaattatggtaga
caaaactcttccacttttagtgcatcaacttcttatttgtgtaataagaaaattgggaaaacgatcttcaatatgcttaccaagct
gtgattccaaatattacgtaaatacacttgcaaaggaggatgtttttagtagcaatttgtactgatggtatggggccaagagatat
atcttagagggagggctgagggtttgaagtccaactcctaagccagtgccagaagagccaaggacaggtacggctgtcatcactta
gacctcaccctgtggagccacaccctagggttggccaatctactcccaggagcagggagggcaggagccagggctgggcataaaag
tcagggcagagccatctattgcttacatttgcttctgacacaactgtgttcactagcaacctcaaacagacaccatggtgcatctg
actcctgaggagaagtctgccgttactgccctgtggggcaaggtgaacgtggatgaagttggtggtgaggccctgggcaggttggt
atcaaggttacaagacaggtttaaggagaccaatagaaactgggcatgtggagacagagaagactcttgggtttctgataggcact
gactctctctgcctattggtctattttcccacccttaggctgctggtggtctacccttggacccagaggttctttgagtcctttgg
ggatctgtccactcctgatgctgttatgggcaaccctaaggtgaaggctcatggcaagaaagtgctcggtgcctttagtgatggcc
tggctcacctggacaacctcaagggcacctttgccacactgagtgagctgcactgtgacaagctgcacgtggatcctgagaacttc
agggtgagtctatgggacgcttgatgttttctttccccttcttttctatggttaagttcatgtcataggaaggggataagtaacag
ggtacagtttagaatgggaaacagacgaatgattgcatcagtgtggaagtctcaggatcgttttagtttcttttatttgctgttca
taacaattgttttcttttgtttaattcttgctttctttttttttcttctccgcaatttttactattatacttaatgccttaacatt
gtgtataacaaaaggaaatatctctgagatacattaagtaacttaaaaaaaaactttacacagtctgcctagtacattactatttg
gaatatatgtgtgcttatttgcatattcataatctccctactttattttcttttatttttaattgatacataatcattatacatat
ttatgggttaaagtgtaatgttttaatatgtgtacacatattgaccaaatcagggtaattttgcatttgtaattttaaaaaatgct
ttcttcttttaatatacttttttgtttatcttatttctaatactttccctaatctctttctttcagggcaataatgatacaatgta
tcatgcctctttgcaccattctaaagaataacagtgataatttctgggttaaggcaatagcaatatctctgcatataaatatttct
gcatataaattgtaactgatgtaagaggtttcatattgctaatagcagctacaatccagctaccattctgcttttattttatggtt
gggataaggctggattattctgagtccaagctaggcccttttgctaatcatgttcatacctcttatcttcctcccacagctcctgg
gcaacgtgctggtctgtgtgctggcccatcactttggcaaagaattcaccccaccagtgcaggctgcctatcagaaagtggtggct
ggtgtggctaatgccctggcccacaagtatcactaagctcgctttcttgctgtccaatttctattaaaggttcctttgttccctaa
gtccaactactaaactgggggatattatgaagggccttgagcatctggattctgcctaataaaaaacatttattttcattgcaatg
atgtatttaaattatttctgaatattttactaaaaagggaatgtgggaggtcagtgcatttaaaacataaagaaatgaagagctag
ttcaaaccttgggaaaatacactatatcttaaactccatgaaagaaggtgaggctgcaaacagctaatgcacattggcaacagccc
ctgatgcatatgccttattcatccctcagaaaaggattcaagtagaggcttgatttggaggttaaagttttgctatgctgtatttt
acattacttattgttttagctgtcctcatgaatgtcttttcactacccatttgcttatcctgcatctctcagccttgactccactc
agttctcttgcttagagataccacctttcccctgaagtgttccttccatgttttacggcgagatggtttctcctcgcctggccact
cagccttagttgtctctgttgtcttatagaggtctacttgaagaaggaaaaacaggggtcatggtttgactgtcctgtgagccctt
cttccctgcctcccccactcacagtgacccggaatctgcagtgctagtctcccggaactatcactctttcacagtctgctttggaa
ggactgggcttagtatgaaaagttaggactgagaagaatttgaaaggcggctttttgtagcttgatattcactactgtcttattac
cctgtcataggcccaccccaaatggaagtcccattcttcctcaggatgtttaagattagcattcaggaagagatcagaggtctgct
ggctcccttatcatgtcccttatggtgcttctggctctgcagttattagcatagtgttaccatcaaccaccttaacttcatttttc
ttattcaatacctaggtaggtagatgctagattctggaaataaaatatgagtctcaagtggtccttgtcctctctcccagtcaaat
tctgaatctagttggcaagattctgaaatcaaggcatataatcagtaataagtgatgatagaagggtatatagaagaattttatta
tatgagagggtgaaaccctcaaaatgaaatgaaatcagacccttgtcttacaccataaacaaaaataaatttgaatgggttaaaga
attaaactaagacctaaaaccataaaaatttttaaagaaatcaaaagaagaaaattctaatattcacgttgcagccgttttttgaa
tttgatatgagaagcaaaggcaacaaaaggaaaaataaagaagtgaggctacatcaaactaaaaaatttccacacaaaaaacaaaa
caatgaacaaatgaaaggtgaaccatgaaatggcatatttgcaaaccaaatatttcttaaatattttggttaatatccaaaatata
taagaaacacagatgattcaataacaaacaaaaaattaaaaataggaaaataaaaaaattaaaaagaagaaaatcctgccatttat
ggcagaattgatgaacctggaggatgtaaaactaagaaaaataagcctgacacaaaaagacaaatactacacaaccttgctcatat
gtgaaacataaaaaagtcactctcatggaaacagacagtagaggtatggtttccaggggttgggggtgggagaatcaggaaactat
tactcaaagggtataaaatttcagttatgtgggatgaataaattctagatatctaatgtacagcatcgtgactgtagttaattgta
ctgtaagtatatttaaaatttgcaaagagagtagatttttttttttttttagatggagttttgctcttgttgtccaggctggagtg
caatggcaagatcttggctcactgcaacctccgcctcctgggttcaagcaaatctcctgcctcagcctcccgagtagctgggatta
caggcatgcgacaccatgcccagctaattttgtatttttagtagagacggggtttctccatgttggtcaggctgatccgcctgcct
cggccacccaaagggctgggattacaggcgtgagccaccgggcctggccgagagtagatcttaaaagcatttaccacaagaaaaag
gtaactatgtgagataatgggtatgttaattagcttgattgtggtaatcatttcacaaggtatacatatattaaaacatcatgttg
tacaccttaaatatatacaatttttatttgtgaatgatacctcaataaagttgaagaataataaaaaagaatagacatcacatgaa
ttaaaaaactaaaaaataaaaaaatgcatcttgatgattagaattgcattcttgatttttcagatacaaatatccatttgactgtt
tactcttttccaaaacaatacaataaattttagcactttatcttcattttccccttcccaatctataattatatatatatatattt
tagatattttgtatagttttactccctagattttctagtgttattattaaatagtgaagaaatgtttacacttatgtacaaaatgt
tttgcatgcttttcttcatttctaacattctctctaagtttattctatttttttctgattatccttaatattatctctttctgctg
gaaatacattgttacttttggtttatctaaaaatggcttcattttcttcattctaaaatcatgttaaattaataccactcatgtgt
aagtaagatagtggaataaatagaaatccaaaaactaaatctcactaaaatataataatgtgatatataaaaatatagcttttaaa
tttagcttggaaataaaaaacaaacagtaattgaacaactatactttttgaaaagagtaaagtgaaatgcttaactgcatatacca
caatcgattacacaattaggtgtgaaggtaaaattcagtcacgaaaaaactagaataaaaatatgggaagacatgtatataatctt
agagataacactgttatttaattatcaacccaaagtagaaactatcaagggagaaataaattcagtcaacaataaaagcatttaag
aagttattctaggctgggagcggtggctcacacctgcaattgcagcactttgggaggcctagacaggcggatcacgacgtcaggag
ttcaagatcagcctggccaacatagtgaaacctcatcgctactaaaaatataaaaacttagcctggcgtggtggcaggcatgtgta
atcccagcaatttgggaggctgaggcaggagaatcgcttgatcctgggaggcagaggttgcagtgagccaagattgtgccactgca
ttccagcccaggtgacagcatgagactccgtcacaaaaaaaaaagaaaaaaaaaaggggggggggagcggtggagccaagatgacc
gaataggaacagctccagtctatagctcccatcgtgagtgacgcagaagacgggtgatttctgcatttccaactgaggtaccaggt
tcatctcacagggaagtgccaggcagtgggtgcaggacagtaggtgcagtgcactgtgcatgagccaaagcagggcgaggcatcac
ctcacccgggaagcacaaggggtcagggaattccctttcctagtcaaagaaaagggtgacagatggcacctggaaaatcgggtcac
tcccgccctaatactgcgctcttccaacaagcttaacaaatggcacaccaggagattatatcccatgcctggctcagagggtccta
cgcccatggagcctcgctcattgctagcacagcagtctgaggtcaaactgcaaggtggcagtgaggctgggggaggggtgcccacc
attgtccaggcttgagcaggtaaacaaagccgcctggaagctcgaactgggtggagcccaccacagctcaaggaggcctgcctgcc
tctgtaggctccacctctaggggcagggcacagacaaacaaaagacaacaagaacctctgcagacttaaatgtccctgtctgacag
ctttgaagagagtagtggttctcccagcacatagcttcagatctgagaacaggcagactgcctcctcaagtgggtccctgaccccc
gagtagcctaactgggaggcatcccccagtaggggcagactgacacctcacatggctggtactcctctaagacaaaacttccagag
gaatgatcaggcagcagcatttgcggttcaccaatatccactgttctgcagccaccgctgttgatacccaggaaaacagcttctgg
agtggacctccagtaaactccaacagacctgcagctgagggtcctgactgttagaaggaaaactaacaaacagaaaggacatccac
accaaaaacccatctgtacatcgccatcatcaaagaccaaaggtagataaaaccataaagatggggaaaaagcagagcagaaaaac
tggacactctaaaaatgagagtgcctctcctcctccaaagtaacgcagctcctcaccagcaatggaacaaagctgggcagagaatg
actttgacgagttgagagaggaaggcttcagaagatcaaactactccaagctaaaggaggaagttcgaacaaacggcaaagaagta
aaaaactttgaaaaaaaattagatgaatggataactagaataaccaatgcacagaagtccttaaaggacctgatggagctgaaaac
caaggcaggagaactacgtgacaaatacacaagcctcagtaaccgatgagatcaactggaagaaagggtatcaatgacgaaagatg
aaatgaatgaaatgaagcatgaagagaagtttagagaaaaaagaataaaaagaaacgaacaaagcctccaagaaatatgggactat
gtgaaaagaccaaatctacatctaattggtgtagctgaaagtgatggggagaatggaaccaagttggaaaacactctgcaggatat
tatccaggagaacttccccaatctagcaaggcaagcccaaattcacattcaggaaatacagagaacgccacaaagatactcctaga
gaaaagcaactccaagacacataactgtcagattcaccaaagttgaaatgaaggaaaaaatgttaagggcagccagagagaaaggt
cgggttacccacaaagggaagcccatcagactaacagctgatctatcggcagaaactctacaagccagaagaaagtgggggccaat
attcaacattgttaaagaaaagaattttcaacccagaatttcatatccagccaaactaagcttcataagtgaaggagaaataaaat
cctttacagacaagcaaatgctgagagattttgtcaccaccaggcctgccctacaagagctcctgaaggaagcactaaacatggaa
aggaacaactagtatcagccactgcaaaaacatgccaaattgtaaagaccatcaaggctaggaagaaactgcatcaacgagcaaaa
taaccagctaacatcataatgacaggatcaaattcatacataacaatactcaccttaaatgtaaataggctaaatgctccaattaa
aagacacagactggcaaattggataaggagtcaagacccatctgtgttctgtattcaggaaacccatctcacgtgcagagacacac
ataggctcgaaataaaaggatggaggaatatctaccaagcaaatggaaaacaaaaaaaggcaggggttgcaatcctagtctctgat
aaaacagattttaaaccaacaaagatcaaaagagacaaagaaggccattacataatggcaaagggatctattcaagaagaagaact
aactatactaaatatatatgcacccaatacaggagcacccagattcataaaacaagtcctgagtgacctacaaagagacttagatg
cccacacaataataatgggagactttaacaccccactgtcaacattagacagatcaacgagacagaaagttaacaaggatatccag
gaattggactcagctctgcaccaagcagacctaatagacatctacagaactctccaccccaaatcaacagaatatacattcttttc
agcaccacaccacacctattccaaaactgaccacatagttggaagtaaagctctcctcagcaaatgtaaaagaacagaaactataa
caaactgtctctcagaccacagtgcaatcaaactagaactcaggattaagaaactcactcaaaaccactcagctacatggaaactg
aacagcctgctcctgaatgactactgggtacataacaaaatgaaggcagaaataaagatgttctttgaaaccaacgagaacaaaga
cacaacacaccagaatctctgagacacattcaaagcagtgtgtagagggaaatttatagcactaaatgcccacaagggaaagcagg
aaagatctaaaattgacaccctaacatcacaattaaaaaactagagaagcaggagcaaacacattcaaaagctaacagaagacaag
aaataactaagatcagagcagaagtgaaggacatagagacacaaaaaaacccttcaaaaaaatcaatgaatccagaagctgttttt
ttgaaaagatcaacaaaattgatagactgctagcaagactaataaagaagaaaagagagaagaatcaaatagacgcaataaaaaat
gacacggggtatcaccactgatcccacagaaatacaaactaccgtcagagaatactataaacacctctacgcaaataaactagaaa
atctagaagaaatggataaattcctcgacacatacactctgccaagactaaaccaggaagaagttgtatctctgaatagaccaata
acaggctctgaaattgaggcaataattaatagcttatcaaccaaaaaaagtccgggaccagtaggattcatagccgaattctacca
gaggtacaaggaggagctggtaccattccttctgaaactattccaatcaatagaaaaagagggaatcctccctaactcattttatg
aggccagcatcatcctgataccaaagcctgacagagacacaacaaaaaaagagaatgttacaccaatatccttgatgaacattgat
gcaaaaatcctcaataaaatactggcaaactgatccaccatgatcaagtgggcttcatccctgccatgcaaggctggttcaacata
cgaaaatcaataaacataatccagcatataaacagaaccaaagacacaaaccatatgattatctcaatagatgcagaaaaggcctt
tgacaaaattcaacaacgcttcatgctaaaaactctcaataaattaggtattgatgggacatatctcaaaataataagagctatct
atgacaaacccacagccaatatcatactgagtggacaaaaactggaagcattccctttgaaaactggcacaaggcagggatgccct
ctctcaccactcctattcaacatagtgttgtaagttctggccagggcaatcaggcaggagaaggaaataaagggcattcaattagg
aaaagaggaagtgaaattgtccctgtttgcagatgacatgattgtatatctagaaaaccccattgtctcagcccaaaatctcctta
agctgataagcaacttcagcaaagtctcaggatataaaatcagtgtgcaaaaatcacaagtattcctatgcaccaataacagacaa
acagagagccaaatcatgagtgaactcccattcacaattgcttcaaagagaataaaatacctaggaatccaacttacaagggatgt
gaaggacctcttcaaggagaactacaaaccactgctcaatgaaataaaagaggatacaaacaaatggaagaacattccatgctcat
gggtaggaagaatcaatatcgtgaaaatggtcatactgcccaaggtaatttatagattcaatgccatccccatcaagctaccaatg
actttcttcacagaactggaaaaaactactttaaagttcatatggaaccaaaaaagagcccacatcaccaaggcaatcctaagcca
aaagaacaaagctggaggcatcacgctacctgacttcaaactatactacaatgctacggtaaccaaaacagcatggtactggtacc
aaaacagagatctagaccaatggaacagaacagagccctcagaaataatgccgcatatctacaactatctgatctttgacaaacct
gagagaaacaagcaatggggaaaggattccctatttaataaatggtgctgggaaaactggctagccatatgtagaaagctgaaact
ggatcccttccttacaccttatacaaaaattaattcaagatggattaaagacttacatgttagacctaaaaccataaaaaccctag
aaaaaaacctaggcaataccattcaggacataggcatgggcaaggacttcatgtctaaaacaccaaaagcaatggcaacaaaagac
aaaatggacaaacgggatctaattaaactaaagagcttctgcacagctaaagaaactaccatcagagtgaacaggcaacctacaaa
atgggagaaaatttttgcaatctactcatctgacaaagggctaatatccagaatctacaatgaactcaaacaaatttacaagaaaa
aacaaacaaccccatcaaaaagtgggcaaaggatatgaacagacacttcgcaaaagaagacatttatgtaatcaaaaaacacatga
aaaaatgctcatcatcactagccatcagagaaatgcaaatcaaaaccacaatgagataccatctcacaccagttagaatggcgatc
attaaaaagtcaggaaacaacaggtgctggagaggatgtggagaaacaggaacaacttttacactgttggtgggactgtaaactag
ttcaaccattgcggaagtcagtgtggcaattcctcaggaatctagaactagaaataccatttgacccagccatcccattactgggt
acatacccaaaggattataaatcatgctgctataaagacacatgcacacgtatgtttattgcagcactattcacaatagcaaagac
ttggaaccaacccaaatgtccaacaacgatagactggattaagaaaatgtggcacatatacaccatggaatactatgcagccataa
aaaatgatgagttcatgtcctttgtagggacatggatgaagctggaaactatcattctcagcaaactatcacaaggagaataaacc
aaacaccgcatgttctcactcataggtgggaattgaacaatgagaacacatggacacatgaagaggaacatcacactctggggact
gttatggggtggggggcaggggcagggatagcactaggagatatacctaatgctaaatgacgagttaatgggtgcagcacaccaac
atggcacatgtatacatatataacaaacctgcatgttgtgcacatgtaccctaaaacttgaagtataataataaaaaaaagttatc
ctattaaaactgatctcacacatccgtagagccattatcaagtctttctctttgaaatagacagaaatttagtgttttctcagtca
gttaac

Five 5′ hypersensitive site (HS) sites (HS1-HS5) and one 3′ HS site have been identified in the human β-globin LCR (Stamatoyannopoulos et al., (2001)). The 5′ HSs 1-4 are Dnase I hypersensitive sites. The HS2 and HS3 elements are the most powerful single elements within the LCR (Ellis et al., EMBO J. (1996), 15:562-568; Collis et al., EMBO J. (1990) 9:233-240), as corroborated by many groups. Deleting HS2 in the context of βYAC in transgenic mice severely affects HS site formation as well as expression of all of the human β-globin genes at every developmental stage (Bungert et al., Mol. Cell Biol. (1999); 19:3062-3072). It was reported that deletion of HS2 minimally reduced the expression of the embryonic εy and βhi globin genes in yolk sac-derived erythrocytes (Ley et al., Ann. N.Y. Acad. Sci. (1998); 850:45-53; Hug et al., Mol. Cell Biol. (1996); 26:2906-2912). HS2 functions primarily as an enhancer.

In certain embodiments, the β-globin LCR region comprises a HS2 region. In non-limiting example, the β-globin LCR region comprises a HS2 region, a HS3 region, and a HS4 region. In certain embodiments, the HS2 region, HS3 region and HS4 region within the β-globin LCR region are contiguous. In one non-limiting embodiment, the β-globin LCR region consisting essentially of a HS2 region, a HS3 region and a HS4 region. In another embodiment, the β-globin LCR region comprises two introduced GATA-1 binding sites at the junction between the HS3 region and the HS4 region. The HS3 region can lie between the HS2 region and the HS4 region. The length and the sequence of the HS2 region can vary. The HS2 region can have a length of from about 400 bp to about 1000 bp, e.g., from about 400 bp to about 500 bp, from about 500 bp to about 600 bp, from about 600 bp to about 700 bp, from about 700 bp to about 800 bp, from about 800 bp to about 900 bp, or from about 900 bp to about 1000 bp. In one non-limiting embodiment, the HS2 region has a length of 860 bp. In one non-limiting example, the HS2 region has the nucleotide sequence set forth in SEQ ID NO:9, which is provided below:

[SEQ ID NO: 9]
GTATATGTGTATATATATATATATATATTCAGGAAATAATATATTCTAGA
ATATGTCACATTCTGTCTCAGGCATCCATTTTCTTTATGATGCCGTTTGA
GGTGGAGTTTTAGTCAGGTGGTCAGCTTCTCCttttttttGCCATCTGCC
CTGTAAGCATCCTGCTGGGGACCCAGATAGGAGTCATCACTCTAGGCTGA
GAACATCTGGGCACACACCCTAAGCCTCAGCATGACTCATCATGACTCAG
CATTGCTGTGCTTGAGCCAGAAGGTTTGCTTAGAAGGTTACACAGAACCA
GAAGGCGGGGGTGGGGCACTGACCCCGACAGGGGCCTGGCCAGAACTGCT
CATGCTTGGACTATGGGAGGTCACTAATGGAGACACACAGAAATGTAACA
GGAACTAAGGAAAAACTGAAGCTTATTTAATCAGAGATGAGATGCTGGAA
GGGATAGAGGGAGCTGAGCTTGTAAAAAGTATAGTAATCATTCAGCAAAT
GGTTTTGAAGCACCTGCTGGATGCTAAACACTATTTTCAGTGCTTGAATC
ATAAATAAGAATAAAACATGTATCTTATTCCCCACAAGAGTCCAAGTAAA
AAATAACAGTTAATTATAATGTGCTCTGTCCCCCAGGCTGGAGTGCAGTG
GCACGATCTCAGCTCACTGCAACCTCCGCCTCCCGGGTTCAAGCAATTCT
CCTGCCTCAGCCACCCTAATAGCTGGGATTACAGGTGCACACCACCATGC
CAGGCTAATTTTTGTACTTTTTGTAGAGGCAGGGTATCACCATGTTGTCC
AAGATGGTCTTGAACTCCTGAGCTCCAAGCAGTCCACCCACCTCAGCCTC
CCAAAGTGCT

In certain embodiments, the HS2 region has a length of about 840 bp. In certain embodiments, the HS2 region has a length of about 650 bp (e.g., 646 bp). In certain embodiments, the HS2 region has a length of about 420 bp (e.g., 423 bp).

The length and the sequence of the HS3 region can vary. The HS3 region can have a length of from about 200 bp to about 1400 bp, e.g., from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, from about 1100 bp to about 1200 bp, from about 1200 bp to about 1300 bp, or from about 1300 bp to about 1400 bp. In certain embodiments, the HS3 region has a length of about 1300 bp. In one non-limiting embodiment, the HS3 region has a length of 1308 bp. In one non-limiting embodiment, the HS3 region has a length of 1301 bp. In one non-limiting example, the HS3 region has the nucleotide sequence set forth in SEQ ID NO:5, which is provided below:

[SEQ ID NO: 5]
AAGCTTTCATTAAAAAAAGTCTAACCAGCTGCATTCGACTTTGACTGCAG
CAGCTGGTTAGAAGGTTCTACTGGAGGAGGGTCCCAGCCCATTGCTAAAT
TAACATCAGGCTCTGAGACTGGCAGTATATCTCTAACAGTGGTTGATGCT
ATCTTCTGGAACTTGCCTGCTACATTGAGACCACTGACCCATACATAGGA
AGCCCATAGCTCTGTCCTGAACTGTTAGGCCACTGGTCCAGAGAGTGTGC
ATCTCCTTTGATCCTCATAATAACCCTATGAGATAGACACAATTATTACT
CTTACTTTATAGATGATGATCCTGAAAACATAGGAGTCAAGGCACTTGCC
CCTAGCTGGGGGTATAGGGGAGCAGTCCCATGTAGTAGTAGAATGAAAAA
TGCTGCTATGCTGTGCCTCCCCCACCTTTCCCATGTCTGCCCTCTACTCA
TGGTCTATCTCTCCTGGCTCCTGGGAGTCATGGACTCCACCCAGCACCAC
CAACCTGACCTAACCACCTATCTGAGCCTGCCAGCCTATAACCCATCTGG
GCCCTGATAGCTGGTGGCCAGCCCTGACCCCACCCCACCCTCCCTGGAAC
CTCTGATAGACACATCTGGCACACCAGCTCGCAAAGTCACCGTGAGGGTC
TTGTGTTTGCTGAGTCAAAATTCCTTGAAATCCAAGTCCTTAGAGACTCC
TGCTCCCAAATTTACAGTCATAGACTTCTTCATGGCTGTCTCCTTTATCC
ACAGAATGATTCCTTTGCTTCATTGCCCCATCCATCTGATCCTCCTCATC
AGTGCAGCACAGGGCCCATGAGCAGTAGCTGCAGAGTCTCACATAGGTCT
GGCACTGCCTCTGACATGTCCGACCTTAGGCAAATGCTTGACTCTTCTGA
GCTCAGTCTTGTCATGGCAAAATAAAGATAATAATAGTGTTTTTTTATGG
AGTTAGCGTGAGGATGGAAAACAATAGCAAAATTGATTAGACTATAAAAG
GTCTCAACAAATAGTAGTAGATTTTATCATCCATTAATCCTTCCCTCTCC
TCTCTTACTCATCCCATCACGTATGCCTCTTAATTTTCCCTTACCTATAA
TAAGAGTTATTCCTCTTATTATATTCTTCTTATAGTGATTCTGGATATTA
AAGTGGGAATGAGGGGCAGGCCACTAACGAAGAAGATGTTTCTCAAAGAA
GCCATTCTCCCCACATAGATCATCTCAGCAGGGTTCAGGAAGATAAAGGA
GGATCAAGGTCGAAGGTAGGAACTAAGGAAGAACACTGGGCAAGTGGATC
C

In certain embodiments, the HS3 region has a length of about 850 bp (e.g., 845 bp). In certain embodiments, the HS3 region has a length of from about 280 bp to about 290 bp (e.g., 280 bp and 287 bp).

Similarly, the length and the sequence of the HS4 region can vary. The HS4 region can have a length of from about 200 bp to about 1200 bp, e.g., from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, or from about 1100 bp to about 1200 bp.

In certain embodiments, the HS4 region has a length of about 1.0 kb or more. In certain embodiments, the HS4 region has a length of about 1.1 kb. In certain embodiments, the HS4 region has a length of about 1150 bp (e.g., 1153 bp). In one non-limiting embodiment, the HS4 region has a length of 1065 bp. In one non-limiting example, the HS4 region has the nucleotide sequence set forth in SEQ ID NO:6, which is provided below:

[SEQ ID NO: 6]
TGAGCCCCTTTTCCTCTAACTGAAAGAAGGAAAAAAAAAATGGAACCCAA
AATATTCTACATAGTTTCCATGTCACAGCCAGGGCTGGGCAGTCTCCTGT
TATTTCTTTTAAAATAAATATATCATTTAAATGCATAAATAAGCAAACCC
TGCTCGGGAATGGGAGGGAGAGTCTCTGGAGTCCACCCCTTCTCGGCCCT
GGCTCTGCAGATAGTGCTATCAAAGCCCTGACAGAGCCCTGCCCATTGCT
GGGCCTTGGAGTGAGTCAGCCTAGTAGAGAGGCAGGGCAAGCCATCTCAT
AGCTGCTGAGTGGGAGAGAGAAAAGGGCTCATTGTCTATAAACTCAGGTC
ATGGCTATTCTTATTCTCACACTAAGAAAAAGAATGAGATGTCTACATAT
ACCCTGCGTCCCCTCTTGTGTACTGGGGCCCCCAAGAGCTCTCTAAAAGT
GATGGCAAAGTCATTGCGCTAGATGCCATCCCATCTATTATAAACCTGCA
TTTGTCTCCACACACCAGTCATGGACAATAACCCTCCTCCCAGGTCCACG
TGCTTGTCTTTGTATAATACTCAAGTAATTTCGGAAAATGTATTCTTTCA
ATCTTGTTCTGTTATTCCTGTTTCAATGGCTTAGTAGAAAAAGTACATAC
TTGTTTTCCCATAAATTGACAATAGACAATTTCACATCAATGTCTATATG
GGTCGTTGTGTTTGCTGTGTTTGCAAAAACTCACAATAACTTTATATTGT
TACTACTCTAAGAAAGTTACAACATGGTGAATACAAGAGAAAGCTATTAC
AAGTCCAGAAAATAAAAGTTATCATCTTGAGGCCTCAGCTTTCTAGGAAT
AATATCAATATTACAAAATTTAATCTAACAATTATGAACAGCAATGAGAT
AATATGTACAAAGTACCCAGACCTATGTGGTAGAGCATCAAGGAAGCGCA
TTGCGGAGCAGTTTTTTGTTTGTTTGTTTTTGTATTCTGTTTCGTGAGGC
AAGGTTTCACTCTGCTGTCCAGGCTGGAGTGCAGTGGCAAGATCATGTCT
CACTGCAGCCTTGAC

In one non-limiting example, the HS4 region has the nucleotide sequence set forth in SEQ ID NO:7, which is provided below:

[SEQ ID NO: 7]
TGAGCCCCTTTTCCTCTAACTGAAAGAAGGAAAAAAAAAATGGAACCCAA
AATATTCTACATAGTTTCCATGTCACAGCCAGGGCTGGGCAGTCTCCTGT
TATTTCTTTTAAAATAAATATATCATTAAATGCATAAATAAGCAAACCCT
GCTCGGGAATGGGAGGGAGAGTCTCTGGAGTCCACCCCTTCTCGGCCCTG
GCTCTGCAGATAGTGCTATCAAAGCCCTGACAGAGCCCTGCCCATTGCTG
GGCCTTGGAGTGAGTCAGCCTAGTAGAGAGGCAGGGCAAGCCATCTCATA
GCTGCTGAGTGGGAGAGAGAAAAGGGCTCATTGTCTATAAACTCAGGTCA
TGGCTATTCTTATTCTCACACTAAGAAAAAGAATGAGATGTCTACATATA
CCCTGCGTCCCCTCTTGTGTACTGGGGCCCCCAAGAGCTCTCTAAAAGTG
ATGGCAAAGTCATTGCGCTAGATGCCATCCCATCTATTATAAACCTGCAT
TTGTCTCCACACACCAGTCATGGACAATAACCCTCCTCCCAGGTCCACGT
GCTTGTCTTTGTATAATACTCAAGTAATTTCGGAAAATGTATTCTTTCAA
TCTTGTTCTGTTATTCCTGTTTCAATGGCTTAGTAGAAAAAGTACATACT
TGTTTTCCCATAAATTGACAATAGACAATTTCACATCAATGTCTATATGG
GTCGTTGTGTTTGCTGTGTTTGCAAAAACTCACAATAACTTTATATTGTT
ACTACTCTAAGAAAGTTACAACATGGTGAATACAAGAGAAAGCTATTACA
AGTCCAGAAAATAAAAGTTATCATCTTGAGGCCTCAGCTTTCTAGGaATA
ATATCAATATTACAAAATTAATCTAACAATTATGAACAGCAATGAGATAA
TATGTACAAAGTACCCAGACCTATGTGGTAGAGCATCAAGGAAGCGCATT
GCGGAGCAGTTTTTTGTTTGTTTGTTTTTGTATTCTGTTTCGTGAGGCAA
GGTTTCACTCTGCTGTCCAGGCTGGAGTGCAGTGGCAAGATCATGTCTCA
CTGCAGCCTTGACAC

In certain embodiments, the HS4 region has a length of less than about 1.0 kb, e.g., less than about 900 bp, less than about 700 bp, less than about 600 bp, or less than about 500 bp. In certain embodiments, the HS4 region has a length of less than about 500 bp. In certain embodiments, the HS4 region has a length of about 450 bp. In one non-limiting embodiment, the HS4 region has a length of about 446 bp. In one non-limiting example, the HS4 region has the nucleotide sequence set forth in SEQ ID NO:8, which is provided below:

[SEQ ID NO: 8]
TGGAACCCAAAATATTCTACATAGTTTCCATGTCACAGCCAGGGCTGGGC
AGTCTCCTGTTATTTCTTTTAAAATAAATATATCATTTAAATGCATAAAT
AAGCAAACCCTGCTCGGGAATGGGAGGGAGAGTCTCTGGAGTCCACCCCT
TCTCGGCCCTGGCTCTGCAGATAGTGCTATCAAAGCCCTGACAGAGCCCT
GCCCATTGCTGGGCCTTGGAGTGAGTCAGCCTAGTAGAGAGGCAGGGCAA
GCCATCTCATAGCTGCTGAGTGGGAGAGAGAAAAGGGCTCATTGTCTATA
AACTCAGGTCATGGCTATTCTTATTCTCACACTAAGAAAAAGAATGAGAT
GTCTACATATACCCTGCGTCCCCTCTTGTGTACTGGGGTCCCCAAGAGCT
CTCTAAAAGTGATGGCAAAGTCATTGCGCTAGATGCCATCCCATCT

In certain embodiments, the HS4 region has a length of about 280 bp (e.g., 283 bp). In certain embodiments, the HS4 region has a length of about 240 bp (e.g., 243 bp).

In certain non-limiting embodiments, the β-globin LCR region comprises a HS2 region having the nucleotide sequence set forth in SEQ ID NO:9, SEQ ID NO:20 or SEQ ID NO:21, a HS3 region having the nucleotide sequence set forth in SEQ ID NO:5, and a HS4 region having the nucleotide sequence set forth in SEQ ID NO:6, SEQ ID NO:7 or SEQ ID NO:8.

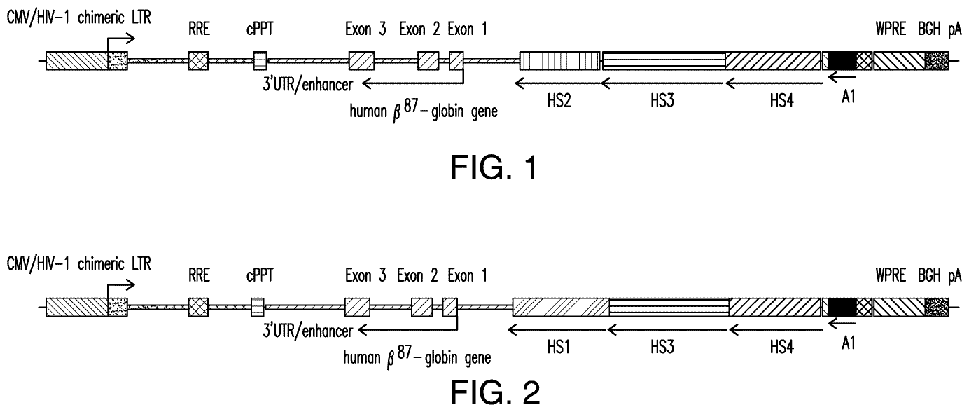

In one non-limiting embodiment, the β-globin LCR region comprises a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7, as shown in FIG. 1.

In another non-limiting embodiment, the β-globin LCR region further comprises a HS1 region, i.e., a β-globin LCR region comprising a HS1 region, a HS2 region, a HS3 region, and a HS4 region. In certain embodiments, the HS1 region, HS2 region, HS3 region and HS4 region within the β-globin LCR region are contiguous. In one non-limiting embodiment, the β-globin LCR region consisting essentially of a HS1 region, a HS2 region, a HS3 region and a HS4 region. In another embodiment, the β-globin LCR region comprises two introduced GATA-1 binding sites at the junction between the HS3 region and the HS4 region.

The length and the sequence of the HS1 region can vary. In certain embodiments, the HS1 region is from about 300 bp to about 1500 bp in length, e.g., from about 300 bp to about 1100 bp in length. In certain embodiments, the HS1 region has a length of about 1.0 kb or more, e.g., about 1.1 kb, about 1.2 kb, about 1.3 kb, about 1.4 kb, or about 1.5 kb. In certain embodiments, the HS1 region has a length of about 1.1 kb. In one non-limiting example, the HS1 region has a length of 1074 bp. In one non-limiting example, the HS1 region has the nucleotide sequence set forth in SEQ ID NO:2, which is provided below:

[SEQ ID NO: 2]
AAGTAAACTTCCACAACCGCAAGCTTATTGAGGCTAAGGCATCTGTGAAG
GAAAGAAACATCTCCTCTAAACCACTATGCTGCTAGAGCCTCTTTTCTGT
ACTCAAGCCTCATTCAGACACTAGTGTCACCAGTCTCCTCATATACCTAT
TGTATTTTCTTCTTCTTGCTGGTTTAGTCATGTTTTCTGGGAGCTTAGGG
GCTTATTTTATTTTGTTTTGTTTTCTAATCAACAGAGATGGGCAAACCCA
TTATTTTTTTCTTTAGACTTGGGATGGTGATAGCTGGGCAGCGTCAGAAA
CTGTGTGTGGATATAGATAAGAGCTCGGACTATGCTGAGCTGTGATGAGG
GAGGGACCTAGCCAAAGGCAGTGAGAGTCAGAATGCTCCTGCTATTGCCT
TCTCAGTCCCCACGCTTGGTTTCTACACAAGTAGATACATAGAAAAGGCT
ATAGGTTAGTGTTTGAGAGTCCTGCATGAGTTAGTTGCTCAGAAATGCCC
GATAAATATGTTATGTGTGTTTATGTATATATATGTTTTATATATATATA
TGTGTGTGTGTGTGTGTGTGTGTGTTGTGTTTACAAATATGTGATTATCA
TCAAAACGTGAGGGCTAAAGTGACCAGATAACTTGCAGGTCCTAGGATAC
CAGGAAAATAAATTACATTCCAAAAATTTAACTGAGACTTTAAAAAAAAA
AAAAAAAAAAAAAAAAAAACCAGTGATCCATGGACACAGGGAGGGGAACA
TCACACACTGGGGCCTGTTGGGGGTGGGGGGCTAGGGGAAGGATAGCATT
AGGAGAAATACCTAATGTAGATGACGGGTTGATGGGTGCAGCAAACCACC
ATGGCACATGTACCCCAGAACTTAAAGCATATTAAAAAAACAGTGATCAT
AAAAGAAGCTCAAATTTAACTATAAGAGACGGAATGGCTCCCACAATTCT
TAACTATAATCTTACAGAATATTCTCATTGAATAGAAGTATGCTTATCAT
TAGAGATTTGGACAGCCAGGAAAGCACAGAAAAAAAAAAAAGGAGCTCTG
TTGCCTTATAGCCTAGAGGTGTTT

In certain embodiments, the HS1 region has a length of less than about 1.0 kb, e.g., from about 400 bp to about 700 bp, from about 400 bp to about 500 bp, from about 500 bp to about 600 bp, from about 600 bp to about 700 bp, from about 700 bp to about 800 bp, from about 800 bp to about 900 bp, or from about 900 bp to about 1.0 kb. In certain embodiments, the HS1 region has a length of less than about 700 bp. In certain embodiments, the HS1 region has a length of about 600 bp. In one non-limiting embodiment, the HS1 region has a length of 602 bp. In one non-limiting example, the HS1 region has the nucleotide sequence set forth in SEQ ID NO:3, which is provided below:

[SEQ ID NO: 3]
GGCATCTGTGAAGGAAAGAAACATCTCCTCTAAACCACTATGCTGCTAGA
GCCTCTTTTCTGTACTCAAGCCTCATTCAGACACTAGTGTCACCAGTCTC
CTCATATACCTATTGTATTTTCTTCTTCTTGCTGGTTTAGTCATGTTTTC
TGGGAGCTTAGGGGCTTATTTTATTTTGTTTTGTTTTCTAATCAACAGAG
ATGGGCAAACCCATTATTTTTTTCTTTAGACTTGGGATGGTGATAGCTGG
GCAGCGTCAGAAACTGTGTGTGGATATAGATAAGAGCTCGGACTATGCTG
AGCTGTGATGAGGGAGGGACCTAGCCAAAGGCAGTGAGAGTCAGAATGCT
CCTGCTATTGCCTTCTCAGTCCCCACGCTTGGTTTCTACACAAGTAGATA
CATAGAAAAGGCTATAGGTTAGTGTTTGAGAGTCCTGCATGAGTTAGTTG
CTCAGAAATGCCCGATAAATATGTTATGTGTGTTTATGTATATATATGTT
TTATATATATATATGTGTGTGTGTGTGTGTGTGTGTGTTGTGTTTACAAA
TATGTGATTATCATCAAAACGTGAGGGCTAAAGTGACCAGATAACTTGCA
GG

In certain embodiments, the HS1 region has a length of less than about 500 bp. In certain embodiments, the HS1 region has a length of about 490 bp. In one non-limiting embodiment, the HS1 region has a length of 489 bp. In one non-limiting example, the HS1 region has the nucleotide sequence set forth in SEQ ID NO:4, which is provided below:

[SEQ ID NO: 4]
GGCATCTGTGAAGGAAAGAAACATCTCCTCTAAACCACTATGCTGCTAGA
GCCTCTTTTCTGTACTCAAGCCTCATTCAGACACTAGTGTCACCAGTCTC
CTCATATACCTATTGTATTTTCTTCTTCTTGCTGGTTTAGTCATGTTTTC
TGGGAGCTTAGGGGCTTATTTTATTTTGTTTTGTTTTCTAATCAACAGAG
ATGGGCAAACCCATTATTTTTTTCTTTAGACTTGGGATGGTGATAGCTGG
GCAGCGTCAGAAACTGTGTGTGGATATAGATAAGAGCTCGGACTATGCTG
AGCTGTGATGAGGGAGGGACCTAGCCAAAGGCAGTGAGAGTCAGAATGCT
CCTGCTATTGCCTTCTCAGTCCCCACGCTTGGTTTCTACACAAGTAGATA
CATAGAAAAGGCTATAGGTTAGTGTTTGAGAGTCCTGCATGAGTTAGTTG
CTCAGAAATGCCCGATAAATATGTTATGTGTGTTTATGT

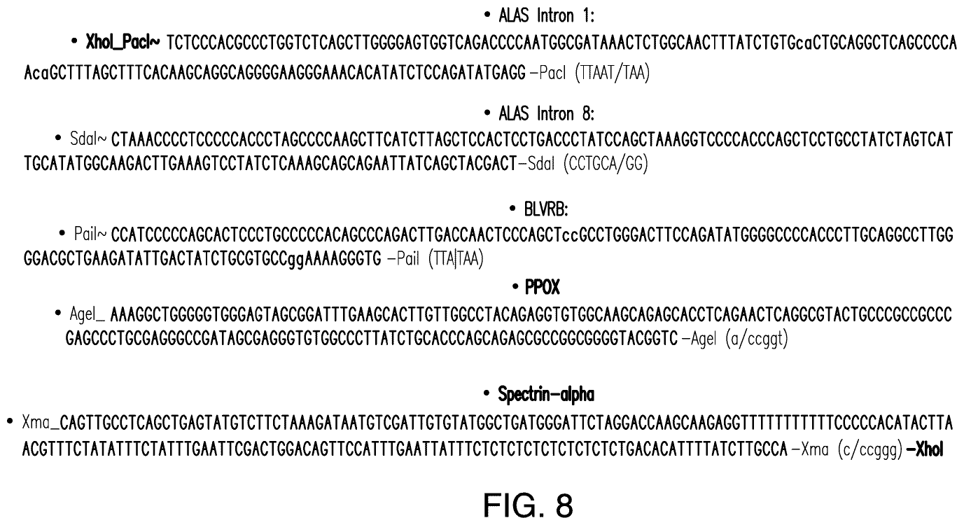

Recent studies have shown that HS2 is not erythroid-specific, but is expressed in other cell lines and lineages (See Example 3 and FIG. 7) and is also present in undifferentiated human embryonic stem cells (Chang et al., Stem cell reviews (2013); 9:397-407). Due to the non-erythroid activity of HS2, HS2-containing globin vectors may pose a risk for their safe use in clinical treatment, e.g., for treating thalassemia and sickle cell patients. In certain embodiments, the β-globin LCR region does not comprise a HS2 region. In certain embodiments, the β-globin LCR region does not comprise a core sequence of HS2. A core sequence of HS2 provides position independent, high level expression. In addition, a core sequence of HS2 sustains the enhancer activity of HS2. For example, the core sequence of HS2 enhances the transcription of a globin gene (e.g., human β-globin gene). Additionally, a core sequence of HS2 comprises one or more binding sites or binding motifs for ubiquitous as well as tissue-specific (e.g., erythroid-specific) proteins (e.g., transcription factors), including, but not limited to, members of AP1 family of proteins (e.g., NF-E2), GATA-1 (also known as “NF-E1” or “NFE1”), Krüppel-like Zn finger proteins (e.g., ubiquitous proteins Sp1 and YY1, and erythroid-restricted factor erythroid Krüppel-like factor (EKLF)), and basic helix-loop-helix (bHLH) proteins (E boxes) (e.g., USF and TAL1). AP1 binding sites are required for enhancement and induction (Moi and Kan (1990); Ney et al., (1990); Talbot and Grosveld (1991)). Furthermore, binding of NF-E2 can cause disruption of in vitro reconstituted chromatin at HS2 (Armstrong and Emerson (1996)). Mutations in the GATA-1 binding sites can cause a reduction in enhancer activity of HS2 in transgenic mice (Caterina et al., (1994)). Although both AP1 (e.g., AP1/NF-E2) and GATA1 binding sites are important for core function, mice lacking these factors do not show impaired globin gene expression (Weiss et al., 1994).

In certain embodiments, the β-globin LCR region does not comprise the full length of a core sequence of HS2. In certain embodiments, the core sequence of a HS2 region is a core sequence of human HS2. In one non-limiting embodiment, the core sequence of human HS2 comprises a tandem pair of binding sites for members of AP1 family of proteins (e.g., NF-E2) (referred to as “AP1/NF-E2” binding sites) (e.g., GCTGAGTCA, and GATGAGTCA), one binding site for Krüppel-like Zn finger proteins (e.g., AGGGTGTGT), one GATA-1 binding site (e.g., CTATCT), and three E boxes (CANNTG, e.g., CAGATG, and CACCTG). In one non-limiting embodiment, the β-globin LCR region does not comprise the full length of a 388 bp core sequence of human HS2, which has the nucleotide sequence set forth in SE ID NO:20 provided below:

[SEQ ID NO: 20]
TAAGCTTCAGTTTTTCCTTAGTTCCTGTTACATTTCTGTGTGTCTCCATT
AGTGACCTCCCATAGTCCAAGCATGAGCAGTTCTGGCCAGGCCCCTGTCG
GGGTCAGTGCCCCACCCCCGCCTTCTGGTTCTGTGTAACCTTCTAAGCAA
ACCTTCTGGCTCAAGCACAGCAATGCTGAGTCATGATGAGTCATGCTGAG
GCTTAGGGTGTGTGCCCAGATGTTCTCAGCCTAGAGTGATGACTCCTATC
TGGGTCCCCAGCAGGATGCTTACAGGGCAGATGGCAAAAAAAAGGAGAAG
CTGACCACCTGACTAAAACTCCACCTCAAACGGCATCATAAAGAAAATGG
ATGCCTGAGACAGAATGTGACATATTCTAGAATATATT

The nucleotide sequence set forth in SEQ ID NO:20 corresponds to nucleotides position 16671 to position 17058 of SEQ ID NO:19 (GenBank Access No.: NG_000007.3). In SEQ ID NO:20, one AP1/NF-E2 binding site having the nucleotide sequence of GCTGAGTCA is located at position 175 to position 183, one AP1/NF-E2 binding site having the nucleotide sequence of GATGAGTCA is located at position 185 to position 193, one binding site for Krüppel-like Zn finger proteins having the nucleotide sequence of AGGGTGTGT is located as position 205 to position 213, two E boxes, each of which have the nucleotide sequence of CAGATG, is located at position 217 to position 222, and position 278 to position 283, one GATA-1 binding site having the nucleotide sequence of CTATCT is located at position 246 to position 251, one E box having the nucleotide sequence of CACCTG is located at position 306 to position 311.

In one non-limiting embodiment, the β-globin LCR region does not comprise the full length of a 387 bp core sequence of human HS2, which has the nucleotide sequence set forth in SEQ ID NO:21 provided below:

[SEQ ID NO: 21]
TAAGCTTCAGTTTTTCCTTAGTTCCTGTTACATTTCTGTGTGTCTCCATT
AGTGACCTCCCATAGTCCAAGCATGAGCAGTTCTGGCCAGGCCCCTGTCG
GGGTCAGTGCCCCACCCCCGCCTTCTGGTTCTGTGTAACCTTCTAAGCAA
ACCTTCTGGCTCAAGCACAGCAATGCTGAGTCATGATGAGTCATGCTGAG
GCTAGGGTGTGTGCCCAGATGTTCTCAGCCTAGAGTGATGACTCCTATCT
GGGTCCCCAGCAGGATGCTTACAGGGCAGATGGCAAAAAAAAGGAGAAGC
TGACCACCTGACTAAAACTCCACCTCAAACGGCATCATAAAGAAAATGGA
TGCCTGAGACAGAATGTGACATATTCTAGAATATATT

In SEQ ID NO:21, one AP1/NF-E2 binding site having the nucleotide sequence of GCTGAGTCA is located at position 175 to position 183, one AP1/NF-E2 binding site having the nucleotide sequence of GATGAGTCA is located at position 185 to position 193, one binding site for Krüppel-like Zn finger proteins having the nucleotide sequence of AGGGTGTGT is located as position 204 to position 212, two E boxes, each of which have the nucleotide sequence of CAGATG, is located at position 216 to position 221, and position 277 to position 282, one GATA-1 binding site having the nucleotide sequence of CTATCT is located at position 245 to position 250, one E box having the nucleotide sequence of CACCTG is located at position 305 to position 310.

In certain embodiments, the β-globin LCR region does not comprise a HS2 region that comprises a core sequence of HS2. A HS2 region that comprises a core sequence of HS2 can vary in length and sequence. In non-limiting examples, a HS2 region that comprises a core sequence of HS2 is from about 400 bp to about 1000 bp, e.g., from about 400 bp to about 500 bp, from about 500 bp to about 600 bp, from about 600 bp to about 700 bp, from about 700 bp to about 800 bp, from about 800 bp to about 900 bp, or from about 900 bp to about 1000 bp, in length. In one non-limiting embodiment, the β-globin LCR region does not comprise a 840 bp HS2 region (e.g., the HS2 region comprised in the globin vector TNS9 disclosed in U.S. Pat. No. 7,541,179). In one non-limiting embodiment, the β-globin LCR region does not comprise a 860 bp HS2 region. In one non-limiting embodiment, the β-globin LCR region does not comprise an about 650 bp HS2 region. In one non-limiting example, the β-globin LCR region does not comprise a 646 bp HS2 region (e.g., the HS2 region comprised in the globin vector LentiGlobin™, also known as “β⁸⁷”). In one non-limiting embodiment, the β-globin LCR region does not comprise an about 420 bp HS2 region. In one non-limiting example, the β-globin LCR region does not comprise a 423 bp HS2 region (e.g., the HS2 region comprised in the globin vector disclosed in Sadelain et al., Proc. Nat'l Acad. Sci. (USA) (1995); 92:6728-6732).

In certain embodiments, the β-globin LCR region does not comprise a HS2 region that sustains the enhancer activity of HS2. In certain embodiments, the β-globin LCR region does not comprise a HS2 region that is capable of enhancing the transcription of a globin gene (e.g., human β-globin gene). In non-limiting examples, the β-globin LCR region does not comprise a HS2 region whose ability to enhance the transcription of a globin gene (e.g., human β-globin gene) is no less than 60%, no less than 70%, no less than 80%, no less than 90%, or no less than 95% in comparison to a native HS2 region.

In certain embodiments, the β-globin LCR region does not comprise a HS2 region that comprises one, two, three, four, five, six or seven of the following binding sites: two (a tandem pair of) AP1/NF-E2 binding sites (e.g., GCTGAGTCA, and GATGAGTCA), one binding site for Krüppel-like Zn finger proteins (e.g., AGGGTGTGT), one GATA-1 binding site (e.g., CTATCT), and three E boxes (CANNTG, e.g., CAGATG, and CACCTG). In certain embodiments, the β-globin LCR region does not comprise a HS2 region that comprises six of the above-described binding sites. For example, in certain embodiments, the β-globin LCR region does not comprise a HS2 region that comprises two AP1/NF-E2 binding sites, one binding site for Krüppel-like Zn finger proteins, one GATA-1 binding site, and two not three E boxes. In certain embodiments, the β-globin LCR region does not comprise a HS2 region that comprises one not two AP1/NF-E2 binding site, one binding site for Krüppel-like Zn finger proteins, one GATA-1 binding site, and three E boxes. In certain embodiments, the β-globin LCR region does not comprise a HS2 region that comprises two AP1/NF-E2 binding sites, one GATA-1 binding site, and three E boxes and does not comprise one binding site for Krüppel-like Zn finger proteins. In certain embodiments, the β-globin LCR region does not comprise a HS2 region that comprises two AP1/NF-E2 binding sites, one binding site for Krüppel-like Zn finger proteins, and three E boxes, and does not comprise one GATA-1 binding site.

In certain embodiments, the β-globin LCR region comprises a HS1 region, a HS3 region, and a HS4 region, and does not comprise a HS2 region. In certain embodiments, the HS1 region, HS3 region and HS4 region within the β-globin LCR region are contiguous. In one non-limiting embodiment, the β-globin LCR region consisting essentially of a HS1 region, a HS3 region and a HS4 region. In another embodiment, the β-globin LCR region comprises two introduced GATA-1 binding sites at the junction between the HS3 region and the HS4 region. The HS3 region can lie between the HS1 region and the HS4 region.

In certain non-limiting embodiments, the β-globin LCR region comprises a HS1 region having the nucleotide sequence set forth in SEQ ID NO:2, SEQ ID NO:3, SEQ ID NO:4, SEQ ID NO:22 or SEQ ID NO:23, a HS3 region having the nucleotide sequence set forth in SEQ ID NO:5, and a HS4 region having the nucleotide sequence set forth in SEQ ID NO:6, SEQ ID NO:7 or SEQ ID NO:8, and the β-globin LCR region does not comprise a HS2 region.

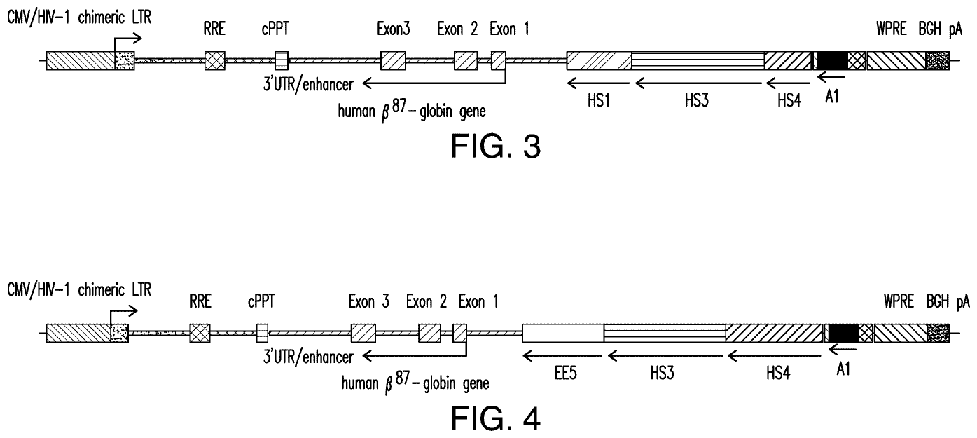

In one non-limiting embodiment, the β-globin LCR region comprises a HS1 region having the nucleotide sequence set forth in SEQ ID NO:2, a HS3 region having the nucleotide sequence set forth in SEQ ID NO:5, and a HS4 region having the nucleotide sequence set forth in SEQ ID NO:6, and the β-globin LCR region does not comprise a HS2 region, as shown in FIG. 2.

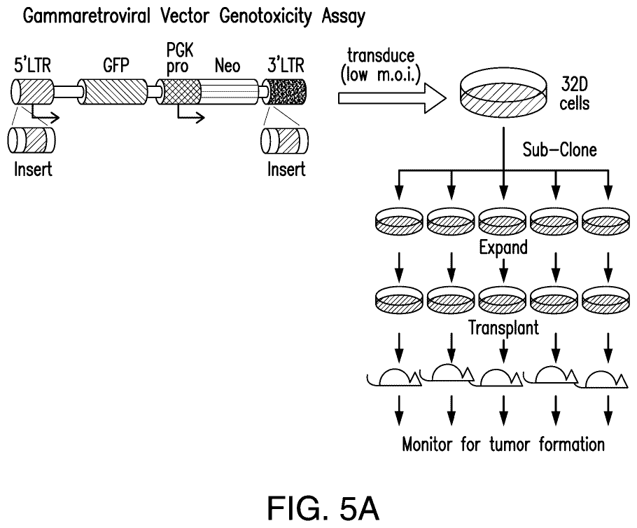

In one non-limiting embodiment, the β-globin LCR region comprises a HS1 region having the nucleotide sequence set forth in SEQ ID NO:3, a HS3 region having the nucleotide sequence set forth in SEQ ID NO:5, and a HS4 region having the nucleotide sequence set forth in SEQ ID NO:8, and the β-globin LCR region does not comprise a HS2 region, as shown in FIG. 3.

In one non-limiting embodiment, the β-globin LCR region comprises a HS1 region having the nucleotide sequence set forth in SEQ ID NO:4, a HS3 region having the nucleotide sequence set forth in SEQ ID NO:5, and a HS4 region having the nucleotide sequence set forth in SEQ ID NO:8, and the β-globin LCR region does not comprise a HS2 region.

In certain embodiments, the β-globin LCR region does not comprise a HS1 region or a HS2 region. In certain embodiments, the β-globin LCR region does not comprise a core sequence of HS1. A core sequence of HS1 sustains the activity of HS1, e.g., enhancer activity, or functioning as a facilitator or regulatory element to tether the enhancer activity of other HS regions, e.g., HS2-4. In addition, a core sequence of HS1 comprises one or more binding sites or binding motifs for ubiquitous as well as tissue-specific (e.g., erythroid-specific) proteins (e.g., transcription factors), including, but not limited to, GATA-1, and Krüppel-like Zn finger proteins (e.g., erythroid-restricted factor EKLF).

In certain embodiments, the β-globin LCR region does not comprise the full length of a core sequence of HS1. In certain embodiments, the core sequence of a HS1 region is a core sequence of human HS1. In one non-limiting embodiment, the core sequence of human HS1 comprises two GATA-1 binding sites (e.g., TTATCT, and CTATCA), and one binding site for EKLF (e.g., CCACACACA). In certain embodiments, the β-globin LCR region does not comprise the full length of a 286 bp core sequence of human HS1. In one non-limiting embodiment, the 286 bp core sequence of human HS1 has the nucleotide sequence set forth in SEQ ID NO:22 provided below:

[SEQ ID NO: 22]
CTGAGCAACTAACTCATGCAGGACTCTCAAACACTAACCTATAGCCTTTT
CTATGTATCTACTTGTGTAGAAACCAAGCGTGGGGACTGAGAAGGCAATA
GCAGGAGCATTCTGACTCTCACTGCCTTTGGCTAGGTCCCTCCCTCATCA
CAGCTCAGCATAGTCCGAGCTCTTATCTATATCCACACACAGTTTCTGAC
GCTGCCCAGCTATCACCATCCCAAGTCTAAAGAAAAAAATAATGGGTTTG
CCCATCTCTGTTGATTAGAAAACAAAACAAAATAAA

In SEQ ID NO:22, one GATA-1 binding site having the nucleotide sequence of TTATCT is located at position 173 to position 178, one GATA-1 binding site having the nucleotide sequence of CTATCA located at position 210 to position 215, and one binding site for EKLF having the nucleotide sequence of CCACACACA is located at position 183 to position 191.

In another non-limiting embodiment, the 286 bp core sequence of human HS1 has the nucleotide sequence set forth in SEQ ID NO:23 provided below:

[SEQ ID NO: 23]
CTGAGCAACTAATCATGCAGGACTCTCAAACACTAACCTATAGCCTTTTC
TATGTATCTACTTGTGTAGAAACCAAGCGTGGGGACTGAGAAGGCAATAG
CAGGAGCATTCTGACTCTCACTGCCTTTAGCTAGGCCCCTCCCTCATCAC
AGCTCAGCATAGTCCTGAGCTCTTATCTATATCCACACACAGTTTCTGAC
GCTGCCCAGCTATCACCATCCCAAGTCTAAAGAAAAAAATAATGGGTTTG
CCCATCTCTGTTGATTAGAAAACAAAACAAAATAAA

The nucleotide sequence set forth in SEQ ID NO:23 corresponds to nucleotides position 21481 to position 21766 of SEQ ID NO:19 (GenBank Access No.: NG_000007.3). In SEQ ID NO:23, one GATA-1 binding site having the nucleotide sequence of TTATCT is located at position 173 to position 178, one GATA-1 binding site having the nucleotide sequence of CTATCA located at position 210 to position 215, and one binding site for EKLF having the nucleotide sequence of CCACACACA is located at position 183 to position 191.

In certain embodiments, the β-globin LCR region does not comprise a HS1 region that comprises a core sequence of HS1. A HS1 region that comprises a core sequence of HS1 can vary in length and sequence. In non-limiting examples, a HS1 region that comprises a core sequence of HS1 is from about 300 bp to about 1200 bp, e.g., from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, or from about 1100 bp to about 1200 bp, in length. In one non-limiting embodiment, the β-globin LCR region does not comprise an about 1.0 kb bp HS1 region. In one non-limiting embodiment, the β-globin LCR region does not comprise an about 1.1 kb HS1 region.

In certain embodiments, the β-globin LCR region does not comprise a HS1 region that sustains the activity of HS1, e.g., enhancer activity, or functioning as a facilitator or regulatory element to tether the enhancer activity of other HS regions, e.g., HS2-4. In certain embodiments, the β-globin LCR region does not comprise a HS1 region that is capable of enhancing the transcription of a globin gene (e.g., human β-globin gene). In non-limiting examples, the β-globin LCR region does not comprise a HS1 region whose ability to enhance the transcription of a globin gene (e.g., human β-globin gene) is no less than 60%, no less than 70%, no less than 80%, no less than 90%, or no less than 95% in comparison to a native HS1 region. In non-limiting examples, the β-globin LCR region does not comprise a HS1 region whose ability to tether the enhancer activity of one or more of HS2-HS4 is no less than 60%, no less than 70%, no less than 80%, no less than 90%, or no less than 95% in comparison to a native HS1 region.

In certain embodiments, the β-globin LCR region does not comprise a HS1 region that comprises one, two, or three of the following binding sites: two GATA-1 binding sites (e.g., TTATCT, and CTATCA), and one binding site for EKLF (e.g., CCACACACA). In certain embodiments, the β-globin LCR region does not comprise a HS1 region that comprises two of the above-described binding sites. For example, in certain embodiments, the β-globin LCR region does not comprise a HS1 region that comprises two GATA-1 binding sites and does not comprise one binding site for EKLF. In certain embodiments, the β-globin LCR region does not comprise a HS1 region that comprises one not two AP1/NF-E2 binding site and one binding site for EKLF.

In certain embodiments, the β-globin LCR region comprises a HS3 region and a HS4 region, and the β-globin LCR region does not comprise a HS1 region or a HS2 region. In certain embodiments, the HS3 region and HS4 region within the β-globin LCR region are contiguous. In one non-limiting embodiment, the β-globin LCR region consisting essentially of a HS3 region and a HS4 region. In another embodiment, the β-globin LCR region comprises two introduced GATA-1 binding sites at the junction between the HS3 region and the HS4 region. The HS3 region can lie between the globin gene or functional portion thereof and the HS4 region.

In certain embodiments, the β-globin LCR region comprises a HS3 region having the nucleotide sequence set forth in SEQ ID NO:5 and a HS4 region having the nucleotide sequence set forth in SEQ ID NO:6, SEQ ID NO:7 or SEQ ID NO:8, and the β-globin LCR region does not comprise a HS1 region or a HS2 region.

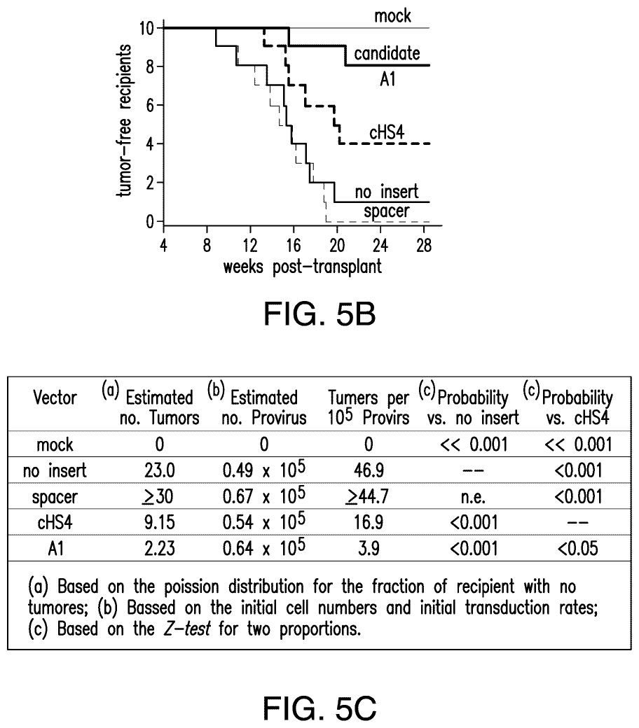

In one non-limiting embodiment, the β-globin LCR region comprises a HS3 region having the nucleotide sequence set forth in SEQ ID NO:5 and a HS4 region having the nucleotide sequence set forth in SEQ ID NO:6, and the β-globin LCR region does not comprise a HS1 region or a HS2 region, as shown in FIG. 4.

Globin Gene

In accordance with the presently disclosed subject matter, the expression cassette comprises a globin gene or a functional portion thereof. The globin gene can be a β-globin gene, a γ-globin gene, or a δ-globin gene. In certain embodiments, the expression cassette comprises a human β-globin gene. In accordance with the presently disclosed subject matter, the human β-globin gene can be a wild-type human β-globin gene, a deleted human β-globin gene comprising one or more deletions of intron sequences, or a mutated human β-globin gene encoding at least one anti-sickling amino acid residue. In one non-limiting embodiment, a presently disclosed expression cassette comprises a wild-type human β-globin gene. In another embodiment, the a presently disclosed expression cassette comprises a human β^{A}-globin gene encoding a threonine to glutamine mutation at codon 87 (β^{A-T87Q}). The glutamine residue at position 87 in the gamma-globin chain augments the anti-sickling activity of the gamma chain relative to the beta chain, while preserving adult oxygen-binding characteristics of the beta chain (Nagel et al., Proc. Natl. Acad. Sci. U.S.A. (1979); 76:670-672). In certain embodiments, a functional portion of a globin gene has at least 80%, at least 90%, at least 95%, or at least 99% identity to a corresponding wild-type reference polynucleotide sequence.

Promoters and Enhancers

In accordance with the presently disclosed subject matter, the expression cassette can further comprise a β-globin promoter. In certain embodiments, the β-globin promoter is positioned between the globin gene or functional portion thereof and the β-globin LCR region. The length and the sequence of the β-globin promoter can vary. In certain embodiments, the β-globin promoter is from about 100 bp to about 1600 bp in length, e.g., from about 200 bp to about 700 bp, from about 100 bp to about 200 bp, from about 200 bp to about 300 bp, from about 300 bp to about 400 bp, from about 400 bp to about 500 bp, from about 500 bp to about 600 bp, from about 600 bp to about 700 bp, from about 700 bp to about 800 bp, from about 800 bp to about 900 bp, from about 900 bp to about 1000 bp, from about 1000 bp to about 1100 bp, from about 1100 bp to about 1200 bp, from about 1200 bp to about 1300 bp, from about 1300 bp to about 1400 bp, from about 1400 bp to about 1500 bp, or from about 1500 bp to about 1600 bp in length. In certain embodiments, the β-globin promoter a human β-globin promoter that is about 130 bp, about 613 bp, about 265 bp, or about 1555 bp, in length. In one embodiment, the β-globin promoter is a human β-globin promoter that is about 613 bp in length. In one non-limiting example, the human β-globin promoter has the nucleotide sequence set forth in SEQ ID NO:10, which is provided below:

[SEQ ID NO: 10]
AAGCAATAGATGGCTCTGCCCTGACTTTTATGCCCAGCCCTGGCTCCTGC
CCTCCCTGCTCCTGGGAGTAGATTGGCCAACCCTAGGGTGTGGCTCCACA
GGGTGAGGTCTAAGTGATGACAGCCGTACCTGTCCTTGGCTCTTCTGGCA
CTGGCTTAGGAGTTGGACTTCAAACCCTCAGCCCTCCCTCTAAGATATAT
CTCTTGGCCCCATACCATCAGTACAAATTGCTACTAAAAACATCCTCCTT
TGCAAGTGTATTTACGTAATATTTGGAATCACAGCTTGGTAAGCATATTG
AAGATCGTTTTCCCAATTTTCTTATTACACAAATAAGAAATTGATGCACT
AAAAGTGGAAGAGTTTTGTCTACCATAATTCAGCTTTGGGATATGTAGAT
GGATCTCTTCCTGCGTCTCCAGAATATGCAAAATACTTACAGGACAGAAT
GGATGAAAACTCTACCTCAGTTCTAAGCATATCTTCTCCTTATTTGGATT
AAAACCTTCTGGTAAGAAAAGAAAAAAAATATATATATATATGTGTATAT
ATACACACATACATATACATATATATGCATTCATTTGTTGTTGTTTTTCT
TAATTTGCTCATG

In one embodiment, the β-globin promoter is a human β-globin promoter that is about 265 bp in length. In one non-limiting example, the human β-globin promoter has the nucleotide sequence set forth in SEQ ID NO:11.

[SEQ ID NO: 11]
AAGCAATAGATGGCTCTGCCCTGACTTTTATGCCCAGCCCTGGCTCCTGC
CCTCCCTGCTCCTGGGAGTAGATTGGCCAACCCTAGGGTGTGGCTCCACA
GGGTGAGGTCTAAGTGATGACAGCCGTACCTGTCCTTGGCTCTTCTGGCA
CTGGCTTAGGAGTTGGACTTCAAACCCTCAGCCCTCCCTCTAAGATATAT
CTCTTGGCCCCATACCATCAGTACAAATTGCTACTAAAAACATCCTCCTT
TGCAAGTGTATTTAC

Additionally or alternatively, a presently disclosed expression cassette can further comprise a human β-globin 3′ enhancer. In certain embodiments, the human β-globin 3′ enhancer is positioned in the upstream of the globin gene or functional portion thereof. In certain embodiments, the β-globin 3′ enhancer is from about 500 bp to about 1000 bp in length, e.g., from about 500 bp to about 600 bp, from about 600 bp to about 700 bp, from about 700 bp to about 800 bp, or from about 800 bp to about 900 bp in length. In one embodiment, the human β-globin 3′ enhancer is about 879 bp in length. In one example, the human β-globin 3′ enhancer has the nucleotide sequence set forth in SEQ ID NO:12.

[SEQ ID NO: 12]
TAGGTATTGAATAAGAAAAATGAAGTTAAGGTGGTTGATGGTAACACTAT
GCTAATAACTGCAGAGCCAGAAGCACCATAAGGGACATGATAAGGGAGCC
AGCAGACCTCTGATCTCTTCCTGAATGCTAATCTTAAACATCCTGAGGAA
GAATGGGACTTCCATTTGGGGTGGGCCTATGATAGGGTAATAAGACAGTA
GTGAATATCAAGCTACAAAAAGCCCCCTTTCAAATTCTTCTCAGTCCTAA
CTTTTCATACTAAGCCCAGTCCTTCCAAAGCAGACTGTGAAAGAGTGATA
GTTCCGGGAGACTAGCACTGCAGATTCCGGGTCACTGTGAGTGGGGGAGG
CAGGGAAGAAGGGCTCACAGGACAGTCAAACCATGCCCCCTGTTTTTCCT
TCTTCAAGTAGACCTCTATAAGACAACAGAGACAACTAAGGCTGAGTGGC
CAGGCGAGGAGAAACCATCTCGCCGTAAAACATGGAAGGAACACTTCAGG
GGAAAGGTGGTATCTCTAAGCAAGAGAACTGAGTGGAGTCAAGGCTGAGA
GATGCAGGATAAGCAAATGGGTAGTGAAAAGACATTCATGAGGACAGCTA
AAACAATAAGTAATGTAAAATACAGCATAGCAAAACTTTAACCTCCAAAT
CAAGCCTCTACTTGAATCCTTTTCTGAGGGATGAATAAGGCATAGGCATC
AGGGGCTGTTGCCAATGTGCATTAGCTGTTTGCAGCCTCACCTTCTTTCA
TGGAGTTTAAGATATAGTGTATTTTCCCAAGGTTTGAACTAGCTCTTCAT
TTCTTTATGTTTTAAATGCACTGACCTCCCACATTCCCTTTTTAGTAAAA
TATTCAGAAATAATTTAAATACATCATTG

Furthermore, a presently disclosed expression cassette can further comprise at least one erythroid-specific enhancer. The presently disclosed expression cassette allows for expression of a globin gene (e.g., human β-globin gene) in erythroid-specific fashion. The erythroid-specific enhancer can enhance the expression of the globin gene in erythroid-specific fashion. For example, the erythroid-specific enhancer lack enhancer activity in non-erythroid tissues. In particularly, for the β-globin LCR region that lacks a HS2 region, which primarily functions as an expression enhancer, the addition of one or more erythroid-specific enhancers can compensate the enhancing activity of a HS2 region. Furthermore, the presently disclosed erythroid-specific enhancers do not decrease or reduce the titer of a vector comprising the expression cassette. The length of the erythroid-specific enhancer can vary, e.g., from about 100 bp to about 200 bp, from about 100 bp to about 120 bp, from about 120 bp to about 140 bp, from about 140 bp to about 200 (e.g., from about 140 bp to about 150 bp, from about 150 bp to about 160 bp, from about 160 bp to about 170 bp, from about 170 bp to about 180 bp, from about 180 bp to about 190 bp, or from about 190 bp to about 200 bp). In certain embodiments, the erythroid-specific enhancer has a length of from about 140 bp to about 200 bp. In one non-limiting embodiment, the erythroid-specific enhancer has a length of 152 bp, which has the nucleotide sequence set forth in SEQ ID NO:13, which is provided below:

[SEQ ID NO: 13]
TCTCCCACGCCCTGGTCTCAGCTTGGGGAGTGGTCAGACCCCAATGGCGA
TAAACTCTGGCAACTTTATCTGTGcaCTGCAGGCTCAGCCCCAAcaGCTT
TAGCTTTCACAAGCAGGCAGGGGAAGGGAAACACATATCTCCAGATATGA
GG

In one non-limiting embodiment, the erythroid-specific enhancer has a length of 157 bp, which has the nucleotide sequence set forth in SEQ ID NO:14, which is provided below:

[SEQ ID NO: 14]
CTAAACCCCTCCCCCACCCTAGCCCCAAGCTTCATCTTAGCTCCACTCCT
GACCCTATCCAGCTAAAGGTCCCCACCCAGCTCCTGCCTATCTAGTCATT
GCATATGGCAAGACTTGAAAGTCCTATCTCAAAGCAGCAGAATTATCAGC
TACGACT

In one non-limiting embodiment, the erythroid-specific enhancer has a length of 141 bp, which has the nucleotide sequence set forth in SEQ ID NO:15, which is provided below:

[SEQ ID NO: 15]
CCATCCCCCAGCACTCCCTGCCCCCACAGCCCAGACTTGACCAACTCCCA
GCTccGCCTGGGACTTCCAGATATGGGGCCCCACCCTTGCAGGCCTTGGG
GACGCTGAAGATATTGACTATCTGCGTGCCggAAAAGGGTG

In one non-limiting embodiment, the erythroid-specific enhancer has a length of 171 bp, which has the nucleotide sequence set forth in SEQ ID NO:16, which is provided below:

[SEQ ID NO: 16]
AAAGGCTGGGGGTGGGAGTAGCGGATTTGAAGCACTTGTTGGCCTACAGA
GGTGTGGCAAGCAGAGCACCTCAGAACTCAGGCGTACTGCCCGCCGCCCG
AGCCCTGCGAGGGCCGATAGCGAGGGTGTGGCCCTTATCTGCACCCAGCA
GAGCGCCGGCGGGGTACGGTC

In one non-limiting embodiment, the erythroid-specific enhancer has a length of 195 bp, which has the nucleotide sequence set forth in SEQ ID NO:17, which is provided below:

[SEQ ID NO: 17]
CAGTTGCCTCAGCTGAGTATGTCTTCTAAAGATAATGTCGATTGTGTATG
GCTGATGGGATTCTAGGACCAAGCAAGAGGTTTTTTTTTTTCCCCCACAT
ACTTAACGTTTCTATATTTCTATTTGAATTCGACTGGACAGTTCCATTTG
AATTATTTCTCTCTCTCTCTCTCTCTGACACATTTTATCTTGCCA

Erythroid-specific enhancers can be identified and determined by any suitable methods known in the art. The erythroid-specific enhancers can be positioned at the 3′ LTR (downstream) or the 5′ LTR (downstream) of the β-globin LCR region. In one embodiment, the at least one erythroid-specific enhancer is positioned in the 5′ LTR of the β-globin LCR region, e.g., the upstream of the HS3 region. The expression cassette can comprise one, two, three, four, or five erythroid-specific enhancers. In one embodiment, the expression cassette comprises one erythroid-specific enhancer. In another embodiment, the expression cassette comprises two erythroid-specific enhancers. In yet another embodiment, the expression cassette comprises three erythroid-specific enhancers. In certain embodiments, the expression cassette comprises four erythroid-specific enhancers. In a non-limiting embodiment, the expression cassette comprises five erythroid-specific enhancers.

Insulators

In accordance with the presently disclosed subject matter, the expression cassette comprises at least one of the above-described insulators. In certain embodiments, a presently disclosed expression cassette comprises at least one insulator comprising the CTCF binding site sequence set forth in SEQ ID NO:18, for example, but not limited to, an insulator comprising SEQ ID NO: 24 or SEQ ID NO:25, such as an insulator having the nucleotide sequence set forth in SEQ ID NO:1 (i.e., insulator A1). In various non-limiting embodiments, the insulator can be incorporated or inserted into one or both LTRs or elsewhere in the region of a presently disclosed expression cassette that integrates into the cellular genome. In one embodiment, the insulator is positioned at the 3′ end of the expression cassette. In one embodiment, the insulator is positioned at the 5′ end of the expression cassette. In one embodiment, the expression cassette comprises two of the insulator having the nucleotide sequence set forth in SEQ ID NO:1, where one insulator is positioned at the 3′ end and the other insulator is positioned at the 5′ end of the expression cassette.

The presently disclosed insulators possess powerful enhancer blocking activity. In certain embodiments, the insulators possess barrier activity in addition to enhancer blocking activity. The presently disclosed insulators substantially decrease the risks of insertional mutagenesis and genotoxicity associated with viral vectors. Furthermore, when a presently disclosed insulator is incorporated into a vector, the insulator does not adversely effect vector titers of the vector. In certain embodiments, the insulators (e.g., insulator A1) increase the in vivo expression of the globin gene or functional portion thereof.

For the purpose of illustration and not limitation, FIGS. 1-4 show recombinant vectors comprising exemplary expression cassettes in accordance with certain embodiments of the presently disclosed subject matter. FIG. 1 shows a recombinant vector comprising a presently disclosed expression cassette that comprises a human β^{A-T87Q} globin gene, which is operably linked to a β-globin LCR region that comprises a 860 bp HS2 region (e.g., one having the nucleotide sequence set forth in SEQ ID NO:9), a 1301 bp HS3 region (e.g., one having the nucleotide sequence set forth in SEQ ID NO:5), and a 1065 bp HS4 region (e.g., one having the nucleotide sequence set forth in SEQ ID NO:7).

FIG. 2 shows one exemplary recombinant vector comprising an expression cassette in accordance with one embodiment of the presently disclosed subject matter. FIG. 2 shows a recombinant vector comprising a presently disclosed expression cassette that comprises a human β^{A-T87Q} globin gene, which is operably linked to a β-globin LCR region that comprises a 1.1 kb HS1 region (e.g., one having the nucleotide sequence set forth in SEQ ID NO:2), a 1301 bp HS3 region (e.g., one having the nucleotide sequence set forth in SEQ ID NO:5), and a 1065 bp HS4 region (e.g., one having the nucleotide sequence set forth in SEQ ID NO:6).

FIG. 3 shows one exemplary recombinant vector comprising an expression cassette in accordance with one embodiment of the presently disclosed subject matter. FIG. 3 shows a recombinant vector comprising a presently disclosed expression cassette that comprises a human β^{A-T87Q} globin gene, which is operably linked to a β-globin LCR region that comprises a 602 bp HS1 region (e.g., one having the nucleotide sequence set forth in SEQ ID NO:3), a 1301 bp HS3 region (e.g., one having the nucleotide sequence set forth in SEQ ID NO: 5), and a 446 bp HS4 region (e.g., one having the nucleotide sequence set forth in SEQ ID NO: 8).

FIG. 4 shows one exemplary recombinant vector comprising an expression cassette in accordance with one embodiment of the presently disclosed subject matter. FIG. 4 shows a recombinant vector comprising a presently disclosed expression cassette that comprises a human β^{A-T87Q} globin gene, which is operably linked to a β-globin LCR region that comprises a 1301 bp HS3 region (e.g., one having the nucleotide sequence set forth in SEQ ID NO:5), and a 1065 bp HS4 region (e.g., one having the nucleotide sequence set forth in SEQ ID NO:6). The expression cassette shown in FIG. 4 also comprises the following five erythroid-specific enhancers (shown as “EE5” in FIG. 4): one erythroid-specific enhancer having the nucleotide sequence set forth in SEQ ID NO:13, one erythroid-specific enhancer having the nucleotide sequence set forth in SEQ ID NO:14, one erythroid-specific enhancer having the nucleotide sequence set forth in SEQ ID NO:15, one erythroid-specific enhancer having the nucleotide sequence set forth in SEQ ID NO: 16, and one erythroid-specific enhancer having the nucleotide sequence set forth in SEQ ID NO: 17.

As shown in FIGS. 1-4, each of the expression cassettes comprise an insulator having the nucleotide sequence set forth in SEQ ID NO:1 (i.e., insulator A1). In addition, as shown in FIGS. 1-4, each of the expression cassettes comprise a 879 bp human β-globin 3′ enhancer, which is positioned upstream of the human β-globin gene. Furthermore, as shown in FIGS. 1-4, each of the recombinant vectors comprise a Woodchuck hepatitis post-regulatory element (WPRE) and a bovine growth hormone polyadenylation signal in the 3′ long terminal repeat (LTR) of the vector (e.g., 3′ to the R region in the 3′ LTR).

### III. Vectors, Nucleases and CRISPR-Cas Systems

The presently disclosed subject matter provides vectors and delivery systems (e.g., a non-naturally occurring or engineered nucleases or a CRISPR-Cas system) comprising the above-described expression cassettes. The vectors and delivery systems are suitable delivery vehicles for the stable introduction of globin gene (e.g., human β-globin) into the genome of a broad range of target cells to increase expression of the globin protein (human β-globin protein) in the cell.

In certain embodiments, the vector is a retroviral vector (e.g., gamma retroviral or lentiviral) that is employed for the introduction or transduction of the above-described expression cassette into the genome of a host cell (e.g., a hematopoietic stem cell, an embryonic stem cell, an induced pluripotent stem cell, or a hemogenic endothelium cell). In certain embodiments, the retroviral vector comprises an expression cassette that comprises one of the above-described insulators, e.g., insulator A1. The insulator can be positioned at the 3′ or the 5′ end of the expression cassette. In one embodiment, the insulator is positioned at the 3′ end of the expression cassette. During reverse transcription and vector integration, the insulator positioned at the 3′ end is copied into the 5′ end of the expression cassette. The resulting topology places copies of the insulator between the genomic regions located at the 5′ LTR and the 3′ LTR of the integrated virus and enhancer activity from the 5′ LTR and internal package promoter, but does not contain the enhancer in the 3′ LTR. This topology can decrease genotoxicity, thereby resulting in decreased tumor formation and increased survival of the animals.

In certain embodiments, the recombinant vector further comprises a Woodchuck hepatitis post-regulatory element (WPRE) in the 3′ long terminal repeat (LTR) of the vector (e.g., 3′ to the R region in the 3′ LTR of the vector). In certain embodiments, the recombinant vector further comprises a bovine growth hormone polyadenylation signal in addition to the WPRE in the 3′ long terminal repeat (LTR) of the vector (e.g., 3′ to the R region in the 3′ LTR of the vector). An essential feature of therapeutic globin vectors is to achieve a high titer, sufficient for effective transduction of patient cells. By virtue of their large cargo, comprising a gene, promoter, enhancers and/or LCR elements, globin lentiviral vectors inherently have low titer, complicating their manufacture and limiting their clinical use. This problem is further compounded by the incorporation of additional genomic elements such as an insulator, which further increase the size of the vector. The WPRE can increase the titer of the recombinant vector. Addition of a bovine growth hormone polyadenylation signal to the WPRE can further increase the titer of the recombinant vector. In certain embodiments, the WPRE and the bovine growth hormone polyadenylation signal are not comprised within the expression cassette, and thus, not transferred to the cells transduced with the recombinant vector. The incorporation of these elements for enhancing the production of globin lentiviral vectors is essential to yield higher titers and hence for the clinical usefulness of the vectors described in this application.

In one non-limiting example, a presently disclosed expression cassette can be cloned into a retroviral vector and expression can be driven from its endogenous promoter, from the retroviral long terminal repeat, or from an alternative internal promoter. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Suitable methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a globin gene (e.g., a human β-globin gene) in a host cell (e.g., hematopoietic stem cells, an embryonic stem cell, or an induced pluripotent stem cell). Preferably, the chosen vector exhibits high efficiency of infection and stable integration and expression (see, e.g., Cayouette et al., Human Gene Therapy (1997); 8:423-430; Kido et al., Current Eye Research (1996); 15:833-844; Bloomer et al., Journal of Virology (1997); 71:6641-6649; Naldini et al., Science (1996); 272:263 267; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy (1990); 15-14; Friedman, Science (1989); 244:1275-1281; Eglitis et al., BioTechniques 6:608-614, 1988; Tolstoshev et al., Current Opinion in Biotechnology (1990); 1:55-61; Sharp, The Lancet (1991); 337:1277-1278; Cornetta et al., Nucleic Acid Research and Molecular Biology (1987) 36:311-322; Anderson, Science (1984); 226:401-409; Moen, Blood Cells (1991); 17:407-416; Miller et al., Biotechnology (1989); 7:980-990; Le Gal La Salle et al., Science (1993); 259:988-990; and Johnson, Chest (1995); 107:775-83S). Retroviral vectors are particularly well developed and have been used in clinical settings (Rosenberg et al., N. Engl. J. Med (1990); 323:370; Anderson et al., U.S. Pat. No. 5,399,346).

The requirement for efficient delivery and integration make retroviral vectors suitable for transducing a presently disclosed expression cassette. Retroviral vectors can be derived from three genera of the retroviridae: the γ-retroviruses (also known as C-type murine retroviruses or oncoretroviruses), the lentiviruses, and the spumaviruses (also known as foamy viruses). Several reviews detailing molecular approaches for the generation of replication-defective retroviral particles are available (Cornetta et al. (2005); Cockrell & Kafri (2007)). The vector itself, which encodes the therapeutic transgene or cDNA, retains the minimal viral sequences needed to enable packaging in viral particles in a packaging cell line, reverse transcription, and integration. The packaging cell expresses the necessary structural proteins and enzymes that are required to assemble an infectious recombinant particle that contains the vector sequence and the machinery needed for its reverse transcription and integration in the transduced cell.

While the manufacturing aspects of all retroviral vector types follow the same general principles, γ-retroviral, lentiviral and spumaviral vectors differ in some of their intrinsic biological properties. Gamma-retroviruses, including the prototypic murine leukaemia viruses (MLV), effectively infect many cell types but are unable to integrate in cells that do not proceed to S phase soon after infection. In contrast, lentiviruses and their vector derivatives can transduce nondividing cells (Follenzi & Naldini, 2002; Salmon & Trono, 2002) owing to their ability to translocate to the nucleus and integrate in the absence of cell division (Lewis & Emerman, 1994; Goff, 2001). Another fundamental attribute of lentiviral vectors is their relative genomic stability, as established for globin lentiviral vectors (May et al., 2000), which contrasts with the genomic instability of MLV-based globin vectors (Leboulch et al., 1994; Sadelain et al., 1995). Lentiviral and foamy vectors further provide a greater packaging capacity (Kumar et al., 2001; Rethwilm, 2007). All three vector types have been used successfully for the transduction of cytokineactivated HSCs (Miyoshi et al., 1999; Josephson et al., 2002; Leurs et al., 2003).

These three vector systems differ in their integration patterns. The integration pattern of retroviruses is semi-random and biased towards genes and their vicinity in approximately two-thirds of all integration events (Schroder et al., 2002; Wu et al., 2003; Mitchell et al., 2004; De Palma et al., 2005; Trobridge et al., 2006). There are however subtle and possibly significant differences in their exact distribution. Gamma-retroviruses have a propensity to integrate upstream of transcribed genes, whereas lentiviruses and lentiviral vectors target the entire transcribed gene sequence. Foamy vectors appear to be less prone to intragenic integration (Trobridge et al., 2006). In one embodiment, the vector comprising the expression cassette is a lentivirus vector. The vectors can be derived from human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), caprine arthritis encephalitis virus (CAEV) and the like. In one non-limiting embodiment, the lentiviral vector is an HIV vector. HIV-based constructs are the most efficient at transduction of human cells.

The semi-random pattern of vector integration exposes patients to the risk of insertional oncogenesis when the vector trans-activates a neighboring oncogene. This may result in clonal expansion (Ott et al, 2006; Cavazzana-Calvo et al, 2010), myelodysplasia (Stein et al, 2010) or leukaemia (Hacein-Bey-Abina et al, 2003, 2008; Howe et al, 2008). Targeted gene delivery strategies, utilizing a non-naturally occurring or engineered nuclease (including, but not limited to, Zinc-finger nuclease (ZNFs), meganuclease, transcription activator-like effector nuclease (TALEN)), or a CRISPR-Cas system, can reduce or even eliminate the concern of insertional oncogenesis that is inherent to the use of retroviral vectors.

Eukaryotic cells utilize two distinct DNA repair mechanisms in response to DNA double strand breaks (DSBs): Homologous recombination (HR) and non-homologous end-joining (NHEJ). The activation of the HR repair machinery depends on the cell cycle status, and it is restricted to the S and G2 phases; in contrast, the NHEJ pathway is active throughout the cell cycle. Mechanistically, HR is an error-free DNA repair mechanism, because it requires a homologous template to repair the damaged DNA strand. On the other hand, NHEJ is a template-independent repair mechanism that is imprecise, due to DNA end processing during repair that leads to insertions or deletions at the DNA break site (Moynahan & Jasin, 2010). Because of its homology-based mechanism, HR has been used as a tool to site-specifically engineer the genome of different species. From a therapeutic perspective, HR has been successfully used to repair mutated genes, thus offering a promising approach to cell-mediated treatment of monogenic diseases (Porteus et al, 2006).

Gene targeting by HR requires the use of two homology arms that flank the transgene/target site of interest. Generally, standard plasmid DNAs have been used to deliver 5-10 kb homology arms along with transgenes for positive and negative selection. This method is commonly used to knock-out/knockin genes in mouse embryonic stem (mES) cells (Capecchi, 2005; FIG. 2B). In human cells, the use of this approach has allowed gene targeting with efficiencies in the order of 10⁻⁶, which are lower than in mES cells and are not therapeutically practical. HR efficiency can be increased by the introduction of DNA-doubled stranded breaks (DSBs) at the target site using specific rare-cutting endonucleases, resulting in over 1,000-fold increase in correct gene targeting (Jasin, 1996). The discovery of this phenomenon prompted the development of methods to create site-specific DSBs in the genome of different species. Various chimeric enzymes have been designed for this purpose over the last decade, namely zinc-finger nucleases (ZFNs), meganucleases, and transcription activator-like effector nucleases (TALENs).

ZFNs are modular chimeric proteins that contain a ZF-based DNA binding domain (DBD) and a FokI nuclease domain (Porteus & Carroll, 2005). DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted in principle. However, as a single ZFN can bind and nick DNA, there is potential for a high number of off-target effects, resulting in the activation of the NHEJ pathway that may either introduce insertions/deletions or integrate the targeting vector in a non-specific manner. Obligate FokI domains that can nick their respective DNA strand only when they form a heterodimer were recently reported (Doyon et al, 2011). The use of such obligate ZFNs can reduce the genotoxic effects of this approach.

Meganucleases (MNs)/homing endonucleases (HEs) are dsDNA nucleases that recognize and cleave large DNA sites (14-40 bp) with low cleavage frequencies in eukaryotic genomes (Paques & Duchateau, 2007). Although this limits the potential target sites, MN-DNA structures have been used as a guide to specifically modify DNA-interacting residues in order to change the MN specificity (Marcaida et al, 2010). I-CreI has been successfully engineered to generate chimeric meganucleases that target the human XPC and RAG1 genes, and they have been shown to stimulate HR activity in mammalian cells with no evident genotoxicity (Redondo et al, 2008; Grizot et al, 2009). The genotoxicity of this approach will need to be compared to that of ZFNs and TALE nucleases.

TALENs are similar ZFN except that the DBD is derived from transcription activator-like effectors (TALEs), which are virulent factors used by phytopathogenic bacteria (Herbers, 1992). The TALE DBD is modular, and it is composed of 34-residue repeats, and its DNA specificity is determined by the number and order of repeats (Herbers, 1992). Each repeat binds a single nucleotide in the target sequence through only two residues (Boch, 2011). The advantage over ZFN technology is the rapid construction of DBDs.

A number of studies have used these chimeric enzymes to stimulate HR for either gene addition or gene repair at their target site (Paques & Duchateau, 2007; Urnov et al, 2010). Porteus designed a ZFN to a half site sequence from the human HBB that surrounds the sickle cell mutation nucleotide (Porteus, 2006). This ZFN targets the sequence and stimulates HR at a chimeric DNA target when combined with a ZFN targeting the Zif268 binding site. There have been recent advances in targeting genes in cord blood CD34⁺ cells. Use of non-integrating lentiviruses to deliver ZFNs and the donor DNA in these cells to target the CCR5 gene was reported in Lombardo et al, 2007. Lombardo et al, 2007 showed gene addition at this locus with correct targeting in 80% of the positively selected cells.

The presently disclosed subject matter provides a non-naturally occurring or engineered nuclease comprising a presently disclosed expression cassette, as described above. Suitable nucleases include, but are not limited to, ZFNs, meganucleases, and TALENs. A presently disclosed nuclease comprises a DNA binding domain and a nuclease cleavage domain. The DNA binding domain of the nuclease can be engineered to bind to a sequence of choice, e.g., a predetermined site. An engineered DNA binding domain can have a distinct binding specificity, compared to a naturally occurring nuclease. Engineering methods include, but are not limited to, rational design and various types of selection. Any suitable cleavage domain can be operatively linked to a DNA-binding domain to form a nuclease. For example, Zinc-finger protein (ZFP) DNA-binding domains can be fused to nuclease cleavage domains to create ZFNs-a functional entity that is able to recognize its intended nucleic acid target through its engineered ZFP DNA binding domain and cause the DNA to be cut near the ZFP binding site via the nuclease activity. See, e.g., Kim et al. Proc Nat'l Acad Sci USA (1996); 93(3):1156-1160. Likewise, TALE DNA-binding domains can be fused to nuclease cleavage domains to create TALENs. See, e.g., U.S. Publication No. 20110301073.

The cleavage domain can be heterologous to the DNA-binding domain, e.g., a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional regions thereof) can be used as a source of cleavage domains and cleavage half-domains.

Similarly, a cleavage half-domain can be derived from the above-described nuclease that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional portions thereof), or each cleavage half-domain can be derived from a different endonuclease (or functional portions thereof).

In certain embodiments, the nuclease comprises an expression cassette that comprises two of the above-described insulators, e.g., two of the insulator having the nucleotide sequence set forth in SEQ ID NO:1. One of the two insulators is positioned at the 3′ end of the expression cassette, and the other insulator is positioned at the 5′ end of the expression cassette.

The presently disclosed subject matter also provides a non-naturally occurring or engineer CRISPR-Cas system comprising the above-described expression cassette. The CRISPR (Clustered Regularly Interspaced Short Palindromic Repeats)-Cas (CRISPR Associated) system is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide a CRISPR-Cas nuclease to a region homologous to the crRNA in the target DNA called a “proto spacer”. The CRISPR-Cas nuclease cleaves the DNA to generate blunt ends at the DSB at sites specified by a 20-nucleotide guide sequence contained within the crRNA transcript. The CRISPR-Cas nuclease requires both the crRNA and the tracrRNA for site specific DNA recognition and cleavage. This system has been engineered such that the crRNA and tracrRNA can be combined into one molecule (the “single guide RNA”); and the crRNA equivalent portion of the single guide RNA can be engineered to guide the CRISPR-Cas nuclease to target any desired sequence (see Jinek et al., Science (2012); 337:816-821). Thus, the CRISPR-Cas system can be engineered to create a DSB at a desired target in a genome. In certain embodiments, the CRISPR-Cas system comprises a CRISPR-Cas nuclease and a single-guide RNA. Suitable examples of CRISPR-Cas nucleases include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These CRISPR-Cas nucleases are known; for example, the amino acid sequence of S. pyogenes Cas9 protein may be found in the SwissProt database under accession number Q99ZW2. In some embodiments, the CRISPR-Cas nuclease has DNA cleavage activity, e.g., Cas9. In certain embodiments, the CRISPR-Cas nuclease is Cas9. The CRISPR-Cas nuclease can direct cleavage of one or both strands at the location of a target sequence (e.g., a genomic safe harbor site). Additionally, the CRISPR-Cas nuclease can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

The presently disclosed nucleases and CRISPR-Cas system allow for targeted delivery of the expression cassette. In certain embodiments, a presently disclosed CRISPR-Cas system or the DNA binding domain of a presently disclosed nuclease binds to a genomic safe harbor site. A nuclease or the CRISPR-Cas system generates a double strand break at the genomic safe harbor site. Genomic safe harbor sites are intragenic or extragenic regions of the human genome that are able to accommodate the predictable expression of newly integrated DNA without adverse effects on the host cell or organism. A useful safe harbor must permit sufficient transgene expression to yield desired levels of the vector-encoded protein or non-coding RNA. A genomic safe harbor site also must not predispose cells to malignant transformation nor alter cellular functions. Methods for identifying genomic safe harbor sites are described in Sadelain et al., “Safe Harbours for the integration of new DNA in the human genome,” Nature Reviews (2012); 12:51-58; Papapetrou et al., “Genomic safe harbors permit high β-globin transgene expression in thalassemia induced pluripotent stem cells” Nat Biotechnol. (2011) January; 29(1):73-8, which are incorporated by reference in their entireties. A presently disclosed genomic safe harbor site meets one or more (one, two, three, four, or five) of the following five criteria: (1) distance of at least 50 kb from the 5′ end of any gene (e.g., from the 5′ end of the gene), (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the human genome. As the most common insertional oncogenesis event is transactivation of neighboring tumor-promoting genes, the first two criteria exclude the portion of the human genome located near promoters of genes, in particular, cancer-related genes, which are genes functionally implicated in human cancers or the human homologs of genes implicated in cancer in model organisms. Proximity to miRNA genes is one exclusion criterion because miRNAs are implicated in the regulation of many cellular processes, including cell proliferation and differentiation. As vector integration within a transcription unit can disrupt gene function through the loss of function of a tumor suppressor gene or the generation of an aberrantly spliced gene product, the fourth (iv) criterion excludes all sites located inside transcribed genes. UCRs, which are regions that are highly conserved over multiple vertebrates and known to be enriched for enhancers and exons, and long non-coding RNAs, are also excluded. In certain embodiments, the genomic safe harbor site is an extragenic genomic safe harbor site. In certain embodiments, the genomic safe harbor site is located on chromosome 1.

The presently disclosed subject matter also provides polynucleotides encoding the above-described nucleases, vectors comprising the polynucleotides encoding the above-described nucleases, polynucleotides encoding the above-described CRISPR-Cas system, and vectors comprising the polynucleotides encoding the above-described CRISPR-Cas system.

The nucleases and polynucleotides encoding these nucleases, and the CRISPR-Cas system and polynucleotides encoding the CRISPR-Cas system can be delivered in vivo or ex vivo by any suitable means. For example, nucleases and CRISPR-Cas system as described herein can be delivered to a cell (e.g., a hematopoietic stem cell, an embryonic stem cell, an induced pluripotent stem cell, or an hemogenic endothelium cell) by a vector comprising polynucleotides encoding the nuclease or the CRISPR-Cas system. Any vectors can be used including, but not limited to, plasmid vectors, retroviral vectors (e.g., γ-retroviral vectors, lentiviral vectors and foamy viral vectors), adenovirus vectors, poxvirus vectors; herpes virus vectors and adena-associated virus vectors, etc. In one embodiment, the vector comprising a polynucleotide encoding an above-described nuclease or an above-described CRISPR-Cas system is a lentiviral vector. In one particular embodiment, the lentiviral vector is a non-integrating lentiviral vector. Examples of non-integrating lentiviral vector are described in Ory et al. (1996) Proc. Natl. Acad. Sci. USA 93:11382-11388; Dull et al., (1998) J. Viral. 72:8463-8471; Zuffery et al. (1998) J. Viral. 72:9873-9880; Follenzi et al., (2000) Nature Genetics 25:217-222; U.S. patent publication No 2009/054985.

Additionally, non-viral approaches can also be employed for the expression of a globin gene in cells. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases. Transient expression may be obtained by RNA electroporation.

### IV. Cells

Genetic modification of cells (e.g., hematopoietic stem cells, embryonic stem cells, induced pluripotent stem cells, and hemogenic endothelium cells) can be accomplished by transducing a substantially homogeneous cell composition with a recombinant DNA or RNA construct (e.g., a vector or a delivery system comprising the above-described expression cassette). The presently disclosed subject matter provides cells transduced with the above-described expression cassettes, cells transduced with the above-described vectors, and cells transduced with the above-described nucleases or with vectors comprising polynucleotides encoding the nucleases, and cell transduced with the above-described CARISPR-Cas system or with vectors comprising polynucleotides encoding the CARISPR-Cas system, which are collectively referred to as “transduced cells”. As described above, the vectors, nucleases and CRISPR-Cas system are employed for transduction of the expression cassette to the cells to express a globin gene (e.g., a human β-globin gene). In certain embodiments, the transduced cells are administered to a subject to treat and/or prevent a hematopoietic disease, disorder, or condition. The presently disclosed insulators can enhance the efficiency of the transduction of the expression cassette to cells.

Suitable transduced cells include, but are not limited to, stem cells, progenitor cells, and differentiated cells. As used herein, the term “progenitor” or “progenitor cells” refers to cells that have the capacity to self-renew and to differentiate into more mature cells. Progenitor cells have a reduced potency compared to pluripotent and multipotent stem cells. Many progenitor cells differentiate along a single lineage, but may also have quite extensive proliferative capacity.

In certain embodiments, the transduced cells are stem cells. Stem cells have the ability to differentiate into the appropriate cell types when administered to a particular biological niche, in vivo. A stem cell is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are sub-classified according to their developmental potential as totipotent, pluripotent, multipotent and oligo/unipotent. As used herein, the term “pluripotent” means the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

In certain embodiments, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells, and/or hematopoietic stem cells. In one embodiment, the transduced cells are hematopoietic stem cells (HSCs). HSCs give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to all blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). When transplanted into lethally irradiated animals or humans, hematopoietic stem and progenitor cells can repopulate the erythroid, neutrophil-macrophage, megakaryocyte and lymphoid hematopoietic cell pool.

HSCs can be isolated or collected from bone marrow, umbilical cord blood, or peripheral blood. HSCs can be identified according to certain phenotypic or genotypic markers. For example, HSCs can be identified by their small size, lack of lineage (lin) markers, low staining (side population) with vital dyes such as rhodamine 123 (rhodamineDULL, also called rholo) or Hoechst 33342, and presence of various antigenic markers on their surface, many of which belong to the cluster of differentiation series (e.g., CD34, CD38, CD90, CD133, CD105, CD45, Terl 19, and c-kit, the receptor for stem cell factor). In one embodiment, the transduced cell is a CD34⁺ HSC.

In one embodiment, the transduced cell is an embryonic stem cell. In another embodiment, the transduced cell is an induced pluripotent stem cell. In yet another embodiment, the transduced cell is a hemogenic endothelium cell.

While HSCs are the natural vehicle for restoring long-term hematopoiesis, their use has some important limitations. The first is their relative scarcity, which can eventually preclude autologous HSC therapy when the harvested cellular product is too small. The second is the difficulty to perform biosafety testing such as integration site analysis and consequently to select cells with chosen integration sites, because adult HSCs cannot be replicated in vitro. The third limitation is that homologous recombination using current technologies is practically impossible thus compromising the advent of gene correction. All of these limitations are ultimately due to the fact that adult HSCs cannot be expanded in vitro without losing their stem cell potency. These limitations explain the critical importance of viral vectors such as gamma-retroviral and lentiviral vectors, which are remarkably quick and efficient in achieving stable gene transfer. This is essential when dealing with HSCs that are only available in limited quantities.

Use of ESs and induced pluripotent stem (iPS) cells for globin gene therapy is disclosed in Moi et al., Haematol Mar. 1, 2008; 93(3):325-330. Embryonic stem (ES) cells are amenable to gene targeting and correction, which requires unlimited in vitro cell division without losing multipotency. Chang et al., Proc Natl Acad Sci USA 2006; 103:1036-40 provided proof of principle of the feasibility of the homologous recombination approach in mice with sickle cell anemia. Takahashi et al. Cell 2006; 126:663-76 reported the successful reprogramming of fibroblasts to an embryonic stem-like state. Cells obtained by this reverse-differentiation process, called induced pluripotent stem (iPS) cells, were produced by exposing embryonic or young adult bulk fibroblast cultures to gamma-retroviral vectors encoding 4 transcription factors, which are physiologically active in the embryonic stem cells, but generally turned off when differentiation progresses. The cultured cells formed colonies similar to ES cell colonies. These findings have been confirmed and extended by others to both mouse and human fibroblasts (Meissner et al., Nat Biotechnol 2007; 25:1177-81; Nakagawa et al., Nat Biotechnol 2007; 26:101-6; Okita et al., Nature 2007; 448:313-7; Park et al., Nature 2007; 451:141-6; Takahashi et al., Nat Protoc 2007; 2:3081-9; Takahashi K et al., Cell 2007; 131:861-72; Wernig et al., Nature 2007; 448:318-24; Yu J et al., Science 2007; 318: 1917-20). Rudolf Jaenisch and co-workers achieved a successful gene therapy in a mouse model of sickle cell disease, using homologous recombination in ES-like iPS cells (Hanna et al., Science 2007; 318:1920-3). The process has so far been mostly applied to fibroblast harvested from a skin biopsy, which are then induced to become iPS by transduction with retroviral vectors that encode four stem cell transcription factors. iPS are amenable to the correction of the SC mutation by standard homologous recombination techniques and can then be differentiated in vitro into unlimited amounts of hematopoietic stem cells. The whole process ends with the autologous transplantation of the corrected HSC into the original mouse donor, which will now be cured of its SC disease. This technique is not only useful for homologous recombination, but can also enhance lentiviral-mediated globin gene transfer for the treatment of β-thalassemia by providing a means to perform detailed integration site analysis and adequate in vitro cell expansion before infusing cells into the recipient.

The cell of the presently disclosed subject matter can be autologous (“self”) or non-autologous (“non-self,” e.g., allogeneic, syngeneic or xenogeneic). As used herein, “autologous” refers to cells from the same subject. As used herein, “allogeneic” refers to cells of the same species that differ genetically to the cell in comparison. As used herein, “syngeneic” refers to cells of a different subject that are genetically identical to the cell in comparison. As used herein, “xenogeneic” refers to cells of a different species to the cell in comparison. In certain embodiments, the cell is autologous, e.g., a cell transduced with the presently disclosed expression cassette is administered to a subject from whom the cell is collected, e.g., the cell is collected from bone marrow, umbilical cord blood, peripheral blood, and/or adipose tissue of the subject. In certain embodiments, the cell is obtained or collected from bone marrow of a subject.

In certain embodiments, prior to transduction with the expression cassette, the cell is pre-stimulated, e.g., in the presence of one or more cytokines (e.g., IL-3, IL-1α, IL-6, Kit ligand (also known as “Stem Cell Factor (SCF)”), and Flt-3 ligand), and/or one or more glycoproteins (e.g., thrombopoietin and fibronectin). In one non-limiting example, the cell is pre-stimulated in the presence of Flt-3 ligand, SCF, thrombopoietin, interleukin-3, and fibronectin. The cell can be pre-stimulated for about 24 hours or longer, e.g., about 48 hours, or about 36 hours. Subsequently, the cell is transduced with a presently disclosed expression cassette, or a vector or another delivery system comprising such expression cassette. Transduction can be performed on a fresh cell, or on a frozen cell. Genomic DNA of the cell is isolated to determine the vector copy number and analyze the integration site or integrated vector structure, e.g., by South blot analysis and/or by Quantitative PCR. For quantification of globin mRNA (e.g., human β-globin transgene analysis), total RNA is extracted from the cell. Quantitative primer extension assay can be used for quantification of globin mRNA.

### V. Compositions and Formulations

The presently disclosed subject matter provides pharmaceutical compositions comprising a presently disclosed transduced cell as described above and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. The pharmaceutically acceptable carrier can be suitable for parenteral (e.g., intravenous, intramuscular, subcutaneous, or intraperitoneal), spinal or epidermal administration (e.g., by injection, infusion or implantation). Depending on the route of administration, the active compound, e.g., the transduced cell, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the invention is contemplated.

The pharmaceutical compositions of the presently disclosed subject matter can further comprise one or more polypeptides, polynucleotides, vectors comprising the same, transduced cells, etc., as described herein, formulated in pharmaceutically-acceptable or physiologically-acceptable solutions for administration to a cell or an animal, either alone, or in combination with one or more other modalities of therapy. If desired, the pharmaceutical compositions of the presently disclosed subject matter can be administered in combination with other agents, including, but not limited to, cytokines, growth factors, hormones, small molecules or various pharmaceutically-active agents. Any additional agents that do not adversely affect the ability of the composition to deliver the intended gene therapy can be included in the compositions.

In the pharmaceutical compositions of the presently disclosed subject matter, formulation of pharmaceutically-acceptable excipients and carrier solutions is well known to those of ordinary skill in the art, as is the development of suitable dosing and treatment regimens for using the particular compositions described herein in a variety of treatment regimens, including, e.g., oral, parenteral, intravenous, intranasal, and intramuscular administration and formulation.

The pharmaceutical compositions of the presently disclosed subject matter can be delivered parenterally (e.g., intravenously, intramuscularly, or intraperitoneally) as described, for example, in U.S. Pat. Nos. 5,543,158; 5,641,515 and 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

The pharmaceutical compositions of the presently disclosed subject matter can be conveniently provided as sterile liquid preparations, e.g., isotonic aqueous solutions, suspensions, emulsions, dispersions, or viscous compositions, which can be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

The compositions can be isotonic, i.e., they can have the same osmotic pressure as blood and lacrimal fluid. The desired isotonicity of the compositions of the presently disclosed subject matter can be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

For parenteral administration in an aqueous solution, for example, the solution should be suitably buffered if necessary and the liquid diluent first rendered isotonic with sufficient saline or glucose. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

In certain embodiments, the compositions can be delivered by intranasal sprays, inhalation, and/or other aerosol delivery vehicles. Methods for delivering genes, polynucleotides, and peptide compositions directly to the lungs via nasal aerosol sprays are described, e.g., in U.S. Pat. Nos. 5,756,353 and 5,804,212. Methods of delivering drugs using lysophosphatidyl-glycerol compounds are described, e.g., in U.S. Pat. No. 5,725,871. Transmucosal drug delivery in the form of a polytetrafluoroethylene support matrix is described, e.g., in U.S. Pat. No. 5,780,045. The compositions of the presently disclosed subject matter can be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the presently disclosed subject matter can comprise one or more repressors and/or activators comprising a combination of any number of polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy.

In certain aspects, the presently disclosed subject matter provides formulations or compositions suitable for the delivery of viral vector systems (i.e., viral-mediated transduction) including, but not limited to, retroviral (e.g., lentiviral) vectors. Exemplary formulations for ex vivo delivery can also include the use of various transfection agents known in the art, such as calcium phosphate, electroporation, heat shock and various liposome formulations (i.e., lipid-mediated transfection). Liposomes are lipid bilayers entrapping a fraction of aqueous fluid. DNA spontaneously associates to the external surface of cationic liposomes (by virtue of its charge) and these liposomes will interact with the cell membrane.

The skilled artisan can readily determine the amount of cells and optional additives, vehicles, and/or carrier in compositions and to be administered in methods of the presently disclosed subject matter. Typically, any additives (in addition to the transduced cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### VI. Uses and Methods

Vectors and other delivery systems (nucleases and CRISPR-Cas systems) comprising the presently disclosed expression cassette provide improved methods of gene therapy. As used herein, the term “gene therapy” refers to the introduction of a polynucleotide into a cell's genome that restores, corrects, or modifies the gene and/or expression of the gene. In various non-limiting embodiments, a presently disclosed vector or other delivery system (e.g., a nuclease or a CRISPR-Cas system) comprises an expression cassette comprising a globin gene or a functional portion thereof that encodes a globin protein (e.g., human R globin protein), which provides curative, preventative, or ameliorative benefits to a subject diagnosed with or that is suspected of having a disease, disorder, or condition of the hematopoietic system. The vector or other delivery systems (e.g., a nuclease and the CRISPR-Cas system) can infect and transduce the cell in vivo, ex vivo, or in vitro. In ex vivo and in vitro embodiments, the transduced cells can then be administered to a subject in need of therapy. The presently disclosed subject matter contemplates that the vectors and other delivery systems (e.g., nucleases or CRISPR-Cas systems), viral particles, and transduced cells of the presently disclosed subject matter are be used to treat, prevent, and/or ameliorate a disease, disorder, or condition of the hematopoietic system in a subject, e.g., a hemoglobinopathy.

As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

As used herein, “thalassemia” refers to a hereditary disorder characterized by defective production of hemoglobin. Examples of thalassemias include α- and β-thalassemia. β-thalassemias are caused by a mutation in the beta globin chain, and can occur in a major or minor form. In the major form of β-thalassemia, children are normal at birth, but develop anemia during the first year of life. The mild form of β-thalassemia produces small red blood cells and the thalassemias are caused by deletion of a gene or genes from the globin chain. α-thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles. Hb Bart syndrome, the most severe form of a thalassemia, results from the loss of all four α-globin alleles. HbH disease is caused by a loss of three of the four [alpha]-globin alleles. In these two conditions, a shortage of [alpha]-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with a thalassemia.

As used herein, the term “sickle cell disease” refers to a group of autosomal recessive genetic blood disorders, which results from mutations in a globin gene and which is characterized by red blood cells that assume an abnormal, rigid, sickle shape. They are defined by the presence of β^{S}-gene coding for a β-globin chain variant in which glutamic acid is substituted by valine at amino acid position 6 of the peptide, and second β-gene that has a mutation that allows for the crystallization of HbS leading to a clinical phenotype. As used herein, the term “sickle cell anemia” refers to a specific form of sickle cell disease in patients who are homozygous for the mutation that causes HbS. Other common forms of sickle cell disease include HbS/β-thalassemia, HbS/HbC and HbS/HbD.

In certain embodiments, gene therapy methods of the presently disclosed subject mater are used to treat, prevent, or ameliorate a hemoglobinopathy that is selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In one non-limiting embodiment, the hemoglobinopathy is β-thalassemia. In another non-limiting embodiment, the hemoglobinopathy is sickle cell anemia

In various non-limiting embodiments, vectors or other delivery systems (e.g., nucleases or CRISPR-Cas systems) comprising a presently disclosed expression cassette are administered by direct injection to a cell, tissue, or organ of a subject in need of gene therapy, in vivo. In various other embodiments, cells are transduced in vitro or ex vivo with vectors or other delivery systems (e.g., nucleases or CRISPR-Cas systems) of the presently disclosed subject matter, and optionally expanded ex vivo. The transduced cells are then administered to a subject in need of gene therapy, e.g., within a pharmaceutical formulation disclosed herein.

The presently disclosed subject matter provides a method of providing a transduced cell to a subject. In various non-limiting embodiments, the method comprises administering (e.g., parenterally) one or more cells (a population of cells) transduced with a presently disclosed expression cassette or a vector or another delivery system (e.g., a nuclease or CRISPR-Cas system) comprising such expression cassette to the subject.

The presently disclosed subject matter provides a method of treating a hemoglobinopathy in a subject. In various non-limiting embodiments, the method comprises administering an effective amount of a presently disclosed transduced cell or a population of the presently disclosed transduced cells (e.g., HSCs, embryonic stem cells, or iPSCs) to the subject.

For treatment, the amount administered is an amount effective in producing the desired effect. An effective amount can be provided in one or a series of administrations. An effective amount can be provided in a bolus or by continuous perfusion. An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

In one non-limiting example, following administration of one or more of the presently disclosed transduced cells, peripheral blood of the subject is collected and hemoglobin levels is measured. A therapeutically relevant level of hemoglobin is produced following administration of one or more of the presently disclosed transduced cells. Therapeutically relevant level of hemoglobin is a level of hemoglobin that is sufficient (1) to improve or correct anemia, (2) to restore the ability of the subject to produce red blood cells containing normal hemoglobin, (3) to correct ineffective erythropoiesis in the subject, (4) to correct extra-medullary hematopoiesis (e.g., splenic and hepatic extra-medullary hematopoiesis), and/or (5) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dL Hb, from about 11.5 g/dL Hb to about 12 g/dL Hb, from about 12 g/dL Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb. In certain embodiments, the therapeutically relevant level of hemoglobin is maintained in the subject for at least about 6 months, for at least about 12 months (or 1 year), for at least about 24 months (or 2 years). In certain embodiments, the therapeutically relevant level of hemoglobin is maintained in the subject for up to about 6 months, for up to about 12 months (or 1 year), for up to about 24 months (or 2 years). In certain embodiments, the therapeutically relevant level of hemoglobin is maintained in the subject for about 6 months, for about 12 months (or 1 year), for about 24 months (or 2 years). In certain embodiments, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain embodiments, the method comprises administering one or more cells transduced with a recombinant vector comprising a presently disclosed expression cassette as described above. The vector copy number of the recombinant vector in the cells that provide for the therapeutically relevant level of hemoglobin (e.g., 9-10 g/dL) in the subject is from about 0.5 to about 2, from about 0.5 to about 1, or from about 1 to about 2 vector copy number per cell. In certain embodiments, the vector copy number of the presently disclosed vector is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 vector copy number per cell.

In certain embodiments, the subject lacks a human leukocyte antigen (HLA)-matched donor. In certain embodiments, the transduced cell is from the same subject. In one embodiment, the transduced cell is from bone marrow of the same subject. Thus, administration of the transduced cells do not incur the risk of graft-versus host disease in the subject. The method does not require immune suppression to prevent graft rejection, e.g., the method does not comprise administering an immunosuppressive agent to the subject.

The present disclosed subject matter also provides a method of increasing the proportion of red blood cells or erythrocytes compared to white blood cells or leukocytes in a subject. In various non-limiting embodiments, the method comprises administering an effective amount of a presently disclosed transduced cell or a population of the presently disclosed transduced cells (e.g., HSCs, embryonic stem cells, or iPSCs) to the subject, wherein the proportion of red blood cell progeny cells of the hematopoietic stem cells are increased compared to white blood cell progeny cells of the hematopoietic stem cells in the subject.

Without wishing to be bound to any particular theory, an important advantage provided by the expression cassette, vectors and other delivery systems (e.g., nucleases and CRISPR-Cas systems), compositions, and methods of the presently disclosed subject is the high efficacy of globin gene therapy that can be achieved by administering populations of cells comprising lower percentages of transduced cells compared to existing methods. This provides important safety advantages associated with reduced chances of deleterious mutation, transformation, or oncogene activation of cellular genes in transduced cells. The transduced cells can be administered as part of a bone marrow or cord blood transplant in an individual that has or has not undergone bone marrow ablative therapy.

One consideration concerning the therapeutic use of the presently disclosed cells transduced with the expression cassette described herein (“transduced cells”) is the quantity of cells necessary to achieve an optimal effect. The quantity of transduced cells to be administered will vary for the subject being treated. In one embodiment, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed transduced cells are administered to a subject. More effective cells may be administered in even smaller numbers. In some embodiments, at least about 1×10⁸ cells/kg, at least about 2×10⁸ cells/kg, at least about 3×10⁸ cells/kg, at least about 4×10⁸ cells/kg, or at least about 5×10⁸ cells/kg of the presently disclosed transduced cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

In various embodiments, the expression cassettes, vectors and other delivery systems (nucleases and CRISPR-Cas systems), compositions, and methods of the presently disclosed subject matter offer improved methods of gene therapy using ex vivo gene therapy and autologous transplantation. Transplantation of cells transduced with the expression cassette or into subjects having a hemoglobinopathy results in long-term correction of the disease.

One or more presently disclosed transduced cells can be administered by any methods known in the art, including, but not limited to, parenteral administration (e.g., intramuscular administration, intravenous administration, subcutaneous administration, or intraperitoneal administration), spinal administration, and epidermal administration. In one non-limiting embodiment, one or more transduced cells are delivered to a subject intravenously. One or more presently disclosed transduced cells can be administered by injection, infusion, or implantation. In one non-limiting embodiment, one or more transduced cells are administered by injection. In another non-limiting embodiment, one or more transduced cells are administered by intravenous injection.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

### VII. Kits

The presently disclosed subject matter provides kits for the treatment or prevention of a hemoglobinopathy. In one embodiment, the kit comprises a therapeutic or prophylactic composition containing an effective amount of a cell transduced with the presently disclosed expression cassette in unit dosage form. In one non-limiting embodiment, the kit comprises one or more expression cassettes disclosed herein. In certain embodiments, the kit comprises one or more vectors comprising an expression cassette disclosed herein. In some embodiments, the kit comprises a sterile container, which can be a box, an ampule, a bottle, a vial, a tube, a bag, a pouch, a blister-pack, or other suitable container forms known in the art. Such containers can be made of plastic, glass, laminated paper, metal foil, or other materials suitable for holding medicaments.

If desired, the transduced cell is provided together with instructions for administering the cell to a subject having or at risk of developing a hemoglobinopathy. The instructions will generally include information about the use of the composition for the treatment or prevention of a hemoglobinopathy. In other embodiments, the instructions include at least one of the following: description of the therapeutic agent; dosage schedule and administration for treatment or prevention of a hemoglobinopathy or symptoms thereof, precautions; warnings; indications; counter-indications; overdosage information; adverse reactions; animal pharmacology; clinical studies; and/or references. Alternatively or additionally, the kit can include instructions for transducing a cell with the one or more expression cassettes and/or vectors comprising such expression cassettes. The instructions may be printed directly on the container (when present), or as a label applied to the container, or as a separate sheet, pamphlet, card, or folder supplied in or with the container.

### EXAMPLES

The practice of the presently disclosed subject matter employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the presently disclosed subject matter, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the expression cassettes, vectors, delivery systems, and therapeutic methods of the presently disclosed subject matter, and are not intended to limit the scope of what the inventors regard as their invention.

### Example 1: Discovery of Novel Insulators

The problems created by insertional mutagenesis of viral vectors are widely known (Nienhuis (2013), Baum et al. (2006), Nienhuis et al. (2006)) as is the evidence that the risks of genotoxicity can be reduced by the use of chromatin insulators (Arumugam et al. (2007), Emery (2011), Evans-Galea et al. (2007), Rivella et al. (2000), Emery et al. (2000), Emery et al. (2002), Yannaki et al. (2002), Hino et al. (2004), Ramezani et al. (2003), Ramezani et al. (2008)). Approaches allowing the efficient identification of enhancer blocking insulators in the human genome have been developed. These new insulators are short, on the average 150 bp, and they do not affect adversely the titers of viral vectors and they are several times more powerful than the insulator cHS4. Genomic approaches were used to discover the most powerful enhancer blocker and barrier insulators of the human genome. For gene therapy of the hemoglobinopathies, powerful enhancers are required to achieve therapeutic levels of globin gene expression. Powerful insulators may thus provide one means to protect the genomic environment from the powerful enhancers of the integrating vectors.

Several studies have demonstrated the ability of the cHS4 insulator to reduce position-effect silencing of gammaretroviral vectors (Evans-Galea et al. (2007), Rivella et al. (2000), Emery et al. (2000), Emery et al. (2002), Yannaki et al. (2002), Hino et al. (2004), Ramezani et al. (2006), Yao et al. (2003), Nishino et al. (2006), Aker et al. (2007), Li and Emery (2008)), and lentiviral vectors (Evans-Galea et al. (2007), Ramezani et al. (2003), Puthenveetil et al. (2004), Arumugam et al. (2007), Bank et al. (2005), Aker et al. (2007), Ma et al. (2003), Chang et al. (2005), Pluta et al. (2005)). Those studies that were appropriately designed demonstrated that inclusion of the 1.2 kb version of the cHS4 insulator increased the likelihood and/or consistency of vector transgene expression in at least some settings (Arumugam et al. (2007), Evans-Galea et al. (2007), Emery et al. (2002), Yannaki et al. (2002), Hino et al. (2004), Ramezani et al. (2006), Aker et al. (2007), Li and Emery (2008), Pluta et al. (2005), Jakobsson et al. (2004)). Nevertheless, the degree of protection afforded by the cHS4 insulator is far from complete. In addition, the inclusion of the 1.2 Kb cHS4 can adversely affect vector titers while the smallest cHS4 core has been proven ineffective (Aker et al. (2007), Jakobsson et al. (2004)).

Effects on genotoxicity were tested using an in vivo assay based on quantitation of tumor formation in mice. Vectors insulated by insulator A1 decreased tumor formation induced by random vector integration in hematopoietic chimeras compared to mice that received uninsulated or cHS4-insulated controls.

To assess effects on vector titers, insulator A1 was introduced into the double-copy region of a third-generation lentiviral vector expressing GFP from a constitutive package promoter, and the viral titers and GFP expression were measured. Insulator A1 did not affect adversely vector GFP expression.

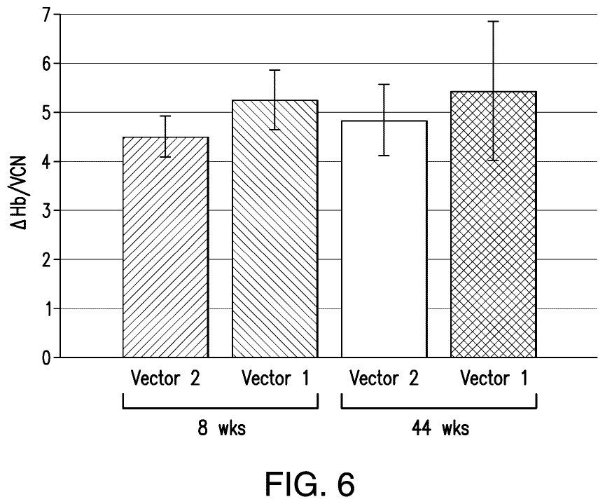

In the in vivo genotoxicity assay, a cell line transduced with gammaretroviral vectors produced tumors after transplantation in mice and allowed quantitation of genotoxic effects by measuring rates of tumor free survival. Effects of an insulator on genotoxicity were quantitated by the number of tumors formed in the mice and the rates of tumor free survival. Insulator A1 was inserted in the proximal portion of the 3′ LTR, from which it is copied into the 5′ LTR during reverse transcription and vector integration. The resulting topology places copies of the insulator between the genomic regions located 5′ and 3′ of the integrated provirus and enhancer activity from the 5′ viral LTR and internal Pgk promoter, but does not contain the enhancer in the 3′ LTR. This can decrease genotoxicity thus resulting in decreased tumor formation and increased survival of the animals. Gamma-retroviral reporter vectors flanked with insulator A1 or control regions were used to transduce the growth factor-dependent cell line 32D, and 10 independent sub-pools for each vector were transplanted into syngeneic C3H/HeJ mice. All 10 mice transplanted with mock-transduced cells remained free of 32D cell-derived tumors, while nearly all mice transplanted with 32D cells transduced with vectors containing no inserts or a 790 bp neutral spacer developed tumors within a median of 16 weeks (FIG. 5B). Flanking this vector with the cHS4 insulator delayed the onset of tumor formation by several weeks, and reduced the frequency of animals that developed tumors to 6 of 10. In contrast, only two of 10 animals developed tumors following transplantation with 32D cells transduced with the vector flanked with insulator A1 (FIG. 5B). The frequency of animals with tumors and the number of vector transduction events in the original sub-pools suggested that flanking the vector with insulator A1 reduced the overall rate of tumor formation 12-fold, from 46.9 tumors per 105 provirus to 3.9 tumors per 105 provirus (FIG. 5C). In comparison, the cHS4 insulator reduced the overall rate of tumor formation 2.8-fold (to 16.9 tumors per 105 provirus), while the neutral spacer had no statistically discernable effect on the rate of tumor formation. These results indicate that the discovered enhancer blocking insulators can decrease substantially the risks of insertional mutagenesis and genotoxicity.

### Example 2: Characterization of Globin Vectors Comprising at Least One Insulator

A presently disclosed expression cassette (designated as “Expression Cassette 1”; as shown in FIG. 1), which comprises insulator A1, and a human β^{A}-globin gene encoding a threonine to glutamine mutation at codon 87 (β^{A-T87Q}) operably linked to a β-globin LCR region comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7, was generated. The rationale for using the variant β chain (β^{A}) is to facilitate the detection of the vector-encoded β-globin gene, distinguishing it from endogenous or transfused beta chains. The glutamine (GLN) residue at position 87 in the γ-globin chain augments the anti-sickling activity of the gamma chain relative to the β chain, while preserving adult oxygen-binding characteristics of the β chain (Nagel et al. (1979)). In Vector 1, a point mutation altering codon 87 (β^{A-T87Q}, or β87) replaces the normal threonine with glutamine and augments anti-sickling activity of the vector-encoded β chain. This 087 chain has been safely used in a patient with HbE-thalassemia (Cavazzana-Calvo et al. (2010)).

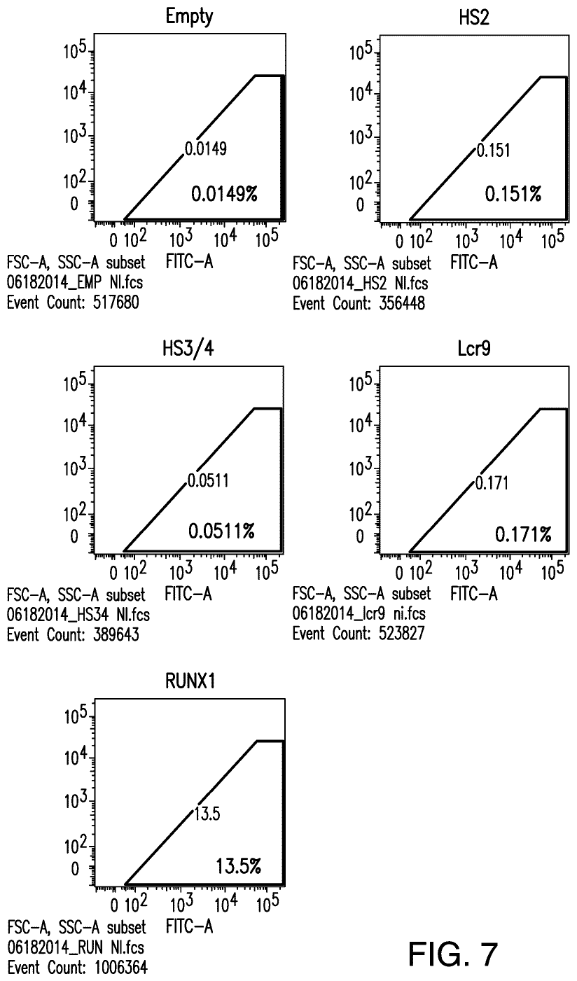

Expression cassette 1 was incorporated or introduced to a lentivirus vector (designated as “Vector 1”). Vector 1 was introduced in bone marrow cells of C57BL/6-Hbb th3/+ mice and transplanted to syngeneic lethally irradiated recipients as previously described (May et al. (2000), May et al. (2002), Lisowski et al. (2007)). The vector titer of V1 was comparable to that of a lentivirus vector comprising an expression cassette lacking insulator A1. The β-globin expression of Vector 1 was compared to that of a lentivirus vector (designated as “Vector 2”) comprising an expression cassette that lacks an insulator and comprises a wild human β-globin gene operably linked to a β-globin LCR region comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:6. In comparison to Vector 2, β-globin expression of Vector 1 normalized to vector copy was equivalent or slightly increased, suggesting an added benefit for in vivo expression provided by the flanking barrier elements, as shown in FIG. 6.

### Example 3: Evaluation of Enhancer Activity in Non-Erythroid K562 Cells

The enhancer activity of HS2 was evaluated in Non-erythroid K562 Cells. As shown in FIG. 7, GFP expression in K562 cells transduced with vectors driven by a minimal promoter linked to no enhancer (“Empty”, HS2, HS3-4, HS2-3-4 or the runx1 enhancer used as positive control (“RUNX1”). Background expression was on the order or 0.01% (“empty), but increased over 10-fold with HS2-3-4 (“Lcr9”, 0.17%). This enhancement was mostly due to HS2 (0.15%) but not HS3-4 (0.05%). All cell lines were comparably transduced (mean vector copy number 2.5). The results support that HS2 but not HS3-HS4 may pose an oncogenic risk in non-erythroid hematopoietic stem and progenitor cells.

### Example 4: Novel Erythroid-Specific Enhancers

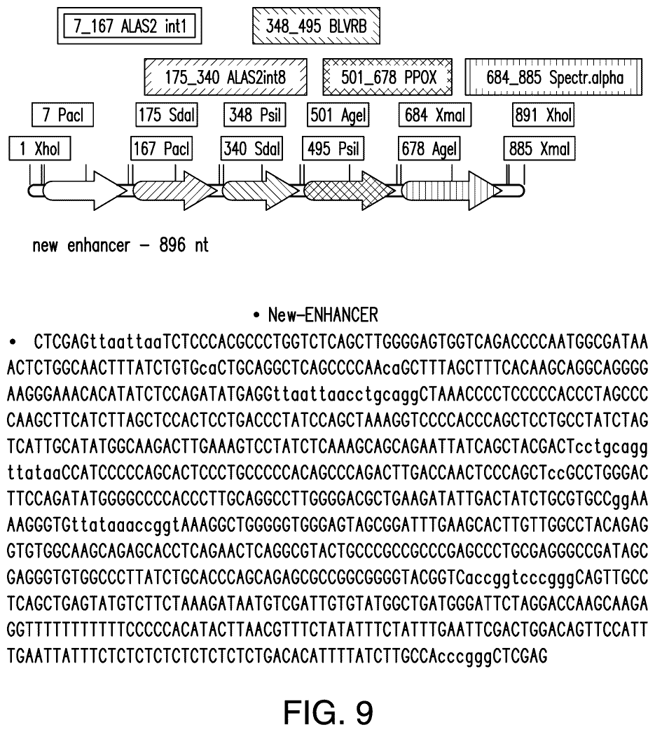

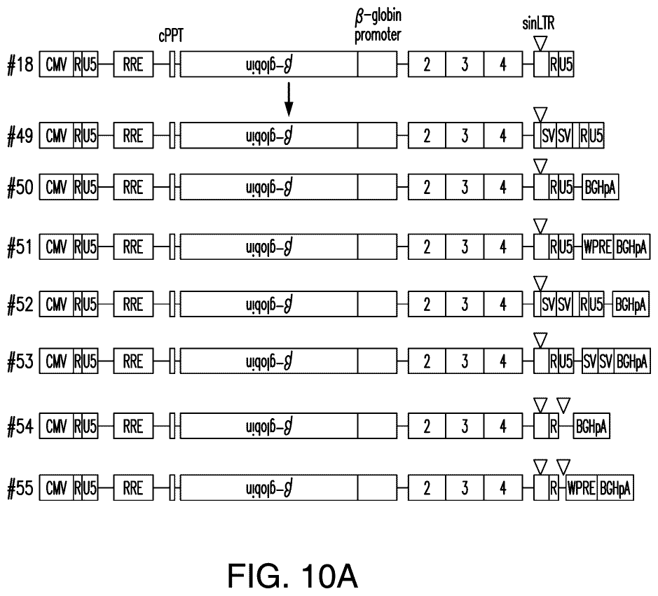

As shown in FIGS. 8 and 9, five erythroid-specific enhancers were substituted for HS2: ALAS Intron 1, ALAS Intron 8, BLVRB, PPOX, and Spectrin-alpha. The inventors have shown that all these five enhancers are powerful enhancers, and lack enhancer activity in non-erythroid tissues, and do not reduce the vector titer.

### Example 5: Increasing Globin Lentiviral Vector Production Through 3′ LTR Modifications

An essential feature of therapeutic globin vectors is to achieve a high titer, sufficient for effective transduction of patient cells. By virtue of their large cargo, comprising a gene, promoter, enhancers and/or LCR elements, globin lentiviral vectors inherently have low titer, complicating their manufacture and limiting their clinical use. This problem is further compounded by the incorporation of additional genomic elements such as an insulator, which further increase the size of the vector.

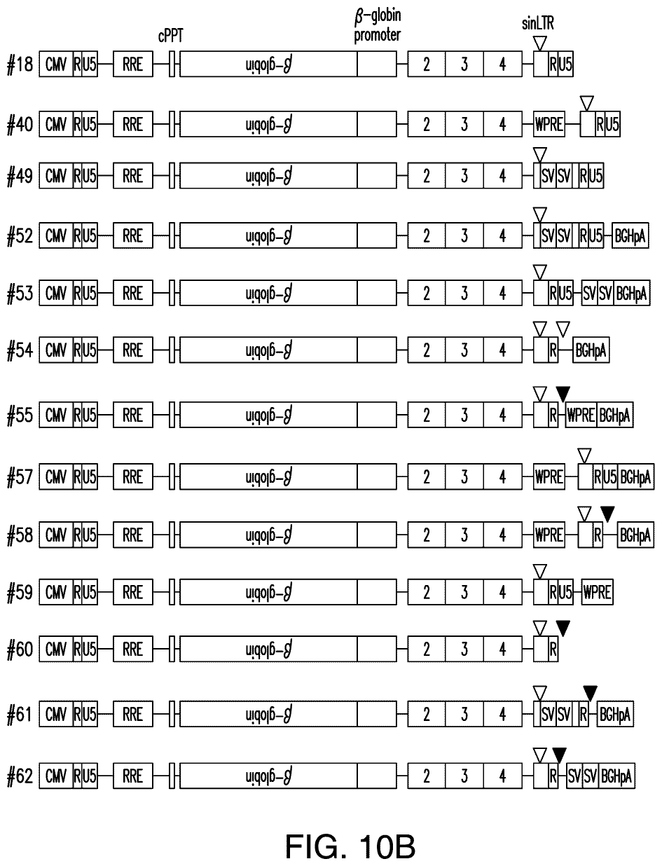

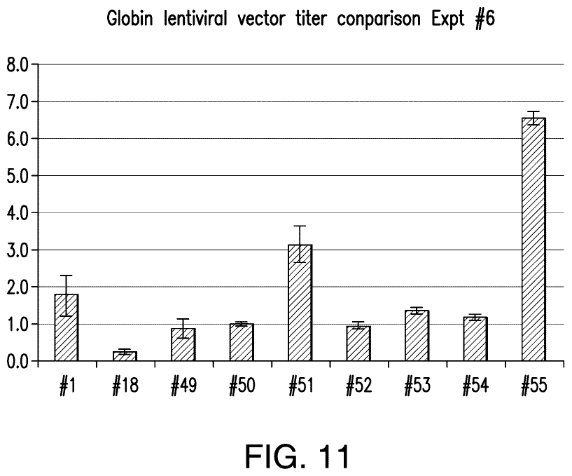

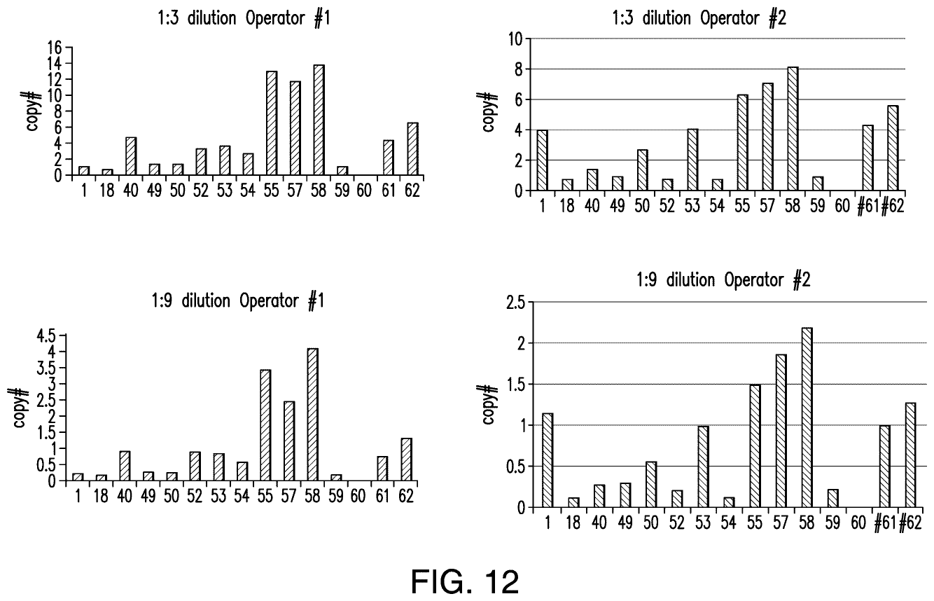

The inventors explored different modifications of the 3′ long terminal repeat (LTR) of globin vectors to increase the titer of globin vectors. Over 62 variations were evaluated, numbered 1 through 62, modeled on a lentivirus vector comprising a human β-globin gene operably linked to a β-globin LCR region comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7. In other words, all of Vectors #1 through Vector 62 comprise a β-globin LCR region comprising a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7. Vector #18 served as a baseline, comprising a standard U3 deletion in the 3′LTR. Vector #1 (not depicted) comprised a full, i.e., wild-type LTR, which cannot be used clinically. Modifications to the 3′LTR are depicted in FIGS. 10A and 10B, and their titers shown in FIGS. 11 and 12 (the Y axis shows the vector copy number of vector stocks manufactured and tested under strictly identical conditions). Titrations were measured in triple replicas, performed in parallel by two operators, and repeated in multiple experiments.

As shown in FIGS. 11 and 12, Vector #55 repeatedly showed a higher titer. This vector comprises a Woodchuck hepatitis post-regulatory element (WPRE) and a bovine growth hormone polyadenylation signal 3′ to the R region in the 3′ LTR. The WPRE element is therefore not transferred to the transduced cells.

The incorporation of these elements for enhancing the production of globin lentiviral vectors is essential to yield higher titers and hence for the clinical usefulness of the vectors described in this application.

### REFERENCES

1. Weatherall, D. J. & Clegg, J. B. The Thalassemia Syndrome. Blackwell Scientific Oxford (1981).
2. Stamatoyannopoulos, G., Nienhuis, A. W., Majerus, P. & Varmus, H. The Molecular Basis of Blood Diseases. WB Saunders, Philadelphia (1994).
3. Weatherall, D. J. Phenotype-genotype relationships in monogenic disease: lessons from the thalassemias. Nat Rev Genet 2, 245-255. (2001).
4. Steinberg, M. H., Forget, B. G., Higgs, D. R. & Nagel, R. L. Molecular Mechanism of β Thalassemia; Bernard G. Forget, (Cambridge University Press, Cambridge, UK, 2001).
5. Cooley, T. B. & Lee, P. A series of cases of splenomegaly in children with anemia and peculiar bone changes. Trans. Am. Pediatr. Soc. 37, 29 (1925).
6. Giardina, P. J. & Grady, R. W. Chelation therapy in beta-thalassemia: an optimistic update. Semin Hematol 38, 360-366. (2001).
7. Giardini, C. & Lucarelli, G. Bone marrow transplantation in the treatment of thalassemia. Current opinion in hematology 1, 170-176. (1994).
8. Boulad, F., Giardina, P., Gillio, A., Kernan, N., Small, T., Brochstein, J., Van Syckle, K., George, D., Szabolcs, P. & O'Reilly, R. J. Bone marrow transplantation for homozygous beta-thalassemia. The Memorial Sloan-Kettering Cancer Center experience. Ann NY Acad Sci 850, 498-502. (1998).
9. Lucarelli, G., Clift, R. A., Galimberti, M., Angelucci, E., Giardini, C., Baronciani, D., Polchi, P., Andreani, M., Gaziev, D., Erer, B., Ciaroni, A., D'Adamo, F., Albertini, F. & Muretto, P. Bone marrow transplantation in adult thalassemic patients. Blood 93, 1164-1167. (1999).
10. Tisdale, J. & Sadelain, M. Toward gene therapy for disorders of globin synthesis. Semin Hematol 38, 382-392 (2001).
11. Pauling, L., Itano, H. A., Singer, S. J. & Wells, I. C. Sickle cell anemia, a molecular disease. Science 110, 543-546 (1949).
12. Swank, R. A. & Stamatoyannopoulos, G. Fetal gene reactivation. Curr Opin Genet Dev 8, 366-370 (1998).
13. Platt, O. S., Orkin, S. H., Dover, G., Beardsley, G. P., Miller, B. & Nathan, D. G. Hydroxyurea enhances fetal hemoglobin production in sickle cell anemia. J Clin Invest 74, 652-656. (1984).
14. Charache, S., Dover, G. J., Moore, R. D., Eckert, S., Ballas, S. K., Koshy, M., Milner, P. F., Orringer, E. P., Phillips, G., Jr., Platt, O. S. & et al. Hydroxyurea: effects on hemoglobin F production in patients with sickle cell anemia. Blood 79, 2555-2565. (1992).
15. Atweh, G. F. & Loukopoulos, D. Pharmacological induction of fetal hemoglobin in sickle cell disease and beta-thalassemia. Semin Hematol 38, 367-373. (2001).
16. Perrine, S. P., Castaneda, S. A., Boosalis, M. S., White, G. L., Jones, B. M. & Bohacek, R. Induction of fetal globin in beta-thalassemia: Cellular obstacles and molecular progress. Ann N Y Acad Sci 1054, 257-265 (2005).
17. Stamatoyannopoulos, G. Prospects for developing a molecular cure for thalassemia. Hematology 10 Suppl 1, 255-257 (2005).
18. Vermylen, C., Cornu, G., Ferster, A., Brichard, B., Ninane, J., Ferrant, A., Zenebergh, A., Maes, P., Dhooge, C., Benoit, Y., Beguin, Y., Dresse, M. F. & Sariban, E. Haematopoietic stem cell transplantation for sickle cell anaemia: the first 50 patients transplanted in Belgium. Bone Marrow Transplant 22, 1-6 (1998).
19. Luzzatto, L. & Goodfellow, P. Sickle cell anaemia. A simple disease with no cure. Nature 337, 17-18 (1989).
20. Sadelain, M. Genetic treatment of the hemoglobinopathies: recombinations and new combinations. Br J Haematol 98, 247-253 (1997).
21. Sadelain, M., Boulad, F., Galanello, R., Giardina, P., Locatelli, F., Maggio, A., Rivella, S., Riviere, I. & Tisdale, J. Therapeutic options for patients with severe beta-thalassemia: the need for globin gene therapy. Hum Gene Ther 18, 1-9 (2007).
22. Borgna-Pignatti, C., Rugolotto, S., De Stefano, P., Zhao, H., Cappellini, M. D., Del Vecchio, G. C., Romeo, M. A., Forni, G. L., Gamberini, M. R., Ghilardi, R., Piga, A. & Cnaan, A. Survival and complications in patients with thalassemia major treated with transfusion and deferoxamine. Haematologica 89, 1187-1193 (2004).
23. Telfer, P. T., Warburton, F., Christou, S., Hadjigavriel, M., Sitarou, M., Kolnagou, A. & Angastiniotis, M. Improved survival in thalassemia major patients on switching from desferrioxamine to combined chelation therapy with desferrioxamine and deferiprone. Haematologica 94, 1777-1778 (2009).
24. Ladis, V., Chouliaras, G., Berdoukas, V., Chatziliami, A., Fragodimitri, C., Karabatsos, F., Youssef, J., Kattamis, A. & Karagiorga-Lagana, M. Survival in a large cohort of Greek patients with transfusion-dependent beta thalassaemia and mortality ratios compared to the general population. European journal of haematology 86, 332-338 (2011).
25. Mancuso, A., Sciarrino, E., Renda, M. C. & Maggio, A. A prospective study of hepatocellular carcinoma incidence in thalassemia. Hemoglobin 30, 119-124 (2006).
26. Persons, D. A. & Tisdale, J. F. Gene therapy for the hemoglobin disorders. Semin Hematol 41, 279-286 (2004).
27. Sadelain, M. Recent advances in globin gene transfer for the treatment of beta-thalassemia and sickle cell anemia. Current opinion in hematology 13, 142-148 (2006).
28. May, C., Rivella, S., Callegari, J., Heller, G., Gaensler, K. M., Luzzatto, L. & Sadelain, M. Therapeutic haemoglobin synthesis in beta-thalassemic mice expressing lentivirus-encoded human beta-globin. Nature 406, 82-86 (2000).
29. May, C., Rivella, S., Chadburn, A. & Sadelain, M. Successful treatment of murine beta-thalassemia intermedia by transfer of the human beta-globin gene. Blood 99, 1902-1908 (2002).
30. Rivella, S., May, C., Chadburn, A., Riviere, I. & Sadelain, M. A novel murine model of Cooley anemia and its rescue by lentiviral-mediated human beta-globin gene transfer. Blood 101, 2932-2939 (2003).
31. Sadelain, M., Boulad, F., Lisowki, L., Moi, P. & Riviere, I. Stem cell engineering for the treatment of severe hemoglobinopathies. Curr Mol Med 8, 690-697 (2008).
32. Bank, A., Dorazio, R. & Leboulch, P. A phase I/II clinical trial of beta-globin gene therapy for beta-thalassemia. Ann NY Acad Sci 1054, 308-316 (2005).
33. Cavazzana-Calvo, M., Payen, E., Negre, O., Wang, G., Hehir, K., Fusil, F., Down, J., Denaro, M., Brady, T., Westerman, K., Cavallesco, R., Gillet-Legrand, B., Caccavelli, L., Sgarra, R., Maouche-Chretien, L., Bernaudin, F., Girot, R., Dorazio, R., Mulder, G. J., Polack, A., Bank, A., Soulier, J., Larghero, J., Kabbara, N., Dalle, B., Gourmel, B., Socie, G., Chretien, S., Cartier, N., Aubourg, P., Fischer, A., Cornetta, K., Galacteros, F., Beuzard, Y., Gluckman, E., Bushman, F., Hacein-Bey-Abina, S. & Leboulch, P. Transfusion independence and HMGA2 activation after gene therapy of human beta-thalassaemia. Nature 467, 318-322 (2010).
34. Braun, C. J., Boztug, K., Paruzynski, A., Witzel, M., Schwarzer, A., Rothe, M., Modlich, U., Beier, R., Gohring, G., Steinemann, D., Fronza, R., Ball, C. R., Haemmerle, R., Naundorf, S., Kuhlcke, K., Rose, M., Fraser, C., Mathias, L., Ferrari, R., Abboud, M R., Al-Herz, W., Kondratenko, I., Marodi, L., Glimm, H., Schlegelberger, B., Schambach, A., Albert, M. H., Schmidt, M., von Kalle, C. & Klein, C. Gene therapy for Wiskott-Aldrich syndrome—long-term efficacy and genotoxicity. Sci Transl Med 6, 227ra233 (2014).
35. Chang, A. H. & Sadelain, M. The genetic engineering of hematopoietic stem cells: the rise of lentiviral vectors, the conundrum of the ltr, and the promise of lineage-restricted vectors. Mol Ther 15, 445-456 (2007).
36. Pawliuk, R., Westerman, K. A., Fabry, M. E., Payen, E., Tighe, R., Bouhassira, E. E., Acharya, S. A., Ellis, J., London, I. M., Eaves, C. J., Humphries, R. K., Beuzard, Y., Nagel, R. L. & Leboulch, P. Correction of sickle cell disease in transgenic mouse models by gene therapy. Science 294, 2368-2371 (2001).
37. Emery, D. W., Chen, H., Li, Q. & Stamatoyannopoulos, G. Development of a condensed locus control region cassette and testing in retrovirus vectors for A gamma-globin. Blood Cells Mol Dis 24, 322-339 (1998).
38. Miccio, A., Cesari, R., Lotti, F., Rossi, C., Sanvito, F., Ponzoni, M., Routledge, S. J., Chow, C. M., Antoniou, M. N. & Ferrari, G. In vivo selection of genetically modified erythroblastic progenitors leads to long-term correction of beta-thalassemia. Proc Natl Acad Sci USA 105, 10547-10552 (2008).
39. Sadelain, M., Wang, C. H., Antoniou, M., Grosveld, F. & Mulligan, R. C. Generation of a high-titer retroviral vector capable of expressing high levels of the human beta-globin gene. Proc Natl Acad Sci USA 92, 6728-6732 (1995).
40. Samakoglu, S., Lisowski, L., Budak-Alpdogan, T., Usachenko, Y., Acuto, S., Di Marzo, R., Maggio, A., Zhu, P., Tisdale, J. F., Riviere, I. & Sadelain, M. A genetic strategy to treat sickle cell anemia by coregulating globin transgene expression and RNA interference. Nat Biotechnol 24, 89-94 (2006).
41. Pestina, T. I., Hargrove, P. W., Jay, D., Gray, J. T., Boyd, K. M. & Persons, D. A. Correction of murine sickle cell disease using gamma-globin lentiviral vectors to mediate high-level expression of fetal hemoglobin. Mol Ther 17, 245-252 (2009).
42. Hanawa, H., Yamamoto, M., Zhao, H., Shimada, T. & Persons, D. A. Optimized lentiviral vector design improves titer and transgene expression of vectors containing the chicken beta-globin locus HS4 insulator element. Mol Ther 17, 667-674 (2009).
43. Arumugam, P. I., Scholes, J., Perelman, N., Xia, P., Yee, J. K. & Malik, P. Improved human beta-globin expression from self-inactivating lentiviral vectors carrying the chicken hypersensitive site-4 (cHS4) insulator element. Mol Ther 15, 1863-1871 (2007).
44. Fraser, P., Pruzina, S., Antoniou, M. & Grosveld, F. Each hypersensitive site of the human beta-globin locus control region confers a different developmental pattern of expression on the globin genes. Genes & development 7, 106-113 (1993).
45. Navas, P. A., Peterson, K. R., Li, Q., Skarpidi, E., Rohde, A., Shaw, S. E., Clegg, C. H., Asano, H. & Stamatoyannopoulos, G. Developmental specificity of the interaction between the locus control region and embryonic or fetal globin genes in transgenic mice with an HS3 core deletion. Molecular and cellular biology 18, 4188-4196 (1998).
46. Li, Q. & Stamatoyannopoulos, G. Hypersensitive site 5 of the human beta locus control region functions as a chromatin insulator. Blood 84, 1399-1401 (1994).
47. Li, Q., Zhang, M., Han, H., Rohde, A. & Stamatoyannopoulos, G. Evidence that DNase I hypersensitive site 5 of the human beta-globin locus control region functions as a chromosomal insulator in transgenic mice. Nucleic Acids Res 30, 2484-2491 (2002).
48. Puthenveetil, G., Scholes, J., Carbonell, D., Qureshi, N., Xia, P., Zeng, L., Li, S., Yu, Y., Hiti, A. L., Yee, J. K. & Malik, P. Successful correction of the human beta-thalassemia major phenotype using a lentiviral vector. Blood 104, 3445-3453 (2004).
49. Wilber, A., Nienhuis, A. W. & Persons, D. A. Transcriptional regulation of fetal to adult hemoglobin switching: new therapeutic opportunities. Blood 117, 3945-3953 (2011).
50. Arumugam, P. I., Higashimoto, T., Urbinati, F., Modlich, U., Nestheide, S., Xia, P., Fox, C., Corsinotti, A., Baum, C. & Malik, P. Genotoxic potential of lineage-specific lentivirus vectors carrying the beta-globin locus control region. Mol Ther 17, 1929-1937 (2009).
51. Chang, K. H., Fang, X., Wang, H., Huang, A., Cao, H., Yang, Y., Bonig, H., Stamatoyannopoulos, J. A. & Papayannopoulou, T. Epigenetic modifications and chromosome conformations of the beta globin locus throughout development. Stem cell reviews 9, 397-407 (2013).
52. Papayannopoulou, T., Priestley, G. V., Rohde, A., Peterson, K. R. & Nakamoto, B. Hemopoietic lineage commitment decisions: in vivo evidence from a transgenic mouse model harboring micro LCR-betapro-LacZ as a transgene. Blood 95, 1274-1282 (2000).
53. Nienhuis, A. W. Development of gene therapy for blood disorders: an update. Blood 122, 1556-1564 (2013).
54. Baum, C., Kustikova, O., Modlich, U., Li, Z. & Fehse, B. Mutagenesis and oncogenesis by chromosomal insertion of gene transfer vectors. Hum Gene Ther 17, 253-263 (2006).
55. Nienhuis, A. W., Dunbar, C. E. & Sorrentino, B. P. Genotoxicity of retroviral integration in hematopoietic cells. Mol Ther 13, 1031-1049 (2006).
56. Emery, D. W. The use of chromatin insulators to improve the expression and safety of integrating gene transfer vectors. Hum Gene Ther 22, 761-774 (2011).
57. Evans-Galea, M. V., Wielgosz, M. M., Hanawa, H., Srivastava, D. K. & Nienhuis, A. W. Suppression of clonal dominance in cultured human lymphoid cells by addition of the cHS4 insulator to a lentiviral vector. Mol Ther 15, 801-809 (2007).
58. Rivella, S., Callegari, J. A., May, C., Tan, C. W. & Sadelain, M. The cHS4 insulator increases the probability of retroviral expression at random chromosomal integration sites. J Virol 74, 4679-4687 (2000).
59. Emery, D. W., Yannaki, E., Tubb, J. & Stamatoyannopoulos, G. A chromatin insulator protects retrovirus vectors from chromosomal position effects. Proc Natl Acad Sci USA 97, 9150-9155 (2000).
60. Emery, D. W., Yannaki, E., Tubb, J., Nishino, T., Li, Q. & Stamatoyannopoulos, G. Development of virus vectors for gene therapy of beta chain hemoglobinopathies: flanking with a chromatin insulator reduces gamma-globin gene silencing in vivo. Blood 100, 2012-2019 (2002).
61. Yannaki, E., Tubb, J., Aker, M., Stamatoyannopoulos, G. & Emery, D. W. Topological constraints governing the use of the chicken HS4 chromatin insulator in oncoretrovirus vectors. Mol Ther 5, 589-598 (2002).
62. Hino, S., Fan, J., Taguwa, S., Akasaka, K. & Matsuoka, M. Sea urchin insulator protects lentiviral vector from silencing by maintaining active chromatin structure. Gene Ther 11, 819-828 (2004).
63. Ramezani, A., Hawley, T. S. & Hawley, R. G. Performance- and safety-enhanced lentiviral vectors containing the human interferon-beta scaffold attachment region and the chicken beta-globin insulator. Blood 101, 4717-4724 (2003).
64. Ramezani, A., Hawley, T. S. & Hawley, R. G. Combinatorial incorporation of enhancer-blocking components of the chicken beta-globin 5′HS4 and human T-cell receptor alpha/delta BEAD-1 insulators in self-inactivating retroviral vectors reduces their genotoxic potential. Stem Cells 26, 3257-3266 (2008).
65. Yannaki, E., Emery, D. W. & Stamatoyannopoulos, G. Gene therapy for beta-thalassaemia: the continuing challenge. Expert reviews in molecular medicine 12, e31 (2010).
66. Persons, D. A. The challenge of obtaining therapeutic levels of genetically modified hematopoietic stem cells in beta-thalassemia patients. Ann NY Acad Sci 1202, 69-74 (2010).
67. Perumbeti, A. & Malik, P. Therapy for beta-globinopathies: a brief review and determinants for successful and safe correction. Ann NY Acad Sci 1202, 36-44 (2010).
68. Johnson, K. D., Grass, J. A., Park, C., Im, H., Choi, K. & Bresnick, E. H. Highly restricted localization of RNA polymerase II within a locus control region of a tissue-specific chromatin domain. Molecular and cellular biology 23, 6484-6493 (2003).
69. Vieira, K. F., Levings, P. P., Hill, M. A., Crusselle, V. J., Kang, S. H., Engel, J. D. & Bungert, J. Recruitment of transcription complexes to the beta-globin gene locus in vivo and in vitro. J Biol Chem 279, 50350-50357 (2004).
70. Levings, P. P., Zhou, Z., Vieira, K. F., Crusselle-Davis, V. J. & Bungert, J. Recruitment of transcription complexes to the beta-globin locus control region and transcription of hypersensitive site 3 prior to erythroid differentiation of murine embryonic stem cells. The FEBS journal 273, 746-755 (2006).
71. Felsenfeld, G. & Groudine, M. Controlling the double helix. Nature 421, 448-453 (2003).
72. Felsenfeld, G. Chromatin as an essential part of the transcriptional mechanism. Nature 355, 219-224 (1992).
73. Brownell, J. E. & Allis, C. D. Special HATs for special occasions: linking histone acetylation to chromatin assembly and gene activation. Curr Opin Genet Dev 6, 176-184 (1996).
74. Kingston, R. E. & Narlikar, G. J. ATP-dependent remodeling and acetylation as regulators of chromatin fluidity. Genes & development 13, 2339-2352 (1999).
75. Tsukiyama, T. & Wu, C. Chromatin remodeling and transcription. Curr Opin Genet Dev 7, 182-191 (1997).
76. Wolffe, A. P., Wong, J. & Pruss, D. Activators and repressors: making use of chromatin to regulate transcription. Genes to cells: devoted to molecular & cellular mechanisms 2, 291-302 (1997).
77. Kadonaga, J. T. Eukaryotic transcription: an interlaced network of transcription factors and chromatin-modifying machines. Cell 92, 307-313 (1998).
78. Struhl, K. Histone acetylation and transcriptional regulatory mechanisms. Genes & development 12, 599-606 (1998).
79. Gross, D. S. & Garrard, W. T. Nuclease hypersensitive sites in chromatin. Annual review of biochemistry 57, 159-197 (1988).
80. Elgin, S. C. Anatomy of hypersensitive sites. Nature 309, 213-214 (1984).
81. Wu, C. The 5′ ends of Drosophila heat shock genes in chromatin are hypersensitive to DNase I. Nature 286, 854-860 (1980).
82. Felsenfeld, G., Boyes, J., Chung, J., Clark, D. & Studitsky, V. Chromatin structure and gene expression. Proc Natl Acad Sci USA 93, 9384-9388 (1996).
83. Burgess-Beusse, B., Farrell, C., Gaszner, M., Litt, M., Mutskov, V., Recillas-Targa, F., Simpson, M., West, A. & Felsenfeld, G. The insulation of genes from external enhancers and silencing chromatin. Proc Natl Acad Sci USA 99 Suppl 4, 16433-16437 (2002).
84. Elgin, S. C. DNAase I-hypersensitive sites of chromatin. Cell 27, 413-415 (1981).
85. McGhee, J. D., Wood, W. I., Dolan, M., Engel, J. D. & Felsenfeld, G. A 200 base pair region at the 5′ end of the chicken adult beta-globin gene is accessible to nuclease digestion. Cell 27, 45-55 (1981).
86. Lowrey, C. H., Bodine, D. M. & Nienhuis, A. W. Mechanism of DNase I hypersensitive site formation within the human globin locus control region. Proc Natl Acad Sci USA 89, 1143-1147 (1992).
87. Adams, C. C. & Workman, J. L. Binding of disparate transcriptional activators to nucleosomal DNA is inherently cooperative. Molecular and cellular biology 15, 1405-1421 (1995).
88. McArthur, M., Gerum, S. & Stamatoyannopoulos, G. Quantification of DNaseI-sensitivity by real-time PCR: quantitative analysis of DNaseI-hypersensitivity of the mouse beta-globin LCR. J Mol Biol 313, 27-34 (2001).
89. Dorschner, M. O., Hawrylycz, M., Humbert, R., Wallace, J. C., Shafer, A., Kawamoto, J., Mack, J., Hall, R., Goldy, J., Sabo, P. J., Kohli, A., Li, Q., McArthur, M. & Stamatoyannopoulos, J. A. High-throughput localization of functional elements by quantitative chromatin profiling. Nat Methods 1, 219-225 (2004).
90. Sabo, P. J., Kuehn, M. S., Thurman, R., Johnson, B. E., Johnson, E. M., Cao, H., Yu, M., Rosenzweig, E., Goldy, J., Haydock, A., Weaver, M., Shafer, A., Lee, K., Neri, F., Humbert, R., Singer, M. A., Richmond, T. A., Dorschner, M. O., McArthur, M., Hawrylycz, M., Green, R. D., Navas, P. A., Noble, W. S. & Stamatoyannopoulos, J. A. Genome-scale mapping of DNase I sensitivity in vivo using tiling DNA microarrays. Nat Methods 3, 511-518 (2006).
91. Sabo, P. J., Hawrylycz, M., Wallace, J. C., Humbert, R., Yu, M., Shafer, A., Kawamoto, J., Hall, R., Mack, J., Dorschner, M. O., McArthur, M. & Stamatoyannopoulos, J. A. Discovery of functional noncoding elements by digital analysis of chromatin structure. Proc Natl Acad Sci USA 101, 16837-16842 (2004).
92. Sabo, P. J., Humbert, R., Hawrylycz, M., Wallace, J. C., Dorschner, M. O., McArthur, M. & Stamatoyannopoulos, J. A. Genome-wide identification of DNaseI hypersensitive sites using active chromatin sequence libraries. Proc Natl Acad Sci USA 101, 4537-4542 (2004).
93. Thurman, R. E., Rynes, E., Humbert, R., Vierstra, J., Maurano, M. T., Haugen, E., Sheffield, N. C., Stergachis, A. B., Wang, H., Vernot, B., Garg, K., John, S., Sandstrom, R., Bates, D., Boatman, L., Canfield, T. K., Diegel, M., Dunn, D., Ebersol, A. K., Frum, T., Giste, E., Johnson, A. K., Johnson, E. M., Kutyavin, T., Lajoie, B., Lee, B. K., Lee, K., London, D., Lotakis, D., Neph, S., Neri, F., Nguyen, E. D., Qu, H., Reynolds, A. P., Roach, V., Safi, A., Sanchez, M. E., Sanyal, A., Shafer, A., Simon, J. M., Song, L., Vong, S., Weaver, M., Yan, Y., Zhang, Z., Zhang, Z., Lenhard, B., Tewari, M., Dorschner, M. O., Hansen, R. S., Navas, P. A., Stamatoyannopoulos, G., Iyer, V. R., Lieb, J. D., Sunyaev, S. R., Akey, J. M., Sabo, P. J., Kaul, R., Furey, T. S., Dekker, J., Crawford, G. E. & Stamatoyannopoulos, J. A. The accessible chromatin landscape of the human genome. Nature 489, 75-82 (2012).
94. Stergachis, A. B., Neph, S., Reynolds, A., Humbert, R., Miller, B., Paige, S. L., Vernot, B., Cheng, J. B., Thurman, R. E., Sandstrom, R., Haugen, E., Heimfeld, S., Murry, C. E., Akey, J. M. & Stamatoyannopoulos, J. A. Developmental fate and cellular maturity encoded in human regulatory DNA landscapes. Cell 154, 888-903 (2013).
95. Neph, S., Stergachis, A. B., Reynolds, A., Sandstrom, R., Borenstein, E. & Stamatoyannopoulos, J. A. Circuitry and dynamics of human transcription factor regulatory networks. Cell 150, 1274-1286 (2012).
96. Maurano, M. T., Humbert, R., Rynes, E., Thurman, R. E., Haugen, E., Wang, H., Reynolds, A. P., Sandstrom, R., Qu, H., Brody, J., Shafer, A., Neri, F., Lee, K., Kutyavin, T., Stehling-Sun, S., Johnson, A. K., Canfield, T. K., Giste, E., Diegel, M., Bates, D., Hansen, R. S., Neph, S., Sabo, P. J., Heimfeld, S., Raubitschek, A., Ziegler, S., Cotsapas, C., Sotoodehnia, N., Glass, I., Sunyaev, S. R., Kaul, R. & Stamatoyannopoulos, J. A. Systematic localization of common disease-associated variation in regulatory DNA. Science 337, 1190-1195 (2012).
97. Stergachis, A. B., Haugen, E., Shafer, A., Fu, W., Vernot, B., Reynolds, A., Raubitschek, A., Ziegler, S., LeProust, E. M., Akey, J. M. & Stamatoyannopoulos, J. A. Exonic transcription factor binding directs codon choice and affects protein evolution. Science 342, 1367-1372 (2013).
98. Neph, S., Vierstra, J., Stergachis, A. B., Reynolds, A. P., Haugen, E., Vernot, B., Thurman, R. E., John, S., Sandstrom, R., Johnson, A. K., Maurano, M. T., Humbert, R., Rynes, E., Wang, H., Vong, S., Lee, K., Bates, D., Diegel, M., Roach, V., Dunn, D., Neri, J., Schafer, A., Hansen, R. S., Kutyavin, T., Giste, E., Weaver, M., Canfield, T., Sabo, P., Zhang, M., Balasundaram, G., Byron, R., MacCoss, M. J., Akey, J. M., Bender, M. A., Groudine, M., Kaul, R. & Stamatoyannopoulos, J. A. An expansive human regulatory lexicon encoded in transcription factor footprints. Nature 489, 83-90 (2012).
99. Ramezani, A., Hawley, T. S. & Hawley, R. G. Stable gammaretroviral vector expression during embryonic stem cell-derived in vitro hematopoietic development. Mol Ther 14, 245-254 (2006).
100. Recillas-Targa, F., Pikaart, M. J., Burgess-Beusse, B., Bell, A. C., Litt, M. D., West, A. G., Gaszner, M. & Felsenfeld, G. Position-effect protection and enhancer blocking by the chicken beta-globin insulator are separable activities. Proc Natl Acad Sci USA 99, 6883-6888 (2002).
101. Gaszner, M. & Felsenfeld, G. Insulators: exploiting transcriptional and epigenetic mechanisms. Nat Rev Genet 7, 703-713 (2006).
102. Wallace, J. A. & Felsenfeld, G. We gather together: insulators and genome organization. Curr Opin Genet Dev 17, 400-407 (2007).
103. Chung, J. H., Bell, A. C. & Felsenfeld, G. Characterization of the chicken beta-globin insulator. Proc Natl Acad Sci USA 94, 575-580 (1997).
104. Bell, A. C., West, A. G. & Felsenfeld, G. The protein CTCF is required for the enhancer blocking activity of vertebrate insulators. Cell 98, 387-396 (1999).
105. Ryu, B. Y., Persons, D. A., Evans-Galea, M. V., Gray, J. T. & Nienhuis, A. W. A chromatin insulator blocks interactions between globin regulatory elements and cellular promoters in erythroid cells. Blood Cells Mol Dis 39, 221-228 (2007).
106. Ryu, B. Y., Evans-Galea, M. V., Gray, J. T., Bodine, D. M., Persons, D. A. & Nienhuis, A. W. An experimental system for the evaluation of retroviral vector design to diminish the risk for proto-oncogene activation. Blood 111, 1866-1875 (2008).
107. Yao, S., Osborne, C. S., Bharadwaj, R. R., Pasceri, P., Sukonnik, T., Pannell, D., Recillas-Targa, F., West, A. G. & Ellis, J. Retrovirus silencer blocking by the cHS4 insulator is CTCF independent. Nucleic Acids Res 31, 5317-5323 (2003).
108. Nishino, T., Tubb, J. & Emery, D. W. Partial correction of murine beta-thalassemia with a gammaretrovirus vector for human gamma-globin. Blood Cells Mol Dis 37, 1-7 (2006).
109. Aker, M., Tubb, J., Groth, A. C., Bukovsky, A. A., Bell, A. C., Felsenfeld, G., Kiem, H. P., Stamatoyannopoulos, G. & Emery, D. W. Extended core sequences from the cHS4 insulator are necessary for protecting retroviral vectors from silencing position effects. Hum Gene Ther 18, 333-343 (2007).
110. Li, C. L. & Emery, D. W. The cHS4 chromatin insulator reduces gammaretroviral vector silencing by epigenetic modifications of integrated provirus. Gene Ther 15, 49-53 (2008).
111. Ma, Y., Ramezani, A., Lewis, R., Hawley, R. G. & Thomson, J. A. High-level sustained transgene expression in human embryonic stem cells using lentiviral vectors. Stem Cells 21, 111-117 (2003).
112. Chang, L. J., Liu, X. & He, J. Lentiviral siRNAs targeting multiple highly conserved RNA sequences of human immunodeficiency virus type 1. Gene Ther 12, 1133-1144 (2005).
113. Pluta, K., Luce, M. J., Bao, L., Agha-Mohammadi, S. & Reiser, J. Tight control of transgene expression by lentivirus vectors containing second-generation tetracycline-responsive promoters. J Gene Med 7, 803-817 (2005).
114. Jakobsson, J., Rosenqvist, N., Thompson, L., Barraud, P. & Lundberg, C. Dynamics of transgene expression in a neural stem cell line transduced with lentiviral vectors incorporating the cHS4 insulator. Experimental cell research 298, 611-623 (2004).
115. Leboulch, P., Huang, G. M., Humphries, R. K., Oh, Y. H., Eaves, C. J., Tuan, D. Y. & London, I. M. Mutagenesis of retroviral vectors transducing human beta-globin gene and beta-globin locus control region derivatives results in stable transmission of an active transcriptional structure. EMBO J 13, 3065-3076 (1994).
116. Kim, T. H., Abdullaev, Z. K., Smith, A. D., Ching, K. A., Loukinov, D. I., Green, R. D., Zhang, M. Q., Lobanenkov, V. V. & Ren, B. Analysis of the vertebrate insulator protein CTCF-binding sites in the human genome. Cell 128, 1231-1245 (2007).
117. Yusufzai, T. M. & Felsenfeld, G. The 5′-HS4 chicken beta-globin insulator is a CTCF-dependent nuclear matrix-associated element. Proc Natl Acad Sci USA 101, 8620-8624 (2004).
118. Phillips, J. E. & Corces, V. G. CTCF: master weaver of the genome. Cell 137, 1194-1211 (2009).
119. Giles, K. E., Gowher, H., Ghirlando, R., Jin, C. & Felsenfeld, G. Chromatin boundaries, insulators, and long-range interactions in the nucleus. Cold Spring Harbor symposia on quantitative biology 75, 79-85 (2010).
120. Barski, A., Cuddapah, S., Cui, K., Roh, T. Y., Schones, D. E., Wang, Z., Wei, G., Chepelev, I. & Zhao, K. High-resolution profiling of histone methylations in the human genome. Cell 129, 823-837 (2007).
121. Wang, H., Maurano, M. T., Qu, H., Varley, K. E., Gertz, J., Pauli, F., Lee, K., Canfield, T., Weaver, M., Sandstrom, R., Thurman, R. E., Kaul, R., Myers, R. M. & Stamatoyannopoulos, J. A. Widespread plasticity in CTCF occupancy linked to DNA methylation. Genome research 22, 1680-1688 (2012).
122. Schmidt, D., Schwalie, P. C., Wilson, M. D., Ballester, B., Goncalves, A., Kutter, C., Brown, G. D., Marshall, A., Flicek, P. & Odom, D. T. Waves of retrotransposon expansion remodel genome organization and CTCF binding in multiple mammalian lineages. Cell 148, 335-348 (2012).
123. Renda, M., Baglivo, I., Burgess-Beusse, B., Esposito, S., Fattorusso, R., Felsenfeld, G. & Pedone, P. V. Critical DNA binding interactions of the insulator protein CTCF: a small number of zinc fingers mediate strong binding, and a single finger-DNA interaction controls binding at imprinted loci. J Biol Chem 282, 33336-33345 (2007).
124. Dickson, J., Gowher, H., Strogantsev, R., Gaszner, M., Hair, A., Felsenfeld, G. & West, A. G. VEZF1 elements mediate protection from DNA methylation. PLoS Genet 6, e1000804 (2010).
125. Li, C. L., Xiong, D., Stamatoyannopoulos, G. & Emery, D. W. Genomic and functional assays demonstrate reduced gammaretroviral vector genotoxicity associated with use of the cHS4 chromatin insulator. Mol Ther 17, 716-724 (2009).
126. Lisowski, L. & Sadelain, M. Locus control region elements HS1 and HS4 enhance the therapeutic efficacy of globin gene transfer in beta-thalassemic mice. Blood 110, 4175-4178 (2007).
127. Nagel, R. L., Bookchin, R. M., Johnson, J., Labie, D., Wajcman, H., Isaac-Sodeye, W. A., Honig, G. R., Schiliro, G., Crookston, J. H. & Matsutomo, K. Structural bases of the inhibitory effects of hemoglobin F and hemoglobin A2 on the polymerization of hemoglobin S. Proc Natl Acad Sci USA 76, 670-672 (1979).
128. Sadelain et al., Proc. Nat'l Acad. Sci. (USA) (1995); 92:6728-6732.
129. Armstrong, J. A., Emerson, B. M., 1996. NFE2 disrupts chromatin structure at human fl-globin locus control region hypersensitive site 2 in vitro. Mol. Cell. Biol. 16, 5634-5644.
130. Caterina, J. J., Ciavatta, D. J., Donze, D., Behringer, R. R., Townes, T. M., 1994. Multiple elements in human fl-globin locus control region 5′ HS2 are involved in enhancer activity and position-independent transgene expression. Nucleic Acids Res. 22, 1006 1011.
131. Moi, P., Kan, Y. W., 1990. Synergistic enhancement of globin gene expression by activator protein-1-like proteins. Proc. Natl. Acad, Sci. USA 87, 9000-9004.
132. Ney, P., Sorrentino, B., McDonagh, K., Nienhuis, A., 1990. Tandem AP-1-binding sites within the human/j-globin dominant control region function as an inducible enhancer in erythroid cells. Genes Dev. 4, 993 1006.
133. Shivdasani, R. A., Rosenblatt, M. F., Zucker-Franklin, D., Jackson, C. W., Hunt, P., Saris, C. J. M., Orkin, S. H., 1995. Transcription factor NF-E2 is required for platelet formation independent of the actions of thrombopoietin/MGDF in megakaryocyte development. Cell 81, 695-704.
134. Talbot, D., Grosveld, F., 1991. The 5′HS2 of the globin locus control region enhances transcription through the interaction of a multimeric complex binding at two functionally distinct NF-E2 binding sites. EMBO J. 10, 1391-1398.
135. Hardison et al., Gene (1997); 205:73-94.
136. Elnitski et al., The Journal of Biological Chemistry (1997); 272(1):369-378; Horak et al., PNAS (2002); 99(5):2924-2929.
137. Shimotsuma et al., Journal of Biological Chemistry (2010); 285(19):14495-14503.

From the foregoing description, it will be apparent that variations and modifications may be made to the presently disclosed subject matter described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

1-110. (canceled)

111. An expression cassette comprising an insulator that comprises the CTCF binding site sequence set forth in SEQ ID NO: 18, and a globin gene or a functional portion thereof operably linked to a β-globin locus control region (LCR) region.

112. The expression cassette of claim 111, wherein the insulator comprises SEQ ID NO: 24, SEQ ID NO: 25, or SEQ ID NO: 1.

113. The expression cassette of claim 111, wherein:
(a) the β-globin LCR region does not comprise a Dnase I hypersensitive site-2 (HS2) region;
(b) the β-globin LCR region does not comprise a core sequence of HS2;
(c) the core sequence of HS2 has the nucleotide sequence set forth in SEQ ID NO:20 or SEQ ID NO: 21;
(d) the β-globin LCR region does not comprise a HS2 region that sustains the enhancer activity of HS2;
(e) the β-globin LCR region comprises a Dnase I hypersensitive site-1 (HS1) region, a Dnase I hypersensitive site-3 (HS3) region, and a Dnase I hypersensitive site-4 (HS4) region;
(f) the HS3 region is positioned between the HS1 region and the HS4 region;
(g) the HS1 region is about 1.1 kb in length;
(h) the HS1 region is about 600 bp in length;
(i) the HS1 region is about 490 bp in length;
(j) the HS3 region is about 1300 bp in length;
(k) the HS4 region is about 1.1 kb in length;
(l) the HS4 region is about 450 bp in length;
(m) the HS1 region has the nucleotide sequence set forth in SEQ ID NO:2, SEQ ID NO:3, or SEQ ID NO:4;
(n) the HS3 region has the nucleotide sequence set forth in SEQ ID NO:5; or
(o) the HS4 region has the nucleotide sequence set forth in SEQ ID NO:6, SEQ ID NO:7, or SEQ ID NO: 8.

114. The expression cassette of claim 111, wherein:
(a) the β-globin LCR region does not comprise a HS1 region;
(b) the β-globin LCR region does not comprise a core sequence of HS1;
(c) the β-globin LCR region does not comprise a HS1 region that sustains the function of HS1;
(d) the β-globin LCR region comprises a HS3 region and a HS4 region;
(e) the β-globin LCR region comprises a HS3 region having the nucleotide sequence set forth in SEQ ID NO:5 and a HS4 region having the nucleotide sequence set forth in SEQ ID NO:6, and the β-globin LCR region does not comprise a HS1 region or a HS2 region;
(f) the core sequence of HS1 has the nucleotide sequence set forth in SEQ ID NO:22 or SEQ ID NO: 23; or
(g) the HS3 region is positioned between the globin gene or functional portion thereof and the HS4 region.

115. The expression cassette of claim 111, wherein:
(a) the β-globin LCR region comprises a HS1 region having the nucleotide sequence set forth in SEQ ID NO:2, a HS3 region having the nucleotide sequence set forth in SEQ ID NO:5, and a HS4 region having the nucleotide sequence set forth in SEQ ID NO:6, and the β-globin LCR region does not comprise a HS2 region;
(b) the β-globin LCR region comprises a HS1 region having the nucleotide sequence set forth in SEQ ID NO:3, a HS3 region having the nucleotide sequence set forth in SEQ ID NO:5, and a HS4 region having the nucleotide sequence set forth in SEQ ID NO:8, and the β-globin LCR region does not comprise a HS2 region; or
(c) the β-globin LCR region comprises a HS1 region having the nucleotide sequence set forth in SEQ ID NO:4, a HS3 region having the nucleotide sequence set forth in SEQ ID NO:5, and a HS4 region having the nucleotide sequence set forth in SEQ ID NO:8, and the β-globin LCR region does not comprise a HS2 region.

116. The expression cassette of claim 111, wherein:
(a) the β-globin LCR region comprises a HS2 region, a HS3 region, and a HS4 region;
(b) the HS2 region is about 860 bp in length;
(c) the HS2 region has the nucleotide sequence set forth in SEQ ID NO:9;
(d) the HS3 region is about 1300 bp in length;
(e) the HS3 region has the nucleotide sequence set forth in SEQ ID NO:5;
(f) the HS4 region is about 1.1 kb in length;
(g) the HS4 region has the nucleotide sequence set forth in SEQ ID NO:7;
(h) the β-globin LCR region comprises a HS2 region having the nucleotide sequence set forth in SEQ ID NO:9, a HS3 region having the nucleotide sequence set forth in SEQ ID NO:5, and a HS4 region having the nucleotide sequence set forth in SEQ ID NO:7; or
(i) the β-globin LCR region further comprises a HS1 region.

117. The expression cassette of claim 111, wherein:
(a) the globin gene is selected from the group consisting of β-globin gene, γ-globin gene, and δ-globin gene;
(b) the globin gene is human β-globin gene;
(c) the human β-globin gene is selected from the group consisting of a wild-type human β-globin gene, a deleted human β-globin gene comprising one or more deletions of intron sequences, and a mutated human β-globin gene encoding at least one anti-sickling amino acid residue; or
(d) the human β-globin gene is human βA-globin gene encoding a threonine to glutamine mutation at codon 87 (βA-T87Q).

118. The expression cassette of claim 111, comprising:
(a) one insulator having the nucleotide sequence set forth in SEQ ID NO: 1;
(b) two of the insulator having the nucleotide sequence set forth in SEQ ID NO: 1;
(c) a β-globin promoter;
(d) a human β-globin 3′ enhancer; or
(e) at least one erythroid-specific enhancer.

119. The expression cassette of claim 111, wherein:
(a) the β-globin promoter is positioned between the globin gene or functional portion thereof and the β-globin LCR region;
(b) the β-globin promoter is a human β-globin promoter that is about 613 bp in length;
(c) the human β-globin promoter has the nucleotide sequence set forth in SEQ ID NO:10;
(d) the β-globin promoter is a human β-globin promoter that is about 265 bp in length;
(e) the human β-globin promoter has the nucleotide sequence set forth in SEQ ID NO:11;
(f) the human β-globin 3′ enhancer is positioned in the upstream of the globin gene or functional portion thereof;
(g) the human β-globin 3′ enhancer is about 879 bp in length;
(h) the human β-globin 3′ enhancer has the nucleotide sequence set forth in SEQ ID NO:12;
(i) the at least one erythroid-specific enhancer is positioned between the globin gene or functional portion thereof and β-globin LCR region;
(j) the at least one erythroid-specific enhancer has a nucleotide sequence selected from the group consisting of SEQ ID NOS: 13, 14, 15, 16 and 17; or
(k) the expression cassette comprises one, two or three erythroid-specific enhancers.

120. A recombinant vector comprising the expression cassette of claim 111, wherein the expression cassette comprises an insulator comprising the nucleotide sequence set forth in SEQ ID NO:1, and the vector further comprising one or both of a Woodchuck hepatitis post-regulatory element (WPRE) and a bovine growth hormone polyadenylation signal in the 3′ long terminal repeat (LTR) of the vector.

121. A cell transduced with the expression cassette of claim 111.

122. The cell of claim 121, wherein the hematopoietic stem cell is a CD34⁺ hematopoietic stem cell.

123. A pharmaceutical composition comprising an effective amount of the cell of claim 121 and a pharmaceutically acceptable carrier.

124. A kit for treating hemoglobinopathy comprising the cell of claim 121.

125. A method of treating a hemoglobinopathy in a subject, comprising administering an effective amount of the cell of claim 121 to the subject, thereby enabling the subject's ability to produce red blood cells containing normal hemoglobin.

126. The method of claim 125, wherein:
(a) the method does not comprise administering an immunosuppressive agent;
(b) the hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease;
(c) the subject is a human;
(d) the cell is from the subject; or
(e) the cell is from bone marrow of the subject.


SEQUENCE LISTING (26 sequences)

SEQ ID NO: 1 - DNA, 305 bp, Homo sapiens
  source 1..305: mol_type = genomic DNA
tccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctag

SEQ ID NO: 2 - DNA, 1074 bp, Homo sapiens
  source 1..1074: mol_type = genomic DNA
aagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcaggtcctaggataccaggaaaataaattacattccaaaaatttaactgagactttaaaaaaaaaaaaaaaaaaaaaaaaaaaaccagtgatccatggacacagggaggggaacatcacacactggggcctgttgggggtggggggctaggggaaggatagcattaggagaaatacctaatgtagatgacgggttgatgggtgcagcaaaccaccatggcacatgtaccccagaacttaaagcatattaaaaaaacagtgatcataaaagaagctcaaatttaactataagagacggaatggctcccacaattcttaactataatcttacagaatattctcattgaatagaagtatgcttatcattagagatttggacagccaggaaagcacagaaaaaaaaaaaaggagctctgttgccttatagcctagaggtgttt

SEQ ID NO: 3 - DNA, 602 bp, Homo sapiens
  source 1..602: mol_type = genomic DNA
ggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgtatatatatgttttatatatatatatgtgtgtgtgtgtgtgtgtgtgtgttgtgtttacaaatatgtgattatcatcaaaacgtgagggctaaagtgaccagataacttgcagg

SEQ ID NO: 4 - DNA, 489 bp, Homo sapiens
  source 1..489: mol_type = genomic DNA
ggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcggactatgctgagctgtgatgagggagggacctagccaaaggcagtgagagtcagaatgctcctgctattgccttctcagtccccacgcttggtttctacacaagtagatacatagaaaaggctataggttagtgtttgagagtcctgcatgagttagttgctcagaaatgcccgataaatatgttatgtgtgtttatgt

SEQ ID NO: 5 - DNA, 1301 bp, Homo sapiens
  source 1..1301: mol_type = genomic DNA
aagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagccattctccccacatagatcatctcagcagggttcaggaagataaaggaggatcaaggtcgaaggtaggaactaaggaagaacactgggcaagtggatcc

SEQ ID NO: 6 - DNA, 1065 bp, Homo sapiens
  source 1..1065: mol_type = genomic DNA
tgagccccttttcctctaactgaaagaaggaaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgaggcctcagctttctaggaataatatcaatattacaaaatttaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgac

SEQ ID NO: 7 - DNA, 1065 bp, Homo sapiens
  source 1..1065: mol_type = genomic DNA
tgagccccttttcctctaactgaaagaaggaaaaaaaaaatggaacccaaaatattctacatagtttccatgtcacagccagggctgggcagtctcctgttatttcttttaaaataaatatatcattaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggcccccaagagctctctaaaagtgatggcaaagtcattgcgctagatgccatcccatctattataaacctgcatttgtctccacacaccagtcatggacaataaccctcctcccaggtccacgtgcttgtctttgtataatactcaagtaatttcggaaaatgtattctttcaatcttgttctgttattcctgtttcaatggcttagtagaaaaagtacatacttgttttcccataaattgacaatagacaatttcacatcaatgtctatatgggtcgttgtgtttgctgtgtttgcaaaaactcacaataactttatattgttactactctaagaaagttacaacatggtgaatacaagagaaagctattacaagtccagaaaataaaagttatcatcttgaggcctcagctttctaggaataatatcaatattacaaaattaatctaacaattatgaacagcaatgagataatatgtacaaagtacccagacctatgtggtagagcatcaaggaagcgcattgcggagcagttttttgtttgtttgtttttgtattctgtttcgtgaggcaaggtttcactctgctgtccaggctggagtgcagtggcaagatcatgtctcactgcagccttgacac

SEQ ID NO: 8 - DNA, 446 bp, Homo sapiens
  source 1..446: mol_type = genomic DNA
tggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattctcacactaagaaaaagaatgagatgtctacatataccctgcgtcccctcttgtgtactggggtccccaagagctctctaaaagtgatggcaaagtcattgcgctagatgccatcccatct

SEQ ID NO: 9 - DNA, 860 bp, Homo sapiens
  source 1..860: mol_type = genomic DNA
gtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgagatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgct

SEQ ID NO: 10 - DNA, 613 bp, Homo sapiens
  source 1..613: mol_type = genomic DNA
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttggaatcacagcttggtaagcatattgaagatcgttttcccaattttcttattacacaaataagaaattgatgcactaaaagtggaagagttttgtctaccataattcagctttgggatatgtagatggatctcttcctgcgtctccagaatatgcaaaatacttacaggacagaatggatgaaaactctacctcagttctaagcatatcttctccttatttggattaaaaccttctggtaagaaaagaaaaaaaatatatatatatatgtgtatatatacacacatacatatacatatatatgcattcatttgttgttgtttttcttaatttgctcatg

SEQ ID NO: 11 - DNA, 265 bp, Homo sapiens
  source 1..265: mol_type = genomic DNA
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 12 - DNA, 879 bp, Homo sapiens
  source 1..879: mol_type = genomic DNA
taggtattgaataagaaaaatgaagttaaggtggttgatggtaacactatgctaataactgcagagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcactgcagattccgggtcactgtgagtgggggaggcagggaagaagggctcacaggacagtcaaaccatgccccctgtttttccttcttcaagtagacctctataagacaacagagacaactaaggctgagtggccaggcgaggagaaaccatctcgccgtaaaacatggaaggaacacttcaggggaaaggtggtatctctaagcaagagaactgagtggagtcaaggctgagagatgcaggataagcaaatgggtagtgaaaagacattcatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattg

SEQ ID NO: 13 - DNA, 152 bp, synthetic construct
  misc_feature 1..152: note = Description of Artificial Sequence: Synthetic polynucleotide
  source 1..152: mol_type = other DNA
tctcccacgccctggtctcagcttggggagtggtcagaccccaatggcgataaactctggcaactttatctgtgcactgcaggctcagccccaacagctttagctttcacaagcaggcaggggaagggaaacacatatctccagatatgagg

SEQ ID NO: 14 - DNA, 157 bp, synthetic construct
  misc_feature 1..157: note = Description of Artificial Sequence: Synthetic polynucleotide
  source 1..157: mol_type = other DNA
ctaaacccctcccccaccctagccccaagcttcatcttagctccactcctgaccctatccagctaaaggtccccacccagctcctgcctatctagtcattgcatatggcaagacttgaaagtcctatctcaaagcagcagaattatcagctacgact

SEQ ID NO: 15 - DNA, 141 bp, synthetic construct
  misc_feature 1..141: note = Description of Artificial Sequence: Synthetic polynucleotide
  source 1..141: mol_type = other DNA
ccatcccccagcactccctgcccccacagcccagacttgaccaactcccagctccgcctgggacttccagatatggggccccacccttgcaggccttggggacgctgaagatattgactatctgcgtgccggaaaagggtg

SEQ ID NO: 16 - DNA, 171 bp, synthetic construct
  misc_feature 1..171: note = Description of Artificial Sequence: Synthetic polynucleotide
  source 1..171: mol_type = other DNA
aaaggctgggggtgggagtagcggatttgaagcacttgttggcctacagaggtgtggcaagcagagcacctcagaactcaggcgtactgcccgccgcccgagccctgcgagggccgatagcgagggtgtggcccttatctgcacccagcagagcgccggcggggtacggtc

SEQ ID NO: 17 - DNA, 195 bp, synthetic construct
  misc_feature 1..195: note = Description of Artificial Sequence: Synthetic polynucleotide
  source 1..195: mol_type = other DNA
cagttgcctcagctgagtatgtcttctaaagataatgtcgattgtgtatggctgatgggattctaggaccaagcaagaggtttttttttttcccccacatacttaacgtttctatatttctatttgaattcgactggacagttccatttgaattatttctctctctctctctctctgacacattttatcttgcca

SEQ ID NO: 18 - DNA, 14 bp, Homo sapiens
  source 1..14: mol_type = genomic DNA
caccaggtggcgct

SEQ ID NO: 19 - DNA, 81706 bp, Homo sapiens
  source 1..81706: mol_type = genomic DNA
ggatcctcacatgagttcagtatataattgtaacagaataaaaaatcaattatgtattcaagttgctagtgtcttaagaggttcacatttttatctaactgattatcacaaaaatacttcgagttacttttcattataattcctgactacacatgaagagactgacacgtaggtgccttacttaggtaggttaagtaatttatccaaaaccacacaatgtagaacctaagctgattcggccatagaaacacaatatgtggtataaatgagacagagggatttctctccttcctatgctgtcagatgaatactgagatagaatatttagttcatctatcacacattaaacgggactttacatttctgtctgttgaagatttgggtgtggggataactcaaggtatcatatccaagggatggatgaaggcaggtgactctaacagaaagggaaaggatgttggcaaggctatgttcatgaaagtatatgtaaaatccacattaagcttctttctgcatgcattggcaatgtttatgaataatgtgtatgtaaaagtgtgctgtatattcaaaagtgtttcatgtgcctaggggtgtcaaatactttgagtttgtaagtatatacttctctgtaatgtgtctgaatatctctatttacttgattctcaataagtaggtatcatagtgaacatctgacaaatgtttgaggaacaatttagtgtttacctattcaccaaaatttattaaatgcctaatctgtatcagatatacaattatctggcgaaatctgtaattcctaatttaaacagctgtgtagcctaattagggataaaggcatgcaaacccataatttgtgtaggttgaaatgagctatagaaaaatgcagtatatttatcagaagtctttagggtcatgaaaaggaatggtcaactgacactgccagggactcatatgtaagagataactaatgtgaagtgactttaaaggagaaattagcagaagttttctttccatgtctcctcatcatgttacaataacggaagagattaaaacaacaaatacatttagacagcaatgtttatcctggttagatgttttaatctaaatctatcttggagtgttaaaatgcatttgctcacctactttaaaatataaatgaaggtaggaacctgtagatacaaaaagttggagaaaaaaagacaataaagatgacaaaaatctattaatccttgatagaaaatgagaagagataaaacactggtttacataaagaaaataagatggatagatagcagatccttataaaagtgataatttgagaaaaaaaatactccatattctgagtttcttcacataaaataatacaaatctgctgtggtaagttacaaagagatagattttttatcattatataaaagatattttaaacagagttatacaacaaaggaacagactatgtcatatattctcacttatcactataaacatctcagaaaaatctgcaaaatcatttcatagcattttaaatagttaggaataatgtagaaaactgaaacagttctaagtttcccacaaacttagagtctcaaatgttgcattacctaacttacctgcaaatattttatacaaatttgcacatgctactctagtcaaaaatatatgtacattatgggtattttctgtgtgtaacttggttctagttgcttctttcagaaatagcctctatttttgatttacctgataaaatcacattcctctccaaagccttctaaatacttccagactaactactttttagtacatctaagaagaaaagagttttgtctcttatccacctctgagtcaaaaagcagcatgtccatcaattggtacatagttcccacagccccacttagctctggattggagttctacttggcattgtttgcaactacatggacgtaaaatgcatggattctcttgaaaaaatgtttctgccatgatgttctctgaaagagactaaccttccctcgctttgcagagaaagactcgtgtaatccttgacaatgtcatctcatctatttattcccatgtctacccatatgtgaccttcatgtctttgctctaagcccctacatcctcaatctacacactaggatagtataaaagtaatagtaataatagtagtaatagtaataacaatacaatgattatggcttatactatacacaagacactgttgatatattatttcatttagtattcacagtaactctgtgcctcaagtactattgtaataccctttaagaggaggaaactgaggcacagggccctaaagtaatattccaagatgaagtggctactaactgacagagggcataattcaactcatgatatttggctctagaatacatgctctgaatcattatacaataataattcatgaggaaacattttttaaagcctaagttatttgctctgaaataagacataatttggggtgagaaagcttagattccatgaagtattacagcatttggtagtctttttgcactccaggtcttatttttactgcttaaacataataaaacatatggttcagtatgcctttgattttacaataatattcctgttatttttggaagcacagggtgtgggataatgctaattactagtgattagtattgagaggtgacagcgtgctggcagtcctcacagccctcgctcgctcttggcgcctcctctgcctgggctcccacattggtggcacttgaggagcccttcagccggccgctgcactgtgggagcccttttctgggctggccaaggccagagccggctccctcagcttgccaggaggtgtggagggacagacgcgggcaggaaccgggctgtgcgccgtgcttgagggagttccgggtgggcatgggctccgaggaccccgcactcggagccgccagccggccccaccggccgcgggcagtgaggggcttagcacctgggccagcagctgctgtgctcaattcctcgccgggccttagctgccttcctgcggggcagggctcgggacctgcagcgcgccatgcctgagcctccccaccttcatgggctcctgtgcggcccgagcctcgccgacgagcgccgccccctgctccagggcacccagtcccatcgaccacccaagggctgaagagtgcgggcgcacggcaggggactggcaggcagctccccctgcagcccaggtgcgggatccactgggtgaagccggctaggctcctgagtttgctggggatgcgaagaacccttatgtctagataagggattgtaaatacaccaattggcactctgtatctagctcaaggtttgtaaacacaccaatcagcaccctgtgtctagctcagggtttgtgaatgcaccaatcaacactctatctagctactctggtggggccttggagaacctttatgtctagctcagggattgtaaatacaccaatcggcagtctgtatctagctcaaggtttgtaaacacaccaatcagcaccctgtgtctagctcagggtttgtgaatgcaccaatcaacactctgtatctagctactctggtggggacgtggagaacctttatgtctagctcagggattgtaaatacaccactcggcagtctgtatctagctcaaggtttgtaaacacaccaatcagcaccctgtgtctagctcagggtttgtgaatgcaccaatcaacactctgtatctagctactctggtggggacttggagaacctttgtgtggacactctgtatctagctaatctggtggggacgtggagaacctttgtgtctagctcatggattgtaaatgcaccaatcagtgccctgtcaaaacagaccactgggctctaccaatcagcaggatgtgggtggggccagataagagaataaaagcaggctgcccgagccagcagtggcaacccgctcgggtccccttccacactgtggaagctttgttctttcgctctttgcaataaatcttgctgctgctcactgtttgggtctacactgcctttatgagctgtaacgctcaccgcgaaggtctgcagcttcactcttgaagccagcgagaccacgaacccaccgggaggaacgaacaactccagaggcgccgccttaagagctggaacgttcactgtgaaggtctgcagcttcactcctgagccagcgagaccacgaacccatcagaaggaagaaactccgaacacatccaaacatcagaacgaacaaactccacacacgcagcctttaagaactgtaacactcaccacgagggtccccggcttcattcttgaagtcagtgaaaccaagaacccaccaattccggacacagtatgtcagaaacaatatgagtcactaaatcaatatacttctcaacaatttccaacagcccttgcaattaacttggccatgtgactggttgtgactaaaataatgtggagataataatgtgttactccctaaggcagagtgcccttctatcattctctttcccttcctctatgtggcagaaagtaaaagattctgaaatgataaagtcaatcacaggaaggcacctggactcctggcccactgcttggaggagagcactcaggaccatgaacatctgactgtgacgtagcaataaagaaacccacgtttcatatgaaactgcttaaaattaatggcacaagtcatgtttttgatgttgcacatttgtctttatttgtggcttgttttgcttccacatcaatccactcaaggcctacattctgctataatgcaatttcaagttctttacaggccgagaaaaatgaatctgaattcctgacctccaaaagtgatcaagatatttttagttcaggctccaaaattttctcattttcataggttttcctcgattgatcattattcatgatttgcaaggaatcattcaatgttttctaaatctattactgcatcctgacacatatgacattttaactatgttccagatttttgaatgaagagtgtaaattttaaatgttttcaccacaaaaaataagtatgtgaagtggtggatttgttaattagccttatttaaccatttaatattgtacacgtacaccaaagcatcatgttgtaccccatgaatacacacaattattatttgtcaatttaaaatgaaataataaaaaataacaaaggcattagcctctgcattgcctttaccggtcatcctcacggtgactaacgcaaaaaacgttctatttcatccttacaaacatccctatctttgatgcctctttgtctagatctctatcccctcctgttttctctacgttatttatatgggtatcatcaccatcctggacaacatcaggacagatatccctcaccaagccaatgttcctctctatgttggctcaaatgtccttgaactttcctttcaccaccctttccacagtcaaaaggatattgtagtttaatgcctcagagttcagcttttaagcttctgacaaattattcttcctctttaggttctcctttatggaatcttctgtactgatggccatgtcctttaactactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaattctcactcttgatgcaacagaccttggaagcatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattgaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcacttgagaggtagaagtgggaggactgcttgagctcaagagtttgatattatcctggacaacatagcaagacctcgtctctacttaaaaaaaaaaaaaaaattagccaggcatgtgatgtacacctgtagtcccagctactcaggaggccgaaatgggaggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggagccagaccacctgagtgattagtgactcagtttctcttagtagttgtatgactcagtttcttcatctgtaaaatggagggttttttaattagtttgtttttgagaaagggtctcactctgtcacccaaatgggagtgtagtggcaaaatctcggctcactgcaacttgcacttcccaggctcaagcggtcctcccacctcaacatcctgagtagctggaaccacaggtacacaccaccatacctcgctaattttttgtatttttggtagagatggggtttcacatgttacacaggatggtctcagactccggagctcaagcaatctgcccacctcagccttccaaagtgctgggattataagcatgattacaggagttttaacaggctcataagattgttctgcagcccgagtgagttaatacatgcaaagagtttaaagcagtgacttataaatgctaactactctagaaatgtttgctagtattttttgtttaactgcaatcattcttgctgcaggtgaaaactagtgttctgtactttatgcccattcatctttaactgtaataataaaaataactgacatttattgaaggctatcagagactgtaattagtgctttgcataattaatcatatttaatactcttggattctttcaggtagatactattattatccccattttactacagttaaaaaaactacctctcaacttgctcaagcatacactctcacacacacaaacataaactactagcaaatagtagaattgagatttggtcctaattatgtctttgctcactatccaataaatatttattgacatgtacttcttggcagtctgtatgctggatgctggggatacaaagatgtttaaatttaagctccagtctctgcttccaaaggcctcccaggccaagttatccattcagaaagcattttttactctttgcattccactgtttttcctaagtgactaaaaaattacactttattcgtctgtgtcctgctctgggatgatagtctgactttcctaacctgagcctaacatccctgacatcaggaaagactacaccatgtggagaaggggtggtggttttgattgctgctgtcttcagttagatggttaactttgtgaagttgaaaactgtggctctctggttgactgttagagttctggcacttgtcactatgcctattatttaacaaatgcatgaatgcttcagaatatgggaatattatcttctggaatagggaatcaagttatattatgtaacccaggattagaagattcttctgtgtgtaagaatttcataaacattaagctgtctagcaaaagcaagggcttggaaaatctgtgagctcctcaccatatagaaagcttttaacccatcattgaataaatccctataggggatttctaccctgagcaaaaggctggtcttgattaattcccaaactcatatagctctgagaaagtctatgctgttaacgttttcttgtctgctaccccatcatatgcacaacaataaatgcaggcctaggcatgactgaaggctctctcataattcttggttgcatgaatcagattatcaacagaaatgttgagacaaactatggggaagcagggtatgaaagagctctgaatgaaatggaaaccgcaatgcttcctgcccattcagggctccagcatgtagaaatctggggctttgtgaagactggcttaaaatcagaagccccattggataagagtagggaagaacctagagcctacgctgagcaggtttccttcatgtgacagggagcctcctgccccgaacttccagggatcctctcttaagtgtttcctgctggaatctcctcacttctatctggaaatggtttctccacagtccagcccctggctagttgaaagagttacccatgcagaggccctcctagcatccagagactagtgcttagattcctactttcagcgttggacaacctggatccacttgcccagtgttcttccttagttcctaccttcgaccttgatcctcctttatcttcctgaaccctgctgagatgatctatgtggggagaatggcttctttgagaaacatcttcttcgttagtggcctgcccctcattcccactttaatatccagaatcactataagaagaatataataagaggaataactcttattataggtaagggaaaattaagaggcatacgtgatgggatgagtaagagaggagagggaaggattaatggacgataaaatctactactatttgttgagaccttttatagtctaatcaattttgctattgttttccatcctcacgctaactccataaaaaaacactattattatctttattttgccatgacaagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagctgctgcagtcaaagtcgaatgcagctggttagactttttttaatgaaagcttagctttcattaaagattaagctcctaagcagggcacagatgaaattgtctaacagcaactttgccatctaaaaaaatctgacttcactggaaacatggaagcccaaggttctgaacatgagaaatttttaggaatctgcacaggagttgagagggaaacaagatggtgaagggactagaaaccacatgagagacacgaggaaatagtgtagatttaggctggaggtaaatgaaagagaagtgggaattaatacttactgaaatctttctatatgtcaggtgccattttatgatatttaataatctcattacatatggtaattctgtgagatatgtattattgaacatactataattaatactaatgataagtaacacctcttgagtacttagtatatgctagaatcaaatttaagtttatcatatgaggccgggcacggtggctcatatatgggattacatgcctgtaatcccagcactttgggaggccaaggcaattggatcacctgaggtcaggagttccagaccagcctggccaacatggtgaaaccccttctctactaaaaaatacaaaaaatcagccaggtgtggtggcacgcgtctataatcccagctactcaggaggctgaggcaggagaatcacttgaacccaggaggtggaggttgcagtgagctaagattgcaccactgcactccagcctaggcgacagagtgagactccatctcaaaaaaaaaaaaagaagtttattatatgaattaacttagttttactcacaccaatactcagaagtagattattacctcatttattgatgaggagcccaatgtacttgtagtgtagatcaacttattgaaagcacaagctaataagtagacaattagtaattagaagtcagatggtctgagctctcctactgtctacattacatgagctcttattaactggggactcgaaaatcaaagacatgaaataatttgtccaagcttacagaaccaccaagtagtaaggctaggatgtagacccagttctgctacctctgaagacagtgttttttccacagcaaaacacaaactcagatattgtggatgcgagaaattagaagtagatattcctgccctgtggcccttgcttcttacttttacttcttgtcgattggaagttgtggtccaagccacagttgcagaccatacttcctcaaccataattgcatttcttcaggaaagtttgagggagaaaaaggtaaagaaaaatttagaaacaacttcagaataaagagattttctcttgggttacagagattgtcatatgacaaattataagcagacacttgagaaaactgaaggcccatgcctgcccaaattaccctttgaccccttggtcaagctgcaactttggttaaagggagtgtttatgtgttatagtgttcatttactcttctggtctaacccattggctccgtcttcatcctgcagtgacctcagtgcctcagaaacatacatatgtttgtctagtttaagtttgtgtgaaattctaactagcgtcaagaactgagggccctaaactatgctaggaatagtgctgtggtgctgtgataggtacacaagaaatgagaagaaactgcagattctctgcatctccctttgccgggtctgacaacaaagtttccccaaattttaccaatgcaagccatttctccatatgctaactactttaaaatcatttggggcttcacattgtctttctcatctgtaaaaagaatggaagaactcattcctacagaactccctatgtcttccctgatgggctagagttcctctttctcaaaaattagccattattgtatttccttctaagccaaagctcagaggtcttgtattgcccagtgacatgcacactggtcaaaagtaggctaagtagaagggtactttcacaggaacagagagcaaaagaggtgggtgaatgagagggtaagtgagaaaagacaaatgagaagttacaacatgatggcttgttgtctaaatatctcctagggaattattgtgagaggtctgaatagtgttgtaaaataagctgaatctgctgccaacattaacagtcaagaaatacctccgaataactgtacctccaattattctttaaggtagcatgcaactgtaatagttgcatgtatatatttatcataatactgtaacagaaaacacttactgaatatatactgtgtccctagttctttacacaataaactaatctcatcctcataattctattagctaatacatattatcatcctatatttcagagacttcaagaagttaagcaacttgctcaagatcatctaagaagtaggtggtatttctgggctcatttggcccctcctaatctctcatggcaacatggctgcctaaagtgttgattgccttaattcatcagggatgggctcatactcactgcagaccttaactggcatcctcttttcttatgtgatctgcctgaccctagtagacttatgaaatttctgatgagaaaggagagaggagaaaggcagagctgactgtgatgagtgatgaaggtgccttctcatctgggtaccagtggggcctctaagactaagtcactctgtctcactgtgtcttagccagttccttacagcttgccctgatgggagatagagaatgggtatcctccaacaaaaaaataaattttcatttctcaaggtccaacttatgttttcttaatttttaaaaaaatcttgaccattctccactctctaaaataatccacagtgagagaaacattcttttcccccatcccataaatacctctattaaatatggaaaatctgggcatggtgtctcacacctgtaatcccagcactttgggaggctgaggtgggtggactgcttggagctcaggagttcaagaccatcttggacaacatggtgataccctgcctctacaaaaagtacaaaaattagcctggcatggtggtgtgcacctgtaatcccagctattagggtggctgaggcaggagaattgcttgaacccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatatatatatatatatacacatatacgtatatatatatatatatatatatttgttgttatcaattgccatagaatgattagttattgtgaatcaaatatttatcttgcaggtggcctctatacctagaagcggcagaatcaggctttattaatacatgtgtatagatttttaggatctatacacatgtattaatatgaaacaaggatatggaagaggaaggcatgaaaacaggaaaagaaaacaaaccttgtttgccattttaaggcacccctggacagctaggtggcaaaaggcctgtgctgttagaggacacatgctcacatacggggtcagatctgacttggggtgctactgggaagctctcatcttaaggatacatctcaggccagtcttggtgcattaggaagatgtaggcaactctgatcctgagaggaaagaaacattcctccaggagagctaaaagggttcacctgtgtgggtaactgtgaaggactacaagaggatgaaaaacaatgacagacagacataatgcttgtgggagaaaaaacaggaggtcaaggggatagagaaggcttccagaagaatggctttgaagctggcttctgtaggagttcacagtggcaaagatgtttcagaaatgtgacatgacttaaggaactatacaaaaaggaacaaatttaaggagaggcagataaattagttcaacagacatgcaaggaattttcagatgaatgttatgtctccactgagcttcttgaggttagcagctgtgagggttttgcaggcccaggacccattacaggacctcacgtatacttgacactgttttttgtattcatttgtgaatgaatgacctcttgtcagtctactcggtttcgctgtgaatgaatgatgtcttgtcagcctacttggtttcgctaagagcacagagagaagatttagtgatgctatgtaaaaacttcctttttggttcaagtgtatgtttgtgatagaaatgaagacaggctacatgatgcatatctaacataaacacaaacattaagaaaggaaatcaacctgaagagtatttatacagataacaaaatacagagagtgagttaaatgtgtaataactgtggcacaggctggaatatgagccatttaaatcacaaattaattagaaaaaaaacagtggggaaaaaattccatggatgggtctagaaagactagcattgttttaggttgagtggcagtgtttaaagggtgatatcagactaaacttgaaatatgtggctaaataactagaatactctttattttttcgtatcatgaatagcagatatagcttgatggccccatgcttggtttaacatccttgctgttcctgacatgaaatccttaatttttgacaaaggggctattcattttcattttatattgggcctagaaattatgtagatggtcctgaggaaaagtttatagcttgtctatttctctctctaacatagttgtcagcacaatgcctaggctataggaagtactcaaagcttgttaaattgaattctatccttcttattcaattctacacatggaggaaaaactcatcagggatggaggcacgcctctaaggaaggcaggtgtggctctgcagtgtgattgggtacttgcaggacgaagggtggggtgggagtggctaaccttccattcctagtgcagaggtcacagcctaaacatcaaattccttgaggtgcggtggctcactcctgtaatcacagcagtttgggacgccaaggtgggcagatcacttgaggtcaggagttggacaccagcccagccaacatagtgaaacctggtctctgcttaaaaatataaaaattagctggacgtggtgacgggagcctgtaatccaactacttgggaggctgaggcaggagaatcgcttgaaccggggaggtggagtttgcactgagcagagatcatgccattgcactccagcctccagagcgagactctgtctaaagaaaaacgaaaacaaacaaacaaacaaacaaacaaaacccatcaaattccctgaccgaacagaattctgtctgattgttctctgacttatctaccattttccctccttaaagaaactgtgaacttccttcagctagaggggcctggctcagaagcctctggtcagcatccaagaaatacttgatgtcactttggctaaaggtatgatgtgtagacaagctccagagatggtttctcatttccatatccacccacccagctttccaattttaaagccaattctgaggtagagactgtgatgaacaaacaccttgacaaaattcaacccaaagactcactttgcctagcttcaaaatccttactctgacatatactcacagccagaaattagcatgcactagagtgtgcatgagtgcaacacacacacacaccaattccatattctctgtcagaaaatcctgttggtttttcgtgaaaggatgttttcagaggctgaccccttgccttcacctccaatgctaccactctggtctaagtcactgtcaccaccacctaaattatagctgttgactcataacaatcttcctgcttctaccactgccccactacaatttcttcccaatatactatccaaattagtcttttcaaaatgtaagtcatatatggtcacctctttgttcaaagtcttctgatagtttcctatatcatttataataaaaccaaatccttacaattctctacaatagttgttcatgcatatattatgtttattacagatacatatatatagctctcatataaataaatatatatatttatgtgtatgtgtgtagagtgttttttcttacaactctatgatgtaggtattattagtgtcccaaattttataatttaggacttctatgatctcatcttttattctccccttcaccgaatctcatcctacattggccttattgatattccttgaaaattctaagcatcttacatctttagggtatttacatttgccattccctatgccctaaatatttaatcatagtttcatataaatgggttcctcatcatctatgggtactctctcaggtgttaactttatagtgaggactttcctgccatactacttaaagtagcgataccctttcaccctgtcctaatcacactctggccttcatttcagttttttttttttctccatagcacctaatctcattggtatataacatgtttcatttgcttatttaatgtcaagctctttccactatcaagtccatgaaaacaggaactttattcctctattctgtttttgtgctgtattcttagcaattttacaattttgaatgaatgaatgagcagtcaaacacatatacaactataattaaaaggatgtatgctgacacatccactgctatgcacacacaaagaaatcagtggagtagagctggaagtgctaagcctgcatagagctagttagccctccgcaggcagagccttgatgggattactgagttctagaattggactcatttgttttgtaggctgagatttgctcttgaaaacttgttctgaccaaaataaaaggctcaaaagatgaatatcgaaaccagggtgttttttacactggaatttataactagagcactcatgtttatgtaagcaattaattgtttcatcagtcaggtaaaagtaaagaaaaactgtgccaaggcaggtagcctaatgcaatatgccactaaagtaaacattatttcataggtgtcagatatggcttattcatccatcttcatgggaaggatggccttggcctggacatcagtgttatgtgaggttcaaaacacctctaggctataaggcaacagagctccttttttttttttctgtgctttcctggctgtccaaatctctaatgataagcatacttctattcaatgagaatattctgtaagattatagttaagaattgtgggagccattccgtctcttatagttaaatttgagcttcttttatgatcactgtttttttaatatgctttaagttctggggtacatgtgccatggtggtttgctgcacccatcaacccgtcatctacattaggtatttctcctaatgctatccttcccctagccccccacccccaacaggccccagtgtgtgatgttcccctccctgtgtccatggatcactggtttttttttgttttttttttttttttaaagtctcagttaaatttttggaatgtaatttattttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagtacagaaaagaggctctagcagcatagtggtttagaggagatgtttctttccttcacagatgccttagcctcaataagcttgcggttgtggaagtttactttcagaacaaactcctgtggggctagaattattgatggctaaaagaagcccgggggagggaaaaatcattcagcatcctcacccttagtgacacaaaacagagggggcctggttttccatatttcctcatgatggatgatctcgttaatgaaggtggtctgacgagatcattgcttcttccatttaagccttgctcacttgccaatcctcagttttaaccttctccagagaaatacacattttttattcaggaaacatactatgttatagtttcaatactaaataatcaaagtactgaagatagcatgcataggcaagaaaaagtccttagctttatgttgctgttgtttcagaatttaaaaaagatcaccaagtcaaggacttctcagttctagcactagaggtggaatcttagcatataatcagaggtttttcaaaatttctagacataagattcaaagccctgcacttaaaatagtctcatttgaattaactctttatataaattgaaagcacattctgaactacttcagagtattgttttatttctatgttcttagttcataaatacattaggcaatgcaatttaattaaaaaaacccaagaatttcttagaattttaatcatgaaaataaatgaaggcatctttacttactcaaggtcccaaaaggtcaaagaaaccaggaaagtaaagctatatttcagcggaaaatgggatatttatgagttttctaagttgacagactcaagttttaaccttcagtgcccatcatgtaggaaagtgtggcataactggctgattctggctttctactcctttttcccattaaagatccctcctgcttaattaacattcacaagtaactctggttgtactttaggcacagtggctcccgaggtcagtcacacaataggatgtctgtgctccaagttgccagagagagagattactcttgagaatgagcctcagccctggctcaaactcacctgcaaacttcgtgagagatgaggcagaggtacactacgaaagcaacagttagaagctaaatgatgagaacacatggactcatagagggaaacaacgcatactggggcctatcagagggtggagggtgagagaaggagaggatcaggaaaaatcactaatggatgctaagcgtaatacctgagtgatgagatcatctatacaacaaacccccttgacattcatttatctatgtaacaaacctgcacatcctgtacatgtacccctgaacttaaaataaaagttgaaaacaagaaagcaacagtttgaacacttgttatggtctattctctcattctttacaattacactagaaaatagccacaggcttcctgcaaggcagccacagaatttatgacttgtgatatccaagtcattcctggataatgcaaaatctaacacaaaatctagtagaatcatttgcttacatctatttttgttctgagaatatagatttagatacataatggaagcagaataatttaaaatctggctaatttagaatcctaagcagctcttttcctatcagtggtttacaagccttgtttatatttttcctattttaaaaataaaaataaagtaagttatttgtggtaaagaatattcattaaagtatttatttcttagataataccatgaaaaacattcagtgaagtgaagggcctactttacttaacaagaatctaatttatataatttttcatactaatagcatctaagaacagtacaatatttgactcttcaggttaaacatatgtcataaattagccagaaagatttaagaaaatattggatgtttccttgtttaaattaggcatcttacagtttttagaatcctgcatagaacttaagaaattacaaatgctaaagcaaacccaaacaggcaggaattaatcttcatcgaatttgggtgtttctttctaaaagtcctttatacttaaatgtcttaagacatacatagattttattttactaattttaattatatagacaataaatgaatattcttactgattactttttctgactgtctaatctttctgatctatcctggatggccataacacttatctctctgaactttgggcttttaatataggaaagaaaagcaataatccatttttcatggtatctcatatgataaacaaataaaatgcttaaaaatgagcaggtgaagcaatttatcttgaaccaacaagcatcgaagcaataatgagactgcccgcagcctacctgacttctgagtcaggatttataagccttgttactgagacacaaacctgggcctttcaatgctataacctttcttgaagctcctccctaccacctttagccataaggaaacatggaatgggtcagatccctggatgcaagccaggtctggaaccataggcagtaaggagagaagaaaatgtgggctctgcaactggctccgagggagcaggagaggatcaaccccatactctgaatctaagagaagactggtgtccatactctgaatgggaagaatgatgggattacccatagggcttgttttagggagaaacctgttctccaaactcttggccttgagatacctggtccttattccttggactttggcaatgtctgaccctcacattcaagttctgaggaagggccactgccttcatactgtggatctgtagcaaattccccctgaaaacccagagctgtatcttaattggttaaaaaaaattatattatctcaacgactgttcttctctgagtagccaagctcagcttggttcaagctacaagcagctgagctgctttttgtctagtcattgttcttttatttcagtggatcaaatacgttctttccaaacctaggatcttgtcttcctaggctatatattttgtcccaggaagtcttaatctggggtccacagaacactagggggctggtgaagtttatagaaaaaaaatctgtatttttacttacatgtaactgaaatttagcattttcttctactttgaatgcaaaggacaaactagaatgacatcatcagtacctattgcatagttataaagagaaaccacagatattttcatactacaccataggtattgcagatctttttgtttttgtttttgtttgagatggagtttcgctcttattgcccaggctggagtgcagtggcatgatttcggctcactgcaacctccccttcctgcattcaagcaattctcctgccttggcctcctgagtagctggggattacaggcacctgccaccatgccagtctaatttttgtatttttagtagagatggggtttcgccatgttggccaggctggtcttgaactcctgacctcagatgatctgcccgccttggcctcctgaagtgctgggattataggtgtgagccaccacgcctggcccattgcagatatttttaattcacatttatctgcatcactacttggatcttaaggtagctgtagacccaatcctagatctaatgctttcataaagaagcaaatataataaatactataccacaaatgtaatgtttgatgtctgataatgatatttcagtgtaattaaacttagcactcctatgtatattatttgatgcaataaaaacatatttttttagcacttacagtctgccaaactggcctgtgacacaaaaaaagtttaggaattcctggttttgtctgtgttagccaatggttagaatatatgctcagaaagataccattggttaatagctaaaagaaaatggagtagaaattcagtggcctggaataataacaatttgggcagtcattaagtcaggtgaagacttctggaatcatgggagaaaagcaagggagacattcttacttgccacaagtgtttttttttttttttttttttatcacaaacataagaaaatataataaataacaaagtcaggttatagaagagagaaacgctcttagtaaacttggaatatggaatccccaaaggcacttgacttgggagacaggagccatactgctaagtgaaaaagacgaagaacctctagggcctgaacatacaggaaattgtaggaacagaaattcctagatctggtggggcaaggggagccataggagaaagaaatggtagaaatggatggagacggaggcagaggtgggcagatcatgaggtcaagagatcgagaccatcctggcaaacatggtgaaatcccgtctctactaaaaataaaaaaattagctgggcatggtggcatgcgcctgtagtcccagctgctcgggaggctgaggcaggagaatcgtttgaacccaggaggcgaaggttgcagtgagctgagatagtgccattgcactccagtctggcaacagagtgagactccgtctcaaaaaaaaaaaaaaaagaaagaaagaaaagaaaaagaaaaaagaaaaaataaatggatgtagaacaagccagaaggaggaactgggctggggcaatgagattatggtgatgtaagggacttttatagaattaacaatgctggaatttgtggaactctgcttctattattcccccaatcattacttctgtcacattgatagttaaataatttctgtgaatttattccttgattctaaaatatgaggataatgacaatggtattataagggcagattaagtgatatagcatgagcaatattcttcaggcacatggatcgaattgaatacactgtaaatcccaacttccagtttcagctctaccaagtaaagagctagcaagtcatcaaaatggggacatacagaaaaaaaaaaggacactagaggaataatataccctgactcctagcctgattaatatatcgattcacttttttctctgtttgatgacaaattctggctttaaataattttaggattttaggcttctcagctcccttcccagtgagaagtataagcaggacagacaggcaagcaagaagagagccccaggcaatactcacaaagtagccaatgtcccctgtggtcatagagaaatgaaaagagagaggattctctggaagcactggatgtaatcttttctgtctgtcctctctagggaatcaccccaaggtactgtactttgggattaaggctttagtcccactgtggactacttgctattctgttcagtttctagaaggaactatgtacggtttttgtctccctagagaaactaaggtacagaagttttgtttacaatgcactccttaagagagctagaactgggtgagattctgttttaacagctttattttcttttccttggccctgtttttgtcactgtcaccacctttaaggcaaatgttaaatgcgctttggctgaaactttttttcctattttgagatttgctcctttatatgaggctttcttggaaaaggagaatgggagagatggatatcattttggaagatgatgaagagggtaaaaaaggggacaaatggaaatttgtgttgcagatagatgaggagccaacaaaaaagagcctcaggatccagcacacattatcacaaacttagtgtccatccatcactgctgaccctctccggacctgactccacccctgagggacacaggtcagccttgaccaatgacttttaagtaccatggagaacagggggccagaacttcggcagtaaagaataaaaggccagacagagaggcagcagcacatatctgcttccgacacagctgcaatcactagcaagctctcaggcctggcatcatggtgcattttactgctgaggagaaggctgccgtcactagcctgtggagcaagatgaatgtggaagaggctggaggtgaagccttgggcaggtaagcattggttctcaatgcatgggaatgaagggtgaatattaccctagcaagttgattgggaaagtcctcaagattttttgcatctctaattttgtatctgatatggtgtcatttcatagactcctcgttgtttacccctggacccagagattttttgacagctttggaaacctgtcgtctccctctgccatcctgggcaaccccaaggtcaaggcccatggcaagaaggtgctgacttcctttggagatgctattaaaaacatggacaacctcaagcccgcctttgctaagctgagtgagctgcactgtgacaagctgcatgtggatcctgagaacttcaaggtgagttcaggtgctggtgatgtgattttttggctttatattttgacattaattgaagctcataatcttattggaaagaccaacaaagatctcagaaatcatgggtcgagcttgatgttagaacagcagacttctagtgagcataaccaaaacttacatgattcagaactagtgacagtaaaggactactaacagcctgaattggcttaacttttcaggaaatcttgccagaacttgatgtgtttatcccagagaattgtattatagaattgtagacttgtgaaagaagaatgaaatttggcttttggtagatgaaagtccatttcaaggaaatagaaatgccttattttatgtgggtcatgataattgaggtttagaaagagatttttgcaaaaaaaataaaagatttgctcaaagaaaaataagacacattttctaaaatatgttaaatttcccatcagtattgtgaccaagtgaaggcttgtttccgaatttgttggggattttaaactcccgctgagaactcttgcagcactcacattctacatttacaaaaattagacaattgcttaaagaaaaacagggagagagggaacccaataatactggtaaaatggggaagggggtgagggtgtaggtaggtagaatgttgaatgtagggctcatagaataaaattgaacctaagctcatctgaattttttgggtgggcacaaaccttggaacagtttgaggtcagggttgtctaggaatgtaggtataaagccgtttttgtttgtttgtttgttttttcatcaagttgttttcggaaacttctactcaacatgcctgtgtgttattttgtcttttgcctaacagctcctgggtaacgtgatggtgattattctggctactcactttggcaaggagttcacccctgaagtgcaggctgcctggcagaagctggtgtctgctgtcgccattgccctggcccataagtaccactgagttctcttccagtttgcaggtgttcctgtgaccctgacaccctccttctgcacatggggactgggcttggccttgagagaaagccttctgtttaataaagtacattttcttcagtaatcaaaaattgcaattttatcttctccatcttttactcttgtgttaaaaggaaaaagtgttcatgggctgagggatggagagaaacataggaagaaccaagagcttccttaagaaatgtatgggggcttgtaaaattaatgtggatgttatgggagaattccaggattccaaggaggatgatatgatggagaaaaatctttatcggggtgggaaaatggttaattaagtggacagagactcctaggcagtttttactgcaccggggaaagaaggagctgttagtggtacctgagaaagcagatttgtggtacatgtcacttttcattaaaaacaaaaacaaaacaaaacaaaacttcatagatatccaagatataggctagaattactattttaatttactcttatttacattttgaagtagctagcttgtcacatgttttatgaaattgatttggagataagatgagtgtgtatcaacaatagcctgctctttccatgaaggattccattatttcatgggttagctgaagctaagacacatgatatcattgtgcattatcttctgatagaatgtaacatgcactaaaataaagttagagttaggacctgagtgggaaagtttttggagagtgtgatgaagactttccgtgggagatagaatactaataaaggcttaaattctaaaaccagcaagctagggcttcgtgacttgcatgaaactggctctctggaagtagaagggagagtaagacatacgtagaggactaggaaagaccagatagtacagggcctggctacaaaaatacaagcttttactatgctattgcaatactaaacgataagcattaggatgttaagtgactcaggaaataagattttgggaaaaagtaatctgcttatgtgcacaaaatggattcaagtttgcagataaaataaaatatggatgatgattcaaggggacagatacaatggttcaaacccaagaggagcagtgagtctgtggaatttgaaggatggacaaaggtggggtgagaaagacatagtattcgactgactgtgggagatgagaaggaagaaggaggtgataaatgactgaaagctcccagactggtgaagataacaggaggaaaccatgcactgacctggtgactctcatgtgtgaagggtagagggatattaacagatttactttttaggaagtgctagattggtcagggagttttgaccttcaggtcttgtgtctttcatatcaaggaacctttgcattttccaagttagagtgccatattttggcaaatataactttattagtaattttatagtgctctcacattgatcagactttttcctgtgaattacttttgaatttggctgtatatatccagaatatgggagagagacaaataattattgtagttgcaggctatcaacaatactggtctctctgagccttataacctttcaatatgcccataaacagagtaaacagggattattcatggcactaaatattttcacctagtcagtcaacaaatgggagcaatgtgcattttttgatacatatttttatatatttatggggtacatgtgatacttacatgcctagaacatgtgatgattaagtctagatatttaggatatccattgctttgagcatttatcatttctatgtattgagaaaatttcaaatcctcatttctagccattttgaaatatataataaatagtaattaactatagtcaccctactcaaatatcaaacattatggcttaatccttctatccaactgtgtttgtacctattaaccaacatctcttaaatcccctcccatacacactcacactttttccagcctctgataactatcattctactctctaccaccatgagacccacttttttagctcccacagatgaataaaaacatgtgatatttgactttctgtatctggcttattttattatctatctctttggcataccaagagtttgtttttgttctgcttcagggctttcaattaacataatgacctctggttccatccatgttgctacaaatgacaagatttcattctttttcatggcaaaatagtactgtgcaaaaatacaattttttaatccgttcatctgttgatagacacttaggttgatcccaaaccttaactattgtgaatagtgcttcaataaacatgagtgtaatgtgtccattggatatactgatttcctttcttttggataaataaccactagtgagattgctggattgtatgatagttctgtttttagtttactgagaaatcttcatactgttttccataatggttgtactattttacattcccaccaacagtgtgtaagaaagagttcccttttctccatatcctcacaaggatctgttattttttgtcttttttgttaatagccgttttaactagagtaagtagatatctcattgtagttttgatttgcatttccctgatcattagtgatgttgagaattttttcatatgtttgttggtcatttgtatatctttttctgagaattgtctgttcatgtccttagcctactttttattgggattgtttgttattttcttgataatctatttgtgttcattttagagcctggatattattcttttgtcagatgtatagattgtgaagattttctcccactctgtgggttgtctgtttattctgcagactcttccttttgccatgcaaaagctctttagtttaatttagtcccagatattttctttgtttttatgtatttgcatttgtgttcttggtcatgaaatcctttcctaagccaatgtgtagaagggtttttccgatgttattttctagaattgttacagtttcagggcttagatttaagtccttgatccatcttgagttgatttttgtataaggtgagagatgaagatccagtttcattctcctacatgtagcttgccagctatccccgcaccatttgttgaatagggtgccctttccccactttatgtttttgtttgctttgtcaaagatcagttggatgtaagtatttgagtttatttctgggttctctattctgttccattggtcgatgtgcctatttgtacaccagcatcatgctgttttggtgactatggccttattgtatagtttgaaatgaggtaatgtaatgccttcagatttgttcttttttttagacttgcttgtttattgggctcttttttggttccataagaattttaggattgttttttctagttctgtgaagactaatggtggtattttgatgggaattgcaatgaatttgtaggttgcttctggcattatggccattttcacaatattgattctacccatctatgagaatggcatgtgtttccatttgtttgtgtcttatatgattactttcagccgtgttttgtagttttccttgtagatgtctttcacctccttggttaggtatatattcctaagtttttgttttgttttgttttgttttttgcagctattgtaaaaggggttgagttcttgattttattctcagcttggtcattgctggtatgtaagaaagcaactcattggtgtacgttaattttgtatccagaaactttgctgaattattttatcagttctagggggttttggaggagtctttagagttttctacatacacaatcatatcatcagcaaacagtgacagtttgactttctctttaacaatttggatgtgctttacttgtttctcttgtctgattgctcttgctaggacttccagtaatatgttaaagagaagtggtgagagtgggtatccttgtctcattccagttttcagacagaatgcttttaactttttcccattcaatataatgttggctgtgtgtttaccatagctggcttttattacattgaggtatgtcctttgtaaaccgattttgctgagttttagtcataaagtgatgttgaattttgttgaatgcagtttctgtggctattgagataatcacatgatttttgtttccaattctctttatgttgtgtatcacacttattgacttgcgtatgttaaaccatccgtgcatccctcgcatgaaacccacttgatcatgggttttgatatgctgtcggatgctattagctagtattttgtcaaggatgttggcatctatgttcatcagggatattgatctgtagtgttttttttttttggttatgttctttcccagttttggtattaaggtgatactggcttcatagaatgatttagggaggattctctctttctctatcttgtagaatactgtcaataggattggtatcaattcttctttgaatgtctggtagaattcagctgtgaatctatctggtcctggacttttttgttgttggtaaatttttattatcatttcagtcttgctgcttattactggtctgttcagggtatctaattcttcctgacttaagctagagccctgtatctttccaggaattcgaacgtctcctttaggttttctagtttatgcatgtaaaggtgttcatagtagccttgaataatcttttgtatttctgtggtatcagtaatagtatctcctgttttgtttctaattgagtttatttgcacttctctcctcttttcttggttaatcttgctaatggtctatcagttttatttatcttttcaaagaaccagctttttatttcatttagcttttgtatttttttgcagttgttttaatttcatttagttctcctcttatcttagttattccctttcttttgctgggttttggttctgtttgtttttgtttctctagtttcttgtggtgtgaccttatattgtctgtctgtcctctttcagactctttgacatcgacatttagggctgtgaactttccttttagcaccatctttgctgtatcctagaggttttgataggttgtgtcactattgtcggtcagttcaagtaattttgttgttcttattatactttaagttctgggatacatgtgcagaatgtgcaggtttgttacataggtatagatgtgccatggtggtttgctgcacccatcaacctgtcatctacattaggtatttcttttaatgttatccctctcctaaccccctcaccccccgacaggccctggtgtgtgatgttcccctccctgtgtccatgtgttctcattgttcaactcccacttatgagtgagaacgtgtggtgtttggtttctctgttcctgtgttagtttgctcagaatgatggtttccaccttcatccatgtccctgcaaagacatgaactcatcatttttatggctgcatagtattccatggtgtatatgtgccacattttctttatccattatatcgctgatggccatttgggttggttccaagtctttgctattgtgaatagtgccacaataaacatacgtgtgcacgtgtctttatagtagaatgatttctaattctttgggtatatacccagtaatgggattgctgggtcaaacagtatttctggttctagatccttgaggaatcgccacactgtcttccacaatggttgaactaatttacacacccatcaacagtgtaaaatttttcctattcttccacatcctctccagcaccttttgtttcctgactttttaataattgccattctaactggcatgagatggtatctcattgtggttttgatttgcatttctctaatgaccagtgatgatgagcttcttttcatgtgtttcttggccacataaatgacttctttagagaagcatctgttcatatcctttgtccactttttgatggggtcgttaggttttttcttgtaaatttgttgaagttctttgtagattttggatgttagccctttgtcagatggatagattgcaaaaattttctcccattctgtaggttgcctgttcactctgatgatagtcttttgctgtgcagaagctctttagtttaattagatcccatatgtcaattttggcctttgttgtcattgcttttgatgttttagtcgtgaatttttgcccatgcctatgtcctgaatggtattgcctaggttatcttctaggatttttatggttttaggttgcacatttaagtctttaatccaccttgagttaatttttgtataaggtgtaaggaaggggtacagtttcagttttatgcatattgctagccagtttttccagcaccatttattaaatagggaattctttctccattgcttttgtgatgtttgtcaaagatcagatggtcgtagatgtgtggcattatttctgaggcttctgttctgttccactggtctatatatctgttttggtaccagtaccatgctgtttttgttactgtagccttgtagtatagtttgaagtcaggtagcatcatgcctccagctttgttctttttgtttaggattgtcttggctatatgggctcttttttgattccatatgacatttaaagtagttttttctaattctttgaaaaaagtcagtggtagcttgatggggatagcattgaatctataaattactttgggcagtatggccattttaaagatattgattctttctatctatgagcatggaatgtttttccatttgtttgtgtcctctcttatttccttgagcagtgagtggtttgtagctctccttgaagaggttcttcacatcccttagaagttgtatttctaggtattttattttattctctttgcagcaattgtgaatgggagttcacccatgatttggctctctgcttgtctattattggtgtataggaacgcttgtgatttctgcacactgattttgtatcttgagactttgctgaagctgtttatcagcttaagattttgggctgagatgacagggtcttctaaatatacaatcatgtcatctgcaaacagagacaatttgacttcctctcttcctatttgaatatgctttatttctttctcttgcctgattgtcctggcgagaacttccaatactatgttgagtaagagtggcgagagggcatccttgtcttgtgccggttttcaaagcaaatgatttttaaatttccatcttgatttcattgttgacccaatgatcattcaggagcaggttatttaatttccctgtatttgcatggttttgaaggttccttttgtagttgatttccaattttattctactgtggtctgagagagtgcttgatataatttcaatttttaaaaatttattgaggcttgttttgtggcatatcatatggcctatcttggagaaagttccatgtgctgatgaatagaatgtgtattctgcagttgttgggtagaatgtcctgtaaatatctgttaagtccatttgttctttaaatccattgtttctttgtagactgtcttgatgacctgcctagtgcagtcagtggagtattgaagtcccccactattattatgttgctgtctagtctagtagtaattgttttataaatttgggatctccagtattagatgcatatatattaagaattgtaatattctcccattggacaagggcttttatcattatatgatgtccctctttgtcttttttaactgctgtttctttaaagtttgttttgtctgacataagaatagctgctttggctcgcttttggtgtccatttgtgtggaatgtcattttccacccctttaccttaagtttatgtgagtccttatgtgttaggtgagtctcctgaaggcggcagataactggttggtgaattcttattcattctgcaattctgtatcttttaagtggagcatttagtccatttacattcaacatcagtattgaggtgtgaggtactattccattcttcgtggtatttgttgcctgtgtatctttttatctgtatttttgttgtatatgtcctatgggatttatgctttaaagaggttctgttttgatgtgcttccagggtttatttcaagatttagagctccttttatcagttcttgtagtgttggcttggtagtgccgaattctctcagcatttgtttttctgaaaaacactgtgtattttcttcatttgtgaagcttagtttcactggatataaaattcttggctgataattgttttgtttaagaaggctgaagatagggccatattcacttctagcttttacggtttctgctgagaaatctgctgttaatctgataggttttctttcataggttacctggtagtttcacctcacagctcttaagattctctttgtctttagataactttggatactctgatgacaatgtacctaggcaatgatatttttgcaatgaatttcccaggtgtttattgagcttcttgtatttggatatctaggtctctagcaaggtgggggaagttttccttgattatttccctggataagttttccaaacttttagatttctcttctttctcaggaatgctgattattcttaggtttgattgtttaacataatcccagatttcttggaggctttgttcatattttcttattcttttttctttgtctttgttggattgggttaattcaaaaactttgtcttcaagctctgaatttcttctgcttggattctattgctgagactttctagagcattttgcatttctataagtgcatccattcatccattgtttcctgaagttttgaatgttttttatttatgctatctctttaactgaagatttctcccctcatttcttgtatcatatttttggtttttttaaaattggacttcaccttcctcggatgcctccttgattagcttaataactgaccttctgaattatttttcaggtaaatcagggatttcttcttggtttggatgcattgctggtgagctagtatgattttttggggggtgttaaagaaccttgtttttcatattaccagagttagttttctggttccttctcacttgggtaggctctgtcagagggaaagtctaggcctcaaggctgagacttttgtcccatgaggtgttcccttgatgtagcacagtcccccttttcctaggcgtggggcttcctgagagccgaactgtagtgattgttatctctcttctggatctagccacccatcaggtctaccagactccaggctggtactggggtttgtctgcacagagtcttgtgacgtgaaccatctgtgggtctctcagccatagatacaaccacctgctccaatggaggtggcagaggatgaaatggactctgtgagggtccttacttttggttgttcaatgcactatttttgtgctggttggcctcctgccaggaggtggcactttctagaaagcatcagcagaggcagtcaggtggtggtggctgggggggctggggcaccctagaactcccaagaatatatgccctttgtcttcagctaccagggtgagtaaggaaggaccatcaggtgggggcaggactagtcgtgtctgagctcagagtctccttgggcaggtctttctgtggctactgtgggaggatgggggtgtagtttccaggtcaatggatttatgttcctaggacaattatggctgcctctgctgtgtcatgcaggtcatcaggaaagtgggggaaagcaagcagtcacgtgacttgcccagctcccatgcaactcaaaaggttggtctcacttccagcgtgcaccctcccccgcaacagcaccgaatctgtttccatgcagtcagtgagcaaggctgagaacttgccccaggctaccagctgcgaaaccaagtagggctgtcctacttccctgccagtggagtctgcacaccaaattcatgtccccccaccaacccccccactgcccagcccctagatctggccaggtggagattttctttttcctgtcatcttttcccagttcctctggcagccctcccaaatgacccctgtgaggcaaggcagaaatggcttcctaggggacccagagagcccacagggcttttcccgctgcttcctctacccctgtattttgcttggccctctaaattgactcagctccaggtaaggtcagaatcttctcctgtggtctagatcttcaggttccccagtgaggatgtgtgtttgggggtagacggtcccccttttccacttccacagtttgggcactcacaatatttggggtgtttcccgggtcctgcaggagcaatctgcttctttcagagggtgtgtgcgttctctcagctttcttgatttatttctgcaggtggttctgcaaaaaaaattcctgatgggagacttcacatgctgctctgtgcatccgagtgggagctgcaatgtacttctgctgcctcccatctgccatcaccctctaatttgtcggtaatatgcatttttaatcaatctttttttctctctctctctttttcttctcccccaaaactatactgccctttgatatcaaggaatcaaggacgtgatgttgaggggtgggcagtggatacactctttaccccttagggagctatatctagatttagatattgccaattcaagataacttaattgaaagcaaattcataatgaatacacacacacacacacacatctgcatgacaagatttttaatagttgaaagaataactaataattgtccacaggcaataagggctttttaagcaaaacagttgtgataaacaggtcattcttagaatagtaatccagccaatagtacaggttgcttagagattatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaagggggctgaacccccttccctggattgcagcacagcagcgaggaaggggctcaacgaagaaaaagtgttccaagctttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaaggaagggaaagtgttccaagctttaggaattcaaggtttagtcaggtgtagcaattctattttattaggaggaatactatttctaatggcacttagcttttcacagcccttgtggatgcctaagaaagtgaaattaatcccatgccctcaagtgtgcagattggtcacagcatttcaagggagagacctcattgtaagactctgggggaggtggggacttaggtgtaagaaatgaatcagcagaggctcacaagtcagcatgagcatgttatgtctgagaaacagaccagcactgtgagatcaaaatgtagtgggaagaatttgtacaacattaattggaaggcttacttaatggaatttttgtatagttggatgttagtgcatctctataagtaagagtttaatatgatggtgttacggacctaatgtttgtgtctcctcaaaattcacatgctgaatccccaactcccaactgaccttatctgtgggggaggcttttgaaaagtaattaggtttagatgagctcataagagcagatccccatcataaaattattttccttatcagaagcagagagacaagccatttctctttcctcccggtgaggacacagtgagaagtccgccatctgcaatccaggaagagaaccctgaccacgagtcagccttcagaaatgtgagaaaaaactctgttgttgaagccacccagtcttttgtattttgttatagcaccttgcactgagtaaggcagatgaagaaggagaaaaaaataagcttgggttttgagtggactacagaccatgtttatctcaggtttgcaaagctcccctcgtcccctatgtttcagtataaaatacctactctactactctcatctataagacccaaataataagcctgcgcccttctctctaactttgatttctcctatttttacttcaacatgctttactctagccttgtaatgtctttacatacagtgaaatgtaaagttctttattctttttttctttctttcttttttctcctcagcctcagaatttggcacatgcccttccttctttcaggaacttctccaacatctctgcctggctccatcatatcataaaggtcccacttcaaatgcagtcactaccgtttcagaatatgcactttctttcttttttgttttttgttttttttaagtcaaagcaaatttcttgagagagtaaagaaataaacgaatgactactgcataggcagagcagccccgagggccgctggttgttccttttatggttatttcttgatgatatgttaaacaagttttggattatttatgccttctctttttaggccatatagggtaactttctgacattgccatggcatttttcttttaatttaatttactgttaccttaaattcaggggtacacgtacaggatatgcaggtttgttttataggtaaaagtgtgccatggttttaatgggttttttttttcttgtaaagttgtttaagtttcttgtttactctggatattaggcctttgtcagaagaatagattggaaaatctttttcccattctgtagattgtctttcgctctgatggtagtttcttttgctgagcaggagctctttagtttaattagattccattggtcaatttttgcttttgctgcaattgcttttcacgctttcatcatgaaatctgtgcccgtgtttatatcatgaatagtattgccttgatttttttctaggctttttatagtttggggtttttcatttaagtctctaatccatctggagttaattttggataaggtataaggaaggagtccagtttcatttttcagcatatggctagccagttctcccccatcatttattaaattgaaaatcctttccccattgcttgcttttgtcaggtttctaaaagaccagatggttgtaggtacaatatgcagtttcttcaagtcatataataccatctgaaatctcttattaattcatttcttttagtatgtatgctggtctcctctgctcactatagtgagggcaccattagccagagaatctgtctgtctagttcatgtaagattctcagaattaagaaaaatggatggcatatgaatgaaacttcatggatgacatatggaatctaatatgtatttgttgaattaatgcataagatgcaacagagagaagttgacaactgcaatgataacctggtattgatgatataagagtctatagatcacagtagaagcaataatcatggaaaacaattggaaatggggaacagccacaaacaagaaagaatcaatacttccaggaaagtgactgcaggtcacttttcctggagcgggtgagagaaaagtggaagttagcagtaactgctgaattcctggttggctgatggaaagatggggcagctgttcactggtacgcagggttttagatgtatgtacctaaggatatgaggtatggcaatgaacagaaattcttttgggaatgagttttagggccattaaaggacatgacctgaagtttcctctgaggccagtccccacaactcaatataaatgtgtttcctgcatatagtcaaagttgccacttctttttcttcatatcatcgatctctgctcttaaagataatcttggttttgcctcaaactgtttgtcactacaaactttccccatgttcctaagtaaaacaggtaactgcctctcaactatatcaagtagactaaaatattgtgtctctaatatcagaaattcagctttaatatattgggtttaactctttgaaatttagagtctccttgaaatacacatgggggtgatttcctaaactttatttcttgtaaggatttatctcaggggtaacacacaaaccagcatcctgaacctctaagtatgaggacagtaagccttaagaatataaaataaactgttcttctctctgccggtggaagtgtgccctgtctattcctgaaattgcttgtttgagacgcatgagacgtgcagcacatgagacacgtgcagcagcctgtggaatattgtcagtgaagaatgtctttgcctgattagatataaagacaagttaaacacagcattagactatagatcaagcctgtgccagacacaaatgacctaatgcccagcacgggccacggaatctcctatcctcttgcttgaacagagcagcacacttctcccccaacactattagatgttctggcataattttgtagatatgtaggatttgacatggactattgttcaatgattcagaggaaatctcctttgttcagataagtacactgactactaaatggattaaaaaacacagtaataaaacccagttttccccttacttccctagtttgtttcttattctgctttcttccaagttgatgctggatagaggtgtttatttctattctaaaaagtgatgaaattggccgggcgcggtggctcacacctgtaatcccagcactttgggaggctgaggtgggcggatcacgaggtcaggagatcaagaccatcctggctaacatggtgaaaccccatctctactaaaaatacaaaaaattagccagagacagtggcgggtgcctgtagtcccagctactcgggaggctgaggcaggagaatggcgtgaacctgggaggcagagcttgcggtgagcagagatcgcgccactgcacactccagcctgggtgacaaagcgagactccatctcaaaaaaaaaaaaaaaaaaaagaaaaagaaagaaagaaagaaaaaaaaactgatgaaattgtgtattcaatgtagtctcaagagaattgaaaaccaagaaaggctgtggcttcttccacataaagcctggatgaataacaggataacacgttgttacattgtcacaactcctgatccaggaattgatggctaagatattcgtaattcttatccttttcagttgtaacttattcctatttgtcagcattcaggttattagcggctgctggcgaagtccttgagaaataaactgcacactggatggtgggggtagtgtaggaaaatggaggggaaggaagtaaagtttcaaattaagcctgaacagcaaagttcccctgagaaggccacctggattctatcagaaactcgaatgtccatcttgcaaaacttccttgcccaaaccccacccctggagtcacaacccacccttgaccaatagattcattttactgagggaggcaaagggctggtcaatagattcatttcactgggagaggcaaagggctgggggccagagaggagaagtaaaaagccacacatgaagcagcaatgcaggcatgcttctggctcatctgtgatcaccaggaaactcccagatctgacactgtagtgcatttcactgctgacaagaaggctgctgccaccagcctgtgaagcaaggttaaggtgagaaggctggaggtgagattctgggcaggtaggtactggaagccgggacaaggtgcagaaaggcagaaagtgtttctgaaagagggattagcccgttgtcttacatagtctgactttgcacctgctctgtgattatgactatcccacagtctcctggttgtctacccatggacctagaggtactttgaaagttttggatatctgggctctgactgtgcaataatgggcaaccccaaagtcaaggcacatggcaagaaggtgctgatctccttcggaaaagctgttatgctcacggatgacctcaaaggcacctttgctacactgagtgacctgcactgtaacaagctgcacgtggaccctgagaacttcctggtgagtagtaagtacactcacgctttcttctttacccttagatatttgcactatgggtacttttgaaagcagaggtggctttctcttgtgttatgagtcagctatgggatatgatatttcagcagtgggattttgagagttatgttgctgtaaataacataactaaaatttggtagagcaaggactatgaataatggaaggccacttaccatttgatagctctgaaaaacacatcttataaaaaattctggccaaaatcaaactgagtgtttttggatgagggaacagaagttgagatagagaaaataacatctttcctttggtcagcgaaattttctataaaaattaatagtcacttttctgcatagtcctggaggttagaaaaagatcaactgaacaaagtagtgggaagctgttaaaaagaggattgtttccctccgaatgatgatggtatacttttgtacgcatggtacaggattctttgttatgagtgtttgggaaaattgtatgtatgtatgtatgtatgtatgtgatgactggggacttatcctatccattactgttccttgaagtactattatcctactttttaaaaggacgaagtctctaaaaaaaaaatgaaacaatcacaatatgttggggtagtgagttggcatagcaagtaagagaaggataggacacaatgggaggtgcagggctgccagtcatattgaagctgatatctagcccataatggtgagagttgctcaaactctggtgaaaaaggatgtaagtgttatatctatttactgcaagtccagcttgaggccttctattcactatgtaccattttcttttttatcttcactccctccccagctcttaggcaacgtgatattgattgttttggcaacccacttcagcgaggattttaccctacagatacaggcttcttggcagtaactaacaaatgctgtggttaatgctgtagcccacaagaccactgagttccctgtccactatgtttgtacctatggtccactatgtttgtacctatgtcccaaaatctcatctcctttagatgggggaggttggggagaagagcagtatcctgcctgctgattcagttcctgcatgataaaaatagaataaagaaatatgctctctaagaaatatcattgtactctttttctgtctttatattttaccctgattcagccaaaaggacgcactatttctgatggaaatgagaatgttggagaatgggagtttaaggacagagaagatactttcttgcaatcctgcaagaaaagagagaactcgtgggtggatttagtggggtagttactcctaggaaggggaaatcgtctctagaataagacaatgtttttacagaaagggaggtcaatggaggtactctttggaggtgtaagaggattgttggtagtgtgtagaggtatgttaggactcaaattagaagttctgtataggctattatttgtatgaaactcaggatatagctcatttggtgactgcagttcacttctacttattttaaacaacatattttttattatttataatgaagtggggatggggcttcctagagaccaatcaagggccaaaccttgaactttctcttaacgtcttcaatggtattaatagagaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtacaagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctgagatttaggaaacagcacgttaagtcaagttgatagaggagaatatggacatttaaaagaggcaggatgatataaaattagggaaactggatgcagagaccagatgaagtaagaaaaatagctatcgttttgagcaaaaatcactgaagtttcttgcatatgagagtgacataataaatagggaaacgtagaaaattgattcacatgtatatatatatatagaactgattagacaaagtctaacttgggtatagtcagaggagcttgctgtaattatattgaggtgatggataaagaactgaagttgatggaaacaatgaagttaagaaaaaaaatcgagtaagagaccattgtggcagtgattgcacagaactggaaaacattgtgaaacagagagtcagagatgacagctaaaatccctgtctgtgaatgaaaagaaggaaatttattgacagaacagcaaatgcctacaagccccctgtttggatctggcaatgaacgtagccattctgtggcaatcacttcaaactcctgtacccaagacccttaggaagtatgtagcaccctcaaacctaaaacctcaaagaaagaggttttagaagatataataccctttcttctccagtttcattaatcccaaaacctctttctcaaagtatttcctctatgtgtccaccccaaagagctcacctcaccatatctcttgagtgggagcacatagataggcggtgctaccatctaacagcttctgaaattcctttgtcatatttttgagtccccactaataacccacaaagcagaataaataccagttgctcatgtacaataatcactcaactgctgtcttgtagcatacattaattaagcacattctttgaataattactgtgtccaaacaatcacactttaaaatctcacacttgtgctatcccttgcccttctgaatgtcactctgtattttaaatgaagagatgagggttgaatttcctgtgttacttattgttcatttctcgatgaggagttttcacattcacctttagtggaaaacacataagtacacatcttacaggaaaaatataccaaactgacatgtagcatgaatgcttgtgcatgtagtcatataaaatcttgtagcaatgtaaacattctctgatatacacatacagatgtgtctatatgtctacacaatttcttatgctccatgaacaaacattccatgcacacataagaacacacactgttacagatgcatacttgagtgcattgacaaaattaccccagtcaatctagagaatttggatttctgcatttgactctgttagctttgtacatgctgttcatttactctgggtgatgtctttccctcattttgccttgtctatcttgtactcatactttaagtcctaacttatatgttatctcaactaagaagctatttttttttaattttaactgggcttaaagccctgtctataaactctgctacaattatgggctctttcttataatatttagtgtttttcctactaatgtacttaatctgctcattgtatattcctaccactaaattttaacctcttttatggtagagacattgtcttgtaaactcttatttccctagtatttggagatgaaaaaaaagattaaattatccaaaattagatctctcttttctacattatgagtattacactatccatagagaagtttgtttgagacctaaactgaggaacctttggttctaaaatgactatgtgatatcttagtatttataggtcatgaggttccttcctctgcctctgctatagtttgattagtcaacaagcatgtgtcatgcatttattcacatcagaatttcatacactaataagacatagtatcagaagtcagtttattagttatatcagttagggtccatcaaggaaaggacaaaccattatcagttactcaacctagaattaaatacagctcttaatagttaattatccttgtattggaagagctaaaatatcaaataaaggacagtgcagaaatctagatgttagtaacatcagaaaacctcttccgccattaggcctagaagggcagaaggagaaaatgtttataccaccagagtccagaaccagagcccataaccagaggtccactggattcagtgagctagtgggtgctccttggagagagccagaactgtctaatgggggcatcaaagtatcagccataaaaaaccataaaaaagactgtctgctgtaggagatccgttcagagagagagagagaccagaaataatcttgcttatgctttccctcagccagtgtttaccattgcagaatgtacatgcgactgaaagggtgaggaaacctgggaaatgtcagttcctcaaatacagagaacactgagggaaggatgagaaataaatgtgaaagcagacatgaatggtaattgacagaaggaaactaggatgtgtccagtaaatgaataattacagtgtgcagtgattattgcaatgattaatgtattgataagataatatgaaaacacagaattcaaacagcagtgaactgagattagaattgtggagagcactggcatttaagaatgtcacacttagaatgtgtctctaggcattgttctgtgcatatatcatctcaatattcattatctgaaaattatgaattaggtacaaagctcaaataatttattttttcaggttagcaagaactttttttttttttttctgagatagagcattgctatggttgcccaggctggagtgcaatggcatgatccaggctcactgcaacatctgcctcccaggttcaagcgattctcctgcctcagcctcccaagtagctggcactacaggcatgtgccaccaccatgcctggctaattttctatttttagtagatagggggtttcaccatgttggtcaggctgatctcgaactcctaacatcaggtgatccaccctcctcggcctctgaaagtgctgggatcacaggcgtgagccaccacacccagccaagaatgtgaattttgtagaaggatataacccatatttctctgaccctagagtccttagtatacctcccataccatgtggctcatcctccttacatacatttcccatctttcaccctaccttttcctttttgtttcagcttttcactgtgtcaaaatctagaaccttatctcctacctgctctgaaaccaacagcaagttgacttccattctaacccacattggcattacactaattaaaatcgatactgagttctaaaatcatcggggattttggggactatgtcttacttcatacttccttgagatttcacattaaatgttggtgttcattaaaggtccttcatttaactttgtattcatcacactcttggattcacagttatatctaaactcttaaatacagcctgtataatcccaattcccaactctgatttctaacctctgacctccaacctcagtgccaaacccatatatcaaacaatgtactgggcttatttatatagatgtcctataggcacctcagactcagcatgggtatttcacttgttatactaaaactgtttctcttccagtgttttccattttagtcattagatagctacttgcccattcaccaaggtcacagattaaaatcatttccctacctctaatcaacagttcgattctgcttcaatttgtccctatctattaatcaccactcttactgcccagtcaggtcctcattgtttcctgaacaagagtagatgctattctttccacttttagaccttatcctggctggatgcggtggctcaggcttgtaaacccagcactttgggaggccaaggcaggcagatcacttgaggtcaggagttcaagaccagcctgaccaacatggtgaaaccccatctctactaaaaatacaaaatcagccgggcgtgtggtgcatgcctgcagtcccagctattcaggtggctgaggcaggagaattgcttgaacccaggaggcagaggttgcggtgagcctagattgcaccattgcactctagcttgggcaatagggatgaaactccatctcagaagagaaaagaaaaaaagaccttattctgttatacaaatcctctcaatgcaatccatatagaataaacatgtaaccagatctcccaatgtgtaaaatcatttcaggtagaacagaattaaagtgaaaagccaagtctttggaattaacagacaaagatcaaataacagtcctcatggccttaagaatttacctaacattttttttagaatcaattttcttatatatgaattggaaacataattcctccctcacaaacacattctaagattttaaggagatattgatgaagtacatcatctgtcatttttaacaggtagtggtagtgattcacacagcacattatgatctgttcttgtatgttctgttccattctgtattcttgacctggttgtattctttctgagctccagatccacatatctaagtacatctttttgcattttacaagagtgcatacaatacaatgtatccaagactgtatttctgattttatcgtaccactaaactcacaaatgtggccctattcttgtgttcacgactgacatcaccgtcatggtccaagtctgataatagaaatggcattgtcactttcttccctactgcaacagaagcccagctatttgtctcccattttctctacttctaaaatacatttcttcactaagtgagaataatcttttaaagacacaaatcaaaccatgccaccacctttcttgaattattcaatatctttcgttggcttccaggttacagaaaaataacttgtaacaaagtttaaaggtcattcatggctcctctctaccctattttataacatttccccttgtgatcagaatctcaggcacatcatccatctttctatatacaaataaagtcatatagtttgaactcacctctggttacttttaatcaaccaaatgctgtaaaatgcatttgtatcgctacgtgttaagcagtagttgattcttttcatttctgtgtaatattctattctttgactataccgtaatttatcaattctactgttggtaagcatttaagtggctaccggtttgaggtttttatgattattgctgtcataagcatttctatacatgtctttggatacacacatgcatgtgtttctgaatatctaaaaatgtaattgctaggtaatagacttatcaagcatccagcatttgtggatactattaaaggttttccaaaggggttatactattgtacagtgtcaccaacagagtttgagtttctattgatccatatcaccaccaaaatttgaactgtcagtcttatctcttctcttgtctcttttttcctcttttttttccttcccttcccctctcttcgtttcttttctctcctcttctcttctttcctctcttcccttccctttctctttctcttccctatcccttctcctctcctctcccctccttttttctcctctcctctccattatttatttttccttcttctcctccatcccttccatcctctctcttcccctcttccttccttcctttctccatttcttcctcctctttccttcaatccttccttttggatatgctcatgggtgtgtatttgtctgccattgtggcattatttgaattcagaaaagagtgaaaaactactgggatcttcattcctgggtctaattccacatttttttttaagaacacatctgtaaaaatgttctgtactagcatattcccaggaacttcgttaaatttaatctggctgaatatggtaaatctacttttcactttgcattctttctttagtcataccataattttaaacattcaaaatatttgtatataatatttgattttatctgtcattaaaatgttaaccttaaaattcatgtttccagaacctatttcaataactggtaaataaacactattcattttttaaatattcttttaatggatatttatttcaatataataaaaaattagagttttattataggaagaatttaccaaaagaaggaggaagcaagcaagtttaaactgcagcaatagatttgtccattccaacctctcaaaattcccttggagacaaaaatctctagaggcaaagaagaactttatattgagtcaacttgttaaaacatctgcttttagataagttttcttagtataaagtgacagaaacaaataagttaaactctaagatacattccactatattagcctaaaacacttctgcaaaaatgaaactaggaggatatttttagaaacaactgctgaaagagatgcggtggggagatatgtagaggagaacagggtttctgagtcaagacacacatgacagaacagccaatctcagggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcaggttggtatcaaggttataagagaggctcaaggaggcaaatggaaactgggcatgtgtagacagagaagactcttgggtttctgataggcactgactctctgtcccttgggctgttttcctaccctcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcagggtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgattaatttcacttatttcatttttttgtccaggtgtgtaagaaggttcctgaggctctacagatagggagcacttgtttattttacaaagagtacatgggaaaagagaaaagcaagggaaccgtacaaggcattaatgggtgacacttctacctccaaagagcagaaattatcaagaactcttgatacaaagataatactggcactgcagaggttctagggaagacctcaaccctaagacatagcctcaagggtaatgctacgattaaactccaacaattactgagaaaataatgtgctcaattaaaggcataatgattactcaagacaatgttatgttgtctttcttcctccttcctttgcctgcacattgtagcccataatactataccccatcaagtgttcctgctccaagaaatagcttcctcctcttacttgccccagaacatctctgtaaagaatttcctcttatcttcccatatttcagtcaagattcattgctcacgtattacttgtgacctctcttgaccccagccacaataaacttctctatactacccaaaaaatctttccaaaccctcccccacaccattttttatatttttatatttttcttatttatttcatgcacacacacacactccgtgctttataagcaattctgcctattctctaccttcttacatgcctactgtgcctcatattaaattcatcaatgggcagaaagaaaatatttattcaagaaaacagtgaatgaatgaacgaatgagtaaatgagtaaatgaaggaatgattattccttgctttagaacttctggaattagaggacaatattaataataccatcgcacagtgtttctttgttgttaatgctacaacatacaaagaggaagcatgcagtaaacaaccgaacagttatttcctttctgatcataggagtaatatttttttccttgagcaccatttttgccataggtaaaattagaaggatttttagaactttctcagttgtatacatttttaaaaatctgtattatatgcatgttgattaattttaaacttacttgaatacctaaacagaatctgttgtttccttgtgtttgaaagtgctttcacagtaactctgtctgtactgccagaatatactgacaatgtgttatagttaactgttttgatcacaacattttgaattgactggcagcagaagctcttttatatccatgtgttttccttaagtcattatacatagtaggcactgagaactctttatatctgaataagatatttaggaaccactggtttacatatcagaagcagagctactcagggcattttggggaagatcactttcacattcctgagcatagggaagttctcataagagtaagatattaaaaggagatacttgtgtggtattcgaaagacagtaagagagattgtagaccttatgatcttgatagggaaaacaaactacattcctttctccaaaagtcaaaaaaaaagagcaaatatagcttactataccttctattcctacaccattagaagtagtcagtgagtctaggcaagatgttggccctaaaaatccaaataccagagaattcatgagaacatcacctggatgggacatgtgccgagcacacacaattactatatgctaggcattgctatcttcatattgaagatgaggaggtcaagagatgaaaaaagacttggcaccttgttgttatattaaaattatttgttagagtagagcttttgtaagagtctaggagtgtgggagctaaatgatgatacacatggacacaaaaaatagatcaacagacacccaggcctacttgagggttgagggtgggaagagggagacgatgaaaaagaacctattgggtattaagttcatcactgagtgatgaaataatctgtacatcaagacccagtgatatgcaatttacctatataacttgtacatgtacccccaaatttaaaatgaaagttaaaacaaagtataggaatggaattaattcctcaagatttggctttaattttatttgataatttatcaaatggttgtttttcttttctcactatggcgttgctttataaactatgttcagtatgtctgaatgaaagggtgtgtgtgtgtgtgaaagagagggagagaggaagggaagagaggacgtaataatgtgaatttgagttcatgaaaatttttcaataaaataatttaatgtcaggagaattaagcctaatagtctcctaaatcatccatctcttgagcttcagagcagtcctctgaattaatgcctacatgtttgtaaagggtgttcagactgaagccaagattctacctctaaagagatgcaatctcaaatttatctgaagactgtacctctgctctccataaattgacaccatggcccacttaatgaggttaaaaaaaagctaattctgaatgaaaatctgagcccagtggaggaaatattaatgaacaaggtgcagactgaaatataaatttttctgtaataattatgcatatactttagcaaagttctgtctatgttgactttattgctttttggtaagaaatacaactttttaaagtgaactaaactatcctatttccaaactattttgtgtgtgtgcggtttgtttctatgggttctggttttcttggagcatttttatttcattttaattaattaattctgagagctgctgagttgtgtttactgagagattgtgtatctgcgagagaagtctgtagcaagtagctagactgtgcttgacctaggaacatatacagtagattgctaaaatgtctcacttggggaattttagactaaacagtagagcatgtataaaaatactctagtcaagtgctgcttttgaaacaaatgataaaaccacactcccatagatgagtgtcatgattttcatggaggaagttaatattcatcctctaagtatacccagactagggccattctgatataaaacattaggacttaagaaagattaatagactggagtaaaggaaatggacctctgtctctctcgctgtctcttttttgaggacttgtgtgtgtgtgtgtgtgtgtgtgtgtgtgtgtgttgtggtcagtggggctggaataaaagtagaatagacctgcacctgctgtggcatccattcacagagtagaagcaagctcacaatagtgaagatgtcagtaagcttgaatagtttttcaggaactttgaatgctgatttagatttgaaactgaggctctgaccataaccaaatttgcactatttattgcttcttgaaacttatttgcctggtatgcctgggcttttgatggtcttagtatagcttgcagccttgtccctgcagggtattatgggtaatagaaagaaaagtctgcgttacactctagtcacactaagtaactaccattggaaaagcaacccctgccttgaagccaggatgatggtatctgcagcagttgccaacacaagagaaggatccatagttcatcatttaaaaaagaaaacaaaatagaaaaaggaaaactatttctgagcataagaagttgtagggtaagtctttaagaaggtgacaatttctgccaatcaggatttcaaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgcatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaaccctcaaaatgaaatgaaatcagacccttgtcttacaccataaacaaaaataaatttgaatgggttaaagaattaaactaagacctaaaaccataaaaatttttaaagaaatcaaaagaagaaaattctaatattcacgttgcagccgttttttgaatttgatatgagaagcaaaggcaacaaaaggaaaaataaagaagtgaggctacatcaaactaaaaaatttccacacaaaaaacaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatggcagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaattctagatatctaatgtacagcatcgtgactgtagttaattgtactgtaagtatatttaaaatttgcaaagagagtagatttttttttttttttagatggagttttgctcttgttgtccaggctggagtgcaatggcaagatcttggctcactgcaacctccgcctcctgggttcaagcaaatctcctgcctcagcctcccgagtagctgggattacaggcatgcgacaccatgcccagctaattttgtatttttagtagagacggggtttctccatgttggtcaggctgatccgcctgcctcggccacccaaagggctgggattacaggcgtgagccaccgggcctggccgagagtagatcttaaaagcatttaccacaagaaaaaggtaactatgtgagataatgggtatgttaattagcttgattgtggtaatcatttcacaaggtatacatatattaaaacatcatgttgtacaccttaaatatatacaatttttatttgtgaatgatacctcaataaagttgaagaataataaaaaagaatagacatcacatgaattaaaaaactaaaaaataaaaaaatgcatcttgatgattagaattgcattcttgatttttcagatacaaatatccatttgactgtttactcttttccaaaacaatacaataaattttagcactttatcttcattttccccttcccaatctataattatatatatatatattttagatattttgtatagttttactccctagattttctagtgttattattaaatagtgaagaaatgtttacacttatgtacaaaatgttttgcatgcttttcttcatttctaacattctctctaagtttattctatttttttctgattatccttaatattatctctttctgctggaaatacattgttacttttggtttatctaaaaatggcttcattttcttcattctaaaatcatgttaaattaataccactcatgtgtaagtaagatagtggaataaatagaaatccaaaaactaaatctcactaaaatataataatgtgatatataaaaatatagcttttaaatttagcttggaaataaaaaacaaacagtaattgaacaactatactttttgaaaagagtaaagtgaaatgcttaactgcatataccacaatcgattacacaattaggtgtgaaggtaaaattcagtcacgaaaaaactagaataaaaatatgggaagacatgtatataatcttagagataacactgttatttaattatcaacccaaagtagaaactatcaagggagaaataaattcagtcaacaataaaagcatttaagaagttattctaggctgggagcggtggctcacacctgcaattgcagcactttgggaggcctagacaggcggatcacgacgtcaggagttcaagatcagcctggccaacatagtgaaacctcatcgctactaaaaatataaaaacttagcctggcgtggtggcaggcatgtgtaatcccagcaatttgggaggctgaggcaggagaatcgcttgatcctgggaggcagaggttgcagtgagccaagattgtgccactgcattccagcccaggtgacagcatgagactccgtcacaaaaaaaaaagaaaaaaaaaaggggggggggagcggtggagccaagatgaccgaataggaacagctccagtctatagctcccatcgtgagtgacgcagaagacgggtgatttctgcatttccaactgaggtaccaggttcatctcacagggaagtgccaggcagtgggtgcaggacagtaggtgcagtgcactgtgcatgagccaaagcagggcgaggcatcacctcacccgggaagcacaaggggtcagggaattccctttcctagtcaaagaaaagggtgacagatggcacctggaaaatcgggtcactcccgccctaatactgcgctcttccaacaagcttaacaaatggcacaccaggagattatatcccatgcctggctcagagggtcctacgcccatggagcctcgctcattgctagcacagcagtctgaggtcaaactgcaaggtggcagtgaggctgggggaggggtgcccaccattgtccaggcttgagcaggtaaacaaagccgcctggaagctcgaactgggtggagcccaccacagctcaaggaggcctgcctgcctctgtaggctccacctctaggggcagggcacagacaaacaaaagacaacaagaacctctgcagacttaaatgtccctgtctgacagctttgaagagagtagtggttctcccagcacatagcttcagatctgagaacaggcagactgcctcctcaagtgggtccctgacccccgagtagcctaactgggaggcatcccccagtaggggcagactgacacctcacatggctggtactcctctaagacaaaacttccagaggaatgatcaggcagcagcatttgcggttcaccaatatccactgttctgcagccaccgctgttgatacccaggaaaacagcttctggagtggacctccagtaaactccaacagacctgcagctgagggtcctgactgttagaaggaaaactaacaaacagaaaggacatccacaccaaaaacccatctgtacatcgccatcatcaaagaccaaaggtagataaaaccataaagatggggaaaaagcagagcagaaaaactggacactctaaaaatgagagtgcctctcctcctccaaagtaacgcagctcctcaccagcaatggaacaaagctgggcagagaatgactttgacgagttgagagaggaaggcttcagaagatcaaactactccaagctaaaggaggaagttcgaacaaacggcaaagaagtaaaaaactttgaaaaaaaattagatgaatggataactagaataaccaatgcacagaagtccttaaaggacctgatggagctgaaaaccaaggcaggagaactacgtgacaaatacacaagcctcagtaaccgatgagatcaactggaagaaagggtatcaatgacgaaagatgaaatgaatgaaatgaagcatgaagagaagtttagagaaaaaagaataaaaagaaacgaacaaagcctccaagaaatatgggactatgtgaaaagaccaaatctacatctaattggtgtagctgaaagtgatggggagaatggaaccaagttggaaaacactctgcaggatattatccaggagaacttccccaatctagcaaggcaagcccaaattcacattcaggaaatacagagaacgccacaaagatactcctagagaaaagcaactccaagacacataactgtcagattcaccaaagttgaaatgaaggaaaaaatgttaagggcagccagagagaaaggtcgggttacccacaaagggaagcccatcagactaacagctgatctatcggcagaaactctacaagccagaagaaagtgggggccaatattcaacattgttaaagaaaagaattttcaacccagaatttcatatccagccaaactaagcttcataagtgaaggagaaataaaatcctttacagacaagcaaatgctgagagattttgtcaccaccaggcctgccctacaagagctcctgaaggaagcactaaacatggaaaggaacaactagtatcagccactgcaaaaacatgccaaattgtaaagaccatcaaggctaggaagaaactgcatcaacgagcaaaataaccagctaacatcataatgacaggatcaaattcatacataacaatactcaccttaaatgtaaataggctaaatgctccaattaaaagacacagactggcaaattggataaggagtcaagacccatctgtgttctgtattcaggaaacccatctcacgtgcagagacacacataggctcgaaataaaaggatggaggaatatctaccaagcaaatggaaaacaaaaaaaggcaggggttgcaatcctagtctctgataaaacagattttaaaccaacaaagatcaaaagagacaaagaaggccattacataatggcaaagggatctattcaagaagaagaactaactatactaaatatatatgcacccaatacaggagcacccagattcataaaacaagtcctgagtgacctacaaagagacttagatgcccacacaataataatgggagactttaacaccccactgtcaacattagacagatcaacgagacagaaagttaacaaggatatccaggaattggactcagctctgcaccaagcagacctaatagacatctacagaactctccaccccaaatcaacagaatatacattcttttcagcaccacaccacacctattccaaaactgaccacatagttggaagtaaagctctcctcagcaaatgtaaaagaacagaaactataacaaactgtctctcagaccacagtgcaatcaaactagaactcaggattaagaaactcactcaaaaccactcagctacatggaaactgaacagcctgctcctgaatgactactgggtacataacaaaatgaaggcagaaataaagatgttctttgaaaccaacgagaacaaagacacaacacaccagaatctctgagacacattcaaagcagtgtgtagagggaaatttatagcactaaatgcccacaagggaaagcaggaaagatctaaaattgacaccctaacatcacaattaaaaaactagagaagcaggagcaaacacattcaaaagctaacagaagacaagaaataactaagatcagagcagaagtgaaggacatagagacacaaaaaaacccttcaaaaaaatcaatgaatccagaagctgtttttttgaaaagatcaacaaaattgatagactgctagcaagactaataaagaagaaaagagagaagaatcaaatagacgcaataaaaaatgacacggggtatcaccactgatcccacagaaatacaaactaccgtcagagaatactataaacacctctacgcaaataaactagaaaatctagaagaaatggataaattcctcgacacatacactctgccaagactaaaccaggaagaagttgtatctctgaatagaccaataacaggctctgaaattgaggcaataattaatagcttatcaaccaaaaaaagtccgggaccagtaggattcatagccgaattctaccagaggtacaaggaggagctggtaccattccttctgaaactattccaatcaatagaaaaagagggaatcctccctaactcattttatgaggccagcatcatcctgataccaaagcctgacagagacacaacaaaaaaagagaatgttacaccaatatccttgatgaacattgatgcaaaaatcctcaataaaatactggcaaactgatccaccatgatcaagtgggcttcatccctgccatgcaaggctggttcaacatacgaaaatcaataaacataatccagcatataaacagaaccaaagacacaaaccatatgattatctcaatagatgcagaaaaggcctttgacaaaattcaacaacgcttcatgctaaaaactctcaataaattaggtattgatgggacatatctcaaaataataagagctatctatgacaaacccacagccaatatcatactgagtggacaaaaactggaagcattccctttgaaaactggcacaaggcagggatgccctctctcaccactcctattcaacatagtgttgtaagttctggccagggcaatcaggcaggagaaggaaataaagggcattcaattaggaaaagaggaagtgaaattgtccctgtttgcagatgacatgattgtatatctagaaaaccccattgtctcagcccaaaatctccttaagctgataagcaacttcagcaaagtctcaggatataaaatcagtgtgcaaaaatcacaagtattcctatgcaccaataacagacaaacagagagccaaatcatgagtgaactcccattcacaattgcttcaaagagaataaaatacctaggaatccaacttacaagggatgtgaaggacctcttcaaggagaactacaaaccactgctcaatgaaataaaagaggatacaaacaaatggaagaacattccatgctcatgggtaggaagaatcaatatcgtgaaaatggtcatactgcccaaggtaatttatagattcaatgccatccccatcaagctaccaatgactttcttcacagaactggaaaaaactactttaaagttcatatggaaccaaaaaagagcccacatcaccaaggcaatcctaagccaaaagaacaaagctggaggcatcacgctacctgacttcaaactatactacaatgctacggtaaccaaaacagcatggtactggtaccaaaacagagatctagaccaatggaacagaacagagccctcagaaataatgccgcatatctacaactatctgatctttgacaaacctgagagaaacaagcaatggggaaaggattccctatttaataaatggtgctgggaaaactggctagccatatgtagaaagctgaaactggatcccttccttacaccttatacaaaaattaattcaagatggattaaagacttacatgttagacctaaaaccataaaaaccctagaaaaaaacctaggcaataccattcaggacataggcatgggcaaggacttcatgtctaaaacaccaaaagcaatggcaacaaaagacaaaatggacaaacgggatctaattaaactaaagagcttctgcacagctaaagaaactaccatcagagtgaacaggcaacctacaaaatgggagaaaatttttgcaatctactcatctgacaaagggctaatatccagaatctacaatgaactcaaacaaatttacaagaaaaaacaaacaaccccatcaaaaagtgggcaaaggatatgaacagacacttcgcaaaagaagacatttatgtaatcaaaaaacacatgaaaaaatgctcatcatcactagccatcagagaaatgcaaatcaaaaccacaatgagataccatctcacaccagttagaatggcgatcattaaaaagtcaggaaacaacaggtgctggagaggatgtggagaaacaggaacaacttttacactgttggtgggactgtaaactagttcaaccattgcggaagtcagtgtggcaattcctcaggaatctagaactagaaataccatttgacccagccatcccattactgggtacatacccaaaggattataaatcatgctgctataaagacacatgcacacgtatgtttattgcagcactattcacaatagcaaagacttggaaccaacccaaatgtccaacaacgatagactggattaagaaaatgtggcacatatacaccatggaatactatgcagccataaaaaatgatgagttcatgtcctttgtagggacatggatgaagctggaaactatcattctcagcaaactatcacaaggagaataaaccaaacaccgcatgttctcactcataggtgggaattgaacaatgagaacacatggacacatgaagaggaacatcacactctggggactgttatggggtggggggcaggggcagggatagcactaggagatatacctaatgctaaatgacgagttaatgggtgcagcacaccaacatggcacatgtatacatatataacaaacctgcatgttgtgcacatgtaccctaaaacttgaagtataataataaaaaaaagttatcctattaaaactgatctcacacatccgtagagccattatcaagtctttctctttgaaatagacagaaatttagtgttttctcagtcagttaac

SEQ ID NO: 20 - DNA, 388 bp, Homo sapiens
  source 1..388: mol_type = genomic DNA
taagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatatt

SEQ ID NO: 21 - DNA, 387 bp, Homo sapiens
  source 1..387: mol_type = genomic DNA
taagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggctagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatatt

SEQ ID NO: 22 - DNA, 286 bp, Homo sapiens
  source 1..286: mol_type = genomic DNA
ctgagcaactaactcatgcaggactctcaaacactaacctatagccttttctatgtatctacttgtgtagaaaccaagcgtggggactgagaaggcaatagcaggagcattctgactctcactgcctttggctaggtccctccctcatcacagctcagcatagtccgagctcttatctatatccacacacagtttctgacgctgcccagctatcaccatcccaagtctaaagaaaaaaataatgggtttgcccatctctgttgattagaaaacaaaacaaaataaa

SEQ ID NO: 23 - DNA, 286 bp, Homo sapiens
  source 1..286: mol_type = genomic DNA
ctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaa

SEQ ID NO: 24 - DNA, 301 bp, Homo sapiens
  source 1..301: mol_type = genomic DNA
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagga

SEQ ID NO: 25 - DNA, 305 bp, Homo sapiens
  source 1..305: mol_type = genomic DNA
ctagccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctactccagggatgctcatccaggaaggaaaggttgcatgctggacacactaaccttgaagaattcttctgtctctctcgtcatttagaaaggaagga

SEQ ID NO: 26 - DNA, 896 bp, synthetic construct
  misc_feature 1..896: note = Description of Artificial Sequence: Synthetic polynucleotide
  source 1..896: mol_type = other DNA
ctcgagttaattaatctcccacgccctggtctcagcttggggagtggtcagaccccaatggcgataaactctggcaactttatctgtgcactgcaggctcagccccaacagctttagctttcacaagcaggcaggggaagggaaacacatatctccagatatgaggttaattaacctgcaggctaaacccctcccccaccctagccccaagcttcatcttagctccactcctgaccctatccagctaaaggtccccacccagctcctgcctatctagtcattgcatatggcaagacttgaaagtcctatctcaaagcagcagaattatcagctacgactcctgcaggttataaccatcccccagcactccctgcccccacagcccagacttgaccaactcccagctccgcctgggacttccagatatggggccccacccttgcaggccttggggacgctgaagatattgactatctgcgtgccggaaaagggtgttataaaccggtaaaggctgggggtgggagtagcggatttgaagcacttgttggcctacagaggtgtggcaagcagagcacctcagaactcaggcgtactgcccgccgcccgagccctgcgagggccgatagcgagggtgtggcccttatctgcacccagcagagcgccggcggggtacggtcaccggtcccgggcagttgcctcagctgagtatgtcttctaaagataatgtcgattgtgtatggctgatgggattctaggaccaagcaagaggtttttttttttcccccacatacttaacgtttctatatttctatttgaattcgactggacagttccatttgaattatttctctctctctctctctctgacacattttatcttgccacccgggctcgag